(19) United States
(12) Patent Application Publication
(10) Pub. No.: US 2025/0270657 A1
(43) Pub. Date: Aug. 28, 2025

(54) Detection of Antibiotic Resistance Genes

(71) Applicant: STRECK, INC., La Vista, NE (US)

(72) Inventors: Stephanie Cossette, Omaha, NE (US); Christopher Connelly, Greina, NE (US); Maria Torres-Gonzalez, Omaha, NE (US); Esther Alao, Omaha, NE (US)

(21) Appl. No.: 17/614,892

(22) PCT Filed: May 29, 2020
(86) PCT No.: PCT/US20/35422

§ 371 Date: Nov. 29, 2021

Related U.S. Application Data
(60) Provisional application No. 62/872,655, filed on Jul. 10, 2019.
(60) Provisional application No. 62/855,684, filed on May 31, 2019.
(60) Provisional application No. 62/855,709, filed on May 31, 2019.

(51) Int. Cl. C12Q 1/689

(52) U.S. Cl. CPC C12Q 1/689; C12Q 2600/16

(57) ABSTRACT

Assays and methods for detecting resistance to beta-lactam antibiotics including detection of multiple β-lactamase family specific gene targets by polymerase chain reaction or microarray. One or more kits including primers and/or probes for identification of β-lactamase genes selected from the group consisting of one or more of the following: MOX-like, FOX-like, ACC-like, ACT/MIR-like, CMY-2-like, DHA-like, CTX-M-14-like, CTX-M-15-like, VIM-like, NDM-like, IMP-like, KPC-like, and OXA-48-like, OXA-51-like, OXA-143-like, OXA-58-like, OXA-23-like, OXA-24/40-like, TEM-like, SHV-like, and GES-like. A kit may also include one or more primers and/or probes for the identification a non-beta lactamase gene family which confers antibiotic resistance, such as the MCR-1 gene.

### CROSS-REFERENCE TO RELATED APPLICATIONS

This application is a U.S. National Phase of International Application No. PCT/US2020/035422, filed May 29, 2020, which claims priority to U.S. Provisional Application Ser. No. 62/855,684, filed May 31, 2019, U.S. Provisional Application Ser. No. 62/855,709, filed May 31, 2019, and U.S. Provisional Application Ser. No. 62/872,655, filed Jul. 10, 2019, the entire disclosures of which are hereby incorporated by reference in their entirety.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “54375_Seqlisting.txt”, which was created on May 29, 2020 and is bytes in size. The subject matter of the Sequence Listing is incorporated herein in its entirety by reference.

### FIELD

The present teachings relate to assays and methods for detecting resistance to antibiotics. The present teachings provide for the detection of family specific gene targets including AmpC β-lactamases, metallo-β-lactamases, carbapenemases, and extended-spectrum β-Lactamases by multiplex real-time polymerase chain reaction.

### BACKGROUND

Bacterial resistance to antibiotics is a major public health issue. This resistance not only presents severe limitations to the ability to control and treat infection, but it also is difficult to identify and characterize in the laboratory. The significant increase in the resistance of pathogenic bacteria over the last 20 years leads to extended periods of hospitalization, high morbidity and high mortality rates.

Enzymatic inactivation is the most common cause of resistance in terms of number of species and of antibiotics involved. As an example, β-lactamases are enzymes expressed by some bacteria. Such enzymes are capable of hydrolyzing the C—N bond of the β-lactam ring structure of a β-lactam antibiotic, effectively inactivating the antibiotic. Despite the existence of several β-lactamase inhibitors, the constant exposure of strains to antibiotics results in constant evolution of β-lactamases.

As a result, it becomes essential to be able to identify such resistant microorganisms and their resistance mechanisms as quickly as possible. Typically, biological samples can be tested for antibiotic resistance, but many test protocols are time consuming and/or limited in the types of resistance they are able to identify. It would therefore be beneficial to provide a test protocol for the simplified identification of resistance for all major β-lactamases.

One approach to the identification of β-lactamases has been to employ oligonucleotide primers specific for nucleic acid characteristic of certain β-lactamases with polymerase chain reaction to identify nucleic acid characteristics of family specific β-lactamase enzymes in samples. See for example, U.S. Pat. Nos. 6,893,846 and 7,476,520, incorporated by reference herein. Another approach has been to employ oligonucleotide primers specific for nucleic acid characteristic of certain AmpC β-lactamases with multiplex polymerase chain reaction to detect the presence or absence of an AmpC β-lactamase gene and to identify nucleic acid characteristic of AmpC β-lactamase genes in samples. Multiplex polymerase chain reaction refers to the use of polymerase chain reaction to amplify several different DNA sequences simultaneously in single or multiple reactions. See for example, U.S. Pat. Nos. 7,045,291 and 7,521,547 incorporated by reference herein.

However, such primers have been limited with regards to the number of β-lactamase gene families or the number of gene targets that may be identified. Furthermore, such primers have been employed mainly with conventional polymerase chain reaction, which typically requires agarose gels to detect and analyze the PCR product(s). The use of agarose gel detection methods based on size discrimination may lead to poor resolution and difficulty in interpreting the data. Conventional polymerase chain reaction also lacks the sensitivity to detect endpoint variability from sample to sample and may not be automated. Real-time polymerase chain reaction allows for monitoring of reaction products as they are formed.

Detection of β-lactamases using real-time polymerase chain reaction and a single primer set may be limited to detection of a single β-lactamase gene family. See for example, US Patent Publication 2007/0248954 incorporated by reference herein. Multiplex real-time polymerase chain reaction has been designed for the identification of many AmpC β-lactamases simultaneously. See Geyer C N, Reisbig M D, Hanson N D. Development of a TaqMan® Multiplex PCR Assay for Detection of Plasmid-Mediated AmpC β-lactamase Genes. Journal of clinical microbiology. 2012 Aug. 15:JCM-02038. The primer/probe combinations in this study, however, have been directed only to AmpC β-lactamases and are limited in the number of gene targets that may be identified.

Multiple factors such as primer and probe design, reaction conditions, and enzyme selection must all be considered when designing a working polymerase chain reaction. This complexity is compounded in multiplex PCR, in which multiple targets are detected simultaneously in the same tube. Balancing the concentrations of primers, probes, and control vectors provided as composite “multiplex PCR” mixes for an assay is a challenging aspect. It is extremely difficult to balance these ratios, as a change of concentration for any of these reagents, corresponding to just one of the genetic targets, may adversely affect detection of any other multiplex target in the reaction mix. If these concentrations are not balanced, one could expect a reduction in efficiency, sensitivity, and specificity. This would reduce confidence in the effectiveness of the assay to correctly identify the gene families identified with the described kits.

Therefore, there is a significant amount of time and technical know-how required to develop these assays into a reliable method. For example, the PCR master mixture, with DNA polymerase, is a customized formulation that permits the final assay to work. Concentrations of DNA polymerase and magnesium may have to be adjusted. The specific concentrations and ranges surrounding DNA polymerase and magnesium are required for the assay to work successfully. In addition to determining concentrations for all reagents, a PCR cycling protocol must be identified that is compatible with all reaction conditions and facilitates real-time multiplex polymerase chain reaction.

### SUMMARY

Accurate and rapid detection of antibiotic resistance is essential for surveillance, epidemiologic tracking, patient therapy, and infection control. Thus, a multiplex PCR based diagnostic assay should provide comprehensive genotypic characterization of β-lactamases and be versatile as well as providing rapid results. The present teachings make it possible to test a sample for the presence of antibiotic resistant microorganisms by identifying any of the major β-lactamases in one test. The present teachings provide for the detection of multiple family-specific β-lactamase gene targets, including but not limited to metallo-β-lactamases, carbapenemases, extended-spectrum β-Lactamases, ampC chromosomal and/or plasmid-mediated AmpC β-lactamases, by multiplex real-time polymerase chain reaction.

The present teachings provide for a kit or kits including one or more primers and/or probes for identification of β-lactamase genes selected from the group consisting of one or more of the following: MOX-like, FOX-like, ACC-like, ACT/MIR-like, CMY-2-like, DHA-like, CTX-M-14-like, CTX-M-15-like, VIM-like, NDM-like, IMP-like, KPC-like, and OXA-48-like, OXA-51-like, OXA-143-like, OXA-58-like, OXA-23-like, OXA-24/40-like, TEM-like, SHV-like, and GES-like. The kit or kits of the present teachings may provide control material for the aforementioned β-lactamase genes. The present teachings provide one or more of the following: primers, probes, controls, assay process and detection strategy for one or more of the following β-lactamases: extended-spectrum β-lactamases (ESBLs), metallo-β-lactamases (MBLs), carbapenem-resistant enterobacteriaceaes (CREs), carbapenem-resistant Acinetobacter (CRAs), and serine-dependent carbapenemases and plasmid-mediated ampC β-lactamases. A kit or kits may also include one or more primers and/or probes for the identification of mobilized colistin-resistant (MCR) genes, a non-beta lactamase gene family that confers antibiotic resistance. The present teachings provide multiplex PCR assays which may test for any combination of these or are directed towards identification of a specific group. The present teachings provide assays with improved clinical sensitivity and analytical specificity of detection. The primer, probes, and control DNA sequences of the present teachings provide both an analytical and commercial advantage as they permit enhanced screening capabilities for detection of a larger number of genetic variants associated with genes conferring resistance to antibiotics in Gram-negative bacteria.

The present teachings provide a kit including one or more primers and/or probes for the identification by polymerase chain reaction, microarray, NGS-based target enrichment, and/or mass spectrometric characterization of one or more β-lactamase genes selected from the group consisting of: CMY, CTX-M, OXA, IMP, VIM, DHA, KPC, MOX, ACC, FOX, EBC, NDM, TEM, SHV, and GES. The present teachings provide for one or more kits including primers and/or probes for identification of β-lactamase genes selected from the group consisting of one or more of the following: MOX-like, FOX-like, ACC-like, EBC-like, CMY-2-like, DHA-like, CTX-M-14-like, CTX-M-15-like, VIM-like, NDM-like, IMP-like, KPC-like, and OXA-48-like, OXA-51-like, OXA-143-like, OXA-58-like, OXA-23-like, OXA-24/40-like, TEM-like, SHV-like, and GES-like. A kit may also include one or more primers and/or probes for the identification of a non-beta lactamase gene family which confers antibiotic resistance. A kit may include one or more primers and/or probes for the identification by polymerase chain reaction or microarray of MCR gene variants. Primers and probes may also be made compatible with next-generation sequencing and mass spectrometry.

In some aspects, the disclosure provides a kit comprising one or more primers and/or one or more probes for the identification of one or more genes associated with antibiotic resistance, wherein the genes are: (A) Imipenem-resistant carbapenemase (IMP), wherein the primers are SEQ ID NO: 296-299 and the probes are SEQ ID NO: 354-356; (B) Mobilized colistin resistance (MCR), wherein the primers are SEQ ID NO: 305-306, 308-309, and 311-312, and the probes are SEQ ID NO: 357-361; (C) Temoniera (TEM), wherein the primers are SEQ ID NO: 314-315; (D) Sulfhydral reagents variable (SHV), wherein the primers are SEQ ID NO: 316-317, and the probe is SEQ ID NO: 362; (E) Guiana extended-spectrum β-lactamase (GES), wherein the primers are SEQ ID NO: 319-320, and the probe is SEQ ID NO: 363; (F) Oxacillinase-type β-lactamase (OXA), wherein the primers are SEQ ID NO: 322-323, 328-329, 331-332, 334-335, and 337-338, and the probes are SEQ ID NO: 364-369, or a combination thereof. In some embodiments, (A) comprises each of the primers having sequences as set out in SEQ ID NO: 296-299 and each of the probes having sequences as set out in SEQ ID NO: 354-356. In some embodiments, a kit of the disclosure comprises (A) and further comprises: (i) primers having SEQ ID NOs: 67-68, 70-71, 73-74, 76-77, 79-80, 89-90, 92-93, 95-96, and 98-99; and (ii) probes having SEQ ID NOs: 69, 72, 75, 78, 81, 87-88, 91, 94, 97, and 100. In some embodiments, the kit further comprises (iii) control sequences having SEQ ID NOs: 261-267 and 269-271. In some embodiments, one or more probes comprises a label. In further embodiments, the label is fluorescein, hexachlorofluorescein, TEX615, Cyanine 5 (Cy5), or a combination thereof. In still further embodiments, SEQ ID NO: 354, as labeled, is as set forth in SEQ ID NO: 300; SEQ ID NO: 355, as labeled, is as set forth in SEQ ID NO: 301; and SEQ ID NO: 356, as labeled, is as set forth in SEQ ID NO: 302. In some embodiments, (B) comprises each of the primers having sequences as set out in SEQ ID NO: 305-306, 308-309, and 311-312, and each of the probes having sequences as set out in SEQ ID NO: 357-361. In some embodiments, a kit of the disclosure comprises (B) and further comprises: (i) primers having SEQ ID NOs: 252, 141, 143, 144, 76, and 77; and (ii) probe having SEQ ID NO: 340. In some embodiments, the kit further comprises (iii) control sequences having SEQ ID NOs: 341-345 and 264. In further embodiments, one or more probes comprises a label. In some embodiments, the label is fluorescein, hexachlorofluorescein, TEX615, Cyanine 5 (Cy5), or a combination thereof. In still further embodiments, SEQ ID NO: 357, as labeled, is as set forth in SEQ ID NO: 303; SEQ ID NO: 358, as labeled, is as set forth in SEQ ID NO: 304; SEQ ID NO: 359, as labeled, is as set forth in SEQ ID NO: 307; SEQ ID NO: 360, as labeled, is as set forth in SEQ ID NO: 310; and SEQ ID NO: 361, as labeled, is as set forth in SEQ ID NO: 313. In some embodiments, (C) comprises each of the primers having sequences as set out in SEQ ID NO: 314-315; (D) comprises each of the primers having sequences as set out in SEQ ID NO: 316-317, and probe having a sequence as set out in SEQ ID NO: 362; and (E) comprises each of the primers having sequences as set out in SEQ ID NO: 319-320, and probe having a sequence as set out in SEQ ID NO: 363. In some embodiments, a kit of the disclosure comprises (C), (D), and (E), and further comprises: (i) primers having SEQ ID NOs: 76 and 77; and (ii) probes having SEQ ID NOs: 148 and 340. In some embodiments, the kit further comprises (iii) control sequences having SEQ ID NOs: 346-348, and 264. In some embodiments, one or more probes comprises a label. In some embodiments, the label is fluorescein, hexachlorofluorescein, TEX615, Cyanine 5 (Cy5), or a combination thereof. In still further embodiments, SEQ ID NO: 362, as labeled, is as set forth in SEQ ID NO: 318; and SEQ ID NO: 363, as labeled, is as set forth in SEQ ID NO: 321. In some embodiments, (F) comprises each of the primers having sequences set out in SEQ ID NO: 322-323, 328-329, 331-332, 334-335, and 337-338, and each of the probes having sequences as set out in SEQ ID NO: 364-369. In some embodiments, a kit of the disclosure comprises (F) and further comprises: (i) primers having SEQ ID NOs: 79-80 and 76-77; and (ii) probes having SEQ ID NOs: 370 and 340. In some embodiments, the kit further comprises (iii) control sequences having SEQ ID NOs: 58, 349-353, and 264. In further embodiments, one or more probes comprises a label. In some embodiments, the label is fluorescein, hexachlorofluorescein, TEX 615, Cyanine 5 (Cy5), or a combination thereof. In still further embodiments, SEQ ID NO: 364, as labeled, is as set forth in SEQ ID NO: 324; SEQ ID NO: 365, as labeled, is as set forth in SEQ ID NO: 330; SEQ ID NO: 366, as labeled, is as set forth in SEQ ID NO: 333; SEQ ID NO: 367, as labeled, is as set forth in SEQ ID NO: 336; SEQ ID NO: 368, as labeled, is as set forth in SEQ ID NO: 339; and SEQ ID NO: 369, as labeled, is as set forth in SEQ ID NO: 340.

In some aspects, the disclosure provides a method of detecting one or more genes associated with antibiotic resistance comprising: (a) amplifying at least a portion of a target nucleic acid from a biological sample using a kit of the disclosure to produce an amplified target nucleic acid; and (b) analyzing the amplified target nucleic acid to detect the one or more genes associated with antibiotic resistance. In some embodiments, the amplifying is performed by polymerase chain reaction (PCR). In further embodiments, the PCR is quantitative real-time PCR. In still further embodiments, the PCR is digital droplet PCR. In some embodiments, at least about 0.1 copy of the target nucleic acid is detected.

In some embodiments, the analyzing is performed by microarray technology. In further embodiments, the analyzing is performed by fluorescence and/or infra-red probe-based detection chemistries. In some embodiments, the biological sample is blood, a blood culture, urine, plasma, feces, a fecal swab, a peri-rectal/peri-anal swab, sputum, and/or a bacterial culture. In some embodiments, the portion of the target nucleic acid that is amplified is from about 25 base pairs to about 2000 base pairs. In further embodiments, the one or more genes associated with antibiotic resistance comprise IMP, MCR, TEM, SHV, GES, and/or OXA. In some embodiments, the one or more genes associated with antibiotic resistance is IMP. In some embodiments, the one or more genes associated with antibiotic resistance is MCR. In some embodiments, the one or more genes associated with antibiotic resistance are TEM, SHV, and GES. In some embodiments, the one or more genes associated with antibiotic resistance is OXA.

Additional aspects and embodiments of the disclosure are described in the following enumerated paragraphs.

Paragraph 1. A kit including one or more primers and/or one or more probes for the identification of one or more genes associated with antibiotic resistance, selected from the group consisting of: CMY, CTX-M, OXA, IMP, VIM, DHA, KPC, MOX, ACC, FOX, ACT/MIR, NDM, mcr-1, mcr-2, mcr-3, mcr-4, mcr-5, TEM, SHV, GES, or a combination thereof.

Paragraph 2. The kit of paragraph 1, wherein the mcr-1 gene target is mcr-1.1, mcr-1.2, mcr-1.3, mcr-1.4, mcr-1.5, mcr-1.6, mcr-1.7, mcr-1.8, mcr-1.9, mcr-1.11, mcr-1.12, mcr-1.13, mcr-1.14, mcr-1.15, or a combination thereof.

Paragraph 3. The kit of paragraph 1 or paragraph 2, wherein the mcr-2 gene target is mcr-2.1.

Paragraph 4. The kit of any one of paragraphs 1-3, wherein the mcr-3 target is mcr-3.1, mcr-3.2, mcr-3.3, mcr-3.4, mcr-3.5, mcr-3.6, mcr-3.7, mcr-3.8, mcr-3.9, mcr-3.10, mcr-3.11, mcr-3.12, mcr-3.13, mcr-3.14, mcr-3.15, mcr-3.16, mcr-3.18, mcr-3.19, mcr-3.20, mcr-3.21, mcr-3.22, mcr-3.23, mcr-3.24, mcr-3.25, or a combination thereof.

Paragraph 5. The kit of any one of paragraphs 1-4, wherein the mcr-4 target is mcr-4.1, mcr-4.2, mcr-4.3, mcr-4.4, mcr-4.5, mcr-4.6, or a combination thereof.

Paragraph 6. The kit of any one of paragraphs 1-5, wherein the mcr-5 target is mcr-5.1, mcr-5.2, mcr-5.3, or a combination thereof.

Paragraph 7. The kit of any one of paragraphs 1-6, wherein the OXA target is OXA-143, OXA-48, OXA-24/40, OXA-58, OXA-51, OXA-23, or a combination thereof.

Paragraph 8. The kit of any one of paragraphs 1-7, further comprising one or more primers and/or one or more probes for the identification of an internal control.

Paragraph 9. The kit of paragraph 8, wherein the internal control is 16S ribosomal RNA (rRNA).

Paragraph 10. A method of detecting one or more genes associated with antibiotic resistance in a sample, comprising: amplifying at least a portion of the one or more genes using a kit of the disclosure; and detecting the amplified portion of the one or more genes.

Paragraph 11. The method of paragraph 10, wherein detecting is carried out by microarray technology, fluorescence detection technology, infrared probe-based detection, intercalating dye-based detection, or nucleic acid sequencing technology.

Paragraph 12. The method of paragraph 10 or paragraph 11, wherein the sample is obtained directly from or extracted directly from a crude biological sample such as blood, blood culture, urine, plasma, feces, a fecal swab, a peri-rectal/peri-anal swab, sputum, or a bacterial culture.

Paragraph 13. The method of any one of paragraphs 10-12, wherein the sample is a purified nucleic acid sample.

Paragraph 14. The method of any one of paragraphs 10-13, wherein the amplifying is carried out by polymerase chain reaction (PCR).

Paragraph 15. The method of paragraph 14, wherein the PCR is real-time PCR, digital droplet PCR, or conventional PCR.

Paragraph 16. The method of paragraph 15, wherein the real-time PCR is quantitative real-time PCR.

Paragraph 17. The method of any one of paragraphs 10-16, wherein the amplifying is performed in multiplex.

Paragraph 18. The method of any one of paragraphs 10-17, wherein the amplified portion of the one or more genes is from about 25 to about 2000 base pairs in length.

### BRIEF DESCRIPTION OF DRAWINGS

FIG. 1 depicts an amplification plot of an exemplary mix 1 of a kit including ampC gene targets.

FIG. 2 depicts an amplification plot of an exemplary mix 2 of a kit including ampC gene targets.

FIG. 3 depicts an amplification plot of an exemplary mix 1 of a kit including β-lactamase gene targets.

FIG. 4 depicts an amplification plot of an exemplary mix 2 of a kit including β-lactamase gene targets.

FIG. 5 depicts an amplification plot of an exemplary mix 3 of a kit including β-lactamase gene targets.

FIG. 6 depicts an amplification plot of an exemplary internal control mix of a kit including MCR gene targets.

FIG. 7 depicts an amplification plot of an exemplary mix 1 of a kit including OXA gene targets.

FIG. 8 depicts an amplification plot of an exemplary mix 2 of a kit including OXA gene targets.

FIG. 9 depicts an amplification plot of an exemplary internal control mix of a kit including SHV-TEM gene targets.

FIG. 10 shows real-time PCR amplification of serially-diluted Multiplex Control Mix of a representative kit of the disclosure for detecting MCR. Standard curves show corresponding efficiencies and correlation coefficients for each target within the control mix, respectively.

FIG. 11 shows real-time PCR amplification of clinical isolates with a representative MCR detection kit of the disclosure (n=90). Representative amplification plots of mcr-positive clinical isolates and positive control for each gene are shown. The internal control (IC) was amplified for all strains tested.

FIG. 12 shows representative data generated by the internal control (IC) utilized in both of the OXA Real-Time PCR mixes.

FIG. 13 shows a direct comparison of amplicons generated by DNA extracted from fresh culture and from stabilized cells. The same bacterial isolate was used in both preparations.

FIG. 14 shows results for the cycle number vs. RFU curve for OXA-58 (FAM, FIG. 14A), OXA-48 (HEX, FIG. 14B), and OXA-24/40 (TEX615, FIG. 14C), respectively.

FIG. 15 shows the amplification of an OXA β-lactamase gene family detected by the OXA Real-Time PCR assay.

FIG. 16 shows the tracking of mcr in the U.S. The map shows where the mcr gene has been reported in U.S. human and food animal sources as of Nov. 2, 2018. Map source from the CDC website.

FIG. 17 shows results from real-time PCR amplification of serially-diluted Multiplex Control Mix of a representative kit of the disclosure for detecting MCR. Standard curves show corresponding efficiencies and correlation coefficients for each target control, respectively.

FIG. 18 shows real-time PCR amplification of a MCR Multiplex Control Mix from a representative kit of the disclosure.

FIG. 19 shows real-time PCR amplification of clinical isolates with a representative kit of the disclosure for detecting MCR (n=90). Representative amplification plots of mcr-positive clinical isolates and positive control for each gene are shown. The internal control (IC) was amplified for all strains tested.

FIG. 20 shows representative data generated by the internal control (IC) utilized in both of the OXA Real-Time PCT mixes.

FIG. 21 shows results from a direct comparison of amplicons generated by DNA extracted from fresh culture and from stabilized cells. The same bacterial isolate was used in both preparations.

FIG. 22 shows representative data generated by the OXA Real-Time PCR Mix #1.

FIG. 23 shows representative data generated by the OXA Real-Time PCR Mix #2.

FIG. 24 shows additional results generated using kits as generally described herein. The table shows targets detected using a representative kit of the disclosure to detect OXA.

FIG. 25 shows additional results generated using kits as generally described herein. The table shows targets detected using a representative kit of the disclosure to detect MCR.

FIG. 26 shows additional results generated using kits as generally described herein. The table shows targets detected using a representative kit of the disclosure to detect TEM/SHV/GES.

FIG. 27 shows additional amplification results of a representative internal control mix of a representative kit of the disclosure including MCR gene targets.

FIG. 28 shows additional amplification results of a representative internal control mix of a representative kit of the disclosure including MCR gene targets.

FIG. 29 shows additional amplification results obtained using a representative mix 1 of a representative kit of the disclosure including OXA gene targets.

FIG. 30 shows additional amplification results obtained using a representative mix 2 of a representative kit of the disclosure including OXA gene targets.

FIG. 31 shows additional amplification results of a representative internal control mix of a representative kit of the disclosure including SHV-TEM gene targets.

FIG. 32 shows additional amplification results of a representative internal control mix of a representative kit of the disclosure including SHV-TEM-GES gene targets.

FIG. 33 shows additional amplification results generated using a representative kit of the disclosure including β-lactamase gene targets.

FIG. 34 shows results using a representative kit of the disclosure in MCR-clinical isolate testing.

FIG. 35 shows results of MCR-Clinical Isolate Testing using a representative kit of the disclosure.

### DETAILED DESCRIPTION

The explanations and illustrations presented herein are intended to acquaint others skilled in the art with the teachings, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

Bacterial resistance to antibiotics poses a global threat to public health and in recent years has shown an increase in mortality rates and the potential to spread through the population. Of these resistance mechanisms, β-Lactamases are enzymes that cleave β-Lactam rings rendering the β-Lactam family of antibiotics ineffective for treatment of clinically-important Gram-negative bacterial infections. Specifically, β-Lactamases confer resistance to penicillins, cephamycins, and, in some cases, carbapenems. β-Lactam-resistant Gram-negative organisms, producing multiple or plasmid-mediated β-lactamases, are difficult to identify phenotypically and necessitate more specific detection methods to identify clinically important β-lactamases. Genetic identification of these resistance mechanisms is critical for active surveillance and infection control. Because these antibiotics are often selected for the management and prevention of infectious disease, the presence and characteristics of specific β-Lactamases play a critical role in selecting the appropriate antibiotic therapy.

AmpC β-lactamases are clinically important cephalosporinases that are resistant to most β-lactam antibiotics. AmpC enzymes are chromosomally encoded in many bacterial species and can be inducible and overexpressed as a consequence of mutation. Overexpression can lead to resistance to most β-lactam antibiotics. The occurrence of transmissible plasmids with acquired genes for AmpC β-lactamases often result in increased β-lactamase production, compared to chromosomally-expressed ampC genes. Additionally, plasmid-mediated AmpC β-lactamases can appear in organisms lacking or having low-level expression of a chromosomal ampC gene. Resistance due to plasmid-mediated AmpC enzymes can be broad in spectrum and often hard to detect. As such, it is clinically useful to detect and discriminate between plasmid-mediated and chromosomally expressed AmpC β-lactamases.

The present teachings relate to assays and methods for detecting Gram-negative bacteria resistant to beta-lactam antibiotics from a biological sample. β-lactam antibiotics are all antibiotic agents that contain a β-lactam ring in their molecular structures. β-lactam antibiotics include penicillins, cephalosporins, carbapenems and monobactams. Antibiotic resistant organisms may produce one or more enzymes known as β-lactamases that provide resistance to β-lactam antibiotics. β-lactamases may confer resistance by the bacteria to antibiotics, which is plasmid-mediated and/or chromosomally expressed making detection difficult.

β-lactamases may be classified based on molecular structure. The four major classes include A to D. Class A, C and D β-lactamases are serine based. Class B β-lactamases, also known as metallo-beta-lactamases, are zinc based.

Extended spectrum β-lactamases (ESBLs) are enzymes that confer bacterial resistance to certain categories of antibiotics, such as third-generation cephalosporins and monobactams. The presence of an ESBL-producing organism in a clinical infection can cause treatment failure if one of the above classes of drugs is used. Detection of ESBLs can be difficult because they have different levels of activity against various cephalosporins. Thus genetic identification of the exact enzyme can facilitate selection of the optimal antimicrobial agent, which is critical to determine the most effective treatment response.

First-generation cephalosporins include cefalexin, cefaloridine, cefalotin, cefazolin, cefadroxil, cefazedone, cefatrizine, cefapirin, cefradine, cefacetrile, cefrodaxine, ceftezole. Second-generation cephalosporins include cefoxitin, cefuroxime, cefamandole, cefaclor, cefotetan, cefonicide, cefotiam, loracarbef, cefmetazole, cefprozil, ceforanide. Third-generation cephalosporins include cefotaxime, ceftazidime, cefsulodine, ceftriaxone, cefmenoxime, latamoxef, ceftizoxime, cefixime, cefodizime, cefetamet, cefpiramide, cefoperazone, cefpodoxime, ceftibuten, cefdinir, cefditoren, ceftriaxone, cefoperazone, cefbuperazone. Fourth-generation cephalosporins include cefepime and cefpirome.

β-lactamase producing bacteria may include Gram-negative bacteria such as those found in the following genera: Pseudomonas, Escherichia, Salmonella, Shigella, Enterobacter, Klebsiella, Serratia, Proteus, Campylobacter, Haemophilus, Morganella, Vibrio, Yersinia, Acinetobacter, Branhamella, Neisseria, Burkholderia, Citrobacter, Hafnia, Edwardsiella, Aeromonas, Moraxella, Pasteurella, Providencia and Legionella.

Antibiotic resistance is intended to mean any type of mechanism which allows a microorganism to render a treatment partially or completely ineffective on the microorganism, guaranteeing its survival. β-lactam antibiotic resistance is intended to mean any type of β-lactamase-based mechanism which allows a microorganism to render a treatment partially or completely ineffective on the microorganism, guaranteeing its survival. For example, wherein the mechanism is related to the expression of an enzyme belonging to the β-lactamase group including extended-spectrum β-lactamase or of an enzyme belonging to the group of class C cephalosporinases.

Biological sample is intended to mean a clinical sample, derived from a specimen of biological fluid, or a food sample, derived from any type of food or drink, or from an agricultural source, such as animals, soil, water, or air, or from a surface such as with a biofilm. This sample may thus be liquid or solid. For example, the biological sample may be a clinical sample of blood, plasma, urine or feces, or of rectal, nose, throat, skin, wound or cerebrospinal fluid specimens.

The present teachings relate to assays and methods for detecting resistance to beta-lactam antibiotics. The present teachings may detect β-lactamase gene targets which are chromosomally encoded and/or plasmid mediated. The present teachings provide for the detection of family specific gene targets relating to β-lactamase genes including AmpC β-lactamases. The β-lactamase genes detected with the present teachings may include those classified into molecular groups A through D. The β-lactamase genes detected with the present teachings may include those classified into functional groups 1 through 3.

The present teachings relate to assays and methods for detecting resistance of one or more gene beta lactamase gene families including like genes. A like gene may be a beta-lactamase that has one or more of the following: similar amino acid sequence, similar function and similar antibiotic susceptibility profiles. A like gene may be considered as like the target gene detected with the present teachings. For example, OXA-48-like enzymes may include: OXA-48, OXA-48b, OXA-162, OXA-163, OXA-181, OXA-199, OXA-204, OXA-232, OXA-244, OXA-245, OXA-24, or a combination thereof.

The present teachings provide one or more primers and/or probes for the identification of one or more β-lactamase genes selected from the group consisting of: CMY, CTX-M, OXA, IMP, VIM, DHA, KPC, MOX, ACC, FOX, EBC, NDM, TEM, SHV, GES, or a combination thereof. The present teachings provide one or more primers and/or probes for the identification of β-lactamase genes selected from the group consisting of one or more of the following: MOX-like, FOX-like, ACC-like, EBC-like, CMY-2-like, DHA-like, CTX-M-14-like, CTX-M-15-like, VIM-like, NDM-like, IMP-like, KPC-like, and OXA-48-like, OXA-51-like, OXA-143-like, OXA-58-like, OXA-23-like, OXA-24/40-like, TEM-like, SHV-like, and GES-like. The present teachings provide one or more primers and/or probes for the identification of a non-beta lactamase gene family which confers antibiotic resistance. For example, one or more primers and/or probes for the identification of MCR gene variants. The primers and/or probes of the present teachings may be included in one or more kits. The one or more kits may be used for identification with any of the following: polymerase chain reaction, microarray, NGS-based target enrichment, and/or mass spectrometric characterization.

Exemplary sequences for primers and probes for of the present teachings are depicted in Table 1. [SEQ. ID NOS 67-260]. Additional primers and probes are disclosed in Tables 2 and 4. Primers and/or probes may be degenerate at any nucleotide position. Primers and/or probes may not be degenerate at any nucleotide position. Any suitable fluorophore and/or quencher and nucleic acid sequence combination may be used. For example, a probe may be labeled with a fluorescent tag at one end and a fluorescent quencher at the other end. For example, a probe may be labeled with a fluorescent tag at one end and a fluorescent quencher at the other end. For example, two fluorescent quenchers may be included at one end or within the probe sequence. It is contemplated that the probe sequences of the present teachings may be labeled with any suitable fluorophore and quencher combinations. For example, any fluorophore of the present teachings may be attached to any probe DNA sequence of the present teachings.

### Labels

As described above and herein throughout, an oligonucleotide of the disclosure may be modified to comprise one or more labels. Labels contemplated herein include a fluorophore, a quencher, a barcode, a mass tag, or a combination thereof. In any of the embodiments of the disclosure, a label may be attached to any probe oligonucleotide sequence disclosed herein. For example and without limitation, the label may be fluorescein, hexachlorofluorescein, TEX 615, and/or TYE™ 665. In some embodiments, the label is FAM, HEX, TEX615, Cy5, or a combination thereof. The fluorophores may excite between 450 nm and 763 nm and emit between 500 nm and 800 nm. An oligonucleotide of the disclosure may comprise one or more quenchers. Quenchers contemplated by the disclosure include but are not limited to Dy Q-425, Q-505, Q-1, Q-2, Q-660, Q-661 (hydrophil), Q-3, Q-700, Q-4; Dabcyl; BHQ 0, 1, 2, 3; ATTO 580Q, 612Q; BBQ-650, Iowa Black® quenchers, Black Hole Quenchers®, or a combination thereof. A mass tag is a tag having a specific mass for use in mass spectrophotometric analysis as described elsewhere herein.

Additional labels contemplated for use by the disclosure are fluorescein (6-FAM; FAM 6 isomer), 6-FAM (NHS Ester), 5-carboxyfluorescein (FAM; 5 isomer), fluorescein dT, FAM-5-EX, Cy3, 2′7′-dimethoxy-4′5-dichloro-6-carboxyfluorescein (JOE, 6-isomer), rhodamine, 6-carboxyrhodamine (R6G), N,N,N′,N′-tetramethyl-6-carboxyrhodamine (TAMRA, 5-isomer), TAMRA 5,6-isomer, TAMRA, 6-isomer (NHS Ester), 6-carboxy-X-rhodamine (ROX), carboxy rhodamine 6G (CR6G), 5/6-isomer, Caroboxy rhodamine 6G (CR6G) 5-isomer, rhodamine 11 OX, MAX (NHS Ester), TET, Cy5, Cy5.5, 4-(4′dimethylaminophenylazo) benzoic acid (DABCYL), CASCADE BLUE® (pyrenyloxytrisulfonic acid), OREGON GREEN™ (2′,7′-difluorofluorescein), Yakima Yellow, HEX, TEX615, TYE665, TYE705, TEXAS RED™ (sulforhodamine 101 acid chloride, NHS Ester), Alexa Fluor®350, 430, 488, 532, 546, 548, 555, 546, 560, 594, 610-X, 610, 620, 633, 647, 660, 700, 750, and 790; Cyanine, Cyanine 3, Cyanine 3.5, Cyanine 5, Cyanine 5.5, Cyanine 7, and 5-(2′-aminoethyl)aminonaphthalene-1-sulfonic acid (EDANS), 5′ IRDye 700, 800, 800CW (NHS Ester), ATTO™ 390, 425, 430LS, 465, 488 (NHS Ester), 495, 514, 520, 532 (NHS Ester), 542 (hydrophil), 550 (NHS Ester), 565 (NHS Ester), Rho101 (NHS Ester), 590 (NHS Ester), 594, 633 (NHS Ester), 647 (NHS Ester), 647N, Rho6G, Rho3B, Rho11, Rho13, Rho14, MB2 (redox), 660, 655 (hydrophil), 665, Oxa12 (lipophil), 680, 700, 725, 740, Methylene Blue, Rhodamine Green-X (NHS Ester), Rhodamine Red-X (NHS Ester), 5-TAMRA (Azide), WellRed D4 Dye, WellRED D3 Dye, WellRed D2 Dye, 6-FAM (Azide), Lightcycler 640 (NHS Ester), Dy750 (NHS Ester), Abberior® FLIP 565, STAR 440SX, STAR 470SX, STAR 488, CAGE 500, STAR 520SXP, CAGE 532, CAGE 552, CAGE 590, STAR 580, STAR 600, CAGE 635, STAR 635P, STAR RED, STAR 635, Pyrene, Dy350 XL, Eterneon 350/430 or 350/455, Dy350, 360 XL, 370 XL, 375 XL, 380 XL, 478, 480 XL, 490, 495, 505, 510 XL, 511 XL, 520XL, 521 XL, 530, 594, 601 XL, 605, 610, 615, 633, 634, 630, 631, 632, 633, 635, 636, 647P, 648P, 650, 654, 652, 649P1, 651, 677, 675, 676, 678, 680, 681, 682, 700, 703, 701, 704, 730, 731, 732, 734, 749P1, 750, 751, 752, 754, 778, 777, 776, 800, 780, 781, 782, and 831; Eterneon 384/480, 393/523, 394/507, 480/635, Green 515 Azide, Yellow 530 Azide, Yellow 550 Azide, Yellow 555 Azide, Orange 580 Azide, Red 600 Azide, Red 630 Azide, RED 645 Azide, Far Red 680 Azide, Sulforhodamine 101, Dy395 XL, 405, 431, 430, 481 XL, 485 XL, 550, 555, 556, 549P1, 574P1, 554, 594, Chromeo 488, 494, 546, 642, Oyster 488, 555, 647, 650, 680, IBApy 493/503, IBApy FL, or a combination thereof.

TABLE 1

| | Primer/Probe Sequence |
|---|---|
| SEQ ID NO. 67 | TGGCCAGAACTGACAGGCAAA |
| SEQ ID NO. 68 | TTTCTCCTGAACGTGGCTGGC |
| SEQ ID NO. 69 | 56-FAM/ACGCTAACT/ZEN/CCAGCATTGGTCTGT/3IABKFQ/ |
| SEQ ID NO. 70 | CCGTCACGCTGTTGTTAGG |
| SEQ ID NO. 71 | GCTGTGTTAATCAATGCCACAC |
| SEQ ID NO. 72 | 5HEX/AACTTGCCG/ZEN/AATTAGAGCRGCAGT/3IABKFQ |
| SEQ ID NO. 73 | CGTTTCGTCTGGATCGCAC |
| SEQ ID NO. 74 | GCTGGGTAAAATAGGTCACC |
| SEQ ID NO. 75 | 5TEX615/TATCATTGGTGGTGCCGTAGTCGC/3IAbRQSp |
| SEQ ID NO. 76 | GAGAGGATGAYCAGCCACAC |
| SEQ ID NO. 77 | CGCCCATTGTSCAATATTCC |
| SEQ ID NO. 78 | 5TYE665/TGAGACACGGTCCAGACTCCTACG/3IAbRQSp |
| SEQ ID NO. 79 | AATCACAGGGCGTAGTTGTG |
| SEQ ID NO. 80 | ACCCACCAGCCAATCTTAGG |
| SEQ ID NO. 81 | 56-FAM/TAGCTTGAT/ZEN/CGCCCTCGATTTGGG/3IABKFQ/ |
| SEQ ID NO. 82 | GCGGAGTTAACTATTGGCTAG |
| SEQ ID NO. 83 | GGCCAAGCTTCTATATTTGCG |
| SEQ ID NO. 84 | 5HEX/TTRTTYGGT/ZEN/GGTTGYTTTRTTAA/3IABKFQ |
| SEQ ID NO. 85 | GCGGAGTTARYTATTGGCTAG |
| SEQ ID NO. 86 | GGCCAAGCYTCTAWATTTGCG |
| SEQ ID NO. 87 | /5HEX/CCGGACGGT/ZEN/CTTGGTAATTTGGGT/3IABKFQ/ |
| SEQ ID NO. 88 | /5HEX/CCGTACGGT/ZEN/TTAGGCAATTTGGGT/3IABKFQ/ |
| SEQ ID NO. 89 | GGCGGCGTTGATGTCCTTCG |
| SEQ ID NO. 90 | CCATTCAGCCAGATCGGCATC |
| SEQ ID NO. 91 | 5TEX615/AGCTCTTCTATCCTGGTGCTGCG/3IAbRQSp |
| SEQ ID NO. 92 | AACTTTCACAGGTGTGCTGGGT |
| SEQ ID NO. 93 | CCGTACGCATACTGGCTTTGC |
| SEQ ID NO. 94 | 56-FAM/AAACCGGGC/ZEN/GATATGCGTCTGTAT/3IABKFQ/ |
| SEQ ID NO. 95 | GTATCGCCGTCTAGTTCTGC |
| SEQ ID NO. 96 | CCTTGAATGAGCTGCACAGTGG |
| SEQ ID NO. 97 | 5HEX/TCGTCGCGG/ZEN/AACCATTCGCTAAA/3IABKFQ/ |
| SEQ ID NO. 98 | GTTTGATCGTCAGGGATGGC |
| SEQ ID NO. 99 | GGCGAAAGTCAGGCTGTG |
| SEQ ID NO. 100 | 5TEX615/CATCAGGACAAGATGGGCGGTATG/3IAbRQSp |
| SEQ ID NO. 101 | GCTGCTCAAGGAGCACAGGAT |
| SEQ ID NO. 102 | CACATTGACATAGGTGTGGTGC |
| SEQ ID NO. 103 | 56-FAM/AGGATGGCA/ZEN/AGGCCCACTATTTCA/3IABKFQ |
| SEQ ID NO. 104 | AACAGCCTCAGCAGCCGGTTA |
| SEQ ID NO. 105 | TTCGCCGCAATCATCCCTAGC |
| SEQ ID NO. 106 | 5HEX/AGCCATTAC/ZEN/GTTCCAGAGTTGCGT/3IABKFQ |
| SEQ ID NO. 107 | GCCGAGGCTTACGGGATCAAG |
| SEQ ID NO. 108 | CAAAGCGCGTAACCGGATTGG |
| SEQ ID NO. 109 | 5TEX615/TCTGCTGAAGTTTRYCGAGGCMAA/3IAbRQSp |
| SEQ ID NO. 110 | AACTTTCACAGGTGTGCTGGGT |
| SEQ ID NO. 111 | CCGTACGCATACTGGCTTTGC |
| SEQ ID NO. 112 | 56-FAM/AAACCGGGC/ZEN/GATATGCGTCTGTAT/3IABKFQ/ |
| SEQ ID NO. 113 | CTGGGTTCTATAAGTAAAACCTTCACCGG |
| SEQ ID NO. 114 | CTTCCACTGCGGCTGCCAGTT |
| SEQ ID NO. 115 | 5HEX/GATGCCATT/ZEN/GCYCGSGGTGAAAT/3IABKFQ |
| SEQ ID NO. 116 | CCGAAGCCTATGGCGTGAAATCC |
| SEQ ID NO. 117 | GCAATGCCCTGCTGGAGCG |
| SEQ ID NO. 118 | 5TEX615/ATGTTGGCCTGAACCCAGCG/3IAbRQSp |
| SEQ ID NO. 119 | AGCACATACAGAATATGTCCCTGC |
| SEQ ID NO. 120 | ACCTGTTAACCAACCTACTTGAGGG |
| SEQ ID NO. 121 | /56-FAM/TTGCAAGACGGACTGGCTTAGACC/3BHQ_1/ |
| SEQ ID NO. 122 | CCTGATCGGATTGGAGAACC |
| SEQ ID NO. 123 | CTACCTCTTGAATAGGCGTAACC |
| SEQ ID NO. 124 | /5TEX615/ACGTCGCGCAAGTTCCTGATAGAC/3IAbRQSp/ |
| SEQ ID NO. 125 | TAGTGACTGCTAATCCAAATCACAG |
| SEQ ID NO. 126 | GCACGAGCAAGATCATTACCATAGC |
| SEQ ID NO. 127 | /5HEX/AGTTATCCAACAAGGCCAAACTCAACA/3BHQ_1/ |
| SEQ ID NO. 128 | AATCACAGGGCGTAGTTGTG |
| SEQ ID NO. 129 | ACCCACCAGCCAATCTTAGG |
| SEQ ID NO. 130 | /5HEX/TAGCTTGATCGCCCTCGATTTGGG/3BHQ_1/ |
| SEQ ID NO. 131 | GTGGGATGGAAAGCCACG |
| SEQ ID NO. 132 | CACTTGCGGGTCTACAGC |
| SEQ ID NO. 133 | /56-FAM/TTACTTTGGGCGAAGCCATGCAAG/3BHQ_1/ |
| SEQ ID NO. 134 | CACCTATGGTAATGCTCTTGC |
| SEQ ID NO. 135 | CTGGAACTGCTGACAATGCC |
| SEQ ID NO. 136 | /5TEX615/TGGGAGAAAGATATGACTTTAGGTGAGGCA/3IAbRQSp/ |
| SEQ ID NO. 137 | CCGTGTATGTTCAGCTAT |
| SEQ ID NO. 138 | CTTATCCATCACGCCTTT |
| SEQ ID NO. 139 | /5TEX615/TATGATGTCGATACCGCCAAATACCA/3IAbRQSp/ |
| SEQ ID NO. 140 | CTGTATGTCAGCGATCAT |
| SEQ ID NO. 141 | GATGCCAGTTTGCTTATCC |
| SEQ ID NO. 142 | /56FAM/AAGTCTGGG/ZEN/TGAGAACGGTGTCTAT/3IABKFQ |
| SEQ ID NO. 143 | CAGTCAGTATGCGAGTTTC |
| SEQ ID NO. 144 | AAAATTCGCCAAGCCATC, |
| SEQ ID NO. 145 | /5HEX/TGCATAAGC/ZEN/CAGTGCGTTTTTATAT/3IABKFQ |
| SEQ ID NO. 146 | AGATCAGTTGGGTGCACG |
| SEQ ID NO. 147 | TGCTTAATCAGTGAGGCACC |
| SEQ ID NO. 148 | /56-FAM/ATGAAGCCA/ZEN/TACCAAACGACGAGC/3IABKFQ/ |
| SEQ ID NO. 149 | CTGGAGCGAAAGATCCACTA |
| SEQ ID NO. 150 | ATCGTCCACCATCCACTG |
| SEQ ID NO. 151 | /5HEX/CCAGATCGG/ZEN/CGACAACGTCACC/3IABKFQ/ |
| SEQ ID NO. 152 | TGGCCAGAACTGACAGGCAAA |
| SEQ ID NO. 153 | TTTCTCCTGAACGTGGCTGGC |
| SEQ ID NO. 154 | 56-FAM/ACGCTAACT/ZEN/CCAGCATTGGTCTGT/3IABKFQ/ |
| SEQ ID NO. 155 | CCGTCACGCTGTTGTTAGG |
| SEQ ID NO. 156 | GCTGTGTTAATCAATGCCACAC |
| SEQ ID NO. 157 | 5HEX/AACTTGCCG/ZEN/AATTAGAGCRGCAGT/3IABKFQ |
| SEQ ID NO. 158 | CGTTTCGTCTGGATCGCAC |
| SEQ ID NO. 159 | GCTGGGTAAAATAGGTCACC |
| SEQ ID NO. 160 | 5TEX615/TATCATTGGTGGTGCCGTAGTCGC/3IAbRQSp |
| SEQ ID NO. 161 | GAGAGGATGAYCAGCCACAC |
| SEQ ID NO. 162 | CGCCCATTGTSCAATATTCC |
| SEQ ID NO. 163 | 5TYE665/TGAGACACGGTCCAGACTCCTACG/3IAbRQSp |
| SEQ ID NO. 164 | AATCACAGGGCGTAGTTGTG |
| SEQ ID NO. 165 | ACCCACCAGCCAATCTTAGG |
| SEQ ID NO. 166 | 56-FAM/TAGCTTGAT/ZEN/CGCCCTCGATTTGGG/13IABKFQ/ |
| SEQ ID NO. 167 | GCGGAGTTAACTATTGGCTAG |
| SEQ ID NO. 168 | GGCCAAGCTTCTATATTTGCG |
| SEQ ID NO. 169 | 5HEX/TTRTTYGGT/ZEN/GGTTGYTTTRTTAA/3IABKFQ |
| SEQ ID NO. 170 | GCGGAGTTARYTATTGGCTAG |
| SEQ ID NO. 171 | GGCCAAGCYTCTAWATTTGCG |
| SEQ ID NO. 172 | /5HEX/CCGGACGGT/ZEN/CTTGGTAATTTGGGT/3IABKFQ/ |
| SEQ ID NO. 173 | /5HEX/CCGTACGGT/ZEN/TTAGGCAATTTGGGT/3IABKFQ |
| SEQ ID NO. 174 | GGCGGCGTTGATGTCCTTCG |
| SEQ ID NO. 175 | CCATTCAGCCAGATCGGCATC |
| SEQ ID NO. 176 | 5TEX615/AGCTCTTCTATCCTGGTGCTGCG/3IAbRQSp |
| SEQ ID NO. 177 | GAGAGGATGAYCAGCCACAC |
| SEQ ID NO. 178 | CGCCCATTGTSCAATATTCC |
| SEQ ID NO. 179 | 5TYE665/TGAGACACGGTCCAGACTCCTACG/3IAbRQSp |
| SEQ ID NO. 180 | AACTTTCACAGGTGTGCTGGGT |
| SEQ ID NO. 181 | CCGTACGCATACTGGCTTTGC |
| SEQ ID NO. 182 | 56-FAM/AAACCGGGC/ZEN/GATATGCGTCTGTAT/3IABKFQ/ |
| SEQ ID NO. 183 | GTATCGCCGTCTAGTTCTGC |
| SEQ ID NO. 184 | CCTTGAATGAGCTGCACAGTGG |
| SEQ ID NO. 185 | 5HEX/TCGTCGCGG/ZEN/AACCATTCGCTAAA/3IABKFQ/ |
| SEQ ID NO. 186 | GTTTGATCGTCAGGGATGGC |
| SEQ ID NO. 187 | GGCGAAAGTCAGGCTGTG |
| SEQ ID NO. 188 | 5TEX615/CATCAGGACAAGATGGGCGGTATG/3IAbRQSp |
| SEQ ID NO. 189 | GAGAGGATGAYCAGCCACAC |
| SEQ ID NO. 190 | CGCCCATTGTSCAATATTCC |
| SEQ ID NO. 191 | 5TYE665/TGAGACACGGTCCAGACTCCTACG/3IAbRQSp |
| SEQ ID NO. 192 | GCTGCTCAAGGAGCACAGGAT |
| SEQ ID NO. 193 | CACATTGACATAGGTGTGGTGC |
| SEQ ID NO. 194 | 56-FAM/AGGATGGCA/ZEN/AGGCCCACTATTTCA/3IABKFQ |
| SEQ ID NO. 195 | AACAGCCTCAGCAGCCGGTTA |
| SEQ ID NO. 196 | TTCGCCGCAATCATCCCTAGC |
| SEQ ID NO. 197 | 5HEX/AGCCATTAC/ZEN/GTTCCAGAGTTGCGT/3IABKFQ |
| SEQ ID NO. 198 | GCCGAGGCTTACGGGATCAAG |
| SEQ ID NO. 199 | CAAAGCGCGTAACCGGATTGG |
| SEQ ID NO. 200 | 5TEX615/TCTGCTGAAGTTTRYCGAGGCMAA/3IAbRQSp |
| SEQ ID NO. 201 | GAGAGGATGAYCAGCCACAC |
| SEQ ID NO. 202 | CGCCCATTGTSCAATATTCC |
| SEQ ID NO. 203 | 5TYE665/TGAGACACGGTCCAGACTCCTACG/3IAbRQSp |
| SEQ ID NO. 204 | AACTTTCACAGGTGTGCTGGGT |
| SEQ ID NO. 205 | CCGTACGCATACTGGCTTTGC |
| SEQ ID NO. 206 | 56-FAM/AAACCGGGC/ZEN/GATATGCGTCTGTAT/3IABKFQ |
| SEQ ID NO. 207 | CTGGGTTCTATAAGTAAAACCTTCACCGG |
| SEQ ID NO. 208 | CTTCCACTGCGGCTGCCAGTT |
| SEQ ID NO. 209 | 5HEX/GATGCCATT/ZEN/GCYCGSGGTGAAAT/3IABKFQ |
| SEQ ID NO. 210 | CCGAAGCCTATGGCGTGAAATCC |
| SEQ ID NO. 211 | GCAATGCCCTGCTGGAGCG |
| SEQ ID NO. 212 | 5TEX615/ATGTTGGCCTGAACCCAGCG/3IAbRQSp |
| SEQ ID NO. 213 | GAGAGGATGAYCAGCCACAC |
| SEQ ID NO. 214 | CGCCCATTGTSCAATATTCC |
| SEQ ID NO. 215 | 5TYE665/TGAGACACGGTCCAGACTCCTACG/3IAbRQSp |
| SEQ ID NO. 216 | AGCACATACAGAATATGTCCCTGC |
| SEQ ID NO. 217 | ACCTGTTAACCAACCTACTTGAGGG |
| SEQ ID NO. 218 | /56-FAM/TTGCAAGACGGACTGGCTTAGACC/3BHQ_1/ |
| SEQ ID NO. 219 | CCTGATCGGATTGGAGAACC |
| SEQ ID NO. 220 | CTACCTCTTGAATAGGCGTAACC |
| SEQ ID NO. 221 | /5TEX615/ACGTCGCGCAAGTTCCTGATAGAC/3IAbRQSp/ |
| SEQ ID NO. 222 | TAGTGACTGCTAATCCAAATCACAG |
| SEQ ID NO. 223 | GCACGAGCAAGATCATTACCATAGC |
| SEQ ID NO. 224 | /5HEX/AGTTATCCAACAAGGCCAAACTCAACA/3BHQ_1/ |
| SEQ ID NO. 225 | GAGAGGATGAYCAGCCACAC |
| SEQ ID NO. 226 | CGCCCATTGTSCAATATTCC |
| SEQ ID NO. 227 | 5TYE665/TGAGACACGGTCCAGACTCCTACG/3IAbRQSp |
| SEQ ID NO. 228 | AATCACAGGGCGTAGTTGTG |
| SEQ ID NO. 229 | ACCCACCAGCCAATCTTAGG |
| SEQ ID NO. 230 | /5HEX/TAGCTTGATCGCCCTCGATTTGGG/3BHQ_1/ |
| SEQ ID NO. 231 | GTGGGATGGAAAGCCACG |
| SEQ ID NO. 232 | CACTTGCGGGTCTACAGC |
| SEQ ID NO. 233 | /56-FAM/TTACTTTGGGCGAAGCCATGCAAG/3BHQ_1/ |
| SEQ ID NO. 234 | CACCTATGGTAATGCTCTTGC, |
| SEQ ID NO. 235 | CTGGAACTGCTGACAATGCC |
| SEQ ID NO. 236 | /5TEX615/TGGGAGAAAGATATGACTTTAGGTGAGGCA/3IAbRQSp/ |
| SEQ ID NO. 237 | GAGAGGATGAYCAGCCACAC |
| SEQ ID NO. 238 | CGCCCATTGTSCAATATTCC |
| SEQ ID NO. 239 | 5TYE665/TGAGACACGGTCCAGACTCCTACG/3IAbRQSp |
| SEQ ID NO. 240 | AGATCAGTTGGGTGCACG |
| SEQ ID NO. 241 | TGCTTAATCAGTGAGGCACC |
| SEQ ID NO. 242 | /56-FAM/ATGAAGCCA/ZEN/TACCAAACGACGAGC/3IABKFQ/ |
| SEQ ID NO. 243 | CTGGAGCGAAAGATCCACTA |
| SEQ ID NO. 244 | ATCGTCCACCATCCACTG |
| SEQ ID NO. 245 | /5HEX/CCAGATCGG/ZEN/CGACAACGTCACC/3IABKFQ/ |
| SEQ ID NO. 246 | GAGAGGATGAYCAGCCACAC |
| SEQ ID NO. 247 | CGCCCATTGTSCAATATTCC |
| SEQ ID NO. 248 | 5TYE665/TGAGACACGGTCCAGACTCCTACG/3IAbRQSp |
| SEQ ID NO. 249 | CCGTGTATGTTCAGCTAT |
| SEQ ID NO. 250 | CTTATCCATCACGCCTTT |
| SEQ ID NO. 251 | /5TEX615/TATGATGTCGATACCGCCAAATACCA/3IAbRQSp/ |
| SEQ ID NO. 252 | CTGTATGTCAGCGATCAT |
| SEQ ID NO. 253 | GATGCCAGTTTGCTTATCC |
| SEQ ID NO. 254 | /56FAM/AAGTCTGGG/ZEN/TGAGAACGGTGTCTAT/3IABKFQ/ |
| SEQ ID NO. 255 | CAGTCAGTATGCGAGTTTC |
| SEQ ID NO. 256 | AAAATTCGCCAAGCCATC |
| SEQ ID NO. 257 | /5HEX/TGCATAAGC/ZEN/CAGTGCGTTTTTATAT/3IABKFQ/ |
| SEQ ID NO. 258 | GAGAGGATGAYCAGCCACAC |
| SEQ ID NO. 259 | CGCCCATTGTSCAATATTCC |
| SEQ ID NO. 260 | 5TYE665/TGAGACACGGTCCAGACTCCTACG/3IAbRQSp |

The present teachings provide a molecular assay. The present teachings may provide a qualitative (i.e., end point) molecular assay for the detection of family-specific KPC, ESBL, MBL, and ampC gene targets. The present teachings may provide a qualitative (i.e., end point) molecular assay for the detection of family-specific plasmid-mediated ampC β-lactamase genes. The present teachings may provide a qualitative (i.e., end point) molecular assay for the detection of OXA gene targets. Fluorescently-labeled DNA probes may be used for detection. The assay of the present teachings may provide for differentiation between a plasmid-mediated ampC β-lactamase gene from a chromosomal ampC β-lactamase gene; provided the two genes are not from the same chromosomal origin. The assay may involve extraction of DNA from bacterial cells. The assay may include subsequent PCR amplification. The assay may include gel-based detection.

In contrast, to traditional phenotypic methods which require 24-48 hours for data, the present teachings may provide for data generation in just hours or one hour. The total time required for DNA extraction, PCR set-up, amplification, and analysis may be around about 2 hours to about 3 hours. The sensitivity of the assay may be about 100%. The specificity of the assay may be about 100%. Therefore, the present teachings provide for fast and reliable detection. Implementation of such rapid assays have a positive impact for infection control and patient care.

The present teachings allow for the detection of multiple β-lactamase gene families. The β-lactamases may include all major β-lactamases including ampC types. For example, the present teachings may allow for identification of up to six to nine β-lactamase gene families. The β-lactamase gene families may include CMY, CTX-Ms, DHA, IMP, KPC, NDM, OXA, VIM, or a combination thereof. The AmpC β-lactamases gene families may include MOX, ACC, FOX, DHA, CMY, EBC, or a combination thereof.

The present teachings provide for a kit which allows for identification of at least nine β-lactamase gene families. The gene families may include: IMP-like, IMP-1-like, NDM-like, OXA-48-like, CTX-M-14-like, CTX-M-15-like, CMY-2-like, DHA-like, VIM-like, and KPC-like. The kit may also include an endogenous internal control (IC) that targets a conserved region common in gram-negative bacteria to reduce false negatives due to PCR inhibition, DNA degradation, or poor extraction. It is contemplated that the endogenous internal control discriminates false negative samples from true negative samples due to but not limited to one or more of PCR inhibition, DNA degradation, and/or poor extraction. The kit may utilize sequence-specific primer pairs for the PCR amplification of each gene family. The kit may utilize fluorescently-labeled, target-specific DNA probes for detection by real-time PCR.

The kit may include one or more multiplex primer-probe mixes containing one or more primers and one or more probes. The multiplex primer-probe mix may be a 10×PCR mix. In one example, the kit includes three multiplex primers-probes mix vials. The mix vials may provide for simultaneous real-time PCR amplification of all targets between three reaction tubes. PCR Mix 1 may amplify a first set of three gene families. For example, CMY-2, CTX-M-14, and CTX-M-15. PCR Mix 2 may amplify a second set of three gene families. For example, OXA-48, IMP, and VIM. PCR mix 3 may amplify a third set of gene families. For example, DHA, KPC, and NDM. The multiplex mix may also include an internal control (IC) in each mix. The kit may include three external DNA control vials or first control mix vial, a second control mix vial and a third control mix vial. The DNA control mix vial may contain synthetic DNA templates of the corresponding multiplex targets. The DNA control mixes may serve as a positive control for each multiplex reaction. The DNA control mix may contain stabilized bacteria with chromosomal or transmissible genetic elements in a sample matrix similar to a patient sample.

The present teachings provide for a kit which allows for identification of at least six plasmid-mediated ampC gene families. The gene families may include: MOX-like, DHA-like, ACC-like, EBC-like, FOX-like, and CMY-2-like. The kit may also include an endogenous internal control (IC) that targets a conserved region common in gram-negative bacteria to reduce false negatives due to PCR inhibition, DNA degradation, or poor extraction. It is contemplated that the endogenous internal control discriminates false negative samples from true negative samples due to but not limited to one or more of PCR inhibition, DNA degradation, and/or poor extraction. The kit may utilize sequence-specific primer pairs for the PCR amplification of each family. The kit may utilize fluorescently-labeled, target-specific DNA probes for detection by real-time PCR.

The kit may include one or more multiplex primer-probe mixes containing one or more primers and one or more probes. The multiplex primer-probe mix may be a 10×PCR mix. In one example, the kit includes two multiplex primers-probes mix vials. The mix vials may provide for simultaneous real-time PCR amplification of all targets between two reaction tubes. PCR Mix 1 may amplify a first set of three gene families. For example, MOX, ACC and FOX. PCR Mix 2 may amplify a second set of three gene families. For example, DHA, EBC and CMY-2. The multiplex mix may also include an internal control (IC) in each mix. The kit may include two external DNA control vials or first control mix vial and a second control mix vial. The DNA control mix vial may contain synthetic DNA templates of the corresponding multiplex targets. The DNA control mixes may serve as a positive control for each multiplex reaction.

The present teachings provide for a kit which allows for identification of at least six OXA carbapenemase gene families. The gene families may include: OXA-23, OXA-24/40, OXA-48, OXA-51, OXA-58, and OXA-143. The gene families may include like gene families. The kit may also include an endogenous internal control (IC) that targets a conserved region common in gram-negative bacteria to reduce false negatives due to PCR inhibition, DNA degradation, or poor extraction. It is contemplated that the endogenous internal control discriminates false negative samples from true negative samples due to but not limited to one or more of PCR inhibition, DNA degradation, and/or poor extraction. The kit may utilize sequence-specific primer pairs for the PCR amplification of each family. The kit may utilize fluorophore-labeled, target-specific DNA probes for detection by real-time PCR.

The kit may include one or more multiplex primer-probe mixes containing one or more primers and one or more probes. The multiplex primer-probe mix may be a 10×PCR mix. In one example, the kit includes two multiplex primers-probes mix vials. The mix vials may provide for simultaneous real-time PCR amplification of all targets between two reaction tubes. PCR Mix 1 may amplify a first set of three gene families. For example, OXA 143, OXA 23 and OXA 51. PCR Mix 2 may amplify a second set of three gene families. For example, OXA 24/40, OXA-48 and OXA-58. The multiplex mix may also include an internal control (IC) in each mix. The kit may include two external DNA control vials or first control mix vial and a second control mix vial. The DNA control mix vial may contain synthetic DNA templates of the corresponding multiplex targets. The DNA control mixes may serve as a positive control for each multiplex reaction.

In addition, the present teachings contemplate that the kit or kits of the present teachings may provide for the detection of a non-beta lactamase gene family. The kit or kits may provide for detection of plasmid-mediated mechanisms of antibiotic resistance for one more types/categories of antibiotics. For example, the kit may also provide for the detection of the MCR-1 gene which confers colistin and polymyxin resistance. The kit or kits may include primer sequences, probe sequences, and a control sequence for detection of one or more non-beta lactamase gene family in addition to beta-lactamase genes. For example, a kit may provide for the detection of ampC genes families and a MCR-1 gene family.

Mcr-1 encodes a member of the family of phosphoethanolamine (PEA) transferases that decorates the lipid A headgroups of lipopolysaccharide with PEA. Modification of lipid A on the 1 and 4′ headgroup positions with PEA or 4-amino-arabinose masks the negatively charged phosphate groups on the bacterial surface, which are involved in interaction with cationic antimicrobial peptides (CAMPs) such as colistin and polymyxin B (Anandan et al., Proceedings of the National Academy of Sciences 114 (9) 2218-2223 (2017)). This modification confers resistance to CAMPs, as well as host innate immune defensins; however, the exact mechanism of resistance is not known.

Furthermore, the present teachings allow for the expansion of the detection of other β-lactamase gene families including TEM, SHV, and GES. The gene families may include like gene families. The kit may also include an endogenous internal control (IC) that targets a conserved region common in Gram-negative bacteria to reduce false negatives due to PCR inhibition, DNA degradation, or poor extraction. It is contemplated that the endogenous internal control discriminates false negative samples from true negative samples due to but not limited to one or more of PCR inhibition, DNA degradation, and/or poor extraction. The kit may utilize sequence-specific primer pairs for the PCR amplification of each family. The kit may utilize fluorescently-labeled, target-specific DNA probes for detection by real-time PCR.

The kit or kits of the present teachings may include synthetic DNA oligonucleotide primers, target-specific DNA probes and DNA controls for the specified gene targets suspended in TE buffer, pH 8.0. Any of the primers of the disclosure may additionally comprise a universal tail (as described e.g., in Vandenbussche et al., PLoS One 11(10):e0164463 (2016); Ebili et al., J. Biomolecular Techniques 28: 97-110 (2017), each incorporated herein by reference in their entirety). The universal tail is a sequence added to the end of a primer (typically the 5′ end) to simplify the process used for DNA sequencing by enabling the use of universal forward and reverse sequencing primers. For example and without limitation, a universal tail contemplated by the disclosure is an M13 tail.

### Modified Oligonucleotides

As used herein, an “oligonucleotide” is an oligomer comprised of nucleotides. An oligonucleotide may be comprised of DNA, RNA modified forms thereof, or a combination thereof.

The term “nucleotide” or its plural as used herein is interchangeable with modified forms as discussed herein and otherwise known in the art. In certain instances, the art uses the term “nucleobase” which embraces naturally occurring nucleotides as well as modifications of nucleotides that can be polymerized. Thus, nucleotide or nucleobase means the naturally occurring nucleobases adenine (A), guanine (G), cytosine (C), thymine (T) and uracil (U) as well as non-naturally occurring nucleobases such as xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C₃-C₆)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which is hereby incorporated by reference in its entirety). In various aspects, oligonucleotides also include one or more “nucleosidic bases” or “base units” which include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include pyrrole, and diazole or triazole derivatives, including those universal bases known in the art.

Oligonucleotides may also include modified nucleobases. A “modified base” is understood in the art to be one that can pair with a natural base (e.g., adenine, guanine, cytosine, uracil, and/or thymine) and/or can pair with a non-naturally occurring base. Exemplary modified bases are described in EP 1 072 679 and WO 97/12896, the disclosures of which are incorporated herein by reference. Modified nucleobases include, without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity of the oligonucleotide and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects, combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Modified oligonucleotides contemplated for use include those wherein both one or more sugar and/or one or more internucleotide linkage of the nucleotide units in the oligonucleotide is replaced with “non-naturally occurring” sugars (i.e., sugars other than ribose or deoxyribose) or internucleotide linkages, respectively. In some aspects, this embodiment contemplates a peptide nucleic acid (PNA). In PNA compounds, the sugar-backbone of an oligonucleotide is replaced with an amide-containing (e.g., peptide bonds between N-(2-aminoethyl)-glycine units) backbone. See, for example U.S. Pat. Nos. 5,539,082; 5,714,331; and 5,719,262, and Nielsen et al., Science, 1991, 254, 1497-1500, the disclosures of which are herein incorporated by reference.

Modified oligonucleotides may also contain one or more substituted sugar groups. In some aspects, a modification of the sugar includes Locked Nucleic Acids (LNAs) in which the 2′-hydroxyl group is linked to the 3′ or 4′ carbon atom of the sugar ring, thereby forming a bicyclic sugar group. The linkage is in certain aspects a methylene (—CH₂—)ₙ group bridging the 2′ oxygen atom and the 4′ carbon atom wherein n is 1 or 2. LNAs and preparation thereof are described in WO 98/39352 and WO 99/14226, the disclosures of which are incorporated herein by reference.

Modified oligonucleotides may also contain a label or a universal tail, each as described herein.

The contents of the kit may be enclosed in vials. For example, the one or more 10×PCR mixes may be comprised of 275 μL. For example, the one or more control mixes may be comprised of 14 μL. For example, the contents of the kit may be sufficient for about 100 reactions total and about 12 reactions of the control DNA mix.

Detection of each target is based on the optical fluorescence of the fluorophore conjugated to each target-specific DNA probe. Any suitable fluorophore and nucleic acid sequence combination may be used. For example, the fluorophores may be selected from the group consisting of: FAM, HEX, TEX615, TYE665, and a combination thereof.

The present teachings provide assays for the detection of β-lactamase gene families from a biological sample. The assays may be included in a kit or kits. The kit may provide for the detection of β-lactamase by various molecular biology technologies and platforms. The kit may include one or more primers and/or probes for the identification by polymerase chain reaction or microarray of one or more β-lactamase genes including CMY, CTX-M, OXA, IMP, VIM, DHA, KPC, MOX, ACC, FOX, EBC, NDM, TEM, SHV, GES, or a combination thereof.

The kit may include one or more primers and/or probes for the identification by polymerase chain reaction or microarray of a non-beta lactamase gene family which confers antibiotic resistance. The kit may include one or more primers and/or probes for the identification by polymerase chain reaction or microarray of one or more MCR genes. The kit may include one or more primers and/or probes for the identification by polymerase chain reaction or microarray of a MCR-1 gene.

The kit may provide for detection of specified targets from crude biological samples such as blood, urine, plasma, feces, sputum, etc. The kit may provide for detection of specified targets directly from or extracted directly from crude biological samples including but not limited to blood, blood cultures, urine, plasma, feces, fecal swabs, peri-rectal/peri-anal swabs, sputum, and bacterial cultures.

The kit may be used for detection of specified targets from purified nucleic acid samples. The kit may be used for any nucleic acid amplification methodology. The kit may be used with conventional polymerase chain reaction. The kit may be used with real-time polymerase chain reaction. The kit may be used with digital droplet polymerase chain reaction. The kit may be used with detection by microarray technology. The kit may be used with fluorescence and/or infra-red probe-based detection chemistries. The kit may be used with intercalating dye-based detection chemistries. The kit may be used for detection of nucleic acid polymerase chain reaction amplicons ranging from 25 base pairs to 2000 base pairs.

The kit may include various reagents. The various reagents may be contained in various vials. The kit may include a primer set or primer sets. The primer set or primer sets may be labeled or unlabeled with a tracking dye or fluorophore. The kit may include probes. The kit may include a primer-probe mix. The kit may include controls. The kit may include magnesium chloride. The kit may include dNTPs. The kit may include DNA polymerase. The kit may include a tracking dye. The kit may include a composition containing a tracking dye. The kit may include a written protocol. The kit may include a customized master mix in a single tube, two tubes, three tubes, or four tubes containing all chemicals and enzymes necessary to run the PCR assay described herein. The kit may include freeze-dried or lyophilized reagents in a single assay tube or multiple assay tubes. The kit may provide for detection of nucleic acid and the kit reagents may be provided in any liquid form, pooled reaction mix, or lyophilized, freeze dried, or cryo-preserved format.

The kit may include a primer set. The primer set may include at least one primer pair. A primer pair may include a forward primer and a reverse primer. The primer set may include one pair of primers. The primer set may include more than one pair of primers. The primer set may include two pairs of primers. The primer set may include three pairs of primers. The primer set may include one to six pairs of primers. The primer set may include one to ten pairs of primers. The primer set may include up to 30 pairs of primers. The primer set may include up to 50 pairs of primers. The primer set may include up to 100 pairs of primers.

The kit may include a primer-probe mix. The primer-probe mix may include a primer set. The primer-probe mix may include one or more probes. Each pair of primers of the primer set may include a probe or set of probes. The primer-probe mix may include a pair of internal control primers. The pair of internal control primers may include a forward primer and a reverse primer. The primer-probe mix may include an internal control probe.

For example, a primer-probe mix may include one or more pairs of primers, one associated probe per primer pair and internal controls including a pair of primers and a probe. Preferably, the primer-probe mix is a multiplex mix including more than one pair of primers, a probe for each primer pair and internal controls. The multiplex mix may be used for the identification of more than one β-lactamase gene family. Each primer pair and probe may detect a different β-lactamase gene family. For example, three primer pairs and their associated three probes may be used for detection of three different β-lactamase gene families.

The DNA concentration range of each primer set in a PCR may be about 1 nM to about 10 μM (10,000 nM). One or more primers may be labeled with a florescent marker as a probe. The DNA concentration of each probe in a PCR may be about 1 nM to about 10,000 nM. The DNA concentration of each probe in a PCR may be about 10 to about 500 nM.

The kit may include at least one control. The kit may include one, two, three or four controls. The kit may include one or more negative controls. The negative control may include nucleic acid known to express a resistance gene other than the target gene of interest. The kit may include one or more positive controls. The one or more positive controls may be internal controls. The positive control may include nucleic acid known to express or contain the resistance gene. The kit may include an endogenous internal control to reduce false negatives due to PCR inhibition, DNA degradation, and/or poor extraction. It is contemplated that the endogenous internal control discriminates false negative samples from true negative samples due to but not limited to one or more of PCR inhibition, DNA degradation, and/or poor extraction. The endogenous internal control may target a conserved nucleotide sequence or sequences common to the Gram-negative bacteria genome. For example, the internal control may detect the 16S rRNA and/or 23S rRNA gene(s). The internal control may detect the 16S and/or 23S rRNA gene for E. Coli, Pseudomonas, Acinetobacter, Klebsiella and Salmonella.

The kit may include control vector in the control vial. One or more pls of the vector control may be added to a 25 μl reaction to get the working concentration. The DNA concentrations for each control vector may be equivalent to 0.1 copy to 2000 copies or 0.0000243 pg/uL to 0.0455 pg/uL. The DNA concentrations for each control vector may be equivalent to 10 copies to 5000 copies or 0.001 pg/uL to 0.5 pg/uL. Control vector concentrations may be as high as 1×10(9) copies and any dilution thereof.

The assays of the present teachings may include the use of magnesium chloride. The kit may include magnesium chloride. The assay may be utilized with a concentration of about 2 mM to about 7 mM MgCl₂. Preferably, the concentration is about 3.0 mM to about 5.5 mM MgCl₂. More preferably, the concentration is 5.0 mM MgCl₂ for an assay for the detection of β-lactamase genes. More preferably the concentration is 5.0 mM MgCl₂ for an assay for the detection of ampC β-lactamase genes. More, preferably, the concentration is 5 mM MgCl₂ for an assay for the detection of OXA genes.

The assays of the present teachings may include the use of DNA polymerase. The kit may include DNA polymerase. The assay may be utilized with a concentration of about 0.25 U/25 μl reaction to about 3 U/25 μl reaction of DNA polymerase. Preferably, the concentration is 1.25 U/25 μl reaction DNA polymerase for an assay for the detection of β-lactamase genes. Preferably the concentration is 1.25 U/25 μl DNA polymerase for an assay for the detection of β-lactamase ampC genes. For example, the present teachings may utilize the PhilisaFAST® DNA polymerase.

The assays and methods of the present teachings may include a PCR cycling protocol. In one example, the cycling protocol comprises (1) 95° C. for 30 s; (2) 95° C. for 1 s; (3) 55° C. for 10 s; (4) 68° C. for 20 s; and repeating steps (2) to (4) for 40 cycles. In one example, the cycling protocol comprises (1) 95° C. for 30 s; (2) 95° C. for 6 s; (3) 66° C. for 10 s; and repeating steps (2) to (3) for 40 cycles. In one example, the cycling protocol includes a hot start of 98° C. for 30 s and 30 cycles of: 98° C. for 5 s, 60° C. for 10 s and 72° C. for 20 s. In one example, the cycling protocol includes using 98° C. for 30 s, followed by 30 cycles of 98° C. for 5 s, 60° C. for 10 s., and 72° C. for 25 s. In one example, the PCR protocols include a detection step where fluorescent signal is measured.

The kit may include one or more of the following: primer, probe and control. A mix of one or more of the following: primer, probe and internal control, may be enclosed in one container. A mix of one or more of the following: primer, probe and internal control, may be enclosed in more than one container. The container may be a vial. In one example, the kit includes 3 DNA control vials and 3 10× primer/probe mix vials. Nine antibiotic resistance gene families and one internal control may be identified with the vials. In one example, the kit includes 2 DNA control vials and 2 10× primer/probe mix vials. Six antibiotic resistance gene families and one internal control may be identified with the vials.

The present teachings allow for detection of the β-lactamase CMY-2 gene family from a biological sample. The present teachings provide for a kit including one or more primers and/or probes for the identification by multiplex real-time polymerase chain reaction of β-lactamase genes including the CMY-2-like gene family. The biological sample may include Gram-negative bacteria such as Escherichia coli, Proteus mirabilis, Klebsiella pneumoniae, Klebsiella oxytoca, Serratia marcescens, Citrobacter freundii and other Citrobacter species.

The CMY-2-like genes detected may include CMY-2, CMY-4, CMY-6, CMY-7, CMY-12, CMY-14, CMY-15, CMY-16, CMY-18, CMY-21, CMY-22, CMY-23, CMY-24, CMY-25, CMY-26, CMY-27, CMY-28, CMY-29, CMY-30, CMY-31, CMY-32, CMY-33, CMY-34, CMY-35, CMY-37, CMY-38, CMY-39, CMY-40, CMY-41, CMY-42, CMY-43, CMY-44, CMY-45, CMY-46, CMY-47, CMY-48, CMY-49, CMY-50, CMY-51, CMY-53, CMY-54, CMY-55, CMY-56, CMY-57, CMY-58, CMY-59, CMY-60, CMY-61, CMY-62, CMY-63, CMY-64, CMY-65, CMY-66, CMY-67, CMY-68, CMY-69, CMY-71, CMY-72, CMY-73, CMY-75, CMY-76, CMY-77, CMY-78, CMY-79, CMY-80, CMY-81, CMY-84, CMY-85, CMY-86, CMY-87, CMY-89, CMY-90, CMY-96, CMY-97, CMY-99, CMY-102, CMY-103, CMY-104, CMY-105, CMY-107, CMY-108, CMY-110, CMY-111, CMY-112, CMY-113, CMY-114, CMY-115, CMY-116, CMY-117, CMY-118, CMY-119, CMY-121, CMY-122, CMY-124, CMY-125, CMY-126, CMY-127, CMY-128, CMY-129, CMY-130, CMY-131, CMY-132, CMY-133, CMY-135, or a combination thereof.

The present teachings allow for the detection of the β-lactamase CTX-M gene family from a biological sample. The present teachings provide for a kit including one or more primers and/or probes for the identification by multiplex real-time polymerase chain reaction of β-lactamase genes including the CTX-M-14-like gene family. The biological sample may include Gram-negative bacteria such as Klebsiella pneumoniae, Enterobacter cloacae, Escherichia coli, Salmonella enterica, Proteus mirabilis and Shigella species. The CTX-M-14-like genes detected may include CTX-M-9, CTX-M-13, CTX-M-14, CTX-M-16, CTX-M-17, CTX-M-19, CTX-M-21, CTX-M-24, CTX-M-27, CTX-M-38, CTX-M-51, CTX-M-64, CTX-M-65, CTX-M-67, CTX-M-82, CTX-M-83, CTX-M-84, CTX-M-85, CTX-M-86, CTX-M-90, CTX-M-93, CTX-M-98, CTX-M-99, CTX-M-102, CTX-M-104, CTX-M-105, CTX-M-110, CTX-M-111, CTX-M-112, CTX-M-113, CTX-M-121, CTX-M-122, CTX-M-123, CTX-M-125, CTX-M-129, CTX-M-130, CTX-M-132, CTX-M-134, CTX-M-147, CTX-M-148, CTX-M-159, or a combination thereof.

The present teachings allow for the detection of the β-lactamase CTX-M gene family from a biological sample. The present teachings provide for a kit including one or more primers and/or probes for the identification by multiplex real-time polymerase chain reaction of β-lactamase genes including the CTX-M-15-like gene family. The biological sample may include Gram-negative bacteria such as Escherichia coli, Klebsiella pneumoniae, Citrobacter freundii, Shigella species and Proteus mirabilis. The CTX-M-15-like genes detected may include CTX-M-1, CTX-M-3, CTX-M-10, CTX-M-15, CTX-M-22, CTX-M-28, CTX-M-29, CTX-M-30, CTX-M-32, CTX-M-37, CTX-M-55, CTX-M-64, CTX-M-71, CTX-M-103, CTX-M-117, CTX-M-123, CTX-M-132, CTX-M-136, CTX-M-138, CTX-M-142, CTX-M-144, CTX-M-155, CTX-M-156, CTX-M-157, CTX-M-158, CTX-M-163, CTX-M-164, CTX-M-166, CTX-M-172, or a combination thereof.

The present teachings allow for the detection of the β-lactamase DHA gene family from a biological sample. The present teachings provide for a kit including one or more primers and/or probes for the identification by multiplex real-time polymerase chain reaction of β-lactamase genes including the DHA-like family. The biological sample may include Gram-negative bacteria such as Klebsiella pneumoniae, Morganella morganii, Escherichia coli, Enterobacter cloacae, Proteus mirabilis and Citrobacter koseri. The DHA-like genes detected may include DHA-1, DHA-2, DHA-5, DHA-6, DHA-7, DHA-9, DHA-10, DHA-12, DHA-13, DHA-14, DHA-15, DHA-16, DHA-17, DHA-18, DHA-19, DHA-20, DHA-21, DHA-22, or a combination thereof.

The present teachings allow for the detection of the β-lactamase IMP gene family from a biological sample. The present teachings provide for a kit including one or more primers and/or probes for the identification by multiplex real-time polymerase chain reaction of β-lactamase genes including the IMP-like family. The biological sample may include Gram-negative bacteria such as Serratia marcescens, Escherichia coli and Pseudomonas aeruginosa. The IMP-like genes detected may include IMP-1, IMP-2, IMP-3, IMP-4, IMP-5, IMP-6, IMP-7, IMP-8, IMP-9, IMP-10, IMP-13, IMP-14, IMP-15, IMP-16, IMP-18, IMP-19, IMP-20, IMP-22, IMP-24, IMP-25, IMP-26, IMP-27, IMP-28, IMP-30, IMP-32, IMP-33, IMP-34, IMP-37, IMP-38, IMP-40, IMP-42, IMP-45, IMP-48, IMP-49, IMP-51, IMP-52, or a combination thereof.

The present teachings allow for the detection of the β-lactamase KPC gene family from a biological sample. The present teachings provide for a kit including one or more primers and/or probes for the identification by multiplex real-time polymerase chain reaction of β-lactamase genes including the KPC-like family. The biological sample may include Gram-negative bacteria such as Klebsiella pneumoniae, Escherichia coli, Enterobacter cloacae and other Enterobacter species, Pseudomonas aeruginosa and Acinetobacter baumannii. The KPC-like genes detected may include KPC-1, KPC-2, KPC-3, KPC-4, KPC-5, KPC-6 KPC-7, KPC-8, KPC-9, KPC-10, KPC-11, KPC-13, KPC-14, KPC-15, KPC-16, KPC-17 KPC-18, KPC-19, KPC-21, KPC-22, KPC-47, KPC-56, KPC-63, KPC-272, KPC-484, KPC-629, KPC-727, KPC-860, or a combination thereof.

The present teachings allow for the detection of the β-lactamase NDM gene family from a biological sample. The present teachings provide for a kit including one or more primers and/or probes for the identification by multiplex real-time polymerase chain reaction of β-lactamase genes including the NDM-like family. The biological sample may include Gram-negative bacteria such as Escherichia coli, Acinetobacter baumannii, Enterobacter cloacae and Klebsiella pneumoniae. The NDM-like genes detected may include NDM-1, NDM-2, NDM-3, NDM-4, NDM-5, NDM-6, NDM-7, NDM-8, NDM-9, NDM-10, NDM-11, NDM-12, NDM-13, NDM-15, NDM-16, NDM-32, or a combination thereof.

The present teachings allow for the detection of the β-lactamase OXA gene family from a biological sample. The present teachings provide for a kit including one or more primers and/or probes for the identification by multiplex real-time polymerase chain reaction of β-lactamase genes including the OXA-48-like family. The biological sample may include Gram-negative bacteria such as Klebsiella pneumoniae, Enterobacter cloacae, Shewanella xiamenensis, Escherichia coli and Serratia marcescens. The OXA-48-like genes detected may include OXA-48, OXA-162, OXA-163, OXA-181, OXA-199, OXA-204, OXA-232, OXA-244, OXA-245, OXA-247, OXA-370, OXA-405, OXA-416, OXA-438, OXA-439, or a combination thereof.

The present teachings provide for a kit including one or more primers and/or probes for the identification by multiplex real-time polymerase chain reaction of β-lactamase genes including one or more of the following: OXA-143-like, OXA-23-like, OXA-51-like, OXA-48-like, OXA-58-like and OXA 24/40-like. The OXA-143-like genes detected may include the following: OXA-143, OXA-182, OXA-231, OXA-253, OXA-255, or a combination thereof. The OXA-23-like genes detected may include the following: OXA-23, OXA-27, OXA-49, OXA-73, OXA-102, OXA-103, OXA-105, OXA-133, OXA-134, OXA-146, OXA-165, OXA-166, OXA-167, OXA-168, OXA-169, OXA-170, OXA-171, OXA-225, OXA-239, or a combination thereof. The OXA-51-like genes detected may include the following: OXA-51, OXA-64, OXA-65, OXA-66, OXA-67, OXA-68, OXA-69, OXA-70, OXA-71, OXA-75, OXA-76, OXA-77, OXA-78, OXA-79, OXA-80, OXA-82, OXA-83, OXA-84, OXA-86, OXA-87, OXA-88, OXA-89, OXA-90, OXA-91, OXA-92, OXA-93, OXA-94 OXA-95, OXA-98, OXA-99, OXA-100, OXA-104, OXA-106, OXA-107, OXA-108, OXA-109, OXA-110, OXA-111, OXA-112, OXA-113, OXA-115, OXA-116, OXA-117, OXA-120, OXA-121, OXA-122, OXA-123, OXA-124, OXA-125, OXA-126, OXA-127, OXA-128, OXA-130, OXA-131, OXA-132, OXA-138, OXA-144, OXA-148, OXA-149, OXA-150, OXA-172, OXA-173, OXA-174, OXA-175, OXA-176, OXA-177, OXA-178, OXA-179, OXA-180, OXA-194, OXA-195, OXA-196, OXA-197, OXA-200, OXA-201, OXA-202, OXA-203, OXA-206, OXA-208, OXA-216, OXA-217, OXA-219, OXA-223, OXA-241, OXA-242, OXA-248, OXA-249, OXA-250, OXA-254, or a combination thereof. The OXA-48-like genes detected may include the following: OXA-48, OXA-48b, OXA-162, OXA-163, OXA-181, OXA-199, OXA-204, OXA-232, OXA-244, OXA-245, OXA-247, or a combination thereof. The OXA-58-like genes may include the following: OXA-58, OXA-96, OXA-97, OXA-164, or a combination thereof. The OXA-40-like genes may include the following: OXA-40, OXA-25, OXA-26, OXA-72, OXA-139, OXA-160, OXA-207, or a combination thereof.

The present teachings allow for the detection of the β-lactamase VIM gene family from a biological sample. The present teachings provide for a kit including one or more primers and/or probes for the identification by multiplex real-time polymerase chain reaction of β-lactamase genes including the VIM-like family. The biological sample may include Gram-negative bacteria such as Klebsiella oxytoca, Citrobacter freundii, Klebsiella pneumoniae, Pseudomonas aeruginosa, Escherichia coli and Enterobacter cloacae. The VIM-like genes detected may include VIM-1, VIM-2, VIM-3, VIM-4, VIM-5, VIM-6, VIM-8, VIM-9, VIM-10, VIM-11, VIM-12, VIM-13, VIM-14, VIM-15, VIM-16, VIM-17, VIM-18, VIM-19, VIM-20, VIM-23, VIM-24, VIM-25, VIM-26, VIM-27, VIM-28, VIM-31, VIM-33, VIM-34, VIM-35, VIM-36, VIM-37, VIM-38, VIM-39, VIM-40, VIM-41, VIM-42, VIM-43, VIM-44, VIM-45, VIM-46, or a combination thereof.

The present teachings allow for the detection of the AmpC R-lactamase MOX gene family from a biological sample. The present teachings provide for a kit including one or more primers and/or probes for the identification by multiplex real-time polymerase chain reaction of R-lactamase genes including the MOX-like family. The biological sample may include Gram-negative bacteria such as Klebsiella pneumoniae, Aeromonas punctata/Aeromonas caviae and other Aeromonas species and Escherichia coli. The MOX-like genes detected may include MOX-1, MOX-2, MOX-3, MOX-4, MOX-5, MOX-6, MOX-7, MOX-8, MOX-10, CMY-1, CMY-8, CMY-9, CMY-10, CMY-11, CMY-19, or a combination thereof.

The present teachings allow for the detection of the AmpC β-lactamase ACC gene family from a biological sample. The present teachings provide for a kit including one or more primers and/or probes for the identification by multiplex real-time polymerase chain reaction of β-lactamase genes including the ACC-like family. The biological sample may include Gram-negative bacteria such as Salmonella enterica, Escherichia coli, Hafnia alvei and Proteus mirabilis. The ACC-like genes detected may include ACC-1, ACC-2, ACC-4, ACC-5, ACC-6, or a combination thereof.

The present teachings allow for the detection of the AmpC β-lactamase FOX gene family from a biological sample. The present teachings provide for a kit including one or more primers and/or probes for the identification by multiplex real-time polymerase chain reaction of β-lactamase genes including the FOX-like family. The biological sample may include Gram-negative bacteria such as Klebsiella pneumoniae and Aeromonas punctata. The FOX-like genes detected may include FOX-1, FOX-2, FOX-3, FOX-4, FOX-5, FOX-6, FOX-7, FOX-8, FOX-9, FOX-10, FOX-12, or a combination thereof.

The present teachings allow for the detection of the AmpC β-lactamase DHA gene family from a biological sample. The present teachings provide for a kit including one or more primers and/or probes for the identification by multiplex real-time polymerase chain reaction of β-lactamase genes including the DHA-like family. The biological sample may include Gram-negative bacteria such as Klebsiella pneumoniae, Morganella morganii, Escherichia coli and Enterobacter cloacae. The DHA-like genes detected may include DHA-1, DHA-2, DHA-5, DHA-6, DHA-7, DHA-9, DHA-10, DHA-12, DHA-13, DHA-14, DHA-15, DHA-16, DHA-17, DHA-18, DHA-19, DHA-20, DHA-21, DHA-22, or a combination thereof.

The present teachings allow for the detection of the AmpC β-lactamase CMY-2 gene family from a biological sample. The present teachings provide for a kit including one or more primers and/or probes for the identification by multiplex real-time polymerase chain reaction of β-lactamase genes including the CMY-2-like family. The biological sample may include Gram-negative bacteria such as Klebsiella pneumoniae, Morganella morganii, Escherichia coli and Enterobacter cloacae. The CMY-2-like genes detected include CMY-2, CMY-4, CMY-6, CMY-7, CMY-12, CMY-14, CMY-15, CMY-16, CMY-18, CMY-21, CMY-22, CMY-23, CMY-24, CMY-25, CMY-26, CMY-27, CMY-28, CMY-29, CMY-30, CMY-31, CMY-32, CMY-33, CMY-34, CMY-35, CMY-37, CMY-38, CMY-39, CMY-40, CMY-41, CMY-42, CMY-43, CMY-44, CMY-45, CMY-46 CMY-47, CMY-48, CMY-49, CMY-50, CMY-51, CMY-53, CMY-54, CMY-55, CMY-56, CMY-57, CMY-58, CMY-59, CMY-60, CMY-61, CMY-62, CMY-63, CMY-64, CMY-65, CMY-66, CMY-67, CMY-68, CMY-69, CMY-71, CMY-72, CMY-73, CMY-75, CMY-76, CMY-77, CMY-78, CMY-79, CMY-80, CMY-81, CMY-84, CMY-85 CMY-86, CMY-87, CMY-89, CMY-90, CMY-96, CMY-97, CMY-99, CMY-102, CMY-103, CMY-104 CMY-105, CMY-107, CMY-108, CMY-110, CMY-111, CMY-112, CMY-113, CMY-114, CMY-115 CMY-116, CMY-117, CMY-118, CMY-119, CMY-121, CMY-122, CMY-124, CMY-125, CMY-126, CMY-127, CMY-128, CMY-129, CMY-130, CMY-131 CMY-132, CMY-133, CMY-135, or a combination thereof.

The present teachings allow for the detection of the AmpC β-lactamase EBC gene family from a biological sample. The present teachings provide for a kit including one or more primers and/or probes for the identification by multiplex real-time polymerase chain reaction of β-lactamase genes including the EBC-like family such as ACT and MIR. The biological sample may include Gram-negative bacteria such as Enterobacter cloacae, Klebsiella pneumoniae, Enterobacter asburiae, Enterobacter kobei, and other Enterobacter species. The EBC-like genes detected may include ACT-1, ACT-2, ACT-5, ACT-8, ACT-13, ACT-14, ACT-15, ACT-16, ACT-17, ACT-18, ACT-20, ACT-21, ACT-23, ACT-24, ACT-25, ACT-27, ACT-29, ACT-30, ACT-31, ACT-32, ACT-33, ACT-34, ACT-35, ACT-36, ACT-37, ACT-38, MIR-1, MIR-2, MIR-3, MIR-4, MIR-6, MIR-7, MIR-8, MIR-9, MIR-10, MIR-11, MIR-12, MIR-13, MIR-14, MIR-15, MIR-16, MIR-17, MIR-18, or a combination thereof.

The present teachings may allow for the detection of the β-lactamase TEM gene family from a biological sample. The present teachings provide for a kit including one or more primers and/or probes for the identification by multiplex real-time polymerase chain reaction of β-lactamase genes including the TEM-like family. The biological sample may include Gram-negative bacteria such as Klebsiella pneumoniae, Enterobacter cloacae, Shewanella xiamenensis, Escherichia coli and Serratia marcescens. The TEM-like genes detected may include TEM-1, TEM-2, TEM-3, TEM-15, TEM-20, TEM-32, TEM-40, TEM-52, TEM-88, TEM-91, TEM-97, TEM-98, TEM-106, TEM-107, TEM-112, TEM-120, TEM-126, TEM-135, TEM-141, TEM-150, TEM-153, TEM-163, TEM-168, TEM-170, TEM-171, TEM-206, TEM-214, TEM-220, or a combination thereof.

The present teachings may allow for the detection of the β-lactamase SHV gene family from a biological sample. The present teachings provide for a kit including one or more primers and/or probes for the identification by multiplex real-time polymerase chain reaction of β-lactamase genes including the SHV-like family. The biological sample may include Gram-negative bacteria such as Klebsiella pneumoniae, Enterobacter cloacae, Shewanella xiamenensis, Escherichia coli and Serratia marcescens. The SHV-like genes detected may include SHV-1, SHV-2, SHV-3, SHV-5, SHV-7, SHV-8, SHV-9, SHV-11, SHV-12, SHV-13, SHV-14, SHV-15, SHV-16, SHV-18, SHV-24, SHV-25, SHV-26, SHV-27, SHV-28, SHV-29, SHV-30, SHV-31, SHV-32, SHV-33, SHV-34, SHV-35, SHV-36, SHV-37, SHV-38, SHV-40, SHV-41, SHV-42, SHV-43, SHV-44, SHV-45, SHV-46, SHV-48, SHV-49, SHV-50, SHV-51, SHV-52, SHV-53, SHV-55, SHV-56, SHV-57, SHV-59, SHV-60, SHV-61, SHV-62, SHV-63, SHV-64, SHV-65, SHV-66, SHV-67, SHV-69, SHV-70, SHV-71, SHV-72, SHV-73, SHV-74, SHV-75, SHV-76, SHV-77, SHV-78, SHV-79, SHV-80, SHV-81, SHV-82, SHV-85, SHV-86, SHV-89, SHV-92, SHV-93, SHV-94, SHV-95, SHV-96, SHV-97, SHV-98, SHV-99, SHV-100, SHV-101, SHV-102, SHV-103, SHV-104, SHV-105, SHV-106, SHV-107, SHV-109, SHV-110, SHV-111, SHV-119, SHV-120, SHV-121, SHV-122, SHV-123, SHV-124, SHV-125, SHV-126, SHV-127, SHV-128, SHV-129, SHV-132, SHV-133, SHV-134, SHV-135, SHV-136, SHV-137, SHV-140, SHV-141, SHV-142, SHV-143, SHV-144, SHV-145, SHV-146, SHV-147, SHV-148, SHV-149, SHV-150, SHV-151, SHV-152, SHV-153, SHV-154, SHV-155, SHV-156, SHV-157, SHV-158, SHV-159, SHV-160, SHV-161, SHV-162, SHV-163, SHV-164, SHV-165, SHV-168, SHV-172, SHV-173, SHV-178, SHV-179, SHV-180, SHV-182, SHV-183, SHV-185, SHV-186, SHV-187, SHV-188, SHV-189, SHV-190, SHV-191, SHV-193, SHV-194, SHV-195, SHV-196, SHV-197, or a combination thereof.

The present teachings may allow for the detection of the GES gene family from a biological sample. The present teachings provide for a kit including one or more primers and/or probes for the identification by multiplex real-time polymerase chain reaction of GES genes including the GES-like family. The GES-like genes detected may include GES-1, GES-2, GES-3, GES-4, GES-5, GES-6, GES-7, GES-8, GES-9, GES-10, GES-11, GES-12, GES-13, GES-14, GES-15, GES-16, GES-17, GES-18, GES-19, GES-20, GES-21, GES-22, GES-23, GES-24, GES-25, GES-26, GES-27, GES-28, GES-29, GES-30, GES-31, GES-32, GES-33, GES-34, GES-35, GES-36, GES-37, GES-39, GES-40, or a combination thereof.

The present teachings may allow for the detection of the MCR gene family from a biological sample. The present teachings provide for a kit including one or more primers and/or probes for the identification by multiplex real-time polymerase chain reaction of MCR genes including the MCR-like family. The MCR-like genes detected may include MCR-1, MCR-1.1, MCR-1.2, MCR-1.3, MCR-1.4, MCR-1.5, MCR-1.6, MCR-1.7, MCR-1.8, MCR-1.9, MCR-1.11, MCR-1.12, MCR-1.13, MCR-1.14, MCR-1.15, and MCR-2, MCR-2.1, MCR-3, MCR-3.1, MCR-3.2, MCR-3.3, MCR-3.4, MCR-3.5, MCR-3.6, MCR-3.7, MCR-3.8, MCR-3.9, MCR-3.10, MCR-3.11, MCR-3.12, MCR-3.13, MCR-3.14, MCR-3.15, MCR-3.16, MCR-3.18, MCR-3.19, MCR-3.20, MCR-3.21, MCR-3.22, MCR-3.23, MCR-3.24, MCR-3.25, MCR-4, MCR-4.1, MCR-4.2, MCR-4.3, MCR-4.4, MCR-4.5, MCR-4.6, MCR-5, MCR-5.1, MCR-5.2, MCR-5.3, or a combination thereof.

The kit of the present teachings may include a mix of at least one primer and/or at least one probe. Primers and/or probes may be degenerate at any nucleotide position. Primers and/or probes may not be degenerate at any nucleotide position. A hydrolysis and/or hybridization probe may be designed for the detection of a specific nucleic acid sequence. Multiple probes may be labeled with a different colored fluorophore. The probe may be labeled with a fluorescent tag at one end and a fluorescent quencher at the other end. Two fluorescent quenchers may be included at one end or within the probe sequence. For example, the fluorophores may be selected from the group consisting of fluorescein, hexachlorofluorescein, TEX615, and TYE™ 665. The fluorophores may excite between 450 nm and 763 nm and emit between 500 nm and 800 nm. For example, the quenchers may be selected from the group consisting of Iowa Black® quenchers and Black Hole Quenchers®. Peak absorbance of each quencher may be at 531 nm, 534 nm, 578 nm, or 656 nm.

Multiple hydrolysis and/or hybridization probes can be added to the same nucleic acid amplification reaction. The selection of the fluorescent labels may depend on the type of hydrolysis and/or hybridization probe used, the number of targets to be detected and the type of thermal cycler used. Preferable combinations of fluorophores and quenchers for multiplex reactions require appropriate excitation wavelengths and little to no overlap in their emission spectra as well as reduction of background fluorescence. It is contemplated that the probe sequences of the present teachings may be labeled with any suitable fluorophore and quencher combinations. For example, any fluorophore of the present teachings may be attached to any probe DNA sequence of the present teachings.

The one or more primers and/or probes may be selected from the group consisting of: TGGCCAGAACTGACAGGCAAA, TTTCTCCTGAACGTGGCTGGC, 56-FAM/ACGCTAACT/ZEN/CCAGCATTGGTCTGT/3IABkFQ/, CCGTCACGCTGTTGTTAGG, GCTGTGTTAATCAATGCCACAC, 5HEX/AACTTGCCG/ZEN/AATTAGAGCRGCAGT/3IABkFQ, CGTTTCGTCTGGATCGCAC, GCTGGGTAAAATAGGTCACC, 5TEX615/TATCATTGGTGGTGCCGTAGTCGC/3IAbRQSp, GAGAGGATGAYCAGCCACAC, CGCCCATTGTSCAATATTCC, 5TYE665/TGAGACACGGTCCAGACTCCTACG/3IAbRQSp, AATCACAGGGCGTAGTTGTG, ACCCACCAGCCAATCTTAGG, 56-FAM/TAGCTTGAT/ZEN/CGCCCTCGATTTGGG/3IABkFQ/, GCGGAGTTAACTATTGGCTAG, GGCCAAGCTTCTATATTTGCG, 5HEX/TTRTTYGGT/ZEN/GGTTGYTTTRTTAA/3IABkFQ, GCGGAGTTARYTATTGGCTAG, GGCCAAGCYTCTAWATTTGCG, /5HEX/CCGGACGGT/ZEN/CTTGGTAATTTGGGT/3IABkFQ/, /5HEX/CCGTACGGT/ZEN/TTAGGCAATTTGGGT/3IABkFQ/, GGCGGCGTTGATGTCCTTCG, CCATTCAGCCAGATCGGCATC, 5TEX615/AGCTCTTCTATCCTGGTGCTGCG/3IAbRQSp, AACTTTCACAGGTGTGCTGGGT, CCGTACGCATACTGGCTTTGC, 56-FAM/AAACCGGGC/ZEN/GATATGCGTCTGTAT/3IABkFQ/, GTATCGCCGTCTAGTTCTGC, CCTTGAATGAGCTGCACAGTGG, 5HEX/TCGTCGCGG/ZEN/AACCATTCGCTAAA/3IABkFQ/, GTTTGATCGTCAGGGATGGC, GGCGAAAGTCAGGCTGTG, 5TEX615/CATCAGGACAAGATGGGCGGTATG/3IAbRQSp, GCTGCTCAAGGAGCACAGGAT, CACATTGACATAGGTGTGGTGC, 56-FAM/AGGATGGCA/ZEN/AGGCCCACTATTTCA/3IABkFQ, AACAGCCTCAGCAGCCGGTTA, TTCGCCGCAATCATCCCTAGC, 5HEX/AGCCATTAC/ZEN/GTTCCAGAGTTGCGT/3IABkFQ, GCCGAGGCTTACGGGATCAAG, CAAAGCGCGTAACCGGATTGG, 5TEX615/TCTGCTGAAGTTTRYCGAGGCMAA/3IAbRQSp, AACTTTCACAGGTGTGCTGGGT, CCGTACGCATACTGGCTTTGC, 56-FAM/AAACCGGGC/ZEN/GATATGCGTCTGTAT/3IABkFQ/, CTGGGTTCTATAAGTAAAACCTTCACCGG, CTTCCACTGCGGCTGCCAGTT, 5HEX/GATGCCATT/ZEN/GCYCGSGGTGAAAT/3IABkFQ, CCGAAGCCTATGGCGTGAAATCC, GCAATGCCCTGCTGGAGCG, 5TEX615/ATGTTGGCCTGAACCCAGCG/3IAbRQSp. Primers and/or probes included in this group may or may not be degenerate at any nucleotide position. [SEQ. ID NOS 67-118]

The kit may include one or more primer-probe multiplex mixes. The primer-probe multiplex mix may include one or more internal controls. The primer-probe multiplex mix and one or more internal controls may be enclosed in one container, such as a vial. The primer-probe multiplex mix and one or more internal controls may be enclosed in more than one container, such as vials.

A primer-probe mix may include sequences for detecting any combination of the following genes: CMY-2-like, CTX-M-14-like, CTX-M-15-like, IMP-like, VIM-like, DHA-like, KPC-like, NDM-like, MOX-like, ACC-like, FOX-like, DHA-like, EBC-like, OXA-143-like, OXA-23-like, OXA-51-like, OXA-48-like, OXA-58-like and OXA-24/40-like.

For example, the kit may include a first primer-probe mix and one or more internal controls in a first vial and a second primer-probe mix and one or more internal controls in a second vial. For example, the kit may include a first primer-probe mix and one or more internal controls in a first vial, a second primer-probe mix and one or more internal controls in a second vial and a third primer-probe mix and one or more internal controls in a third vial. Each vial may contain different mixtures. Each vial may contain the same mixture.

The kit may include at least one control DNA mix. The kit may include one or more DNA control mixes. The kit may include exactly two control DNA mixes. The kit may include exactly three control DNA mixes. The DNA control mix may include at least one DNA sequence corresponding to at least one gene family and at least one internal control DNA sequence. The DNA control mix may be enclosed in one container, such as a vial. The DNA control mix may be enclosed in more than one container, such as vials.

For example, the kit may include a first DNA control mix in a first vial and a second DNA control mix in a second vial. For example, the kit may include a first DNA control mix in a first vial, a second DNA control mix in a second vial and a third DNA control mix in a third vial. Each vial may contain different mixtures. Each vial may contain the same mixture.

In one example, the kit includes three primer-probe multiplex mix vials including internal controls and three DNA control mix vials. The three primer-probe multiplex mixes may provide for identification of up to nine antibiotic resistance genes and internal controls. A first primer-probe mix may include sequences for detecting gene families which are CMY-2-like, CTX-M-14-like, CTX-M-15-like and internal controls. A second primer-probe mix may include sequences for detecting gene families which are OXA-48-like, IMP-like, VIM-like and internal controls. A third primer-probe mix may include sequences for detecting gene families which are DHA-like, KPC-like, NDM-like and internal controls. The one or more DNA control mixes may be plasmid or vector controls. A first DNA control mix may include DNA sequences for CMY-2, CTX-M-14, CTX-M-15 and an internal control DNA sequence. A second DNA control mix may include DNA sequences for OXA-48, IMP, VIM and an internal control DNA sequence. A third DNA control mix may include DNA sequences for DHA, KPC, NDM and an internal control DNA sequence.

It is contemplated that the combination of gene families may vary. For example, a primer-probe mix may include sequences for detecting any combination of the following genes: CMY-2-like, CTX-M-14-like, CTX-M-15-like, and OXA-48-like, IMP-like, VIM-like, DHA-like, KPC-like and NDM-like. It is further contemplated that additional β-lactamase gene targets may be included in the primer-probe mix or mixes.

The first primer-probe mix may include one or more primers and/or probes selected from the group consisting of: TGGCCAGAACTGACAGGCAAA, TTTCTCCTGAACGTGGCTGGC, 56FAM/ACGCTAACT/ZEN/CCAGCATTGGTCTGT/3IABkFQ/, CCGTCACGCTGTTGTTAGG, GCTGTGTTAATCAATGCCACAC, 5HEX/AACTTGCCG/ZEN/AATTAGAGCRGCAGT/3IABkFQ, CGTTTCGTCTGGATCGCAC, GCTGGGTAAAATAGGTCACC and 5TEX615/TATCATTGGTGGTGCCGTAGTCGC/3IAbRQSp. The first primer-probe mix may include one or more internal controls selected from the group consisting of: GAGAGGATGAYCAGCCACAC, CGCCCATTGTSCAATATTCC and 5TYE665/TGAGACACGGTCCAGACTCCTACG/3IAbRQSp. A primer-probe mix may include a combination of the one or more said group of primers and/or probes and the one or more said group of internal controls. The primer-probe mix including internal controls may be a multiplex mix. (SEQ. ID NOS: 152-163)

The kit may include a first, second and third primer and/or probe mix, the first primer and/or probe mix including one or more primers and/or probes selected from the group consisting of: TGGCCAGAACTGACAGGCAAA, TTTCTCCTGAACGTGGCTGGC, 56-FAM/ACGCTAACT/ZEN/CCAGCATTGGTCTGT/3IABkFQ/, CCGTCACGCTGTTGTTAGG, GCTGTGTTAATCAATGCCACAC, 5HEX/AACTTGCCG/ZEN/AATTAGAGCRGCAGT/3IABkFQ, CGTTTCGTCTGGATCGCAC, GCTGGGTAAAATAGGTCACC, 5TEX615/TATCATTGGTGGTGCCGTAGTCGC/3IAbRQSp, GAGAGGATGAYCAGCCACAC, CGCCCATTGTSCAATATTCC, and 5TYE665/TGAGACACGGTCCAGACTCCTACG/3IAbRQSp. Primers and/or probes included in this group may or may not be degenerate at any nucleotide position. [SEQ. ID NOS 152-163]

The second primer-probe mix may include one or more primers and/or probes selected from the group consisting of: AATCACAGGGCGTAGTTGTG, ACCCACCAGCCAATCTTAGG, 56-FAM/TAGCTTGAT/ZEN/CGCCCTCGATTTGGG/3IABkFQ/, GCGGAGTTAACTATTGGCTAG, GGCCAAGCTTCTATATTTGCG, 5HEX/TTRTTYGGT/ZEN/GGTTGYTTTRTTAA/3IABkFQ, GCGGAGTTARYTATTGGCTAG, GGCCAAGCYTCTAWATTTGCG, /5HEX/CCGGACGGT/ZEN/CTTGGTAATTTGGGT/3IABkFQ/, /5HEX/CCGTACGGT/ZEN/TTAGGCAATTTGGGT/3IABkFQ, GGCGGCGTTGATGTCCTTCG, CCATTCAGCCAGATCGGCATC and 5TEX615/AGCTCTTCTATCCTGGTGCTGCG/3IAbRQSp. The second primer-probe mix may include one or more internal controls selected from the group consisting of: GAGAGGATGAYCAGCCACAC, CGCCCATTGTSCAATATTCC, and 5TYE665/TGAGACACGGTCCAGACTCCTACG/3IAbRQSp. A primer-probe mix may include a combination of the one or more said group of primers and/or probes and the one or more said group of internal controls. The primer-probe mix including internal controls may be a multiplex mix. (SEQ. ID NOS: 164-179)

The kit may include a first, second, and third primer and/or probe mix, the second primer and/or probe mix including one or more primers and/or probes selected from the group consisting of: AATCACAGGGCGTAGTTGTG, ACCCACCAGCCAATCTTAGG, 56-FAM/TAGCTTGAT/ZEN/CGCCCTCGATTTGGG/13IABkFQ/, GCGGAGTTAACTATTGGCTAG, GGCCAAGCTTCTATATTTGCG, 5HEX/TTRTTYGGT/ZEN/GGTTGYTTTRTTAA/3IABkFQ, GCGGAGTTARYTATTGGCTAG, GGCCAAGCYTCTAWATTTGCG, /5HEX/CCGGACGGT/ZEN/CTTGGTAATTTGGGT/3IABkFQ/, /5HEX/CCGTACGGT/ZEN/TTAGGCAATTTGGGT/3IABkFQ, GGCGGCGTTGATGTCCTTCG, CCATTCAGCCAGATCGGCATC, 5TEX615/AGCTCTTCTATCCTGGTGCTGCG/3IAbRQSp, GAGAGGATGAYCAGCCACAC, CGCCCATTGTSCAATATTCC, and 5TYE665/TGAGACACGGTCCAGACTCCTACG/3IAbRQSp. Primers and/or probes included in this group may or may not be degenerate at any nucleotide position. (SEQ. ID NOS: 164-179)

The third primer-probe mix may include one or more primers and/or probes selected from the group consisting of: AACTTTCACAGGTGTGCTGGGT, CCGTACGCATACTGGCTTTGC, 56-FAM/AAACCGGGC/ZEN/GATATGCGTCTGTAT/3IABkFQ/, GTATCGCCGTCTAGTTCTGC, CCTTGAATGAGCTGCACAGTGG, 5HEX/TCGTCGCGG/ZEN/AACCATTCGCTAAA/3IABkFQ/, GTTTGATCGTCAGGGATGGC, GGCGAAAGTCAGGCTGTG and 5TEX615/CATCAGGACAAGATGGGCGGTATG/3IAbRQSp. The third primer-probe mix may include one or more internal controls selected from the group consisting of: GAGAGGATGAYCAGCCACAC, CGCCCATTGTSCAATATTCC and 5TYE665/TGAGACACGGTCCAGACTCCTACG/3IAbRQSp. A primer-probe mix may include a combination of the one or more said group of primers and/or probes and the one or more said group of internal controls. The primer-probe mix including internal controls may be a multiplex mix. (SEQ. ID NOS: 180-191)

The kit may include a first, second and third primer and/or probe mix, the third primer and/or probe mix including one or more primers and/or probes selected from the group consisting of: AACTTTCACAGGTGTGCTGGGT, CCGTACGCATACTGGCTTTGC, 56-FAM/AAACCGGGC/ZEN/GATATGCGTCTGTAT/3IABkFQ/, GTATCGCCGTCTAGTTCTGC, CCTTGAATGAGCTGCACAGTGG, 5HEX/TCGTCGCGG/ZEN/AACCATTCGCTAAA/3IABkFQ/, GTTTGATCGTCAGGGATGGC, GGCGAAAGTCAGGCTGTG, 5TEX615/CATCAGGACAAGATGGGCGGTATG/3IAbRQSp, GAGAGGATGAYCAGCCACAC, CGCCCATTGTSCAATATTCC, and 5TYE665/TGAGACACGGTCCAGACTCCTACG/3IAbRQSp. Primers and/or probes included in this group may or may not be degenerate at any nucleotide position. (SEQ. ID NOS: 180-191)

A first DNA control mix may include one or more sequences selected from the group consisting of: TGGCCAGAACTGACAGGCAAACAGTGGCAGGGTATCCGCCTGCTGCACTTAGCCACCTAT ACGGCAGGCGGCCTACCGCTGCAGATCCCCGATGACGTTAGGGATAAAGCCGCATTACTG CATTTTTATCAAAACTGGCAGCCGCAATGGACTCCGGGCGCTAAGCGACTTTACGCTAACT CCAGCATTGGTCTGTTTGGCGCGCTGGCGGTGAAACCCTCAGGAATGAGTTACGAAGAGG CAATGACCAGACGCGTCCTGCAACCATTAAAACTGGCGCATACCTGGATTACGGTTCCGCA GAACGAACAAAAAGATTATGCCTGGGGCTATCGCGAAGGGAAGCCCGTACACGTTTCTCC GGGACAACTTGACGCCGAAGCCTATGGCGTGAAATCCAGCGTTATTGATATGGCCCGCTG GGTTCAGGCCAACATGGATGCCAGCCACGTTCAGGAGAAA, CCGTCACGCTGTTGTTAGGAAGTGTGCCGCTGTATGCGCAAACGGCGGACGTACAGCAAA AACTTGCCGAATTAGAGCGGCAGTCGGGAGGCAGACTGGGTGTGGCATTGATTAACACAG C, and CGTTTCGTCTGGATCGCACTGAACCTACGCTGAATACCGCCATTCCCGGCGACCCGAGAG ACACCACCACGCCGCGGGCGATGGCGCAGACGTTGCGTCAGCTTACGCTGGGTCATGCG CTGGGCGAAACCCAGCGGGCGCAGTTGGTGACGTGGCTCAAAGGCAATACGACCGGCGC AGCCAGCATTCGGGCCGGCTTACCGACGTCGTGGACTGTGGGTGATAAGACCGGCAGCG GCGACTACGGCACCACCAATGATATTGCGGTGATCTGGCCGCAGGGTCGTGCGCCGCTG GTTCTGGTGACCTATTTTACCCAGC. The first DNA control mix may include the following internal control sequence: GAGAGGATGACCAGCCACACTGGAACTGAGACACGGTCCAGACTCCTACGGGAGGCAGC AGTGGGGAATATTGCACAATGGGCG. A DNA control mix may include a combination of the one or more said group of sequences and the said internal control sequence. A DNA control mix may include any combination of sequences from the first control mix, the second control mix, the third control mix and the internal control sequence. (SEQ. ID NOS: 261-264)

A second DNA control mix may include one or more sequences selected from the group consisting of: AATCACAGGGCGTAGTTGTGCTCTGGAATGAGAATAAGCAGCAAGGATTTACCAATAATCT TAAACGGGCGAACCAAGCATTTTTACCCGCATCTACCTTTAAAATTCCCAATAGCTTGATCG CCCTCGATTTGGGCGTGGTTAAGGATGAACACCAAGTCTTTAAGTGGGATGGACAGACGC GCGATATCGCCACTTGGAATCGCGATCATAATCTAATCACCGCGATGAAATATTCAGTTGT GCCTGTTTATCAAGAATTTGCCCGCCAAATTGGCGAGGCACGTATGAGCAAGATGCTACAT GCTTTCGATTATGGTAATGAGGACATTTCGGGCAATGTAGACAGTTTCTGGCTCGACGGTG GTATTCGAATTTCGGCCACGGAGCAAATCAGCTTTTTAAGAAAGCTGTATCACAATAAGTTA CACGTATCGGAGCGCAGCCAGCGTATTGTCAAACAAGCCATGCTGACCGAAGCCAATGGT GACTATATTATTCGGGCTAAAACTGGATACTCGACTAGAATCGAACCTAAGATTGGCTGGT GGGT, GCGGAGTTAGTTATTGGCTAGTTAAAAATAAAATTGAAGTTTTTTATCCCGGCCCGGGGCA CACTCAAGATAACGTAGTGGTTTGGTTACCTGAAAAGAAAATTTTATTCGGTGGTTGTTTTG TTAAACCGGACGGTCTTGGTAATTTGGGTGACGCAAATTTAGAAGCTTGGCC and GGCGGCGTTGATGTCCTTCGGGCGGCTGGGGTGGCAACGTACGCATCACCGTCGACACG CCGGCTAGCCGAGGTAGAGGGGAACGAGATTCCCACGCACTCTCTAGAAGGACTCTCATC GAGCGGGGACGCAGTGCGCTTCGGTCCAGTAGAACTCTTCTATCCTGGTGCTGCGCATTC GACCGACAACTTAGTTGTGTACGTCCCGTCTGCGAGTGTGCTCTATGGTGGTTGTGCGATT CATGAGTTGTCACGCACGTCTGCGGGGAACGTGGCCGATGCCGATCTGGCTGAATGG.

The second DNA control mix may include the following internal control sequence: GAGAGGATGACCAGCCACACTGGAACTGAGACACGGTCCAGACTCCTACGGGAGGCAGC AGTGGGGAATATTGCACAATGGGCG. A DNA control mix may include a combination of the one or more said group of sequences and the said internal control sequence. A DNA control mix may include any combination of sequences from the first control mix, the second control mix, the third control mix and the internal control sequence. (SEQ. ID NOS: 265-268)

A third DNA control mix may include one or more sequences selected from the group consisting of: AACTTTCACAGGTGTGCTGGGTGCGGTTTCTGTGGCGAAAAAAGAGATGGCGCTGAATGA TCCGGCGGCAAAATACCAGCCGGAGCTGGCTCTGCCGCAGTGGAAGGGGATCACATTGC TGGATCTGGCTACCTATACCGCAGGCGGACTGCCGTTACAGGTGCCGGATGCGGTAAAAA GCCGTGCGGATCTGCTGAATTTCTATCAGCAGTGGCAGCCGTCCCGGAAACCGGGCGATA TGCGTCTGTATGCAAACAGCAGTATCGGCCTGTTTGGTGCTCTGACCGCAAACGCGGCGG GGATGCCGTATGAGCAGTTGCTGACTGCACGGATCCTGGCACCGCTGGGGTTATCTCACA CCTTTATTACTGTGCCGGAAAGTGCGCAAAGCCAGTATGCGTACGG, GTATCGCCGTCTAGTTCTGCTGTCTTGTCTCTCATGGCCGCTGGCTGGCTTTTCTGCCACC GCGCTGACCAACCTCGTCGCGGAACCATTCGCTAAACTCGAACAGGACTTTGGCGGCTCC ATCGGTGTGTACGCGATGGATACCGGCTCAGGCGCAACTGTAAGTTACCGCGCTGAGGAG CGCTTCCCACTGTGCAGCTCATTCAAGG and GTTTGATCGTCAGGGATGGCGGCCGCGTGCTGGTGGTCGATACCGCCTGGACCGATGAC CAGACCGCCCAGATCCTCAACTGGATCAAGCAGGAGATCAACCTGCCGGTCGCGCTGGC GGTGGTGACTCACGCGCATCAGGACAAGATGGGCGGTATGGACGCGCTGCATGCGGCGG GGATTGCGACTTATGCCAATGCGTTGTCGAACCAGCTTGCCCCGCAAGAGGGGATGGTTG CGGCGCAACACAGCCTGACTTTCGCC. The third DNA control mix may include the following internal control sequence: GAGAGGATGACCAGCCACACTGGAACTGAGACACGGTCCAGACTCCTACGGGAGGCAGC AGTGGGGAATATTGCACAATGGGCG. A DNA control mix may include a combination of the one or more said group of sequences and the said internal control sequence. A DNA control mix may include any combination of sequences from the first control mix, the second control mix, the third control mix and the internal control sequence. (SEQ. ID NOS: 269-272)

In one example, the kit includes two primer-probe multiplex mix vials including internal controls and two DNA control mix vials. The two primer-probe multiplex mixes may provide for identification of up to six antibiotic resistance genes and internal controls. A first primer-probe mix may include sequences for detecting gene families which are MOX-like, ACC-like, FOX-like and internal controls. A second primer-probe mix may include sequences for detecting gene families which are DHA-like, ACT/MIR-like, CMY-2-like and internal controls. A first DNA control mix may include DNA sequences for MOX, ACC, FOX and an internal control DNA sequence. A second DNA control mix may include DNA sequences for DHA, ACT/MIR, CMY-2 and an internal control DNA sequence.

It is contemplated that the combination of gene families may vary. For example, a primer-probe mix may include sequences for detecting any combination of the following genes: MOX-like, ACC-like, FOX-like, DHA-like, ACT/MIR-like and CMY-2-like. It is further contemplated that additional β-lactamase gene targets may be included in the primer-probe mix or mixes.

The first primer-probe mix may include one or more primers and/or probes selected from the group consisting of: GCTGCTCAAGGAGCACAGGAT, CACATTGACATAGGTGTGGTGC, 56-FAM/AGGATGGCA/ZEN/AGGCCCACTATTTCA/3IABkFQ, AACAGCCTCAGCAGCCGGTTA, TTCGCCGCAATCATCCCTAGC, 5HEX/AGCCATTAC/ZEN/GTTCCAGAGTTGCGT/3IABkFQ, GCCGAGGCTTACGGGATCAAG, CAAAGCGCGTAACCGGATTGG and 5TEX615/TCTGCTGAAGTTTRYCGAGGCMAA/3IAbRQSp. The first primer-probe mix may include one or more internal controls selected from the group consisting of: GAGAGGATGAYCAGCCACAC, CGCCCATTGTSCAATATTCC and 5TYE665/TGAGACACGGTCCAGACTCCTACG/3IAbRQSp. A primer-probe mix may include a combination of the one or more said group of primers and/or probes and the one or more said group of internal controls. The primer-probe mix including internal controls may be a multiplex mix. (SEQ. ID NOS: 192-203)

The second primer-probe mix may include one or more primers and/or probes selected from the group consisting of: AACTTTCACAGGTGTGCTGGGT, CCGTACGCATACTGGCTTTGC, 56-FAM/AAACCGGGC/ZEN/GATATGCGTCTGTAT/3IABkFQ, CTGGGTTCTATAAGTAAAACCTTCACCGG, CTTCCACTGCGGCTGCCAGTT, 5HEX/GATGCCATT/ZEN/GCYCGSGGTGAAAT/3IABkFQ, CCGAAGCCTATGGCGTGAAATCC, GCAATGCCCTGCTGGAGCG, and

5TEX615/ATGTTGGCCTGAACCCAGCG/3IAbRQSp. The second primer-probe mix may include one or more internal controls selected from the group consisting of: GAGAGGATGAYCAGCCACAC, CGCCCATTGTSCAATATTCC and 5TYE665/TGAGACACGGTCCAGACTCCTACG/3IAbRQSp. A primer-probe mix may include a combination of the one or more said group of primers and/or probes and the one or more said group of internal controls. The primer-probe mix including internal controls may be a multiplex mix. (SEQ. ID NOS: 204-215)

The kit may include exactly two primer and/or probe mixes, a first primer and/or probe mix including one or more primers and/or probes selected from the group consisting of: GCTGCTCAAGGAGCACAGGAT, CACATTGACATAGGTGTGGTGC, 56-FAM/AGGATGGCA/ZEN/AGGCCCACTATTTCA/3IABkFQ, AACAGCCTCAGCAGCCGGTTA, TTCGCCGCAATCATCCCTAGC, 5HEX/AGCCATTAC/ZEN/GTTCCAGAGTTGCGT/3IABkFQ, GCCGAGGCTTACGGGATCAAG, CAAAGCGCGTAACCGGATTGG, 5TEX615/TCTGCTGAAGTTTRYCGAGGCMAA/3IAbRQSp, GAGAGGATGAYCAGCCACAC, CGCCCATTGTSCAATATTCC, and 5TYE665/TGAGACACGGTCCAGACTCCTACG/3IAbRQSp; and a second primer and/or probe mix including one or more primers and/or probes selected from the group consisting of: AACTTTCACAGGTGTGCTGGGT, CCGTACGCATACTGGCTTTGC, 56-FAM/AAACCGGGC/ZEN/GATATGCGTCTGTAT/3IABkFQ, CTGGGTTCTATAAGTAAAACCTTCACCGG, CTTCCACTGCGGCTGCCAGTT, 5HEX/GATGCCATT/ZEN/GCYCGSGGTGAAAT/3IABkFQ, CCGAAGCCTATGGCGTGAAATCC, GCAATGCCCTGCTGGAGCG, 5TEX615/ATGTTGGCCTGAACCCAGCG/3IAbRQSp, GAGAGGATGAYCAGCCACAC, CGCCCATTGTSCAATATTCC, and 5TYE665/TGAGACACGGTCCAGACTCCTACG/3IAbRQSp. Primers and/or probes included in this group may or may not be degenerate at any nucleotide position. (SEQ. ID NOS: 192-215)

A first DNA control mix may include one or more sequences selected from the group consisting of: GCTGCTCAAGGAGCACAGGATCCCGGGCATGGCGGTGGCCGTGCTCAAGGATG GCAAGGCCCACTATTTCAATTACGGGGTGGCCAACCGGGAGAGCGGGGCCAGCGTCAGC GAGCAGACCCTGTTCGAGATAGGATCCGTGAGCAAGACCCTGACTGCGACCCTGGGGGC CTATGCGGTGGTCAAGGGAGCGATGCAGCTGGATGACAAGGCGAGCCGGCACGCGCCCT GGCTCAAGGGATCCGTCTTTGACAGCATCACCATGGGGGAGCTTGCCACCTACAGCGCCG GAGGCCTGCCACTGCAATTCCCCGAGGAGGTGGATTCATCCGAGAAGATGCGCGCCTACT ACCGCCAGTGGGCCCCTGTCTATTCGCCGGGCTCCCATCGCCAGTACTCCAACCCCAGCA TAGGGCTGTTCGGCCACCTGGCGGCGAGCAGCCTGAAGCAGCCATTTGCCCAGTTGATG GAGCAGACCCTGCTGCCCGGGCTCGGCATGCACCACACCTATGTCAATGTG, AACAGCCTCAGCAGCCGGTTACGGAAAATACGTTATTTGAAGTGGGTTCGCTGAGTAAAAC GTTTGCTGCCACCTTGGCGTCCTATGCGCAGGTGAGCGGTAAGCTGTCTTTGGATCAAAG CGTTAGCCATTACGTTCCAGAGTTGCGTGGCAGCAGCTTTGACCACGTTAGCGTACTCAAT GTGGGCACGCATACCTCAGGCCTACAGCTATTTATGCCGGAAGATATTAAAAATACCACAC AGCTGATGGCTTATCTAAAAGCATGGAAACCTGCCGATGCGGCTGGAACCCATCGCGTTTA TTCCAATATCGGTACTGGTTTGCTAGGGATGATTGCGGCGAA and GCCGAGGCTTACGGGATCAAGACCGGCTCGGCGGATCTGCTGAAGTTTACCGAGGCCAA CATGGGGTATCAGGGAGATGCCGCGCTAAAAACGCGGATCGCGCTGACCCATACCGGTTT CTACTCGGTGGGAGACATGACTCAGGGGCTGGGTTGGGAGAGCTACGCCTATCCGTTGAC CGAGCAGGCGCTGCTGGCGGGCAACTCCCCGGCGGTGAGCTTCCAGGCCAATCCGGTTA CGCGCTTTG. The first DNA control mix may include the following internal control sequence: GAGAGGATGACCAGCCACACTGGAACTGAGACACGGTCCAGACTCCTACGGGAGGCAGC AGTGGGGAATATTGCACAATGGGCG. A DNA control mix may include a combination of the one or more said group of sequences and the said internal control sequence. A DNA control mix may include any combination of sequences from the first control mix, the second control mix and the internal control sequence. (SEQ. ID NOS: 273-276)

A second DNA control mix may include one or more sequences selected from the group consisting of: AACTTTCACAGGTGTGCTGGGTGCGGTTTCTGTGGCGAAAAAAGAGATGGCGCTGAATGA TCCGGCGGCAAAATACCAGCCGGAGCTGGCTCTGCCGCAGTGGAAGGGGATCACATTGC TGGATCTGGCTACCTATACCGCAGGCGGACTGCCGTTACAGGTGCCGGATGCGGTAAAAA GCCGTGCGGATCTGCTGAATTTCTATCAGCAGTGGCAGCCGTCCCGGAAACCGGGCGATA TGCGTCTGTATGCAAACAGCAGTATCGGCCTGTTTGGTGCTCTGACCGCAAACGCGGCGG GGATGCCGTATGAGCAGTTGCTGACTGCACGGATCCTGGCACCGCTGGGGTTATCTCACA CCTTTATTACTGTGCCGGAAAGTGCGCAAAGCCAGTATGCGTACGG, TCGGTAAAGCCGATGTTGCGGCGAACAAACCCGTCACCCCGCAAACCCTGTTTGAGCTGG GCTCTATAAGTAAAACCTTCACCGGCGTACTGGGCGGCGATGCCATTGCCCGGGGTGAAA TAGCGCTGGGCGATCCGGTAGCAAAATACTGGCCTGAGCTCACGGGCAAGCAGTGGCAG GGCATTCGCATGCTGGATCTGGCAACCTATACCGCAGGCGGTCTGCCGTTACAGGTGCCG GATGAGGTCACGGATACCGCCTCTCTGCTGCGCTTTTATCAAAACTGGCAGCCGCAGTGG AAG and CCGAAGCCTATGGCGTGAAATCCAGCGTTATTGATATGGCCCGCTGGGTTCAGGCCAACA TGGATGCCAGCCACGTTCAGGAGAAAACGCTCCAGCAGGGCATTGC. The second DNA control mix may include the following internal control sequence: GAGAGGATGACCAGCCACACTGGAACTGAGACACGGTCCAGACTCCTACGGGAGGCAGC AGTGGGGAATATTGCACAATGGGCG. A DNA control mix may include a combination of the one or more said group of sequences and the said internal control sequence. A DNA control mix may include any combination of sequences from the first control mix, the second control mix and the internal control sequence. (SEQ. ID NOS: 277-280)

In one example, the kit includes two primer-probe multiplex mix vials including internal controls and two DNA control mix vials. The two primer-probe multiplex mixes may provide for identification of up to six antibiotic resistance genes and internal controls. In some embodiments, a first primer-probe mix includes sequences for detecting gene families which are OXA-143-like, OXA-24/40-like, OXA-48-like and internal controls. A first primer-probe mix may include sequences for detecting gene families which are OXA-143-like, OXA-23-like, OXA-51-like and internal controls. In some embodiments, a second primer-probe mix includes sequences for detecting gene families which are OXA-58-like, OXA-51-like, OXA-23-like and internal controls. A second primer-probe mix may include sequences for detecting gene families which are OXA-48-like, OXA-58-like, OXA-24/40-like and internal controls. In some embodiments, a first DNA control mix includes DNA sequences for OXA-143, OXA-24/40, OXA-481 and an internal control DNA sequence. A first DNA control mix may include DNA sequences for OXA-143, OXA-23, OXA-51 and an internal control DNA sequence. In some embodiments, a second DNA control mix may include DNA sequences for OXA-58, OXA-51 and OXA-23 and an internal control DNA sequence. A second DNA control mix may include DNA sequences for OXA-48, OXA-58 and OXA 24/40 and an internal control DNA sequence.

It is contemplated that the combination of gene families may vary. For example, a primer-probe mix may include sequences for detecting any combination of the following genes: OXA-143-like, OXA-23-like, OXA-51-like, OXA-48-like, OXA-58-like and OXA-24/40-like. It is further contemplated that additional β-lactamase gene targets may be included in the primer-probe mix or mixes.

The first primer-probe mix may include one or more primers and/or probes selected from the group consisting of: AGCACATACAGAATATGTCCCTGC, ACCTGTTAACCAACCTACTTGAGGG, /56-FAM/TTGCAAGACGGACTGGCTTAGACC/3BHQ_1/, CCTGATCGGATTGGAGAACC, CTACCTCTTGAATAGGCGTAACC, /5TEX615/ACGTCGCGCAAGTTCCTGATAGAC/3IAbRQSp/, TAGTGACTGCTAATCCAAATCACAG, GCACGAGCAAGATCATTACCATAGC, /5HEX/AGTTATCCAACAAGGCCAAACTCAACA/3BHQ_1/. [SEQ. ID NOS 119-127] The first primer-probe mix may include one or more internal controls selected from the group consisting of: GAGAGGATGAYCAGCCACAC, CGCCCATTGTSCAATATTCC and 5TYE665/TGAGACACGGTCCAGACTCCTACG/3IAbRQSp. A primer-probe mix may include a combination of the one or more said group of primers and/or probes and the one or more said group of internal controls. The primer-probe mix including internal controls may be a multiplex mix.

The second primer-probe mix may include one or more primers and/or probes selected from the group consisting of: AATCACAGGGCGTAGTTGTG, ACCCACCAGCCAATCTTAGG, /5HEX/TAGCTTGATCGCCCTCGATTTGGG/3BHQ_1/, GTGGGATGGAAAGCCACG, CACTTGCGGGTCTACAGC, /56-FAM/TTACTTTGGGCGAAGCCATGCAAG/3BHQ_1/, CACCTATGGTAATGCTCTTGC, CTGGAACTGCTGACAATGCC, /5TEX615/TGGGAGAAAGATATGACTTTAGGTGAGGCA/3IAbRQSp/. (SEQ. ID NOS: 128-136) The second primer-probe mix may include one or more internal controls selected from the group consisting of: GAGAGGATGAYCAGCCACAC, CGCCCATTGTSCAATATTCC and 5TYE665/TGAGACACGGTCCAGACTCCTACG/3IAbRQSp. A primer-probe mix may include a combination of the one or more said group of primers and/or probes and the one or more said group of internal controls. The primer-probe mix including internal controls may be a multiplex mix.

The kit may include exactly two primer and/or probe mixes, a first primer and/or probe mix including one or more primers and/or probes selected from the group consisting of: AGCACATACAGAATATGTCCCTGC, ACCTGTTAACCAACCTACTTGAGGG, /56-FAM/TTGCAAGACGGACTGGCTTAGACC/3BHQ_1/, CCTGATCGGATTGGAGAACC, CTACCTCTTGAATAGGCGTAACC, /5TEX615/ACGTCGCGCAAGTTCCTGATAGAC/3IAbRQSp/, TAGTGACTGCTAATCCAAATCACAG, GCACGAGCAAGATCATTACCATAGC, /5HEX/AGTTATCCAACAAGGCCAAACTCAACA/3BHQ_1/, GAGAGGATGAYCAGCCACAC, CGCCCATTGTSCAATATTCC and 5TYE665/TGAGACACGGTCCAGACTCCTACG/3IAbRQSp; and a second primer and/or probe mix including one or more primers and/or probes selected from the group consisting of: AATCACAGGGCGTAGTTGTG, ACCCACCAGCCAATCTTAGG, /5HEX/TAGCTTGATCGCCCTCGATTTGGG/3BHQ_1/, GTGGGATGGAAAGCCACG, CACTTGCGGGTCTACAGC, /56-FAM/TTACTTTGGGCGAAGCCATGCAAG/3BHQ_1/, CACCTATGGTAATGCTCTTGC, CTGGAACTGCTGACAATGCC, /5TEX615/TGGGAGAAAGATATGACTTTAGGTGAGGCA/3IAbRQSp/, GAGAGGATGAYCAGCCACAC, CGCCCATTGTSCAATATTCC and 5TYE665/TGAGACACGGTCCAGACTCCTACG/3IAbRQSp. Primers and/or probes included in this group may or may not be degenerate at any nucleotide position. (SEQ. ID NOS: 216-239)

A first DNA control mix may include one or more sequences selected from the group consisting of: AGCACATACAGAATATGTCCCTGCATCAACATTTAAGATGCTAAATGCCTTAATTGGACTAG AAAATCATAAAGCTACAACAACTGAGATTTTCAAATGGGACGGTAAAAAGAGATCTTATCCC ATGTGGGAAAAAGATATGACTTTAGGTGATGCCATGGCACTTTCAGCAGTTCCTGTATATCA AGAACTTGCAAGACGGACTGGCTTAGACCTAATGCAAAAAGAAGTTAAACGGGTTGGTTTT GGTAATATGAACATTGGAACACAAGTTGATAACTTCTGGTTGGTTGGCCCCCTCAAGATTA CACCAATACAAGAGGTTAATTTTGCCGATGATTTTGCAAATAATCGATTACCCTTTAAATTAG AGACTCAAGAAGAAGTTAAAAAAATGCTTCTGATTAAAGAATTCAATGGTAGTAAAATTTATG CAAAAAGCGGCTGGGGAATGGATGTAACCCCTCAAGTAGGTTGGTTAACAGGT, CCTGATCGGATTGGAGAACCAGAAAACGGATATTAATGAAATATTTAAATGGAAGGGCGAG AAAAGGTCATTTACCGCTTGGGAAAAAGACATGACACTAGGAGAAGCCATGAAGCTTTCTG CAGTCCCAGTCTATCAGGAACTTGCGCGACGTATCGGTCTTGATCTCATGCAAAAAGAAGT AAAACGTATTGGTTTCGGTAATGCTGAAATTGGACAGCAGGTTGATAATTTCTGGTTGGTAG GACCATTAAAGGTTACGCCTATTCAAGAGGTAG and TAGTGACTGCTAATCCAAATCACAGCGCTTCAAAATCTGATGAAAAAGCAGAGAAAATTAAA AATTTATTTAACGAAGTACACACTACGGGTGTTTTAGTTATCCAACAAGGCCAAACTCAACA AAGCTATGGTAATGATCTTGCTCGTGC. The first DNA control mix may include the following internal control sequence: GAGAGGATGACCAGCCACACTGGAACTGAGACACGGTCCAGACTCCTACGGGAGGCAGC AGTGGGGAATATTGCACAATGGGCG. A DNA control mix may include a combination of the one or more said group of sequences and the said internal control sequence. A DNA control mix may include any combination of sequences from the first control mix, the second control mix and the internal control sequence. (SEQ. ID NOS: 281-284)

A second DNA control mix may include one or more sequences selected from the group consisting of: AATCACAGGGCGTAGTTGTGCTCTGGAATGAGAATAAGCAGCAAGGATTTACCAATAATCT TAAACGGGCGAACCAAGCATTTTTACCCGCATCTACCTTTAAAATTCCCAATAGCTTGATCG CCCTCGATTTGGGCGTGGTTAAGGATGAACACCAAGTCTTTAAGTGGGATGGACAGACGC GCGATATCGCCACTTGGAATCGCGATCATAATCTAATCACCGCGATGAAATATTCAGTTGT GCCTGTTTATCAAGAATTTGCCCGCCAAATTGGCGAGGCACGTATGAGCAAGATGCTACAT GCTTTCGATTATGGTAATGAGGACATTTCGGGCAATGTAGACAGTTTCTGGCTCGACGGTG GTATTCGAATTTCGGCCACGGAGCAAATCAGCTTTTTAAGAAAGCTGTATCACAATAAGTTA CACGTATCGGAGCGCAGCCAGCGTATTGTCAAACAAGCCATGCTGACCGAAGCCAATGGT GACTATATTATTCGGGCTAAAACTGGATACTCGACTAGAATCGAACCTAAGATTGGCTGGT GGGT, GTGGGATGGAAAGCCACGTTTTTTTAAAGCATGGGACAAAGATTTTACTTTGGGCGAAGCC ATGCAAGCATCTACAGTGCCTGTATATCAAGAATTGGCACGTCGTATTGGTCCAAGCTTAAT GCAAAGTGAATTGCAACGTATTGGTTATGGCAATATGCAAATAGGCACGGAAGTTGATCAA TTTTGGTTGAAAGGGCCTTTGACAATTACACCTATACAAGAAGTAAAGTTTGTGTATGATTT AGCCCAAGGGCAATTGCCTTTTAAACCTGAAGTTCAGCAACAAGTGAAAGAGATGTTGTAT GTAGAGCGCAGAGGGGAGAATCGTCTATATGCTAAAAGTGGCTGGGGAATGGCTGTAGAC CCGCAAGTG, CACTTGCGGGTCTACAGCCATTCCCCAGCCACTTTTAGCATATAGACGATTCTCCCCTCTG CGCTCTACATACAACATCTCTTTCACTTGTTGCTGAACTTCAGGTTTAAAAGGCAATTGCCC TTGGGCTAAATCATACACAAACTTTACTTCTTGTATAGGTGTAATTGTCAAAGGCCCTTTCA ACCAAAATTGATCAACTTCCGTGCCTATTTGCATATTGCCATAACCAATACGTTGCAATTCA CTTTGCATTAAGCTTGGACCAATACGACGTGCCAATTCTTGATATACAGGCACTGTAGATG CTTGCATGGCTTCGCCCAAAGTAAAATCTTTGTCCCATGCTTTAAAAAAACGTGGCTTTCCA TCCCAC, and CACCTATGGTAATGCTCTTGCACGAGCAAATAAAGAATATGTCCCTGCATCAACATTTAAGA TGCTAAATGCTTTAATCGGGCTAGAAAATCATAAAGCAACAACAAATGAGATTTTCAAATGG GATGGTAAAAAAAGAACTTATCCTATGTGGGAGAAAGATATGACTTTAGGTGAGGCAATGG CATTGTCAGCAGTTCCAG. The second DNA control mix may include the following internal control sequence: GAGAGGATGACCAGCCACACTGGAACTGAGACACGGTCCAGACTCCTACGGGAGGCAGC AGTGGGGAATATTGCACAATGGGCG. A DNA control mix may include a combination of the one or more said group of sequences and the said internal control sequence additional β-lactamase. A DNA control mix may include any combination of sequences from the first control mix, the second control mix and the internal control sequence. (SEQ. ID NOS: 285-289)

In one example, the kit includes one primer-probe multiplex mix vials including internal control and one DNA control mix vial. A primer-probe mix may include sequences for detecting MCR gene families and internal control.

The primer-probe mix may include primers and/or probes selected from the group consisting of: CCGTGTATGTTCAGCTAT, CTTATCCATCACGCCTTT, /5TEX615/TATGATGTCGATACCGCCAAATACCA/3IAbRQSp/, CTGTATGTCAGCGATCAT, GATGCCAGTTTGCTTATCC, /56-FAM/AAGTCTGGG/ZEN/TGAGAACGGTGTCTAT/3IABkFQ/, CAGTCAGTATGCGAGTTTC, AAAATTCGCCAAGCCATC, and/5HEX/TGCATAAGC/ZEN/CAGTGCGTTTTTATAT/3IABkFQ/. The primer-probe mix may include one or more internal controls selected from the group consisting of: GAGAGGATGAYCAGCCACAC, CGCCCATTGTSCAATATTCC and 5TYE665/TGAGACACGGTCCAGACTCCTACG/3IAbRQSp. The primer-probe mix may include a combination of the one or more said group of primers and/or probes and the one or more said group of internal controls. The primer-probe mix including internal controls may be a multiplex mix. (SEQ. ID NOS: 137-145)

A DNA control mix may include one or more sequences selected from the group consisting of ATGATGCAGCATACTTCTGTGTGGTACCGACGCTCGGTCAGTCCGTTTGTTCTTGTGGGAG TGTTGCCGTTTTCTTGACCGCGACCGCCAATCTTACCTTTTTTGATAAAATCAGCCAAACCT ATCCCATCGCGGACAATCTCGGCTTTGTGCTGACGATCGCTGTCGTGCTCTTTGGCGCGA TGCTACTGATCACCACGCTGTTATCATCGTATCGCTATGTGCTAAAGCCTGTGTTGATTTTG CTATTAATCATGGGCGCGGTGACCAGTTATTTTACTGACACTTATGGCACGGTCTATGATAC GACCATGCTCCAAAATGCCCTACAGACCGACCAAGCCGAGACCAAGGATCTATTAAACGC AGCGTTTATCATGCGTATCATTGGTTTGGGTGTGCTACCAAGTTTGCTTGTGGCTTTTGTTA AGGTGGATTATCCGACTTGGGGCAAGGGTTTGATGCGCCGATTGGGCTTGATCGTGGCAA GTCTTGCGCTGATTTTACTGCCTGTGGTGGCGTTCAGCAGTCATTATGCCAGTTTCTTTCG CGTGCATAAGCCGCTGCGTAGCTATGTCAATCCGATCATGCCAATCTACTCGGTGGGTAAG CTTGCCAGTATTGAGTATAAAAAAGCCAGTGCGCCAAAAGATACCATTTATCACGCCAAAG ACGCGGTACAAGCAACCAAGCCTGATATGCGTAAGCCACGCCTAGTGGTGTTCGTCGTCG GTGAGACGGCACGCGCCGATCATGTCAGCTTCAATGGCTATGAGCGCGATACTTTCCCAC AGCTTGCCAAGATCGATGGCGTGACCAATTTTAGCAATGTCACATCGTGCGGCACATCGAC GGCGTATTCTGTGCCGTGTATGTTCAGCTATCTGGGCGCGGATGAGTATGATGTCGATACC GCCAAATACCAAGAAAATGTGCTGGATACGCTGGATCGCTTGGGCGTAAGTATCTTGTGGC GTGATAATAATTCGGACTCAAAAGGCGTGATGGATAAGCTGCCAAAAGCGCAATTTGCCGA TTATAAATCCGCGACCAACAACGCCATCTGCAACACCAATCCTTATAACGAATGCCGCGAT GTCGGTATGCTCGTTGGCTTAGATGACTTTGTCGCTGCCAATAACGGCAAAGATATGCTGA TCATGCTGCACCAAATGGGCAATCACGGGCCTGCGTATTTTAAGCGATATGATGAAAAGTT TGCCAAATTCACGCCAGTGTGTGAAGGTAATGAGCTTGCCAAGTGCGAACATCAGTCCTTG ATCAATGCTTATGACAATGCCTTGCTTGCCACCGATGATTTCATCGCTCAAAGTATCCAGTG GCTGCAGACGCACAGCAATGCCTATGATGTCTCAATGCTGTATGTCAGCGATCATGGCGAA AGTCTGGGTGAGAACGGTGTCTATCTACATGGTATGCCAAATGCCTTTGCACCAAAAGAAC AGCGCAGTGTGCCTGCATTTTTCTGGACGGATAAGCAAACTGGCATCACGCCAATGGCAA CCGATACCGTCCTGACCCATGACGCGATCACGCCGACATTATTAAAGCTGTTTGATGTCAC CGCGGACAAAGTCAAAGACCGCACCGCATTCATCCGCTGA and ATGACATCACATCACTCTTGGTATCGCTATTCTATCAATCCTTTTGTGCTGATGGGTTTGGT GGCGTTATTTTTGGCAGCGACAGCGAACCTGACATTTTTTGAAAAAGCGATGGCGGTCTAT CCTGTATCGGATAACTTAGGCTTTATCATCTCAATGGCGGTGGCGGTGATGGGTGCTATGC TACTGATTGTCGTGCTGTTATCCTATCGCTATGTGCTAAAGCCTGTCCTGATTTTGCTACTG ATTATGGGTGCGGTGACGAGCTATTTTACCGATACTTATGGCACGGTCTATGACACCACCA TGCTCCAAAATGCCATGCAAACCGACCAAGCCGAGTCTAAGGACTTGATGAATTTGGCGTT TTTTGTGCGAATTATCGGGCTTGGCGTGTTGCCAAGTGTGTTGGTCGCAGTTGCCAAAGTC AATTATCCAACATGGGGCAAAGGTCTGATTCAGCGTGCGATGACATGGGGTGTCAGCCTT GTGCTGTTGCTTGTGCCGATTGGACTATTTAGCAGTCAGTATGCGAGTTTCTTTCGGGTGC ATAAGCCAGTGCGTTTTTATATCAACCCGATTACGCCGATTTATTCGGTGGGTAAGCTTGC CAGTATCGAGTACAAAAAAGCCACTGCGCCAACAGACACCATCTATCATGCCAAAGACGCC GTGCAGACCACCAAGCCGAGCGAGCGTAAGCCACGCCTAGTGGTGTTCGTCGTCGGTGA GACGGCGCGTGCTGACCATGTGCAGTTCAATGGCTATGGCCGTGAGACTTTCCCGCAGCT TGCCAAAGTTGATGGCTTGGCGAATTTTAGCCAAGTGACATCGTGTGGCACATCGACGGC GTATTCTGTGCCGTGTATGTTCAGCTATTTGGGTCAAGATGACTATGATGTCGATACCGCC AAATACCAAGAAAATGTGCTAGATACGCTTGACCGCTTGGGTGTGGGTATCTTGTGGCGTG ATAATAATTCAGACTCAAAAGGCGTGATGGATAAGCTACCTGCCACGCAGTATTTTGATTAT AAATCAGCAACCAACAATACCATCTGTAACACCAATCCCTATAACGAATGCCGTGATGTCG GTATGCTTGTCGGGCTAGATGACTATGTCAGCGCCAATAATGGCAAAGATATGCTCATCAT GCTACACCAAATGGGCAATCATGGGCCGGCGTACTTTAAGCGTTATGATGAGCAATTTGCC AAATTCACCCCCGTGTGCGAAGGCAACGAGCTTGCCAAATGCGAACACCAATCACTCATCA ATGCCTATGACAATGCGCTACTTGCGACTGATGATTTTATCGCCAAAAGCATCGATTGGCT AAAAACGCATGAAGCGAACTACGATGTCGCCATGCTCTATGTCAGTGACCACGGCGAGAG CTTGGGCGAAAATGGTGTCTATCTGCATGGTATGCCAAATGCCTTTGCACCAAAAGAACAG CGAGCTGTGCCTGCGTTTTTTTGGTCAAATAATACGACATTCAAGCCAACTGCCAGCGATA CTGTGCTGACGCATGATGCGATTACGCCAACACTGCTTAAGCTGTTTGATGTCACAGCGGG CAAGGTCAAAGACCGCGCGGCATTTATCCAGTAA. The DNA control mix may include the following internal control sequence: GAGAGGATGACCAGCCACACTGGAACTGAGACACGGTCCAGACTCCTACGGGAGGCAGC AGTGGGGAATATTGCACAATGGGCG. A DNA control mix may include a combination of the one or more said group of sequences and the said internal control sequence. (SEQ. ID NOS: 290-292)

In one example, the kit includes one primer-probe multiplex mix vial including internal control and one DNA control mix vial. A primer-probe mix may include sequences for detecting TEM-like and SHV-like gene families and internal control.

The primer-probe mix may include primers and/or probes selected from the group consisting of: AGATCAGTTGGGTGCACG, TGCTTAATCAGTGAGGCACC, /56-FAM/ATGAAGCCA/ZEN/TACCAAACGACGAGC/3IABkFQ/, CTGGAGCGAAAGATCCACTA, ATCGTCCACCATCCACTG, and/5HEX/CCAGATCGG/ZEN/CGACAACGTCACC/3IABkFQ/. The primer-probe mix may include one or more internal controls selected from the group consisting of: GAGAGGATGAYCAGCCACAC, CGCCCATTGTSCAATATTCC and 5TYE665/TGAGACACGGTCCAGACTCCTACG/3IAbRQSp. The primer-probe mix may include a combination of the one or more said group of primers and/or probes and the one or more said group of internal controls. The primer-probe mix including internal controls may be a multiplex mix. (SEQ. ID NOS: 240-248)

A DNA control mix may include one or more sequences selected from the group consisting of: AGATCAGTTGGGTGCACGAGTGGGTTACATCGAACTGGATCTCAACAGCGGTAAGATCCTT GAGAGTTTTCGCCCCGAAGAACGTTTTCCAATGATGAGCACTTTTAAAGTTCTGCTATGTG GTGCGGTATTATCCCGTGTTGACGCCGGGCAAGAGCAACTCGGTCGCCGCATACACTATT CTCAGAATGACTTGGTTGAGTACTCACCAGTCACAGAAAAGCATCTTACGGATGGCATGAC AGTAAGAGAATTATGCAGTGCTGCCATAACCATGAGTGATAACACTGCGGCCAACTTACTT CTGACAACGATCGGAGGACCGAAGGAGCTAACCGCTTTTTTGCACAACATGGGGGATCAT GTAACTCGCCTTGATCGTTGGGAACCGGAGCTGAATGAAGCCATACCAAACGACGAGCGT GACACCACGACGCCTGCAGCAATGGCAACAACGTTGCGCAAACTATTAACTGGCGAACTA CTTACTCTAGCTTCCCGGCAACAATTAATAGACTGGATGGAGGCGGATAAAGTTGCAGGAC CACTTCTGCGCTCGGCCCTTCCGGCTGGCTGGTTTATTGCTGATAAATCTGGAGCCAGTGA GCGTGGGTCTCGCGGTATCATTGCAGCACTGGGGCCAGATGGTAAGCCCTCCCGTATCGT AGTTATCTACACGACGGGGAGTCAGGCAACTATGGATGAACGAAATAGACAGATCGCTGA GATAGGTGCCTCACTGATTAAGCA and CTGGAGCGAAAGATCCACTATCGCCAGCAGGATCTGGTGGACTACTCGCCGGTCAGCGAA AAACACCTTGCCGACGGCATGACGGTCGGCGAACTCTGCGCCGCCGCCATTACCATGAGC GATAACAGCGCCGCCAATCTGCTGCTGGCCACCGTCGGCGGCCCCGCAGGATTGACTGC CTTTTTGCGCCAGATCGGCGACAACGTCACCCGCCTTGACCGCTGGGAAACGGAACTGAA TGAGGCGCTTCCCGGCGACGCCCGCGACACCACTACCCCGGCCAGCATGGCCGCGACCC TGCGCAAGCTGCTGACCAGCCAGCGTCTGAGCGCCCGTTCGCAACGGCAGCTGCTGCAG TGGATGGTGGACGAT. The DNA control mix may include the following internal control sequence: GAGAGGATGACCAGCCACACTGGAACTGAGACACGGTCCAGACTCCTACGGGAGGCAGC AGTGGGGAATATTGCACAATGGGCG. A DNA control mix may include a combination of the one or more said group of sequences and the said internal control sequence. (SEQ. ID NOS: 293-295)

The primer-probe multiplex mix may comprise different oligonucleotide sequences. An oligonucleotide sequence may be utilized as a primer. An oligonucleotide sequence may be utilized as a probe. An oligonucleotide sequence may be utilized as an internal control sequence. The oligonucleotide concentration of a primer and/or probe sequence may range from 0.05 μM to 60 μM. For example, the oligonucleotide concentration of a primer and/or probe sequence may range from 3 μM to 8 μM. For example, the oligonucleotide concentration of an internal control sequence may range from 2 μM to 6 μM. For example, the oligonucleotide concentration of an internal control sequence may range from 2 μM to 8 μM. The vial oligonucleotide concentrations may be prepared as a 10× stock solution.

The synthetic gene size of a DNA control sequence may be from about 84 bp to about 533 bp. The concentration of a DNA control sequence may be about 25 ng/μl. The concentration of a DNA control sequence may be from 0.033 ng/μL to about 0.5 ng/μl.

The present teachings provide methods for detection of β-lactamase gene families from a biological sample. Preferably, the sample includes Gram-negative bacteria. The method may include sample processing. The method may include extracting DNA from the sample. The method may include extracting RNA from the sample. The method may include the use of assays of the present teachings. The assays may be included in a kit or kits. The method may include employing the kit of the present teachings for the detection of multiple β-lactamase gene families from a biological sample.

The method may include employing the kit for analysis of nucleic acid contained in a clinical sample. The method may include employing the kit for analysis of DNA extracted from a clinical sample. The method may include employing the kit for analysis of DNA extracted from an overnight bacterial culture of a clinical sample.

The method may include amplifying a targeted DNA sequence by real-time polymerase reaction. The method may include amplifying several targeted DNA sequences by multiplex real-time polymerase reaction. The method may include analyzing the amplified sequences or amplicons. The method may include detecting the presence or absence of β-lactamase genes. The method may include detecting the presence or absence of ampC β-lactamase genes. The method may include identifying up to six β-lactamase gene families. The method may include identifying up to nine β-lactamase gene families. The method may include identifying up to fifteen β-lactamase gene families. The method may include identifying up to twenty β-lactamase gene families. The method may include identifying from about six to about thirty β-lactamase gene families. The method may include analyzing collected data.

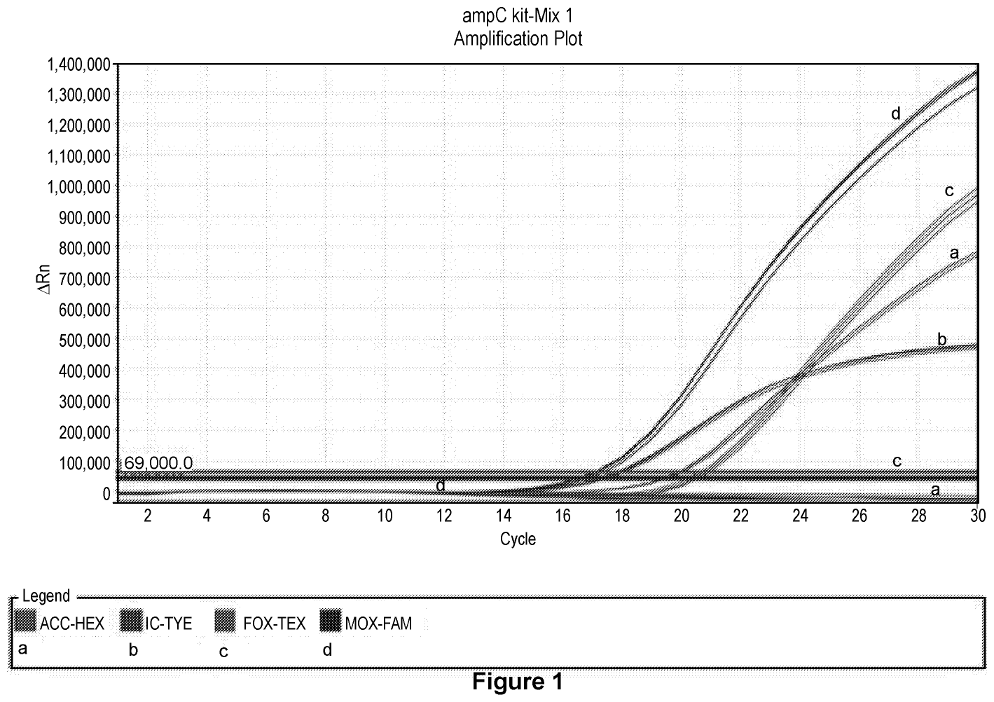

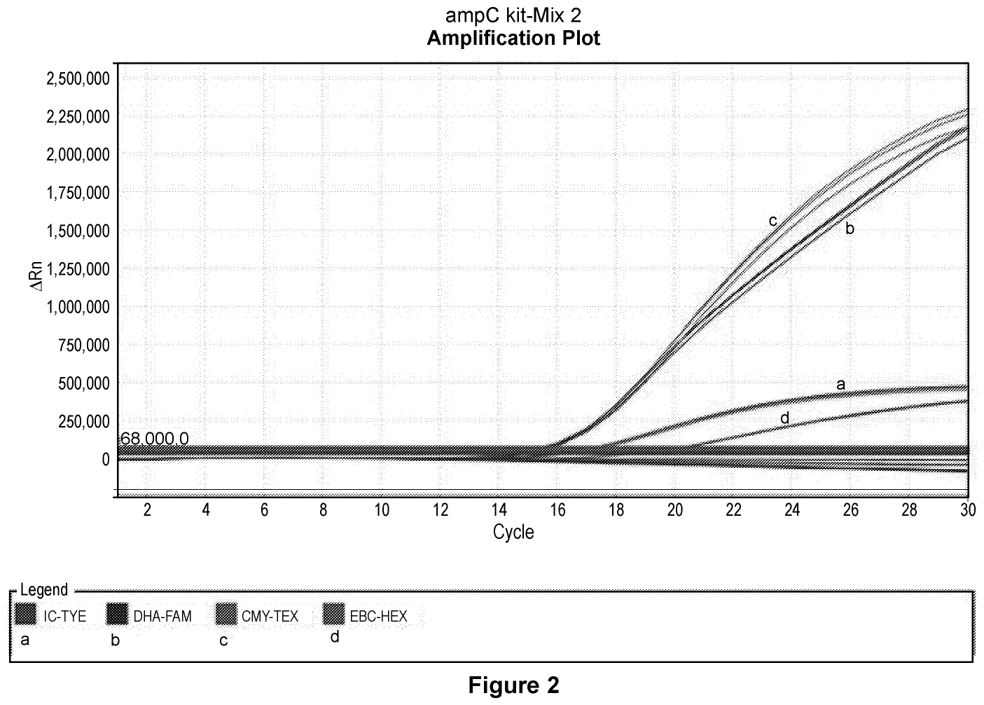

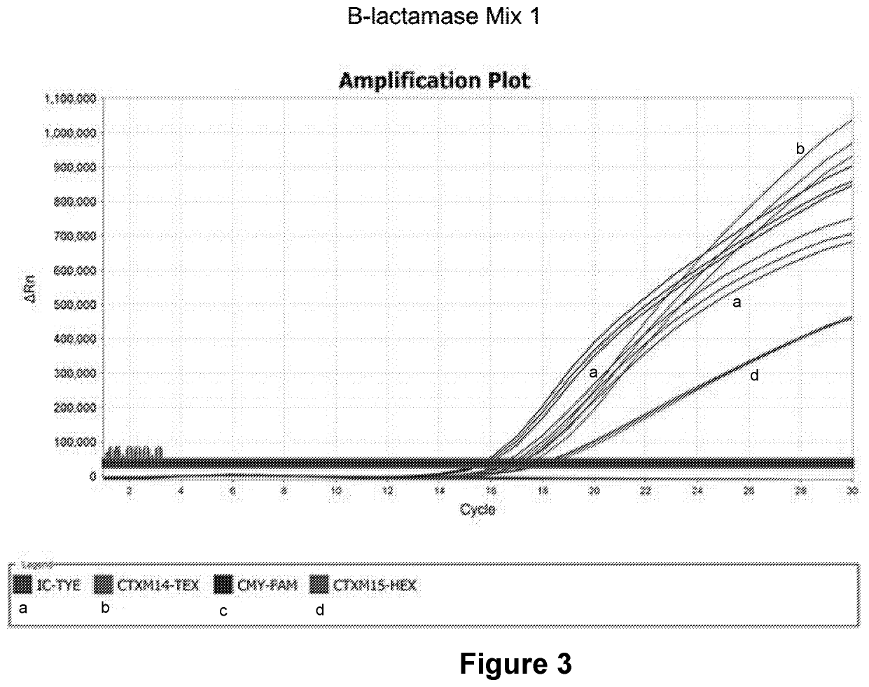

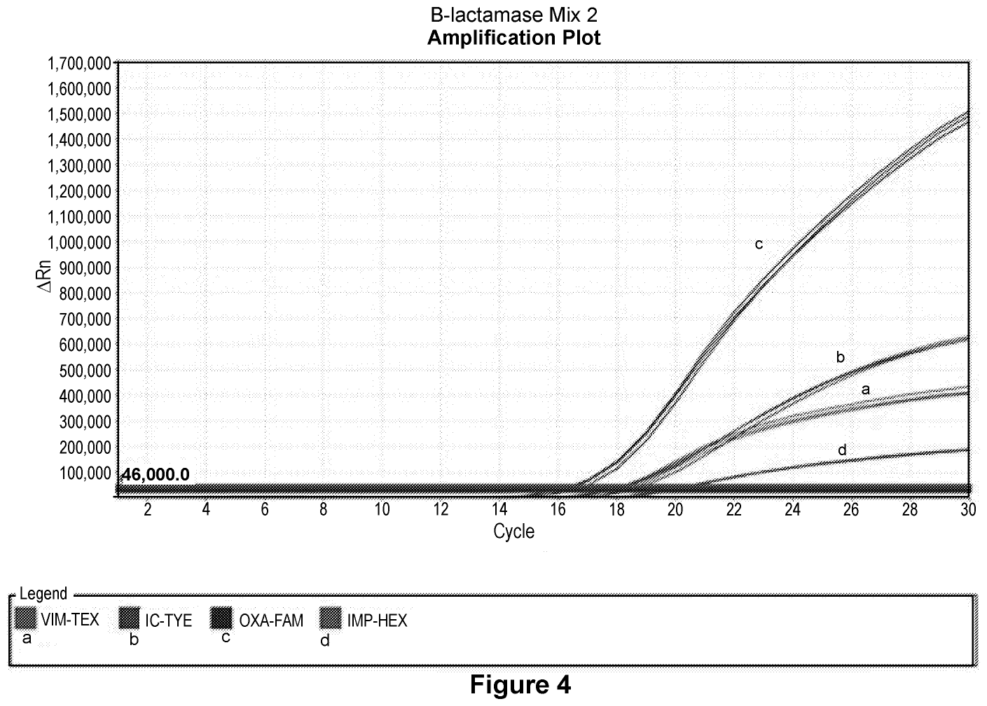

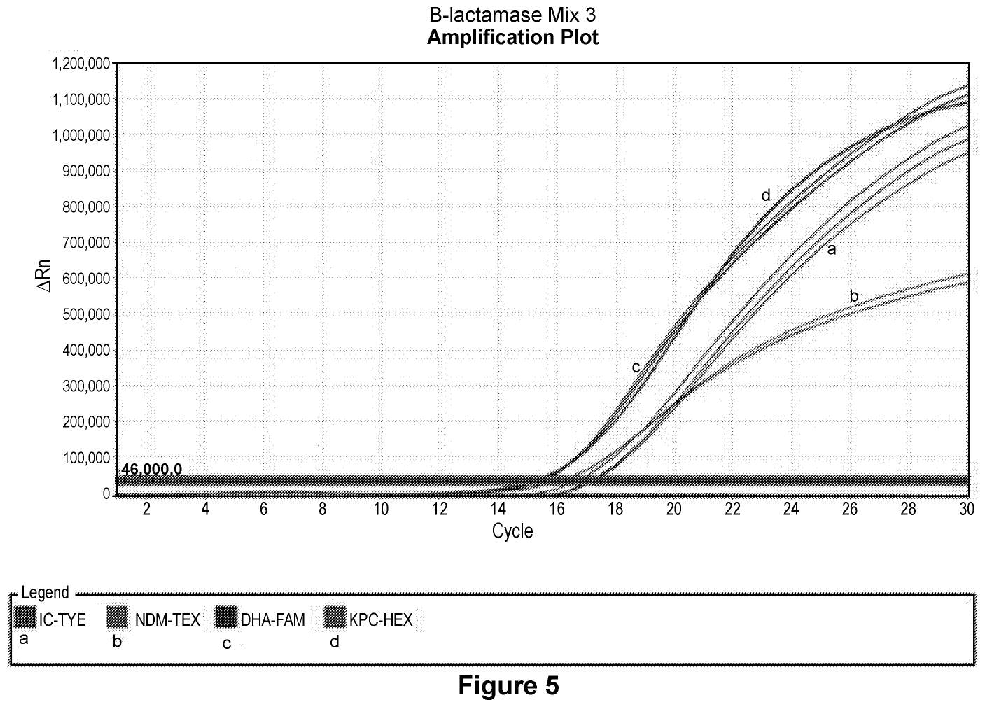

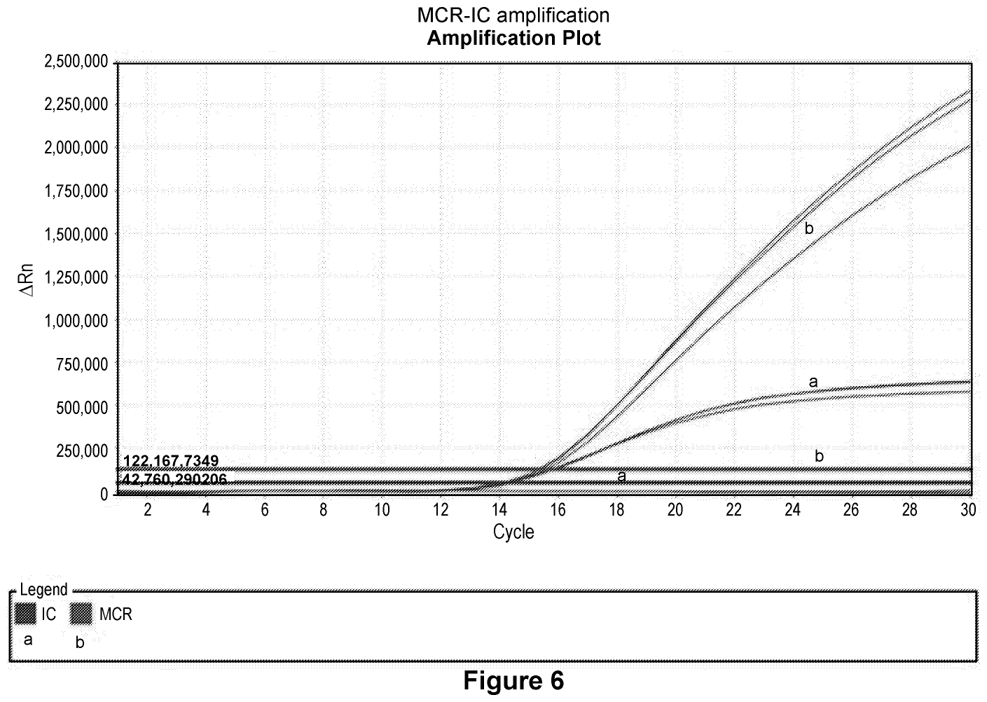

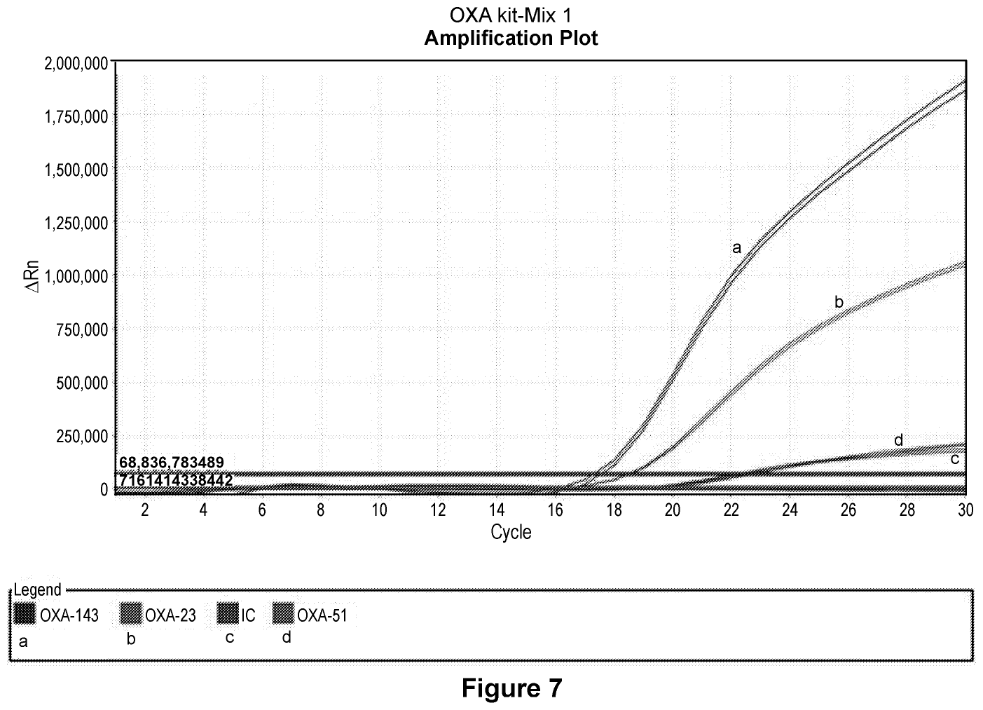

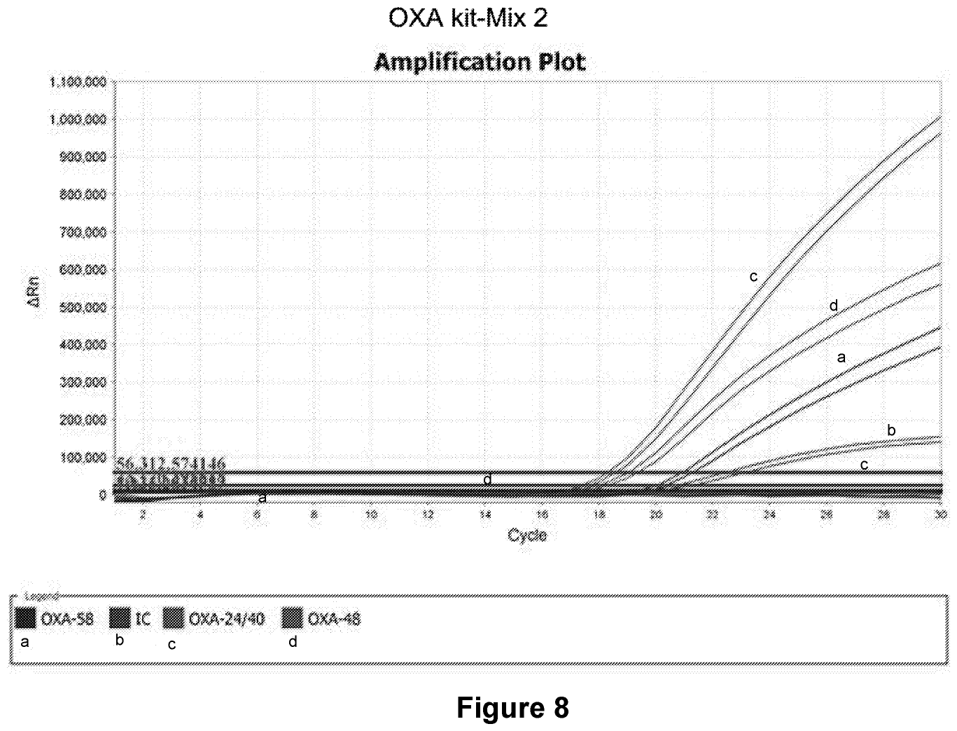

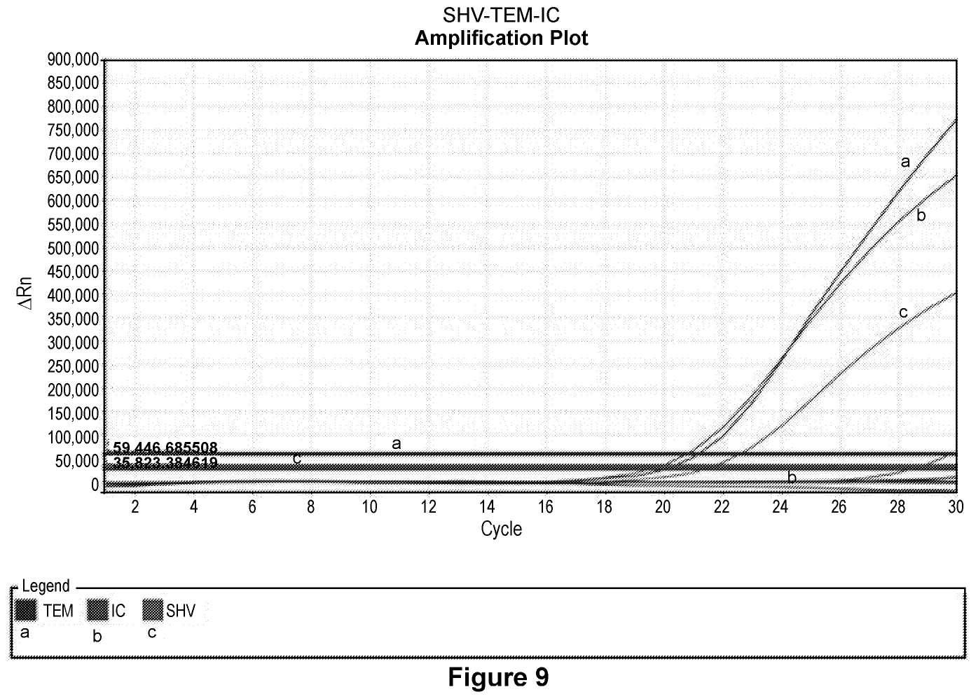

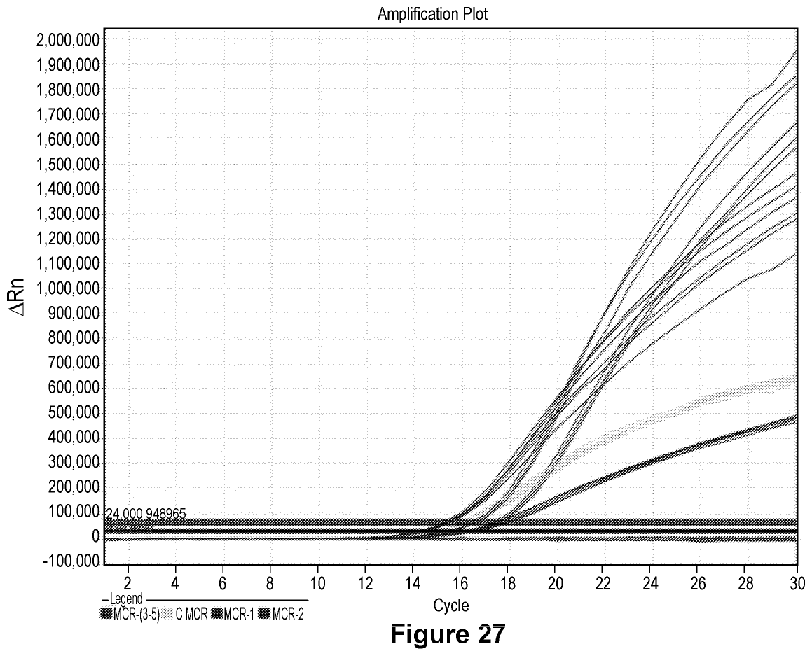

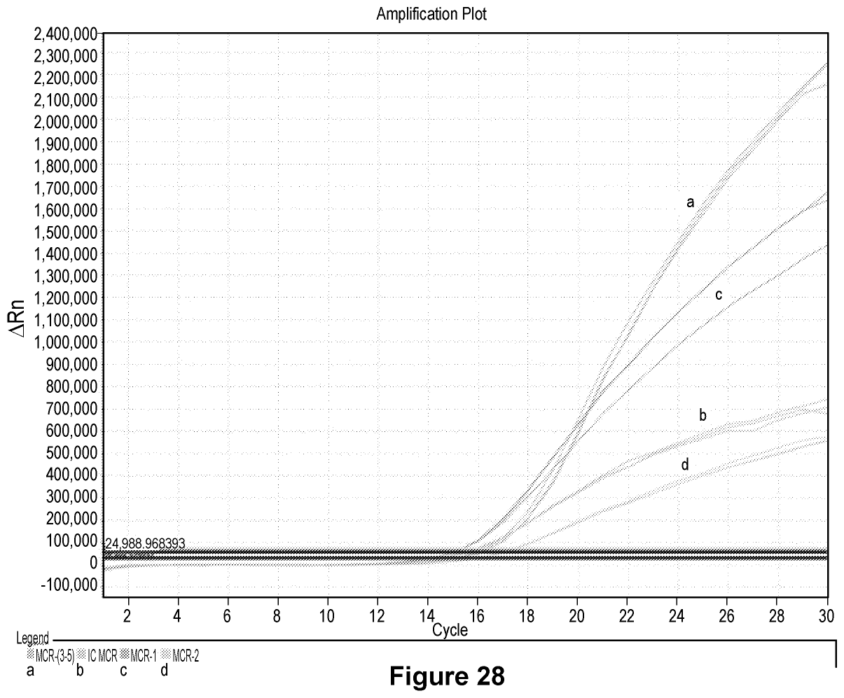

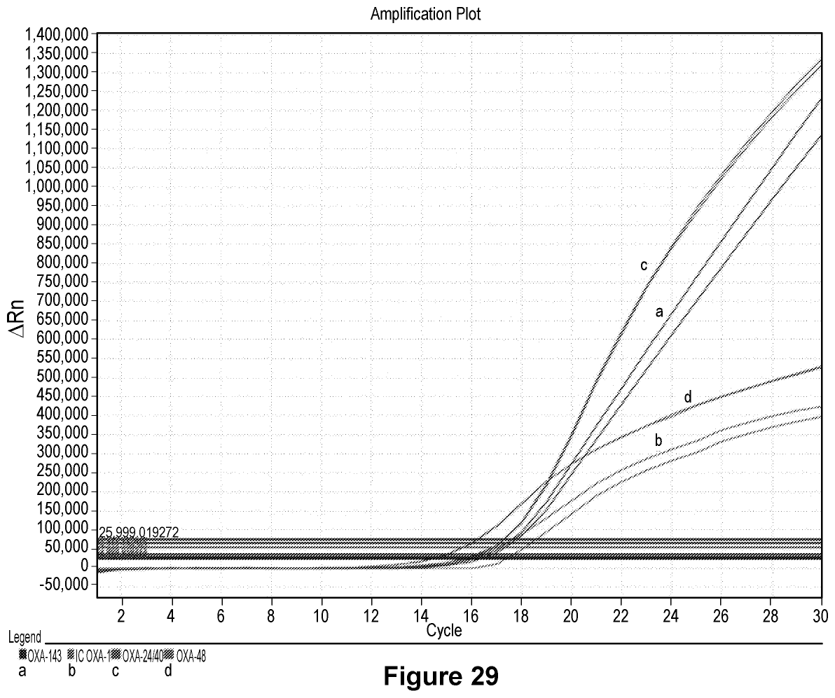

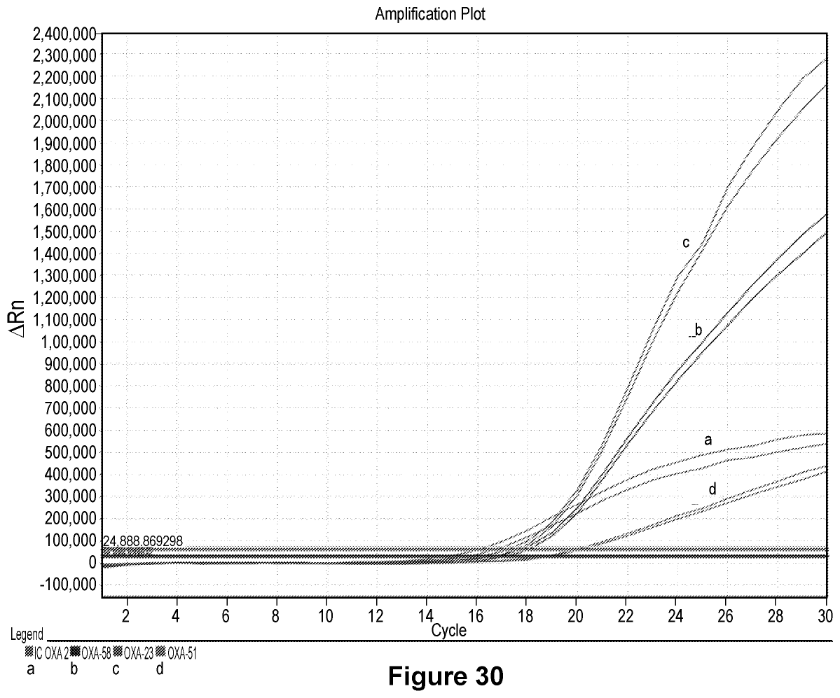

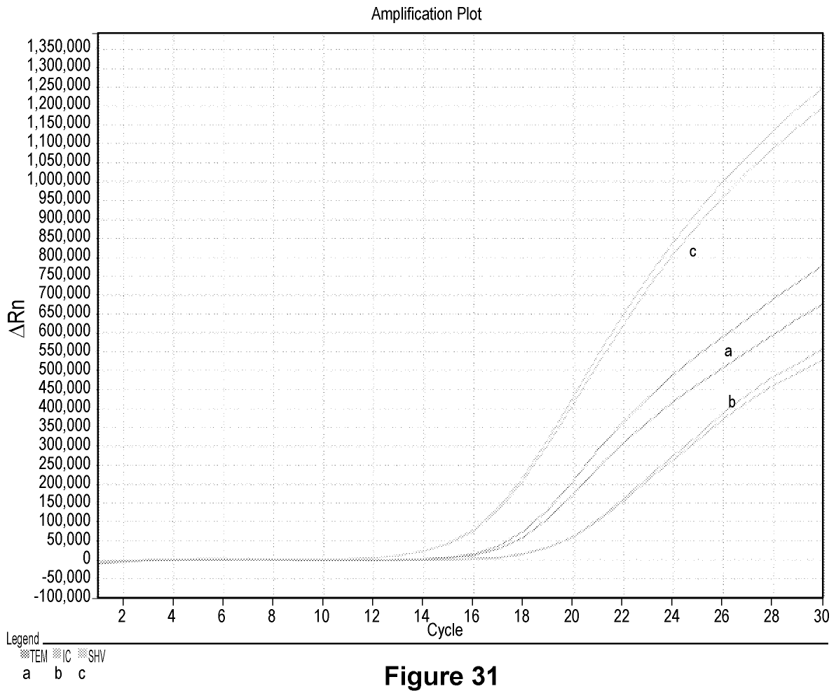

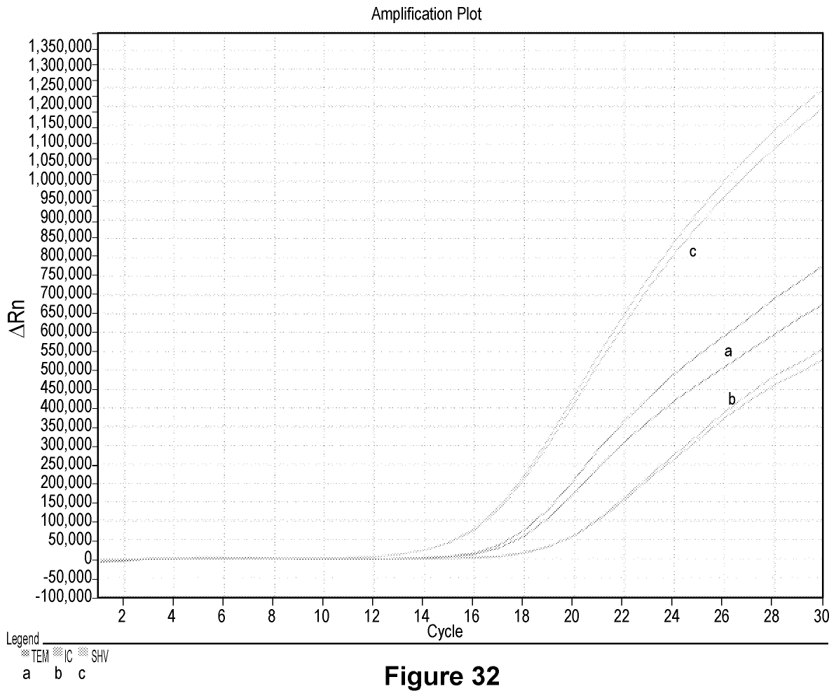

Examples of real-time PCR amplification curves obtained on the ABI QS7 Flex-Real-Time System for some of the multiplex mixes described herein are shown in FIGS. 1-9. FIG. 1 depicts an amplification plot of an exemplary mix 1 including ampC gene targets. FIG. 2 depicts an amplification plot of an exemplary mix 2 including ampC gene targets. FIG. 3 depicts an amplification plot of an exemplary mix 1 including β-lactamase gene targets. FIG. 4 depicts an amplification plot of an exemplary mix 2 including β-lactamase gene targets. FIG. 5 depicts an amplification plot of an exemplary mix 3 including β-lactamase gene targets. FIG. 6 depicts an amplification plot of an exemplary internal control mix including MCR gene targets. FIG. 7 depicts an amplification plot of an exemplary mix 1 including OXA gene targets. FIG. 8 depicts an amplification plot of an exemplary mix 2 including OXA gene targets. FIG. 9 depicts an amplification plot of an exemplary internal control mix including SHV-TEM gene targets. Additional results generated using representative kits of the disclosure are shown in FIGS. 27-32.

The method may include using one or more oligonucleotide primers that are complementary to at least a portion of the nucleic acid sequence of interest. The method may include annealing several pairs of primers to different target DNA sequences. The method may include annealing primer/probe sequences to bacterial nucleic acid sequences comprising targeted antibiotic resistant gene family variants of β-lactamases. The primer and/or probe sequences may anneal with 100% specificity to the target gene variants. The primer and/or probe sequences may anneal with about 95% specificity to the target gene variants. The primer and/or probe sequences may anneal with about 90% to about 100% specificity to the target gene variants. The primer and/or probe sequences may anneal with about 80% to about 100% specificity to the target gene variants.

The method may include using temperature mediated DNA polymerase. The method may include using fluorescent dyes. The method may include the using sequence specific DNA probes including oligonucleotides labeled with a reporter. The method may include using a microarray.

The method may include using a thermal cycler. For example, the kit of the present teachings may be utilized with the following PCR systems: Streck ZULU RT™ PCR System, Applied Biosystems (ABI) QuantStudio 7 (QS7) Flex Real-Time System, ABI 7500 Real-Time PCR System, QIAGEN Rotor-Gene® Q, and CFX96 Touch™ Real-Time PCR Detection System, Applied Biosystems™ 7500 Fast Dx Real-Time PCR Instrument, Roche LightCycler® 480 I and II, and Cepheid SmartCycler®. It is contemplated that any detection system capable of detecting the multiplex fluorescent signal provided in the kit of the present teachings may be suitable.

The method may include real-time monitoring of qPCR reaction products. The probes may generate a signal when hydrolyzed by the DNA polymerase causing liberation of a detectable fluorescent signal. The real-time monitoring method may employ fluorescence at different wavelengths. The method may include the use of DNA-intercalating fluorescent dyes. The method may include the use of a target specific nucleotide probe labeled with a fluorescent tag at one end. The other end of the hybridization probe may be labeled with a fluorescent quencher. Fluorescent hybridization probes generate a fluorescence signal only when they bind to their target and enable real-time of monitoring of nucleic acid amplification assays.

Surprisingly, some DNA targets detected with these kits, allow for amplification of regions of DNA much larger than the conventional wisdom within the real-time PCR field. For example, most amplicons would traditionally be between 50 to 150 base pairs in size. The present teachings allow for successfully amplified amplicons up to 553 base pairs by real-time PCR.

There may be one or more benefits to detecting larger amplicons. Larger amplicons may, in some cases, provide greater specificity for a specific antibiotic resistance gene family. Detection of larger amplicons may permit detection of an increased number of gene variants within a given resistance gene family. Detection of larger amplicons may also allow confirmation by agarose gel electrophoresis since the molecular sizes of each gene that is detected can be resolved from one another.

The efficiency of detection for each target in a dilution series may be measured for amplicons between 25 base pairs and 2000 base pairs. The efficiency of the PCR for amplicons within this size range may be from 80% to 110%. More specifically, the efficiency of the reactions may be from 90% to 105%. The coefficient of determination may be from 0.98 to 1.1. More specifically, the coefficient of determination may be from 0.99 to 1.0. The limit of detection may be from 0.1 copies to 1×10¹⁰ copies.

Alternate sequences for primer, probes, and DNA controls for β-lactamase gene targets of the present teachings are depicted in Table 2 and Table 3. (SEQ. ID NOS: 1-48 and SEQ. ID NOS: 49-66)

Primers and/or probes may be degenerate at any nucleotide position. Primers and/or probes may not be degenerate at any nucleotide position. Any suitable fluorophore and/or quencher and nucleic acid sequence combination may be used. For example, a probe may be labeled with a fluorescent tag at one end and a fluorescent quencher at the other end. For example, a probe may be labeled with a fluorescent tag at one end and a fluorescent quencher at the other end. For example, two fluorescent quenchers may be included at one end or within the probe sequence. It is contemplated that the probe sequences of the present teachings may be labeled with any suitable fluorophore and quencher combinations. For example, any fluorophore of the present teachings may be attached to any probe DNA sequence of the present teachings.

### Additional Kits

Additional kits provided by the disclosure include the following.

In some aspects, the disclosure provides a kit comprising one or more primers and/or one or more probes for the identification of one or more genes associated with antibiotic resistance, wherein the genes are: (A) Imipenem-resistant carbapenemase (IMP), wherein the primers are SEQ ID NO: 296-299 and the probes are SEQ ID NO: 354-356; (B) Mobilized colistin resistance (MCR), wherein the primers are SEQ ID NO: 305-306, 308-309, and 311-312, and the probes are SEQ ID NO: 357-361; (C) Temoniera (TEM), wherein the primers are SEQ ID NO: 314-315; (D) Sulfhydral reagents variable (SHV), wherein the primers are SEQ ID NO: 316-317, and the probe is SEQ ID NO: 362; (E) Guiana extended-spectrum β-lactamase (GES), wherein the primers are SEQ ID NO: 319-320, and the probe is SEQ ID NO: 363; (F) Oxacillinase-type β-lactamase (OXA), wherein the primers are SEQ ID NO: 322-323, 328-329, 331-332, 334-335, and 337-338, and the probes are SEQ ID NO: 364-369, or a combination thereof.

In some embodiments, a kit of the disclosure comprises one or more primers and/or one or more probes for the identification of an imipenem-resistant carbapenemase (IMP) gene, wherein the primers are as set out in SEQ ID NO: 296-299 and the probes are as set out in SEQ ID NO: 354-356, wherein the kit further comprises: (i) primers having SEQ ID NOs: 67-68, 70-71, 73-74, 76-77, 79-80, 89-90, 92-93, 95-96, and 98-99; and (ii) probes having SEQ ID NOs: 69, 72, 75, 78, 81, 87-88, 91, 94, 97, and 100. In some embodiments, the kit further comprises (iii) control sequences having SEQ ID NOs: 261-267 and 269-271. In some embodiments, one or more probes comprises a label. In further embodiments, the label is fluorescein, hexachlorofluorescein, TEX615, TYE™ 665, or a combination thereof. In still further embodiments, SEQ ID NO: 354, as labeled, is as set forth in SEQ ID NO: 300; SEQ ID NO: 355, as labeled, is as set forth in SEQ ID NO: 301; and SEQ ID NO: 356, as labeled, is as set forth in SEQ ID NO: 302. In some aspects, the disclosure provides a kit comprising one or more primers and/or one or more probes for the identification of an imipenem-resistant carbapenemase (IMP) gene, the kit comprising primers having sequences as set out in SEQ ID NOs: 296-299, 67-68, 70-71, 73-74, 76-77, 79-80, 89-90, 92-93, 95-96, and 98-99; probes having sequences as set out in SEQ ID NOs: 354-356, 69, 72, 75, 78, 81, 87-88, 91, 94, 97, and 100; and control sequences having sequences as set out in SEQ ID NOs: 261-267 and 269-271.

In some embodiments, a kit of the disclosure comprises one or more primers and/or one or more probes for the identification of a mobilized colistin resistance (MCR) gene, wherein the primers are as set out in SEQ ID NO: 305-306, 308-309, and 311-312, and the probes are as set out in SEQ ID NO: 357-361, wherein the kit further comprises: (i) primers having SEQ ID NOs: 252, 141, 143, 144, 76, and 77; and (ii) a probe having SEQ ID NO: 340. In some embodiments, the kit further comprises (iii) control sequences having SEQ ID NOs: 341-345 and 264. In further embodiments, one or more probes comprises a label. In some embodiments, the label is fluorescein, hexachlorofluorescein, TEX615, TYE™ 665, or a combination thereof. In still further embodiments, SEQ ID NO: 357, as labeled, is as set forth in SEQ ID NO: 303; SEQ ID NO: 358, as labeled, is as set forth in SEQ ID NO: 304; SEQ ID NO: 359, as labeled, is as set forth in SEQ ID NO: 307; SEQ ID NO: 360, as labeled, is as set forth in SEQ ID NO: 310; and SEQ ID NO: 361, as labeled, is as set forth in SEQ ID NO: 313. In some aspects, the disclosure provides a kit comprising one or more primers and/or one or more probes for the identification of a mobilized colistin resistance (MCR) gene, the kit comprising primers having sequences as set out in SEQ ID NOs: 305-306, 308-309, 311-312, 252, 141, 143, 144, 76, and 77; probes having sequences as set out in SEQ ID NOs: 357-361, 340; and control sequences having sequences as set out in SEQ ID NOs: 341-345 and 264.

In some embodiments, a kit of the disclosure comprises one or more primers and/or one or more probes for the identification of (i) a temoniera (TEM) gene, wherein the primers are as set out in SEQ ID NO: 314-315; (ii) a sulfhydral reagents variable (SHV) gene, wherein the primers are as set out in SEQ ID NO: 316-317, and the probe is as set out in SEQ ID NO: 362; and (iii) a Guiana extended-spectrum β-lactamase (GES) gene, wherein the primers are as set out in SEQ ID NO: 319-320, and the probe is as set out in SEQ ID NO: 363, wherein the kit further comprises: (i) primers having SEQ ID NOs: 76 and 77; and (ii) probes having SEQ ID NOs: 148 and 340. In some embodiments, the kit further comprises (iii) control sequences having SEQ ID NOs: 346-348, and 264. In some embodiments, one or more probes comprises a label. In some embodiments, the label is fluorescein, hexachlorofluorescein, TEX615, TYE™ 665, or a combination thereof. In still further embodiments, SEQ ID NO: 362, as labeled, is as set forth in SEQ ID NO: 318; and SEQ ID NO: 363, as labeled, is as set forth in SEQ ID NO: 321. In some aspects, the disclosure provides a kit comprising one or more primers and/or one or more probes for the identification of (i) a temoniera (TEM) gene, (ii) a sulfhydral reagents variable (SHV) gene, and (iii) a Guiana extended-spectrum β-lactamase (GES) gene, the kit comprising primers having sequences as set out in SEQ ID NOs: 314-315, 316-317, 319-320, wherein the kit further comprises primers having sequences as set out in SEQ ID NOs: 76 and 77, probes having sequences as set out in SEQ ID NOs: 148 and 340, and control sequences having sequences as set out in SEQ ID NOs: 346-348, and 264.

In some embodiments, a kit of the disclosure comprises one or more primers and/or one or more probes for the identification of an oxacillinase-type β-lactamase (OXA) gene, wherein the primers are as set out in SEQ ID NO: 322-323, 328-329, 331-332, 334-335, and 337-338, and the probes are as set out in SEQ ID NO: 364-369, wherein the kit further comprises: (i) primers having SEQ ID NOs: 79-80 and 76-77; and (ii) probes having SEQ ID NOs: 370 and 340. In some embodiments, the kit further comprises (iii) control sequences having SEQ ID NOs: 58, 349-353, and 264. In further embodiments, one or more probes comprises a label. In some embodiments, the label is fluorescein, hexachlorofluorescein, TEX 615, TYE™ 665, or a combination thereof. In still further embodiments, SEQ ID NO: 364, as labeled, is as set forth in SEQ ID NO: 324; SEQ ID NO: 365, as labeled, is as set forth in SEQ ID NO: 330; SEQ ID NO: 366, as labeled, is as set forth in SEQ ID NO: 333; SEQ ID NO: 367, as labeled, is as set forth in SEQ ID NO: 336; SEQ ID NO: 368, as labeled, is as set forth in SEQ ID NO: 339; and SEQ ID NO: 369, as labeled, is as set forth in SEQ ID NO: 340. In some aspects, the disclosure provides a kit comprising one or more primers and/or one or more probes for the identification of an oxacillinase-type β-lactamase (OXA) gene, the kit comprising primers having sequences as set out in SEQ ID NOs: 322-323, 328-329, 331-332, 334-335, and 337-338, 79-80, and 76-77; probes having sequences as set out in 364-369, 370, and 340; and control sequences having sequences as set out in SEQ ID NOs: 58, 349-353, and 264.

In some aspects, a kit of the disclosure comprises the components as set out in Table 9.

TABLE 9
IMP detection kit.

| | |
|---|---|
| | SEQ |
| Sequence (5′-3′) | ID NO |

PCR Mix 1

| | |
|---|---|
| TGGCCAGAACTGACAGGCAAA | 67 |
| TTTCTCCTGAACGTGGCTGGC | 68 |
| 56-FAM/ACGCTAACT/ZEN/CCAGCATTGGTCTGT/3IABKFQ/ | 69 |
| CCGTCACGCTGTTGTTAGG | 70 |
| GCTGTGTTAATCAATGCCACAC | 71 |
| 5HEX/AACTTGCCG/ZEN/AATTAGAGCRGCAGT/3IABKFQ | 72 |
| CGTTTCGTCTGGATCGCAC | 73 |
| GCTGGGTAAAATAGGTCACC | 74 |
| 5TEX615/TATCATTGGTGGTGCCGTAGTCGC/3IAbRQSp | 75 |
| GAGAGGATGAYCAGCCACAC | 76 |
| CGCCCATTGTSCAATATTCC | 77 |
| 5TYE665/TGAGACACGGTCCAGACTCCTACG/3IAbRQSp | 78 |

PCR Mix 2

| | |
|---|---|
| AATCACAGGGCGTAGTTGTG | 79 |
| ACCCACCAGCCAATCTTAGG | 80 |
| 56-FAM/TAGCTTGAT/ZEN/CGCCCTCGATTTGGG/3IABKFQ/ | 81 |
| TTTATCCAGGCCCAGGGCACA | 296 |
| TTTTTTATCCAGGCCCAGGG | 297 |
| ACGGGGTTAGTTATTGGCTGG | 298 |
| GGCCAAGCTTCTAAATTTGCG | 299 |
| 5HEX/CCGGACGGT/ZEN/CTTGGTAATTTGGGT/3IABKFQ | 87 |
| 5HEX/CCGTACGGT/ZEN/TTAGGCAATTTGGGT/3IABKFQ | 88 |
| 5HEX/CCTCACGGC/ZEN/CTTGGTAATTTGGGT/3IABKFQ | 300 |
| 5HEX/ACCGTATGG/ZEN/TCTAGGTAATTTGGGTG/3IABKFQ | 301 |
| 5HEX/CCTCACGGT/ZEN/CTTGGCAATTTAGGT/3IABKFQ | 302 |
| GGCGGCGTTGATGTCCTTCG | 89 |
| CCATTCAGCCAGATCGGCATC | 90 |
| 5TEX615/AGCTCTTCTATCCTGGTGCTGCG/3IAbRQSp | 91 |
| GAGAGGATGAYCAGCCACAC | 76 |
| CGCCCATTGTSCAATATTCC | 77 |
| 5TYE665/TGAGACACGGTCCAGACTCCTACG/3IAbRQSp | 78 |

PCR Mix 3

| | |
|---|---|
| AACTTTCACAGGTGTGCTGGGT | 92 |
| CCGTACGCATACTGGCTTTGC | 93 |
| 56-FAM/AAACCGGGC/ZEN/GATATGCGTCTGTAT/3IABKFQ | 94 |
| GTATCGCCGTCTAGTTCTGC | 95 |
| CCTTGAATGAGCTGCACAGTGG | 96 |
| 5HEX/TCGTCGCGG/ZEN/AACCATTCGCTAAA/3IABKFQ/ | 97 |
| GTTTGATCGTCAGGGATGGC | 98 |
| GGCGAAAGTCAGGCTGTG | 99 |
| 5TEX615/CATCAGGACAAGATGGGCGGTATG/3IAbRQSp | 100 |
| GAGAGGATGAYCAGCCACAC | 76 |
| CGCCCATTGTSCAATATTCC | 77 |
| 5TYE665/TGAGACACGGTCCAGACTCCTACG/3IAbRQSp | 78 |

Control Mix 1

| | |
|---|---|
| TGGCCAGAACTGACAGGCAAACAGTGGCAGGGTATCCGCCTGCTGCACTTAGCCACCT | 261 |
| ATACGGCAGGCGGCCTACCGCTGCAGATCCCCGATGACGTTAGGGATAAAGCCGCATT | |
| ACTGCATTTTTATCAAAACTGGCAGCCGCAATGGACTCCGGGCGCTAAGCGACTTTACG | |
| CTAACTCCAGCATTGGTCTGTTTGGCGCGCTGGCGGTGAAACCCTCAGGAATGAGTTA | |
| CGAAGAGGCAATGACCAGACGCGTCCTGCAACCATTAAAACTGGCGCATACCTGGATT | |
| ACGGTTCCGCAGAACGAACAAAAAGATTATGCCTGGGGCTATCGCGAAGGGAAGCCCG | |
| TACACGTTTCTCCGGGACAACTTGACGCCGAAGCCTATGGCGTGAAATCCAGCGTTATT | |
| GATATGGCCCGCTGGGTTCAGGCCAACATGGATGCCAGCCACGTTCAGGAGAAA | |
| CCGTCACGCTGTTGTTAGGAAGTGTGCCGCTGTATGCGCAAACGGCGGACGTACAGCA | 262 |
| AAAACTTGCCGAATTAGAGCGGCAGTCGGGAGGCAGACTGGGTGTGGCATTGATTAAC | |
| ACAGC | |
| CGTTTCGTCTGGATCGCACTGAACCTACGCTGAATACCGCCATTCCCGGCGACCCGAG | 263 |
| AGACACCACCACGCCGCGGGCGATGGCGCAGACGTTGCGTCAGCTTACGCTGGGTCA | |
| TGCGCTGGGCGAAACCCAGCGGGCGCAGTTGGTGACGTGGCTCAAAGGCAATACGAC | |
| CGGCGCAGCCAGCATTCGGGCCGGCTTACCGACGTCGTGGACTGTGGGTGATAAGAC | |
| CGGCAGCGGCGACTACGGCACCACCAATGATATTGCGGTGATCTGGCCGCAGGGTCG | |
| TGCGCCGCTGGTTCTGGTGACCTATTTTACCCAGC | |
| GAGAGGATGACCAGCCACACTGGAACTGAGACACGGTCCAGACTCCTACGGGAGGCA | 264 |
| GCAGTGGGGAATATTGCACAATGGGCG | |

Control Mix 2

| | |
|---|---|
| AATCACAGGGCGTAGTTGTGCTCTGGAATGAGAATAAGCAGCAAGGATTTACCAATAAT | 265 |
| CTTAAACGGGCGAACCAAGCATTTTTACCCGCATCTACCTTTAAAATTCCCAATAGCTTG | |
| ATCGCCCTCGATTTGGGCGTGGTTAAGGATGAACACCAAGTCTTTAAGTGGGATGGACA | |
| GACGCGCGATATCGCCACTTGGAATCGCGATCATAATCTAATCACCGCGATGAAATATT | |
| CAGTTGTGCCTGTTTATCAAGAATTTGCCCGCCAAATTGGCGAGGCACGTATGAGCAAG | |
| ATGCTACATGCTTTCGATTATGGTAATGAGGACATTTCGGGCAATGTAGACAGTTTCTGG | |
| CTCGACGGTGGTATTCGAATTTCGGCCACGGAGCAAATCAGCTTTTTAAGAAAGCTGTA | |
| TCACAATAAGTTACACGTATCGGAGCGCAGCCAGCGTATTGTCAAACAAGCCATGCTGA | |
| CCGAAGCCAATGGTGACTATATTATTCGGGCTAAAACTGGATACTCGACTAGAATCGAA | |
| CCTAAGATTGGCTGGTGGGT | |
| GCGGAGTTAGTTATTGGCTAGTTAAAAATAAAATTGAAGTTTTTTATCCCGGCCCGGGG | 266 |
| CACACTCAAGATAACGTAGTGGTTTGGTTACCTGAAAAGAAAATTTTATTCGGTGGTTGT | |
| TTTGTTAAACCGGACGGTCTTGGTAATTTGGGTGACGCAAATTTAGAAGCTTGGCC | |
| GGCGGCGTTGATGTCCTTCGGGCGGCTGGGGTGGCAACGTACGCATCACCGTCGACA | 267 |
| CGCCGGCTAGCCGAGGTAGAGGGGAACGAGATTCCCACGCACTCTCTAGAAGGACTCT | |
| CATCGAGCGGGGACGCAGTGCGCTTCGGTCCAGTAGAACTCTTCTATCCTGGTGCTGC | |
| GCATTCGACCGACAACTTAGTTGTGTACGTCCCGTCTGCGAGTGTGCTCTATGGTGGTT | |
| GTGCGATTCATGAGTTGTCACGCACGTCTGCGGGGAACGTGGCCGATGCCGATCTGGC | |
| TGAATGG | |
| GAGAGGATGACCAGCCACACTGGAACTGAGACACGGTCCAGACTCCTACGGGAGGCA | 264 |
| GCAGTGGGGAATATTGCACAATGGGCG | |

Control Mix 3

| | |
|---|---|
| AACTTTCACAGGTGTGCTGGGTGCGGTTTCTGTGGCGAAAAAAGAGATGGCGCTGAAT | 269 |
| GATCCGGCGGCAAAATACCAGCCGGAGCTGGCTCTGCCGCAGTGGAAGGGGATCACA | |
| TTGCTGGATCTGGCTACCTATACCGCAGGCGGACTGCCGTTACAGGTGCCGGATGCGG | |
| TAAAAAGCCGTGCGGATCTGCTGAATTTCTATCAGCAGTGGCAGCCGTCCCGGAAACC | |
| GGGCGATATGCGTCTGTATGCAAACAGCAGTATCGGCCTGTTTGGTGCTCTGACCGCA | |
| AACGCGGGGGGGATGCCGTATGAGCAGTTGCTGACTGCACGGATCCTGGCACCGCTG | |
| GGGTTATCTCACACCTTTATTACTGTGCCGGAAAGTGCGCAAAGCCAGTATGCGTACGG | |
| GTATCGCCGTCTAGTTCTGCTGTCTTGTCTCTCATGGCCGCTGGCTGGCTTTTCTGCCA | 270 |
| CCGCGCTGACCAACCTCGTCGCGGAACCATTCGCTAAACTCGAACAGGACTTTGGCGG | |
| CTCCATCGGTGTGTACGCGATGGATACCGGCTCAGGCGCAACTGTAAGTTACCGCGCT | |
| GAGGAGCGCTTCCCACTGTGCAGCTCATTCAAGG | |
| GTTTGATCGTCAGGGATGGCGGCCGCGTGCTGGTGGTCGATACCGCCTGGACCGATG | 271 |
| ACCAGACCGCCCAGATCCTCAACTGGATCAAGCAGGAGATCAACCTGCCGGTCGCGCT | |
| GGCGGTGGTGACTCACGCGCATCAGGACAAGATGGGGGGTATGGACGCGCTGCATGC | |
| GGCGGGGATTGCGACTTATGCCAATGCGTTGTCGAACCAGCTTGCCCCGCAAGAGGG | |
| GATGGTTGCGGCGCAACACAGCCTGACTTTCGCC | |
| GAGAGGATGACCAGCCACACTGGAACTGAGACACGGTCCAGACTCCTACGGGAGGCA | 264 |
| GCAGTGGGGAATATTGCACAATGGGCG | |

In some aspects, a kit of the disclosure comprises the components as set out in Table 10.

TABLE 10
MCR detection kit.

| | |
|---|---|
| | SEQ |
| Sequence (5′-3′) | ID NO |

PCR Mix

| | |
|---|---|
| CTG TAT GTC AGC GAT CAT | 252 |
| GAT GCC AGT TTG CTT ATC C | 141 |
| 5′/56-FAM/AAG TCT GGG/ZEN/TGA GAA CGG/3IABKFQ/3′ | 303 |
| CAG TCA GTA TGC GAG TTT C | 143 |
| AAA ATT CGC CAA GCC ATC | 144 |
| 5′/5HEX/TGC ATA AGC/ZEN/CAG TGC GTT TTT/3IABKFQ/-3′ | 304 |
| 5′-GAT GTT CGT TCG TGT GGG AC-3′ | 305 |
| 5′-GAA GGA CAA CCT CGT CAT AGC AT-3′ | 306 |
| 5′-/5TEX615/AAA GGC GTC TGC GAC CGA GT/3IAbRQSp/-3′ | 307 |
| 5′-AGG CGT TAC ATT GTC CCT ACC-3′ | 308 |
| 5′-AGC ACG GOG AGG ATC ATA-3′ | 309 |
| 5′-/5TEX615/TCT GCC CGC CCC ATT CGT GAA AAC/3IAbRQSp/-3′ | 310 |
| 5′-GAC TAC GAC GAA CGC CAG ATT-3′ | 311 |
| 5′-TCG GTA GCT TGC GGG ATA G-3′ | 312 |
| 5′-/5TEX615/AGT CGG GCT GTA AAG GCG TCT GT/3IAbRQSp/3′ | 313 |
| 5′-GAG AGG ATG AYC AGC CAC AC-3′ | 76 |
| 5′-CGC CCA TTG TSC AAT ATT CC-3′ | 77 |
| 5′-/5Cy5/TGA GAC ACG/TAO/GTC CAG ACT CCT ACG/3IAbRQSp/-3′ | 340 |

Control Mix

| | |
|---|---|
| CTGTATGTCAGCGATCATGGCGAAAGTCTGGGTGAGAACGGTGTCTATCTACATGGTAT | 341 |
| GCCAAATGCCTTTGCACCAAAAGAACAGCGCAGTGTGCCTGCATTTTTCTGGACGGATA | |
| AGCAAACTGGCATC | |
| CAGTCAGTATGCGAGTTTCTTTCGGGTGCATAAGCCAGTGCGTTTTTATATCAACCCGAT | 342 |
| TACGCCGATTTATTCGGTGGGTAAGCTTGCCAGTATCGAGTACAAAAAAGCCACTGCGC | |
| CAACAGACACCATCTATCATGCCAAAGACGCCGTGCAGCCACCAAGCCGAGCGAGCGT | |
| AAGCCACGCCTAGTGGTGTTCGTCGTCGGTGAGACGGCGCGTGCTGACCATGTGCAGT | |
| TCAATGGCTATGGCCGTGAGACTTTCCCGCAGCTTGCCAAAGTTGATGGCTTGGCGAAT | |
| TTT | |
| TTTAATGATGTTCGTTCGTGTGGGACTGCAACCGCTGTATCCGTCCCCTGCATGTTCTC | 343 |
| CAATATGGGGAGAAAGGAGTTTGATGATAATCGCGCTCGCAATAGCGAGGGCCTGCTA | |
| GATGTGTTGCAAAAAACGGGGATCTCCATTTTTTGGAAGGAGAACGATGGAGGCTGCAA | |
| AGGCGTCTGCGACCGAGTACCTAACATCGAAATCGAACCAAAGGATCACCCTAAGTTCT | |
| GCGATAAAAACACATGCTATGACGAGGTTGTCCTTCAAGACCTC | |
| TGAGTTAAGGCGTTACATTGTCCCTACCTATTTTGTCAGTAGTGCATCTAAATATCTCAAT | 344 |
| GAGCACTATTTGCAGACGCCCATGGAATACCAACAACTTGGCCTAGATGCGAAGAATGC | |
| CAGTCGTAACCCGAACACTAAACCTAACTTATTAGTGGTTGTTGTGGGTGAAACTGCGC | |
| GCTCAATGAGCTATCAATATTATGGATATAACAAGCCAACCAATGCTCATACCCAAAATC | |
| AGGGGCTGATTGCGTTTAACGATACTAGCTCATGCGGCACGGCCACGGCGGTGTCTCT | |
| ACCCTGTATGTTTTCACGAATGGGGGGGGCAGACTATGATCCTCGCCGTGCTAATGCTC | |
| TCGGCGCGACTACGACGAACGCCAGATTCGTCGGCGCGAGTCCGTGCTGCACGTTTTA | 345 |
| AACCGTAGTGACGTCAACATTCTCTGGCGCGATAACCAGTCGGGCTGTAAAGGCGTCT | |
| GTGATGGACTGCCCTTTGAAAACCTGTCTTCGGCAGGCCATCCCACACTGTGCCATGG | |
| CGTGCGCTGCCTGGATGAAATTCTGCTCGAAGGGTTGGCCGAGAAGATAACAACAAGC | |
| CGCAGCGATATGCTGATCGTTCTGCATATGCTGGGCAATCACGGCCCAGCGTATTTCCT | |
| GCGCTATCCCGCAAGCTACCGACGCTGG | |
| GAGAGGATGACCAGCCACACTGGAACTGAGACACGGTCCAGACTCCTACGGGAGGCA | 264 |
| GCAGTGGGGAATATTGCACAATGGGCG | |

In some aspects, a kit of the disclosure comprises the components as set out in Table 11.

TABLE 11
TEM-SHV-GES detection kit.

| | |
|---|---|
| | SEQ |
| Sequence (5′-3′) | ID NO |

PCR Mix

| | |
|---|---|
| 5′-CGG TCG CCG CAT ACA CTA TT-3′ | 314 |
| 5′-CAG TGC TGC AAT GAT ACC GC-3′ | 315 |
| 5′-/56-FAM/ATG AAG CCA/ZEN/TAC CAA ACG ACG AGC/3IABKFQ/-3′ | 148 |
| 5′-CGC CAT TAC CAT GAG CGA TAA-3′ | 316 |
| 5′-GGA AGC GCC TCA TTC AGT T-3′ | 317 |
| 5′-/5HEX/ACA ACG TCA/ZEN/CCC GCC TTG AC/3IABKFQ/-3′ | 318 |
| 5′-GCT GAT CGG AAA CCA AAC GG-3′ | 319 |
| 5′-ACT TGA CCG ACA GAG GCA AC-3′ | 320 |
| 5′-/5TEX615/AAA CCA ATG TCG TTC CGG CCC/3IAbRQSp/-3′ | 321 |
| 5′-GAG AGG ATG AYC AGC CAC AC-3′ | 76 |
| 5′-CGC CCA TTG TSC AAT ATT CC-3′ | 77 |
| 5′-/5Cy5/TGA GAC ACG/TAO/GTC CAG ACT CCT ACG/3IAbRQSp/-3′ | 340 |

Control Mix

| | |
|---|---|
| CGGTCGCCGCATACACTATTCTCAGAATGACTTGGTTGAGTACTCACCAGTCACAGAAA | 346 |
| AGCATCTTACGGATGGCATGACAGTAAGAGAATTATGCAGTGCTGCCATAACCATGAGT | |
| GATAACACTGCGGCCAACTTACTTCTGACAACGATCGGAGGACCGAAGGAGCTAACCG | |
| CTTTTTTGCACAACATGGGGGATCATGTAACTCGCCTTGATCGTTGGGAACCGGAGCTG | |
| AATGAAGCCATACCAAACGACGAGCGTGACACCACGACGCCTGCAGCAATGGCAACAA | |
| CGTTGCGCAAACTATTAACTGGCGAACTACTTACTCTAGCTTCCCGGCAACAATTAATAG | |
| ACTGGATGGAGGCGGATAAAGTTGCAGGACCACTTCTGCGCTCGGCCCTTCCGGCTGG | |
| CTGGTTTATTGCTGATAAATCTGGAGCCAGTGAGCGTGGGTCTCGCGGTATCATTGCAG | |
| CACTG | |
| CGCCATTACCATGAGCGATAACAGCGCCGCCAATCTGCTGCTGGCCACCGTCGGCGGC | 347 |
| CCCGCAGGATTGACTGCCTTTTTGCGCCAGATCGGCGACAACGTCACCCGCCTTGACC | |
| GCTGGGAAACGGAACTGAATGAGGCGCTTCC | |
| GCTGATCGGAAACCAAACGGGAGACGCGACACTACGAGCGGGTTTTCCTAAAGATTGG | 348 |
| GTTGTTGGAGAGAAAACTGGTACCTGCGCCAACGGGGGCCGGAACGACATTGGTTTTT | |
| TTAAAGCCCAGGAGAGAGATTACGCTGTAGCGGTGTATACAACGGCCCCGAAACTATC | |
| GGCCGTAGAACGTGACGAATTAGTTGCCTCTGTCGGTCAAGT | |
| GAGAGGATGACCAGCCACACTGGAACTGAGACACGGTCCAGACTCCTACGGGAGGCA | 264 |
| GCAGTGGGGAATATTGCACAATGGGCG | |

In some aspects, a kit of the disclosure comprises the components as set out in Table 12.

TABLE 12
OXA detection kit.

| | |
|---|---|
| | SEQ |
| Sequence (5′-3′) | ID NO |

PCR Mix 1

| | |
|---|---|
| 5′-GGT AAT AAC CTG GTA CGA GCA CAT AC-3′ | 322 |
| 5′-ACC AAA ACC AAC CCG TTT AAC TTC T-3′ | 323 |
| 5′-/56-FAM/CCA TGG CAC/ZEN/TTT CAG CAG TTC CTG T/3IABKFQ/-3′ | 324 |
| 5′- AAT CAC AGG GCG TAG TTG TG-3′ | 79 |
| 5′-ACC CAC CAG CCA ATC TTA GG-3′ | 80 |
| 5′-/5HEX/TAG CTT GAT/ZEN/CGC CCT CGA TTT GGG/3IABKFQ/-3′ | 370 |
| 5′-CTT AGC ACC TAT GGT AAT GCT CTT GC-3′ | 328 |
| 5′-TTC TGC ATT AGC TCT AGG CCA G-3′ | 329 |
| 5′-/5TEX615/ACT TTA GGT GAG GCA ATG GCA TTG TCA GC/3IAbRQSp/-3′ | 330 |
| 5′-GAG AGG ATG AYC AGC CAC AC-3′ | 76 |
| 5′-CGC CCA TTG TSC AAT ATT CC-3′ | 77 |
| 5′-/5Cy5/TGA GAC ACG/TAO/GTC CAG ACT CCT ACG/3IAbRQSp/-3′ | 340 |

PCR Mix 2

| | |
|---|---|
| 5′-CAT CGA TCA GAA TGT TCA AGC GC-3′ | 331 |
| 5′-CCA ATA CGA CGT GCC AAT TCT TG-3′ | 332 |
| 5′-/56-FAM/TGG CAC GCA/ZEN/TTT AGA CCG AGC AAA AAC AG/3IABKFQ/-3′ | 333 |
| 5′-CTC GTG CTT CGA CCG AGT ATG-3′ | 334 |
| 5′-TTA ACC AGC CTA CTT GTG GGT-3′ | 335 |
| 5′-/5HEX/CCT GCT TCG /ZEN/ACC TTC AAA ATG CTT AAT GCT/3IABKFQ/-3′ | 336 |
| 5′-TAC AGA ATA TGT GCC AGC CTC TAC-3′ | 337 |
| 5′-GCA TGA GAT CAA GAC CGA TAC GTC-3′ | 338 |
| 5′-/5TEX615/TGC CCT GAT CGG ATT GGA GAA CCA/3IAbRQSp/-3′ | 339 |
| 5′-GAG AGG ATG AYC AGC CAC AC-3′ | 76 |
| 5′-CGC CCA TTG TSC AAT ATT CC-3′ | 77 |
| 5′-/5Cy5/TGA GAC ACG/TAO/GTC CAG ACT CCT ACG/3IAbRQSp/-3′ | 340 |

Control Mix 1

| | |
|---|---|
| TATGGTAATAACCTGGTACGAGCACATACAGAATATGTCCCTGCGTCAACATTTAAGATG | 349 |
| CTAAATGCCTTAATTGGATTAGAAAATCATAAAGCTACAACAACTGAGATTTTCAAATGG | |
| GATGGTAAAAAAAGATCTTATCCTATGTGGGAAAAAGATATGACTTTAGGTGATGCCATG | |
| GCACTTTCAGCAGTTCCTGTATATCAAGAACTTGCAAGACGGACTGGCTTAGATCTAAT | |
| GCAAAAAGAAGTTAAACGGGTTGGTTTTGGTAAT | |
| AATCACAGGGCGTAGTTGTGCTCTGGAATGAGAATAAGCAGCAAGGATTTACCAATAAT | 58 |
| CTTAAACGGGCGAACCAAGCATTTTTACCCGCATCTACCTTTAAAATTCCCAATAGCTTG | |
| ATCGCCCTCGATTTGGGCGTGGTTAAGGATGAACACCAAGTCTTTAAGTGGGATGGACA | |
| GACGCGCGATATCGCCACTTGGAATCGCGATCATAATCTAATCACCGCGATGAAATATT | |
| CAGTTGTGCCTGTTTATCAAGAATTTGCCCGCCAAATTGGCGAGGCACGTATGAGCAAG | |
| ATGCTACATGCTTTCGATTATGGTAATGAGGACATTTCGGGCAATGTAGACAGTTTCTGG | |
| CTCGACGGTGGTATTCGAATTTCGGCCACGGAGCAAATCAGCTTTTTAAGAAAGCTGTA | |
| TCACAATAAGTTACACGTATCGGAGCGCAGCCAGCGTATTGTCAAACAAGCCATGCTGA | |
| CCGAAGCCAATGGTGACTATATTATTCGGGCTAAAACTGGATACTCGACTAGAATCGAA | |
| CCTAAGATTGGCTGGTGGGT | |
| AATCTTAGCACCTATGGTAATGCTCTTGCACGAGCAAATAAAGAATATGTCCCTGCATCA | 350 |
| ACATTTAAGATGCTAAATGCTTTAATCGGGCTAGAAAATCGCGGGCAACAACAAATGAG | |
| ATTTTCAAATGGGATGGTAAAAAAAGAACTTATCCTATGTGGGAGAAAGATATGACTTTA | |
| GGTGAGGCAATGGCATTGTCAGCAGTTCCAGTATATCAAGAGCTTGCAAGACGGACTG | |
| GCCTAGAGCTAATGCAGAAAGAAGTAAAGC | |
| GAGAGGATGACCAGCCACACTGGAACTGAGACACGGTCCAGACTCCTACGGGAGGCA | 264 |
| GCAGTGGGGAATATTGCACAATGGGCG | |
| Control Mix 2 | |
| TCAATCATCGATCAGAATGTTCAAGCGCTTTTTAATGAAATCTCAGCTGATGCTGTGTTT | 351 |
| GTCACATATGATGGTCAAAATATTAAAAAATATGGCACGCATTTAGACCGAGCAAAAACA | |
| GCTTATATTCCTGCATCTACATTTAAAATTGCCAATGCACTAATTGGTTTAGAAAATCATA | |
| AAGCAACATCTACAGAAATATTTAAGTGGGATGGAAAGCCACGTTTTTTTAAAGCATGGG | |
| ACAAAGATTTTACTTTGGGCGAAGCCATGCAAGCATCTACAGTGCCTGTATATCAAGAAT | |
| TGGCACGTCGTATTGGTCCAAGCTTAATGCA | |
| GTAATGATCTTGCTCGTGCTTCGACCGAGTATGTACCTGCTTCGACCTTCAAAATGCTTA | 352 |
| ATGCTTTGATCGGCCTTGAGCACCATAAGGCAACCACCACAGAAGTATTTAAGTGGGAC | |
| GGGCAAAAAAGGCTATTCCCAGAATGGGAAAAGGACATGACCCTAGGCGATGCTATGA | |
| AAGCTTCCGCTATTCCGGTTTATCAAGATTTAGCTCGTCGTATTGGACTTGAACTCATGT | |
| CTAAGGAAGTGAAGCGTGTTGGTTATGGCAATGCAGATATCGGTACCCAAGTCGATAAT | |
| TTTTGGCTGGTGGGTCCTTTAAAAATTACTCCTCAGCAAGAGGCACAGTTTGCTTACAAG | |
| CTAGCTAATAAAACGCTTCCATTTAGCCCAAAAGTCCAAGATGAAGTGCAATCCATGTTA | |
| TTCATAGAAGAAAAGAATGGAAATAAAATATACGCAAAAAGTGGTTGGGGATGGGATGT | |
| AGACCCACAAGTAGGCTGGTTAACTGGATGGG | |
| CAAATACAGAATATGTGCCAGCCTCTACATTTAAAATGTTGAATGCCCTGATCGGATTGG | 353 |
| AGAACCAGAAAACGGATATTAATGAAATATTTAAATGGAAGGGCGAGAAAAGGTCATTTA | |
| CCGCTTGGGAAAAAGACATGACACTAGGAGAAGCCATGAAGCTTTCTGCAGTCCCAGT | |
| CTATCAGGAACTTGCGCGACGTATCGGTCTTGATCTCATGCAAAAAG | |
| GAGAGGATGACCAGCCACACTGGAACTGAGACACGGTCCAGACTCCTACGGGAGGCA | 264 |
| GCAGTGGGGAATATTGCACAATGGGCG | |

TABLE 2

| | Primer/ | |
|---|---|---|
| | Probe | Sequence |
| SEQ ID NO. 1 | MOX F′ | AGA CCC TGT TCG AGA TAG |
| SEQ ID NO. 2 | MOX R′ | ATG GTG ATG CTG TCA AAG |
| SEQ ID NO. 3 | MOX-FAM | 5′-56-FAM-CGT GAG CAA GAC CCT GAC TG-3′BHQ1 |
| SEQ ID NO. 4 | FOX F′ | ACT ATT TCA ACT ATG GGG TT |
| SEQ ID NO. 5 | FOX R′ | TTG TCA TCC AGC TCA AAG |
| SEQ ID NO. 6 | FOX-TEX | 5′-Tex615-TGA CCG CAG CAT AGG CAC-3′BHQ-2 |
| SEQ ID NO. 7 | EBC F′ | GTG GCG GTG ATT TAT GAG |
| SEQ ID NO. 8 | EBC R′ | CGG TGA AGG TTT TAC TTA TAG AA |
| SEQ ID NO. 9 | EBC-HEX | 5′-5HEX/CAGCCGCAC/ZEN/TACTTCACCT/-3′BHQ-1 |
| SEQ ID NO. 10 | DHA F′ | TGCGTACGGTTATGAGAACAA |
| SEQ ID NO. 11 | DHA R′ | CCCAGCGCAGCATATCTT |
| SEQ ID NO. 12 | DHA-FAM | ATGCGGAATCTTACGGCGTGGAAT |
| SEQ ID NO. 13 | CMY F′ | TCC AGC GTT ATT GAT ATG G |
| SEQ ID NO. 14 | CMY R′ | CAT CTC CCA GCC TAA TCC |
| SEQ ID NO. 15 | CMY-TEX | 5′TexRd-XN/ACATATCGCCAATACGCCAGT/3IAbRQSp/-3′ |
| SEQ ID NO. 16 | ACC F′ | GCCGCTGATGCAGAAGAATA |
| SEQ ID NO. 17 | ACC R′ | TTT GCC GCT AAC CCA TAG TT |
| SEQ ID NO. 18 | ACC-HEX | 5′-/5HEX/TCA CTG CGA/ZEN/CCG ACA TAC CG/3IABKFQ/-3′ |
| SEQ ID NO. 19 | IC F′ | GAG AGG ATG ACC AGC CAC AC |
| SEQ ID NO. 20 | IC R′ | AGT ACT TTA CAA CCC GAA GGC |
| SEQ ID NO. 21 | IC-TYE | 5′-/5TYE665/TGA GAC ACG GTC CAG ACT CCT ACG G/3BHQ_2/-3′ |
| SEQ ID NO. 22 | CTX-M-14 F′ | 5′-TTGGTGACGTGGCTCAAA-3′ |
| SEQ ID NO. 23 | CTX-M-14 R′ | 5′-ATATCATTGGTGGTGCCGTAG-3′ |
| SEQ ID NO. 24 | CTX-M-14- | 5′-/56-FAM/CGTGGACTG/ZEN/TGGGTGATAAGACCG/3IABKFQ/-3′ |
| | FAM | |
| SEQ ID NO. 25 | CTX-M-15 F′ | 5′-GTCACGCTGTTGTTAGGAAGT-3′ |
| SEQ ID NO. 26 | CTX-M-15 R′ | 5′-TAATCAATGCCACACCCAGTC-3′ |
| SEQ ID NO. 27 | CTX-M-15- | 5′-/5TEX615/AACTTGCCGAATTAGAGCGGCAGT/3BHQ_2/-3′ |
| | TEX615 | |
| SEQ ID NO. 28 | OXA48-F′ | 5′-AGCAGCAAGGATTTACCAATAATC-3′ |
| SEQ ID NO. 29 | OXA48-R′ | 5′-CGTCTGTCCATCCCACTTAAA-3′ |
| SEQ ID NO. 30 | OXA48-HEX | 5′-/5HEX/TAGCTTGAT/ZEN/CGCCCTCGATTTGGG/3IABKFQ/-3′ |
| SEQ ID NO. 31 | CMY F′ | 5′-TCCAGCGTTATTGATATGG-3′ |
| SEQ ID NO. 32 | CMY R′ | 5′-CATCTCCCAGCCTAATCC-3′ |
| SEQ ID NO. 33 | CMY-TxR | 5′-/5TexRd-XN/ACATATCGCCAATACGCCAGT/3IAbRQSp/-3′ |
| SEQ ID NO. 34 | NDM F′ | 5′-TTTGATCGTCAGGGATGGC-3′ |
| SEQ ID NO. 35 | NDM R′ | 5′-CAGGTTGATCTCCTGCTTGAT-3′ |
| SEQ ID NO. 36 | NDM-HEX | 5′-/5HEX/AGACCGCCC/ZEN/AGATCCTCAACTG/3IABKFQ/-3′ |
| SEQ ID NO. 37 | KPC F′ | 5′-CGCTAAACTCGAACAGGACTT-3′ |
| SEQ ID NO. 38 | KPC R′ | 5′-TAACTTACAGTTGCGCCTGAG-3′ |
| SEQ ID NO. 39 | KPC-FAM | 5′-/5TYE665/ATCGGTGTGTACGCGATGGATACC/3BHQ_2/-3′ |
| SEQ ID NO. 40 | VIM F′ | 5′-CATTCGACCGACAACTTAG-3′ |
| SEQ ID NO. 41 | VIM R′ | 5′-CGTGCGTGACAACTCAT-3′ |
| SEQ ID NO. 42 | VIM-TEX | 5′-/5TEX615/TGTGCTCTATGGTGGTTGTGCGAT/3BHQ_2/-3′ |
| SEQ ID NO. 43 | DHA F′ | 5′-TGCGTACGGTTATGAGAACAA-3′ |
| SEQ ID NO. 44 | DHA R′ | 5′-CCCAGCGCAGCATATCTT-3′ |
| SEQ ID NO. 45 | DHA-FAM | 5′-/56-FAM/ATGCGGAAT/ZEN/CTTACGGCGTGAAAT/3IABKFQ-3′ |
| SEQ ID NO. 46 | IMP F′ | 5′-ACGTAGTGGTTTGGTTACCTG-3′ |
| SEQ ID NO. 47 | IMP R′ | 5′-AAGCTTCTAAATTTGCGTCACC-3′ |
| SEQ ID NO. 48 | IMP-TYE705 | 5′-/5HEX/TTTGTTAAA/ZEN/CCGGACGGTCTTGGT/3IABKFQ/-3′ |

TABLE 3

| | DNA | |
|---|---|---|
| | Control | Sequence |
| SEQ ID NO. 49 | MOX | AACCGGGAGAGCGGGGCCAGCGTCAGCGAGCAGACCCTGTTCGAGATAGG |
| | | ATCCGTGAGCAAGACCCTGACTGCGACCCTGGGGGCCTATGCGGTGGTCA |
| | | AGGGAGCGATGCAGCTGGATGACAAGGCGAGCCGGCACGCGCCCTGGCTC |
| | | AAGGGATCCGTCTTTGACAGCATCACCATGGGGGAGCTTGCCACCTACAGC |
| SEQ ID NO. 50 | FOX | GGGGATGGCGGTCGCCGTGCTGAAAGATGGCAAGGCCCACTATTTCAACTA |
| | | TGGGGTTGCCAACCGCGAGAGTGGTCAGCGCGTCAGCGAGCAGACCCTGT |
| | | TCGAGATTGGCTCGGTCAGCAAGACCCTGACCGCGACCCTCGGTGCCTATG |
| | | CTGCGGTCAAGGGGGGCTTTGAGCTGGATGACAAGGTGAGCCAGCACGCC |
| | | CCCTGGCTCAAAGGTTCCGCCTTTGATGGTGTGACCAT |
| SEQ ID NO. 51 | EBC | GGACCGTTACGCCGCTGATGAAAGCGCAGGCCATTCCGGGTATGGCGGTG |
| | | GCGGTGATTTATGAGGGTCAGCCGCACTACTTCACCTTCGGTAAAGCCGAT |
| | | GTTGCGGCGAACAAACCTGTCACTCCACAAACCTTGTTCGAACTGGGTTCTA |
| | | TAAGTAAAACCTTCACCGGCGTACTCGGTGGCGATGCCATTGCTCGCGGTG |
| | | AAATATCGCTGGGCGA |
| SEQ ID NO. 52 | DHA | GACTGCACGGATCCTGGCACCGCTGGGGTTATCTCACACCTTTATTACTGTG |
| | | CCGGAAAGTGCGCAAAGCCAGTATGCGTACGGTTATGAGAACAAAAAACCG |
| | | GTCCGCGTGTCGCCGGGACAGCTTGATGCGGAATCTTACGGCGTGGAATCC |
| | | GCCTCAAAAGATATGCTGCGCTGGGCGGAAATGAATATGGAGCCGTCACGG |
| | | GCCGGTAATGCGGAT |
| SEQ ID NO. 53 | CMY | GCCTGTACACGTTTCTCCGGGACAACTTGACGCCGAAGCCTATGGCGTGAA |
| | | ATCCAGCGTTATTGATATGGCCCGCTGGGTTCAGGTCAACATGGACGCCAG |
| | | CCGCGTTCAGGAGAAAACGCTCCAGCAGGGCATTGCGCTTGCGCAGTCTCG |
| | | CTACTGGCGTATTGGCGATATGTACCAGGGATTAGGCTGGGAGATGCTGAA |
| | | CTGGCCGCTGAAAGCTGATTCGATCATCAACGGTAGCGACAGCAAA |
| | | GTGGCATTGG |
| SEQ ID NO. 54 | ACC | GAGAGCAAAATTAAAGACACCGTTGATGACCTGATCCAGCCGCTGATGCAG |
| | | AAGAATAATATTCCCGGTATGTCGGTCGCAGTGACCGTCAACGGTAAAAACT |
| | | ACATTTATAACTATGGGTTAGCGGCAAAACAGCCTCAGCAGCCGGTT |
| SEQ ID NO. 55 | IC | AGCTTGTTGGTGGGGTAACGGCTCACCAAGGCGACGATCCCTAGCTGGTCT |
| | | GAGAGGATGACCAGCCACACTGGAACTGAGACACGGTCCAGACTCCTACGG |
| | | GAGGCAGCAGTGGGGAATATTGCACAATGGGCGCAAGCCTGATGCAGCCAT |
| | | GCCGCGTGTATGAAGAAGGCCTTCGGGTTGTAAAGTACTTTCAGCGGGGAG |
| | | GAAGGGAGTAAAGTTAATACCTTTGCTCATTGACGTTACCCGCAGAAGAAGC |
| | | ACCGGCTAACTCCG |
| SEQ ID NO. 56 | CTX-M- | CGTTTCGTCTGGATCGCACTGAACCTACGCTGAATACCGCCATTCCCGGCG |
| | 14 | ACCCGAGAGACACCACCACGCCGCGGGCGATGGCGCAGACGTTGCGTCAG |
| | | CTTACGCTGGGTCATGCGCTGGGCGAAACCCAGCGGGCGCAGTTGGTGAC |
| | | GTGGCTCAAAGGCAATACGACCGGCGCAGCCAGCATTCGGGCCGGCTTAC |
| | | CGACGTCGTGGACTGTGGGTGATAAGACCGGCAGCGGCGACTACGGCACC |
| | | ACCAATGATATTGCGGTGATCTGGCCGCAGGGTCGTGCGCCGCTGGTTCTG |
| | | GTGACCTATTTTACCCAGC |
| SEQ ID NO. 57 | CTX-M- | CCGTCACGCTGTTGTTAGGAAGTGTGCCGCTGTATGCGCAAACGGCGGACG |
| | 15 | TACAGCAAAAACTTGCCGAATTAGAGCGGCAGTCGGGAGGCAGACTGGGTG |
| | | TGGCATTGATTAACACAGC |
| SEQ ID NO. 58 | OXA | AATCACAGGGCGTAGTTGTGCTCTGGAATGAGAATAAGCAGCAAGGATTTAC |
| | | CAATAATCTTAAACGGGCGAACCAAGCATTTTTACCCGCATCTACCTTTAAAA |
| | | TTCCCAATAGCTTGATCGCCCTCGATTTGGGCGTGGTTAAGGATGAACACCA |
| | | AGTCTTTAAGTGGGATGGACAGACGCGCGATATCGCCACTTGGAATCGCGA |
| | | TCATAATCTAATCACCGCGATGAAATATTCAGTTGTGCCTGTTTATCAAGAAT |
| | | TTGCCCGCCAAATTGGCGAGGCACGTATGAGCAAGATGCTACATGCTTTCG |
| | | ATTATGGTAATGAGGACATTTCGGGCAATGTAGACAGTTTCTGGCTCGACGG |
| | | TGGTATTCGAATTTCGGCCACGGAGCAAATCAGCTTTTTAAGAAAGCTGTAT |
| | | CACAATAAGTTACACGTATCGGAGCGCAGCCAGCGTATTGTCAAACAAGCCA |
| | | TGCTGACCGAAGCCAATGGTGACTAATTATTCGGGCTAAAACTGGATACTCG |
| | | ACTAGAATCGAACCTAAGATTGGCTGGCTGGGT |
| SEQ ID NO. 59 | IC | CGGAGTTAGCCGGTGCTTCTTCTGCGGGTAACGTCAATGAGCAAAGGTATTA |
| | | ACTTTACTCCCTTCCTCCCCGCTGAAAGTACTTTACAACCCGAAGGCCTTCTT |
| | | CATACACGCGGCATGGCTGCATCAGGCTTGCGCCCATTGTGCAATATTCCC |
| | | CACTGCTGCCTCCCGTAGGAGTCTGGACCGTGTCTCAGTTCCAGTGTGGCT |
| | | GGTCATCCTCTCAGACCAGCTAGGGATCGTCGCCTTGGTGAGCCGTTACCC |
| | | CACCAACAAGCT |
| SEQ ID NO. 60 | CMY | GCCTGTACACGTTTCTCCGGGACAACTTGACGCCGAAGCCTATGGCGTGAA |
| | | ATCCAGCGTTATTGATATGGCCCGCTGGGTTCAGGTCAACATGGACGCCAG |
| | | CCGCGTTCAGGAGAAAACGCTCCAGCAGGGCATTGCGCTTGCGCAGTCTCG |
| | | CTACTGGCGTATTGGCGATATGTACCAGGGATTAGGCTGGGAGATGCTGAA |
| | | CTGGCCGCTGAAAGCTGATTCGATCATCAACGGTAGCGACAGCAAAGTGGC |
| | | ATTGG |
| SEQ ID NO. 61 | NDM | GGCGAAAGTCAGGCTGTGTTGCGCCGCAACCATCCCCTCTTGCGGGGCAA |
| | | GCTGGTTCGACAACGCATTGGCATAAGTCGCAATCCCCGCCGCATGCAGCG |
| | | CGTCCATACCGCCCATCTTGTCCTGATGCGCGTGAGTCACCACCGCCAGCG |
| | | CGACCGGCAGGTTGATCTCCTGCTTGATCCAGTTGAGGATCTGGGCGGTCT |
| | | GGTCATCGGTCCAGGCGGTATCGACCACCAGCACGCGGCCGCCATCCCTG |
| | | ACGATCAAAC |
| SEQ ID NO. 62 | KPC | GTATCGCCGTCTAGTTCTGCTGTCTTGTCTCTCATGGCCGCTGGCTGGCTTT |
| | | TCTGCCACCGCGCTGACCAACCTCGTCGCGGAACCATTCGCTAAACTCGAA |
| | | CAGGACTTTGGCGGCTCCATCGGTGTGTACGCGATGGATACCGGCTCAGGC |
| | | GCAACTGTAAGTTACCGCGCTGAGGAGCGCTTCCCACTGTGCAGCTCATTC |
| | | AAGG |
| SEQ ID NO. 63 | VIM | CCATTCAGCCAGATCGGCATCGGCCACGTTCCCCGCAGACGTGCGTGACAA |
| | | CTCATGAATCGCACAACCACCATAGAGCACACTCGCAGACGGGACGTACAC |
| | | AACTAAGTTGTCGGTCGAATGCGCAGCACCAGGATAGAAGAGTTCTACTGG |
| | | ACCGAAGCGCACTGCGTCCCCGCTCGAGTCCTTCTAGAGAGTGCGTGGGAA |
| | | TCTCGTTCCCCTCTACCTCGGCTAGCCGGCGTGTCGACGGTGATGCGTACG |
| | | TTGCCACCCCAGCCGCCCGAAGGACATCAACGCCGCC |
| SEQ ID NO. 64 | DHA | GACTGCACGGATCCTGGCACCGCTGGGGTTATCTCACACCTTTATTACTGTG |
| | | CCGGAAAGTGCGCAAAGCCAGTATGCGTACGGTTATGAGAACAAAAAACCG |
| | | GTCCGCGTGTCGCCGGGACAGCTTGATGCGGAATCTTACGGCGTGAAATCC |
| | | GCCTCAAAAGATATGCTGCGCTGGGCGGAAATGAATATGGAGCCGTCACGG |
| | | GCCGGTAATGCGGAT |
| SEQ ID NO. 65 | IC | CGGAGTTAGCCGGTGCTTCTTCTGCGGGTAACGTCAATGAGCAAAGGTATTA |
| | | ACTTTACTCCCTTCCTCCCCGCTGAAAGTACTTTACAACCCGAAGGCCTTCTT |
| | | CATACACGCGGCATGGCTGCATCAGGCTTGCGCCCATTGTGCAATATTCCC |
| | | CACTGCTGCCTCCCGTAGGAGTCTGGACCGTGTCTCAGTTCCAGTGTGGCT |
| | | GGTCATCCTCTCAGACCAGCTAGGGATCGTCGCCTTGGTGAGCCGTTACCC |
| | | CACCAACAAGCT |
| SEQ ID NO. 66 | IMP | GCGGAGTTAGTTATTGGCTAGTTAAAAATAAAATTGAAGTTTTTTATCCCGGC |
| | | CCGGGGCACACTCAAGATAACGTAGTGGTTTGGTTACCTGAAAAGAAAATTT |
| | | TATTCGGTGGTTGTTTTGTTAAACCGGACGGTCTTGGTAATTTGGGTGACGC |
| | | AAATTTAGAAGCTTGGCC |

TABLE 4
Primer/Probe Sequences

| Kit | 5′-3′ Sequence | SEQ ID NO |
|---|---|---|
| BID | TTTATCCAGGCCCAGGGCACA | 296 |
| BID | TTTTTTATCCAGGCCCAGGG | 297 |
| BID | ACGGGGTTAGTTATTGGCTGG | 298 |
| BID | GGCCAAGCTTCTAAATTTGCG | 299 |
| BID | 5HEX/CCTCACGGC/ZEN/CTTGGTAATTTGGGT/3IABKFQ | 300 |
| BID | CCTCACGGCCTTGGTAATTTGGGT | 354 |
| BID | 5HEX/ACCGTATGG/ZEN/TCTAGGTAATTTGGGTG/3IABKFQ | 301 |
| BID | ACCGTATGGTCTAGGTAATTTGGGTG | 355 |
| BID | 5HEX/CCTCACGGT/ZEN/CTTGGCAATTTAGGT/3IABKFQ | 302 |
| BID | CCTCACGGTCTTGGCAATTTAGGT | 356 |
| MCR | 56-FAM/AAG TCT GGG/ZEN/TGA GAA CGG/3IABKFQ | 303 |
| MCR | AAGTCTGGGTGAGAACGG | 357 |
| MCR | 5′/5HEX/TGC ATA AGC/ZEN/CAG TGC GTT TTT/3IABKFQ/-3′ | 304 |
| MCR | TGCATAAGCCAGTGCGTTTTT | 358 |
| MCR | 5′-GAT GTT CGT TCG TGT GGG AC-3′ | 305 |
| MCR | 5′-GAA GGA CAA CCT CGT CAT AGC AT-3′ | 306 |
| MCR | 5′-/5TEX615/AAA GGC GTC TGC GAC CGA GT/3IABRQSP/-3′ | 307 |
| MCR | AAAGGCGTCTGCGACCGAGT | 359 |
| MCR | 5′-AGG CGT TAC ATT GTC CCT ACC-3′ | 308 |
| MCR | 5′-AGC ACG GCG AGG ATC ATA-3′ | 309 |
| MCR | 5′-/5Tex615/TCT GCC CGC CCC ATT CGT GAA AAC/3IABRQSP/-3′ | 310 |
| MCR | TCTGCCCGCCCCATTCGTGAAAAC | 360 |
| MCR | 5′-GAC TAC GAC GAA CGC CAG ATT-3′ | 311 |
| MCR | 5′-TCG GTA GCT TGC GGG ATA G-3′ | 312 |
| MCR | 5′-/5TEX615/AGT CGG GCT GTA AAG GCG TCT GT/3IABRQSP/3′ | 313 |
| MCR | AGTCGGGCTGTAAAGGCGTCTGT | 361 |
| TSG | 5′-CGG TCG CCG CAT ACA CTA TT-3′ | 314 |
| TSG | 5′-CAG TGC TGC AAT GAT ACC GC-3′ | 315 |
| TSG | 5′-CGC CAT TAC CAT GAG CGA TAA-3′ | 316 |
| TSG | 5′-GGA AGC GCC TCA TTC AGT T -3′ | 317 |
| TSG | 5′-/5HEX/ACA ACG TCA/ZEN/CCC GCC TTG AC/3IABKFQ/-3′ | 318 |
| TSG | ACAACGTCACCCGCCTTGAC | 362 |
| TSG | 5′-GCTGATCGGAAACCAAACGG-3′ | 319 |
| TSG | 5′-ACTTGACCGACAGAGGCAAC-3′ | 320 |
| TSG | 5′-/TEX615/AAACCAATGTCGTTCCGGCCC/3IABKFQ/-3′ | 321 |
| TSG | AAACCAATGTCGTTCCGGCCC | 363 |
| OXA | 5′-GGT AAT AAC CTG GTA CGA GCA CAT AC-3′ | 322 |
| OXA | 5′-ACC AAA ACC AAC CCG TTT AAC TTC T-3′ | 323 |
| OXA | 5′-/56-FAM/CCA TGG CAC/ZEN/TTT CAG CAG TTC CTG T/3IABKFQ/-3′ | 324 |
| OXA | CCATGGCACTTTCAGCAGTTCCTGT | 364 |
| OXA | 5′-AAT CAC AGG GCG TAG TTG TG-3′ | 79 |
| OXA | 5′-ACC CAC CAG CCA ATC TTA GG-3′ | 80 |
| OXA | 5′-/5HEX/TAG CTT GAT CGC CCT CGA TTT GGG/3BHQ_1/-3′ | 130 |
| OXA | 5′-CTT AGC ACC TAT GGT AAT GCT CTT GC-3′ | 328 |
| OXA | 5′-TTC TGC ATT AGC TCT AGG CCA G-3′ | 329 |
| OXA | 5′-/5TEX615/ACT TTA GGT GAG GCA ATG GCA TTG TCA GC/3IAbRQSp/- | 330 |
| | 3′ | |
| OXA | ACTTTAGGTGAGGCAATGGCATTGTCAGC | 365 |
| OXA | 5′-CAT CGA TCA GAA TGT TCA AGC GC-3′ | 331 |
| OXA | 5′-CCA ATA CGA CGT GCC AAT TCT TG-3′ | 332 |
| OXA | 5′-/56-FAM/TGG CAC GCA/ZEN/TTT AGA CCG AGC AAA AAC | 333 |
| | AG/3IABKFQ/-3′ | |
| OXA | TGGCACGCATTTAGACCGAGCAAAAACAG | 366 |
| OXA | 5′-CTC GTG CTT CGA CCG AGT ATG-3′ | 334 |
| OXA | 5′-TTA ACC AGC CTA CTT GTG GGT-3′ | 335 |
| OXA | 5′-/5HEX/CCT GCT TCG/ZEN/ACC TTC AAA ATG CTT AAT | 336 |
| | GCT/3IABKFQ/-3′ | |
| OXA | CCTGCTTCGACCTTCAAAATGCTTAATGCT | 367 |
| OXA | 5′-TAC AGA ATA TGT GCC AGC CTC TAC-3′ | 337 |
| OXA | 5′-GCA TGA GAT CAA GAC CGA TAC GTC-3′ | 338 |
| OXA | 5′-/5TEX615/TGC CCT GAT CGG ATT GGA GAA CCA/3IAbRQSp/-3′ | 339 |
| OXA | TGCCCTGATCGGATTGGAGAACCA | 368 |
| | 5′-/5Cy5/TGA GAC ACG/TAO/GTC CAG ACT CCT ACG/3IAbRQSp/-3′ | 340 |
| | TGAGACACGGTCCAGACTCCTACG | 369 |

TABLE 5
Control Sequences

| Kit | Sequence | SEQ ID NO: |
|---|---|---|
| OXA | TATGGTAATAACCTGGTACGAGCACATACAGAATATGTCCCTGCGTCAACAT | 349 |
| | TTAAGATGCTAAATGCCTTAATTGGATTAGAAAATCATAAAGCTACAACAACT | |
| | GAGATTTTCAAATGGGATGGTAAAAAAAGATCTTATCCTATGTGGGAAAAAG | |
| | ATATGACTTTAGGTGATGCCATGGCACTTTCAGCAGTTCCTGTATATCAAGA | |
| | ACTTGCAAGACGGACTGGCTTAGATCTAATGCAAAAAGAAGTTAAACGGGT | |
| | TGGTTTTGGTAAT | |
| OXA | AATCTTAGCACCTATGGTAATGCTCTTGCACGAGCAAATAAAGAATATGTCC | 350 |
| | CTGCATCAACATTTAAGATGCTAAATGCTTTAATCGGGCTAGAAAATCGCGG | |
| | GCAACAACAAATGAGATTTTCAAATGGGATGGTAAAAAAAGAACTTATCCTA | |
| | TGTGGGAGAAAGATATGACTTTAGGTGAGGCAATGGCATTGTCAGCAGTTC | |
| | CAGTATATCAAGAGCTTGCAAGACGGACTGGCCTAGAGCTAATGCAGAAAG | |
| | AAGTAAAGC | |
| OXA | TCAATCATCGATCAGAATGTTCAAGCGCTTTTTAATGAAATCTCAGCTGATG | 351 |
| | CTGTGTTTGTCACATATGATGGTCAAAATATTAAAAAATATGGCACGCATTTA | |
| | GACCGAGCAAAAACAGCTTATATTCCTGCATCTACATTTAAAATTGCCAATG | |
| | CACTAATTGGTTTAGAAAATCATAAAGCAACATCTACAGAAATATTTAAGTGG | |
| | GATGGAAAGCCACGTTTTTTTAAAGCATGGGACAAAGATTTTACTTTGGGCG | |
| | AAGCCATGCAAGCATCTACAGTGCCTGTATATCAAGAATTGGCACGTCGTAT | |
| | TGGTCCAAGCTTAATGCA | |
| OXA | GTAATGATCTTGCTCGTGCTTCGACCGAGTATGTACCTGCTTCGACCTTCAA | 352 |
| | AATGCTTAATGCTTTGATCGGCCTTGAGCACCATAAGGCAACCACCACAGA | |
| | AGTATTTAAGTGGGACGGGCAAAAAAGGCTATTCCCAGAATGGGAAAAGGA | |
| | CATGACCCTAGGCGATGCTATGAAAGCTTCCGCTATTCCGGTTTATCAAGAT | |
| | TTAGCTCGTCGTATTGGACTTGAACTCATGTCTAAGGAAGTGAAGCGTGTTG | |
| | GTTATGGCAATGCAGATATCGGTACCCAAGTCGATAATTTTTGGCTGGTGG | |
| | GTCCTTTAAAAATTACTCCTCAGCAAGAGGCACAGTTTGCTTACAAGCTAGC | |
| | TAATAAAACGCTTCCATTTAGCCCAAAAGTCCAAGATGAAGTGCAATCCATG | |
| | TTATTCATAGAAGAAAAGAATGGAAATAAAATATACGCAAAAAGTGGTTGGG | |
| | GATGGGATGTAGACCCACAAGTAGGCTGGTTAACTGGATGGG | |
| OXA | CAAATACAGAATATGTGCCAGCCTCTACATTTAAAATGTTGAATGCCCTGAT | 353 |
| | CGGATTGGAGAACCAGAAAACGGATATTAATGAAATATTTAAATGGAAGGG | |
| | CGAGAAAAGGTCATTTACCGCTTGGGAAAAAGACATGACACTAGGAGAAGC | |
| | CATGAAGCTTTCTGCAGTCCCAGTCTATCAGGAACTTGCGCGACGTATCGG | |
| | TCTTGATCTCATGCAAAAAG | |
| MCR | CTGTATGTCAGCGATCATGGCGAAAGTCTGGGTGAGAACGGTGTCTATCTA | 341 |
| | CATGGTATGCCAAATGCCTTTGCACCAAAAGAACAGCGCAGTGTGCCTGCA | |
| | TTTTTCTGGACGGATAAGCAAACTGGCATC | |
| MCR | CAGTCAGTATGCGAGTTTCTTTCGGGTGCATAAGCCAGTGCGTTTTTATATC | 342 |
| | AACCCGATTACGCCGATTTATTCGGTGGGTAAGCTTGCCAGTATCGAGTAC | |
| | AAAAAAGCCACTGCGCCAACAGACACCATCTATCATGCCAAAGACGCCGTG | |
| | CAGCCACCAAGCCGAGCGAGCGTAAGCCACGCCTAGTGGTGTTCGTCGTC | |
| | GGTGAGACGGCGCGTGCTGACCATGTGCAGTTCAATGGCTATGGCCGTGA | |
| | GACTTTCCCGCAGCTTGCCAAAGTTGATGGCTTGGCGAATTTT | |
| MCR | TTTAATGATGTTCGTTCGTGTGGGACTGCAACCGCTGTATCCGTCCCCTGC | 343 |
| | ATGTTCTCCAATATGGGGAGAAAGGAGTTTGATGATAATCGCGCTCGCAAT | |
| | AGCGAGGGCCTGCTAGATGTGTTGCAAAAAACGGGGATCTCCATTTTTTGG | |
| | AAGGAGAACGATGGAGGCTGCAAAGGCGTCTGCGACCGAGTACCTAACAT | |
| | CGAAATCGAACCAAAGGATCACCCTAAGTTCTGCGATAAAAACACATGCTAT | |
| | GACGAGGTTGTCCTTCAAGACCTC | |
| MCR | TGAGTTAAGGCGTTACATTGTCCCTACCTATTTTGTCAGTAGTGCATCTAAA | 344 |
| | TATCTCAATGAGCACTATTTGCAGACGCCCATGGAATACCAACAACTTGGCC | |
| | TAGATGCGAAGAATGCCAGTCGTAACCCGAACACTAAACCTAACTTATTAGT | |
| | GGTTGTTGTGGGTGAAACTGCGCGCTCAATGAGCTATCAATATTATGGATAT | |
| | AACAAGCCAACCAATGCTCATACCCAAAATCAGGGGCTGATTGCGTTTAAC | |
| | GATACTAGCTCATGCGGCACGGCCACGGCGGTGTCTCTACCCTGTATGTTT | |
| | TCACGAATGGGGGGGGCAGACTATGATCCTCGCCGTGCTAATGCTC | |
| MCR | TCGGCGCGACTACGACGAACGCCAGATTCGTCGGCGCGAGTCCGTGCTGC | 345 |
| | ACGTTTTAAACCGTAGTGACGTCAACATTCTCTGGCGCGATAACCAGTCGG | |
| | GCTGTAAAGGCGTCTGTGATGGACTGCCCTTTGAAAACCTGTCTTCGGCAG | |
| | GCCATCCCACACTGTGCCATGGCGTGCGCTGCCTGGATGAAATTCTGCTCG | |
| | AAGGGTTGGCCGAGAAGATAACAACAAGCCGCAGCGATATGCTGATCGTTC | |
| | TGCATATGCTGGGCAATCACGGCCCAGCGTATTTCCTGCGCTATCCCGCAA | |
| | GCTACCGACGCTGG | |
| TSG | CGGTCGCCGCATACACTATTCTCAGAATGACTTGGTTGAGTACTCACCAGT | 346 |
| | CACAGAAAAGCATCTTACGGATGGCATGACAGTAAGAGAATTATGCAGTGC | |
| | TGCCATAACCATGAGTGATAACACTGCGGCCAACTTACTTCTGACAACGATC | |
| | GGAGGACCGAAGGAGCTAACCGCTTTTTTGCACAACATGGGGGATCATGTA | |
| | ACTCGCCTTGATCGTTGGGAACCGGAGCTGAATGAAGCCATACCAAACGAC | |
| | GAGCGTGACACCACGACGCCTGCAGCAATGGCAACAACGTTGCGCAAACT | |
| | ATTAACTGGCGAACTACTTACTCTAGCTTCCCGGCAACAATTAATAGACTGG | |
| | ATGGAGGCGGATAAAGTTGCAGGACCACTTCTGCGCTCGGCCCTTCCGGC | |
| | TGGCTGGTTTATTGCTGATAAATCTGGAGCCAGTGAGCGTGGGTCTCGCGG | |
| | TATCATTGCAGCACTG | |
| TSG | CGCCATTACCATGAGCGATAACAGCGCCGCCAATCTGCTGCTGGCCACCG | 347 |
| | TCGGCGGCCCCGCAGGATTGACTGCCTTTTTGCGCCAGATCGGCGACAAC | |
| | GTCACCCGCCTTGACCGCTGGGAAACGGAACTGAATGAGGCGCTTCC | |
| TSG | GCTGATCGGAAACCAAACGGGAGACGCGACACTACGAGCGGGTTTTCCTA | 348 |
| | AAGATTGGGTTGTTGGAGAGAAAACTGGTACCTGCGCCAACGGGGGCCGG | |
| | AACGACATTGGTTTTTTTAAAGCCCAGGAGAGAGATTACGCTGTAGCGGTG | |
| | TATACAACGGCCCCGAAACTATCGGCCGTAGAACGTGACGAATTAGTTGCC | |
| | TCTGTCGGTCAAGT | |

### Multiplex

As described herein, a kit of the disclosure may include one or more multiplex primer-probe mixes containing one or more primers and one or more probes. The disclosure also contemplates that any of the kits of the disclosure may be combined to detect multiple nucleic acid sequences (e.g., target genes) simultaneously (i.e., to perform a multiplex reaction). Thus, in some embodiments, one or more primers and one or more probes of a kit of the disclosure may be added to the components of a second kit of the disclosure in a reaction to detect an additional target nucleic acid sequence. Strictly by way of example, if a kit is being used to detect the OXA target gene, then one or more primers and one or more probes from a second kit may be added to the reaction (e.g., added to the same tube or added to a different well of the same microtiter plate) so that another target gene (e.g., MCR) is also detected.

Thus, combinations of components from kits as disclosed herein is contemplated.

In some aspects, a kit of the disclosure for use in detecting the OXA gene target comprises: (A) 2 multiplexed primer/probe mixes (10×PCR Mix 1-2), wherein the mix amplifies OXA-23, OXA-51, OXA-143, OXA-48, OXA-58, and OXA-24/40, as well as the internal control (16S rRNA gene); (B) 2 multiplexed positive control DNA mixes; and (C) premixed 2×PCR master mix vials (Supermix). A representative kit for detecting an OXA gene target is shown in Table 12.

In some aspects, a kit of the disclosure for use in detecting the mcr gene target comprises: (A) 1 multiplexed primer/probe mix (10×PCR Mix), wherein the mix amplifies MCR-1, MCR-2 and MCR-3-5, as well as the internal control (16S rRNA gene); (B) 1 multiplexed positive control DNA mix; and (C) premixed 2×PCR master mix vial (Supermix). A representative kit for detecting an mcr gene target is shown in Table 10.

In some aspects, a kit of the disclosure for use in detecting the TEM/SHV/GES targets comprises: (A) 1 multiplexed primer/probe mix (10×PCR mix), wherein the mix amplifies the ESBL targets TEM, SHV, and GES, as well as the internal control (16S rRNA gene); (B) 1 multiplexed positive control DNA mix; and (C) premixed 2×PCR master mix vial (Supermix). A representative kit for detecting TEM/SHV/GES gene targets is shown in Table 11.

The sequence listing including SEQ ID NOS 1-370 is hereby incorporated by reference for all purposes.

### EXAMPLES

### MCR Related Examples

The following examples are provided to illustrate, but not to limit, the subject matter described herein.

### Example 1

This example illustrates the real-time PCR amplification of serial dilutions of the MCR Control mix targets. A serial dilution was performed to show the efficiency of the developed assays.

Oligonucleotides. The MCR multiplex PCR assay was designed to detect 5 mcr gene families: mcr-1, mcr-2, mcr-3, mcr-4, and mcr-5. Representative sequences for each mcr gene family was accessed using the NCBI GenBank Database and these sequences were used to construct alignments for each of the five gene families using UniPro's UGENE software. The alignments were used to design primers and fluorophore-labelled hydrolysis probes that inclusively detect as many variants within each gene family, while differentiating between each family. For the particular experiments disclosed herein, the oligonucleotides (both primers and probes) were synthesized by IDT (Integrated DNA Technologies, Coralville, Iowa). A concentration gradient ranging from 100 nM-800 nM was performed in advance to determine the optimum concentration for each oligonucleotide set.

PCR. PCR was performed on the BioRad CFX-96 thermal cycler (Bio-Rad, Hercules, CA) using the operational settings summarized in Table 6. Utilizing the determined optimum oligonucleotide concentration and varying the amount of spiked-in copies of the targeted gene sequences, the efficiencies of the developed assays were calculated.

TABLE 6
PCR Cycling Conditions

| Step | Temp (° C.) | Duration (sec) | Cycles |
|---|---|---|---|
| Enzyme Activation | 98 | 30 | Hold |
| Denature | 98 | 5 | |
| Anneal | 60 | 10 | 30 |
| Extension/Capture | 72 | 10 | |

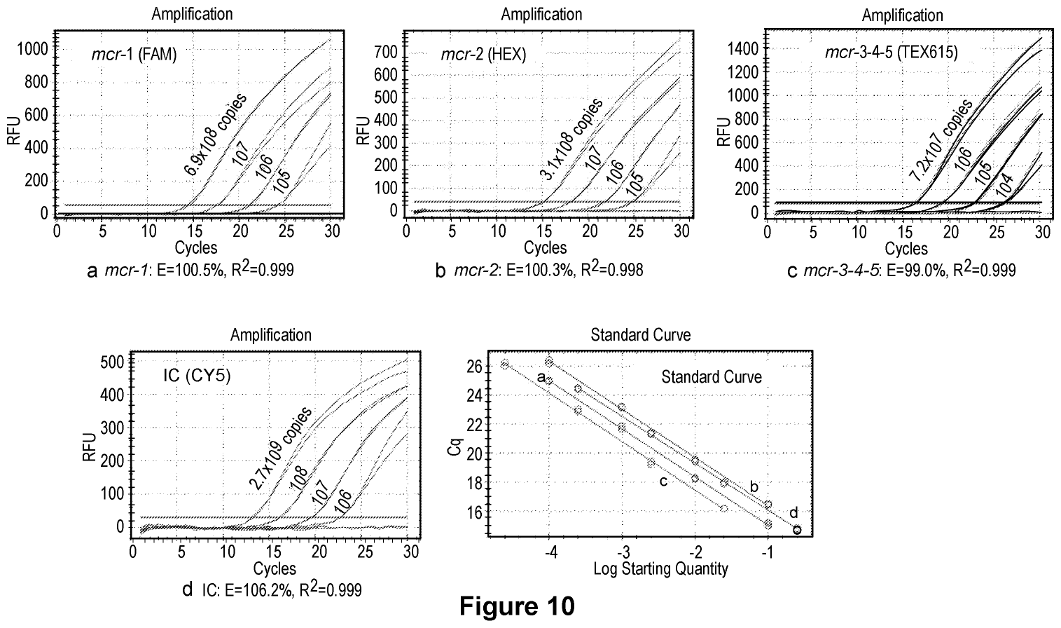

Experimental Results. FIG. 10A illustrates the results for the mcr-1 gene, using the FAM fluorescent label. The cycle number vs. relative fluorescence unit (RFU) curves resulting using 6.9×10⁸, 6.9×10⁷, 6.9×10⁶, and 6.9×10⁵ copy amounts are shown. This experiment resulted in an efficiency of 100.5%, with an R² value of 0.999.

FIG. 10B illustrates the results for the mcr-2 gene, using the HEX fluorescent label. The cycle number vs. RFU curves resulting using 3.1×10⁸, 3.1×10⁷, 3.1×10⁶, and 3.1×10⁵ copy amounts are shown. This experiment resulted in an efficiency of 100.3%, with an R² value of 0.998.

FIG. 10C illustrates the results for the mcr-3, mcr-4, and mcr-5 gene, using the TEX615 fluorescent label. The cycle number vs. RFU curves resulting using 7.2×10⁷, 7.2×10⁶, 7.2×10⁵, and 7.2×10⁴ copy amounts are shown. This experiment resulted in an efficiency of 99%, with an R² value of 0.999.

FIG. 10D illustrates the results for the Internal Control, using the CY5 fluorescent label. The cycle number vs. RFU curves resulting using 2.7×10⁹, 2.7×10⁸, 2.7×10⁷, and 2.7×10⁶ copy amounts are shown. This experiment resulted in an efficiency of 106.2%, with an R² value of 0.999.

FIG. 10E illustrates the straight-line standard curve of each of these results.

The results in FIG. 10 demonstrate the excellent efficiency of the multiplex MCR PCR assays with efficiencies of each target was greater than 99% which fall within the minimum information necessary for evaluating qPCR experiments (e.g. the MIQE guidelines).

### Example 2

This example illustrates the performance of primers and probes of a representative MCR Kit of the disclosure used to detect MCR nucleic acids extracted and quantified from 90 clinical isolates as measured by quantification cycle (Cq) value.

PCR. Real-time PCR was performed using the BioRad CFX-96 thermal cycler and the cycling conditions of Table 6 on 90 isolate samples which were obtained from the CDC's Antibiotic Resistance (AR) isolate bank panel with new or novel antibiotic resistance. Nucleic acid was isolated using the DNeasy Blood and Tissue Kit (Qiagen, Hilden, Germany) or Exiprep Dx Bacteria Genomic DNA kit (Bioneer, Daejeon, Korea) per the manufacturer's instructions.

The PCR assay mixture contains the following components:
Master Mix Solution A: Includes the MCR gene families, 18-specific and internal control primers and probes, Streck 2× Supermix (PCR Buffer, HotstartTaq DNA Polymerase (New England Biolabs, Ipswich, MA), MgCl, dNTP Mixture). 24 uL of Master A reagent mix is used for each 25 uL PCR Reaction.
Sample: 1 uL of the extracted isolate sample.

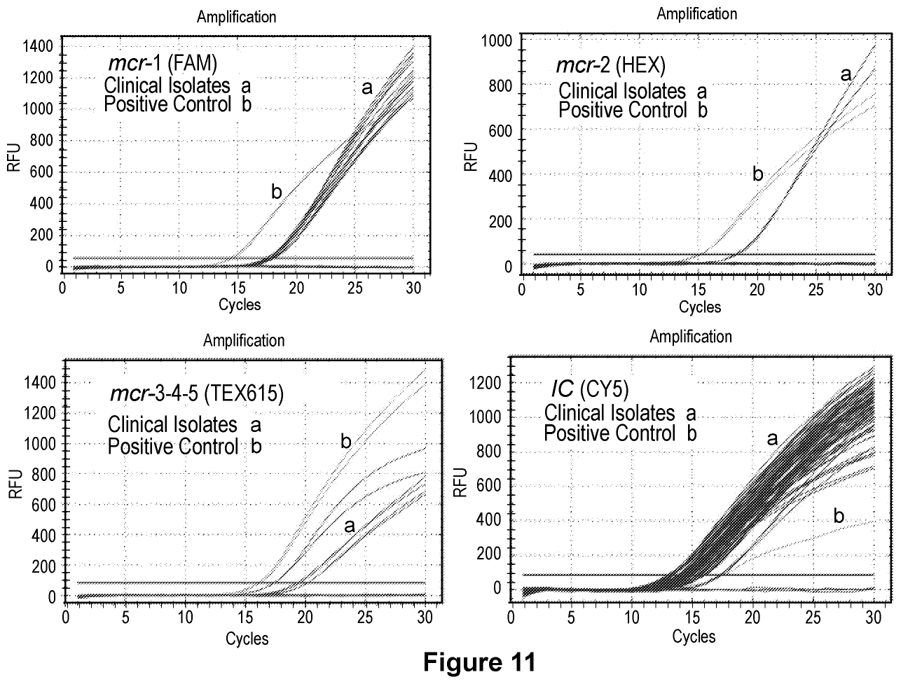

FIGS. 11A-11D illustrate the cycle number vs. RFU curves for mcr-1, mcr-2, mcr-3-4-5, and the internal control experiments.

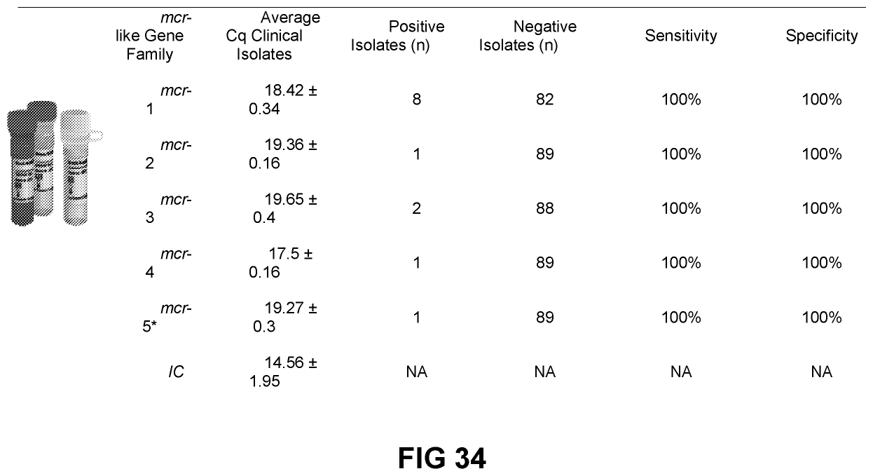

Table 7 and FIG. 34 show the average Cqs and the sensitivity and specificity in each MCR gene family. With both sensitivity and specificity at 100% for all tests, these results illustrate the ability of each specific MCR gene family primer and probe set to specifically differentiate between each gene family without any cross reactivity or false positives.

TABLE 7
Representative kit of the disclosure, MCR-Clinical Isolate Testing

| mcr- | Average | | | | |
|---|---|---|---|---|---|
| like | Cq | Positive | Negative | | |
| Gene | Clinical | Isolates | Isolates | | |
| Family | Isolates | (n) | (n) | Sensitivity | Specificity |
| mcr-1 | 18.42 ± | 8 | 82 | 100% | 100% |
| | 0.34 | | | | |
| mcr-2 | 19.36 ± | 1 | 89 | 100% | 100% |
| | 0.16 | | | | |
| mcr-3 | 19.65 ± | 2 | 88 | 100% | 100% |
| | 0.4 | | | | |
| mcr-4 | 17.5 ± | 1 | 89 | 100% | 100% |
| | 0.16 | | | | |
| mcr-5* | 19.27 ± | 1 | 89 | 100% | 100% |
| | 0.3 | | | | |
| IC | 14.56 ± | NA | NA | NA | NA |
| | 1.95 | | | | |

These experiments show that a representative MCR kit of the disclosure identifies and differentiates mcr-1, mcr-2, and mcr-3-4-5 gene families with 100% sensitivity and 100% specificity. The test detected up to 48 out of 51 mcr allelic variants within the described gene families. As such, the kit represents a surveillance tool to track the spread of mobilized colistin resistance.

### OXA Related Examples

### Example 3

This example illustrates the ability of the OXA kit to identify gene variants from DNA isolated from both fresh bacterial culture and stabilized bacterial culture samples.

Oligonucleotides. The OXA multiplex PCR assay is designed to detect a total of 224 OXA-like variants (six gene families) without cross-reactivity between the OXA subgroups. Representative sequences for each OXA gene family was accessed using the NCBI GenBank Database and these sequences were used to construct alignments for each of the five gene families using UniPro's UGENE software. The alignments were used to design primers and fluorescently labelled hydrolysis probes that inclusively detect as many variants within each gene family, while differentiating between each family. For the particular experiments disclosed herein, the oligonucleotides (both primers and probes) were synthesized by IDT (Integrated DNA Technologies, Coralville, Iowa).

PCR. Real-time PCR was performed using the Bio-Rad CFX96 Touch (BioRad laboratories, Hercules, California) on nucleic acid extracted from bacterial isolates or from plasmid-based control sequences with either the QuickGene DNA tissue kit S (FujiFilm Wako Chemicals Europe, Neuss, Germany) or the Qiagen DNeasy Blood and Tissue kit (Qiagen, Hilden, Germany) in an assay to detect Carbapenem-hydrolyzing class D (CDHL) β-lactamase DNA. Operational settings are those summarized in Table 6.

The assay is comprised of two 25 μL reactions both containing 12.5 μL of a customized formula of Luna Universal qPCR Master Mix (New England BioLabs, Ipswitch, Massachusetts) 1 μL of sample, and 2.5 μL of a Master mix containing the primers and probes for the internal control and the primers and probes for OXA-143, OXA-48, OXA-24/40 (mix #1), or the primers and probes for OXA-58, OXA-51, OXA-23 (mix #2).

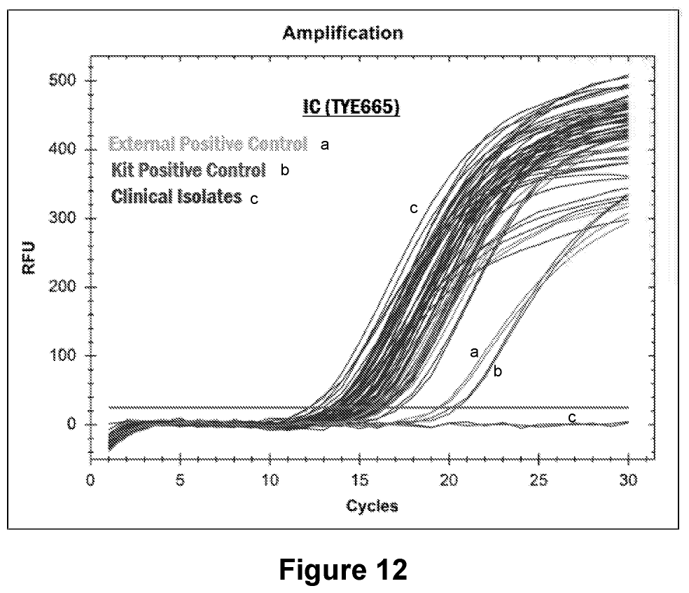

FIG. 12 illustrates representative data using the internal control utilized in both OXA real-time PCR mixes.

Cell Stabilization. Bacterial cells were stabilized using methods as disclosed in U.S. Patent Application Publication No. 2019017774, incorporated herein by reference in its entirety.

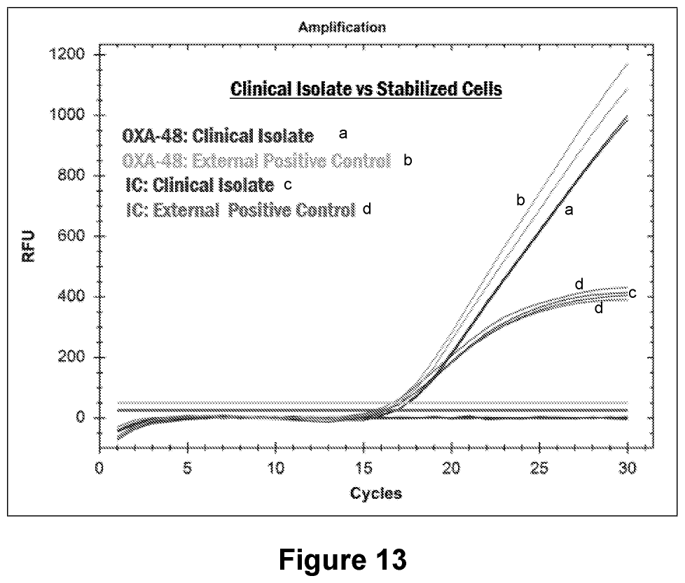

FIG. 13 shows a direct comparison of amplicons generated from DNA extracted from fresh culture and from stabilized cells. The lack of a meaningful difference shows that the stabilization methods utilized for cell sample does not interfere with the ability of the OXA kit to detect the presence of the OXA gene variants.

### Example 4

This example shows the performance of primers and probes in a representative OXA Kit of the disclosure used to detect OXA nucleic acids extracted and quantified from clinical isolates. Nucleic acid was isolated using the QuickGene DNA tissue kit S (FujiFilm Wako Chemicals Europe, Neuss, Germany) or the Qiagen DNeasy Blood and Tissue kit (Qiagen, Hilden, Germany).

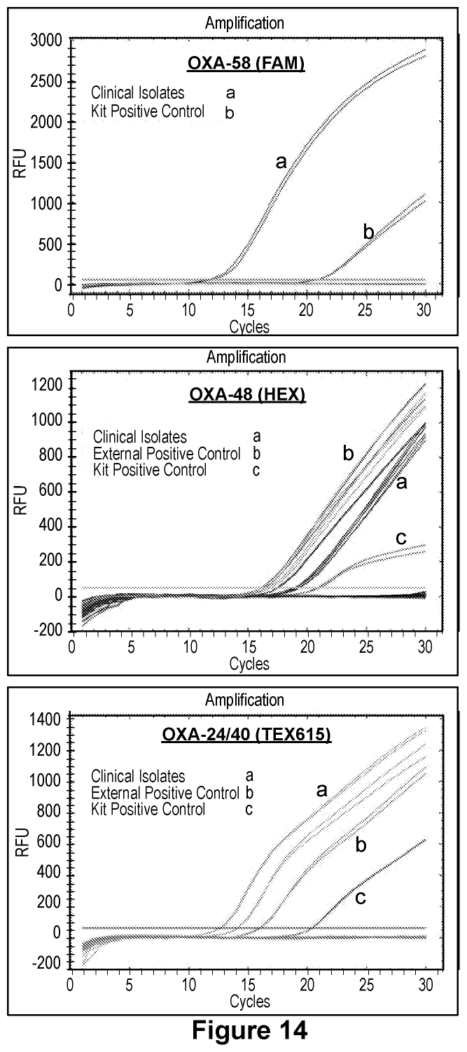

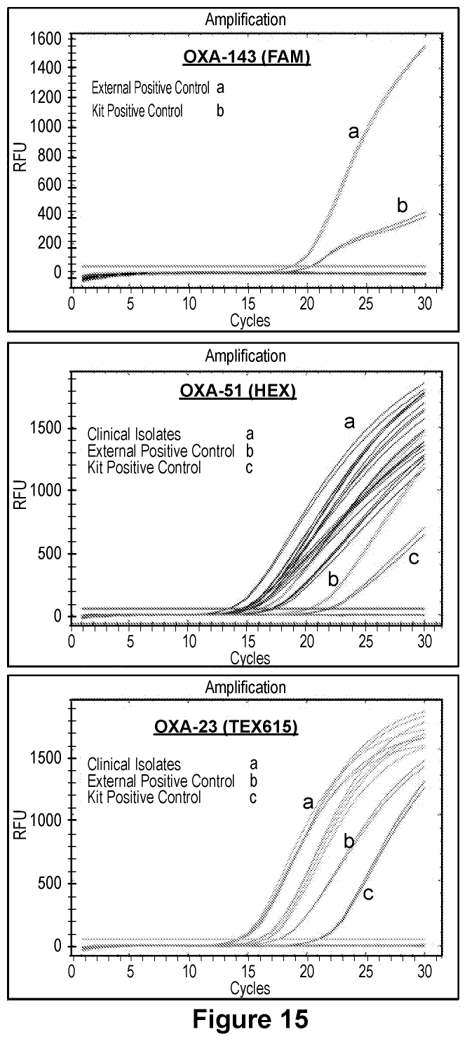

FIG. 14A-14C shows results for the cycle number vs. RFU curve for OXA-58 (FAM, FIG. 14A), OXA-48 (HEX, FIG. 14B), and OXA-24/40 (TEX615, FIG. 14C), respectively. FIG. 15A-15C shows results for cycle number vs. RFU curve for OXA-143 (FAM, FIG. 15A), OXA-51 (HEX, FIG. 15B), and OXA-23 (TEX615, FIG. 15C). The numerical results for these experiments, including average Cq for internal controls, external controls and clinical isolates are recorded in Table 8.

TABLE 8
Summary of results. Average quantification cycle (Cq) values ± the standard deviation
are shown for each target source across each of the targeted gene families. The data
set (n) in each of the Average Cq columns represents the number of PCR reactions used
to generate these values, all specimens and controls were run in duplicate.
Table 8: OXA Real-Time PCR Assay Summary

| OXA-like | | Average Cq | | Positive | Negative | | |
|---|---|---|---|---|---|---|---|
| Gene | Average Cq | External | Average Cq | Isolates | Isolates | | |
| Family | Positive Control | Positive Control | Clinical Isolates | (n) | (n) | Sensitivity | Specificity |
| OXA-143 | 20.23 ± 0.13 (n = 4) | 18.50 ± 0.49 (n = 4) | NA | 0 | 54 | 100% | 100% |
| OXA-48 | 20.89 ± 0.14 (n = 4) | 16.54 ± 0.26 (n = 8) | 17.39 ± 1.49 (n = 16) | 8 | 46 | 100% | 100% |
| OXA-24/40 | 21.13 ± 0.15 (n = 4) | 16.74 ± 0.20 (n = 4) | 13.93 ± 0.72 (n = 4) | 2 | 52 | 100% | 100% |
| OXA-58 | 20.82 ± 0.15 (n = 4) | NA | 11.86 ± 0.21 (n = 2) | 1 | 53 | 100% | 100% |
| OXA-51 | 21.86 ± 0.15 (n = 4) | 18.20 ± 2.20 (n = 8) | 14.87 ± 0.70 (n = 18) | 9 | 45 | 100% | 100% |
| OXA-23 | 20.90 ± 0.17 (n = 4) | 17.57 ± 0.08 (n = 4) | 14.25 ± 0.15 (n = 4) | 2 | 52 | 100% | 100% |
| IC | 20.09 ± 0.27 (n = 8) | 16.74 ± 1.65 (n = 32) | 14.32 ± 1.11 (n = 215) | NA | NA | NA | NA |

*Four of the isolates that generated positive results are omitted from these calculations due to pending confirmatory testing.

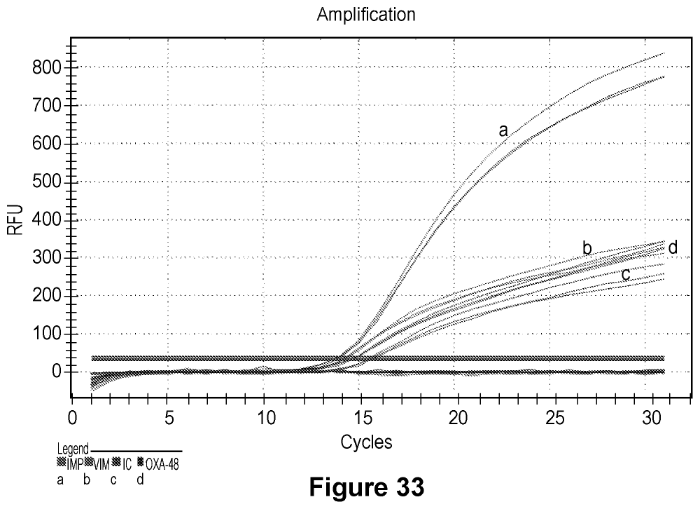

These experiments show that a representative OXA detection kit of the disclosure identifies and differentiates the six OXA gene families with 100% sensitivity and 100% specificity. As such, the kit represents a surveillance tool to track the spread of mobilized OXA-based antibiotic resistance. Additional results generated using a representative kit of the disclosure including β-lactamase gene targets are shown in FIG. 33.

### Example 5

Recent increases in colistin-resistant infections led the CDC to launch an urgent public health response for mcr surveillance. Colistin is a last resort antibiotic that is more utilized due to increases in carbapenem-resistant infections. Since mcr-1 was first reported, more mcr gene variants have been identified, but few screening tools have been developed to rapidly detect mcr-positive samples. To improve surveillance for mcr genes, a multiplex real-time PCR assay is described using a representative MCR detection kit of the disclosure that detected mcr gene families 1 through 5 in less than 45 minutes.

### Materials and Methods

This study utilized sequence-specific primers and probes for real-time PCR-based detection of mobilized colistin resistance mcr variants. An internal control (IC), targeting a conserved region in Gram-negative bacteria, was also included in the multiplex mix to discriminate false negative samples. Positive DNA controls were included with the multiplex assay. Data was generated using the Bio-Rad CFX96 Touch™ Real-time PCR Detection System.

### Results

A representative MCR kit of the disclosure was optimized to amplify mcr families 1 through 5. Amplification of serial dilutions of target controls generated PCR efficiencies 99% and correlation coefficients 0.998. The sensitivity and specificity of the assay was evaluated using 90 clinical isolates and determined to be 100%. Internal control DNA were detected in 100% of samples and within 20 PCR cycles.

### Conclusions

A representative MCR kit of the disclosure provided a rapid amplification and detection strategy to monitor plasmid-mediated colistin resistance genes. The data demonstrated a sensitive and specific assay, with no observed cross-reactivity with previously characterized clinical isolates from Gram-negative organisms. The results demonstrated this assay can serve as a screening tool for surveillance of mcr-mediated colistin resistance, thereby improving antimicrobial stewardship practices to minimize mcr gene dissemination into the community.

Global increase in carbapenemase-producing Enterobacteriaceae has resulted in increased use of colistin.

Colistin is considered a last resort antibiotic for multi-drug resistant Gram-negative bacteria.

Mobilized colistin resistance (mcr) gene mcr-1 was first reported in 2015 in Escherichia coli strain isolated from pigs and humans in China.

During the last 4 years mcr genes have been detected in over 30 countries and other types of bacteria such as Klebsiella pneumoniae and Salmonella enterica.

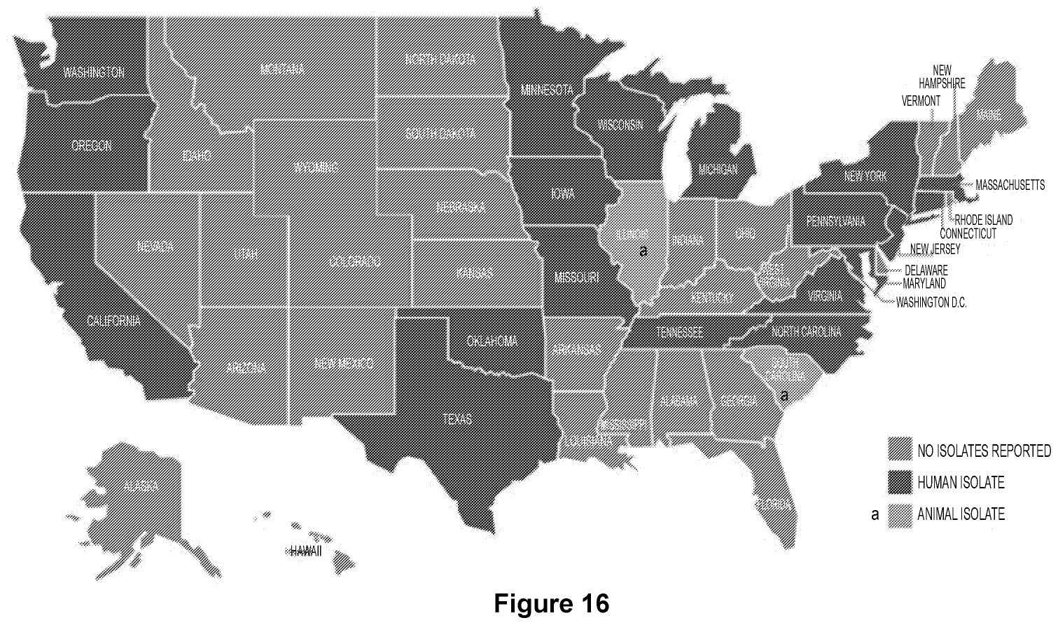

According to the CDC, in the U.S. alone, over 50 human isolates have been reported across 19 states (FIG. 16).

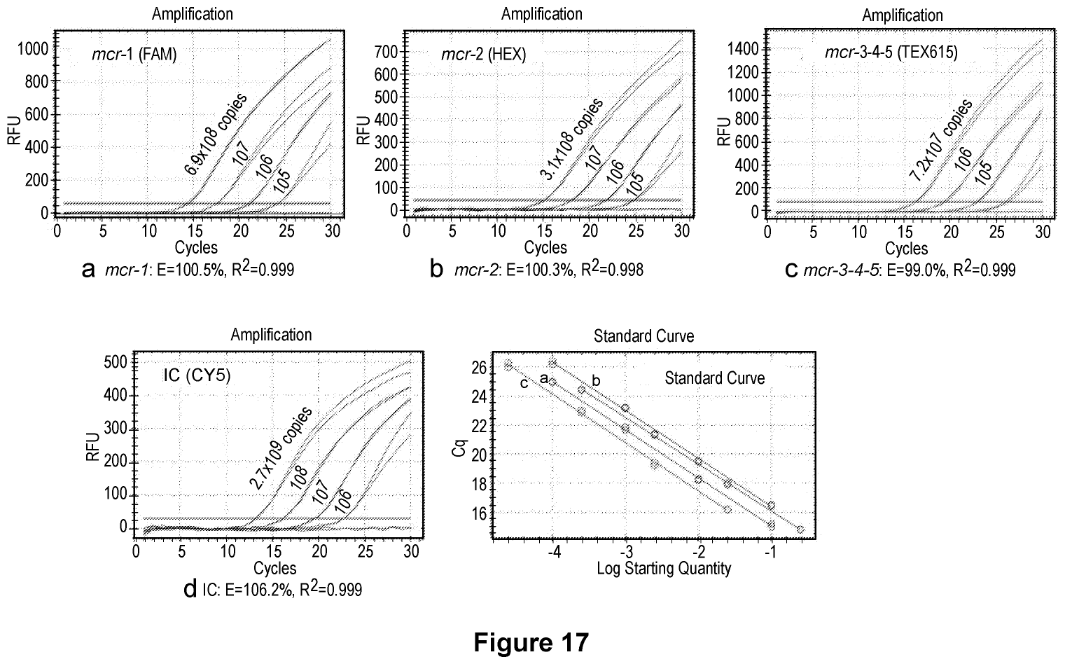

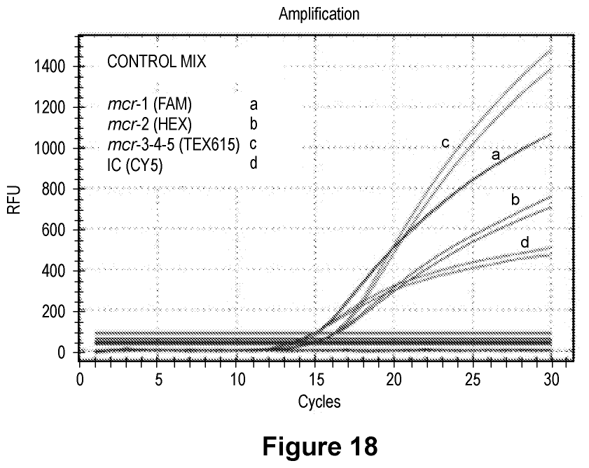

The results in FIG. 17 show the amplification of serial dilutions of MCR Control Mix targets from a representative kit of the disclosure. Standard curves are shown for each target control. PCR efficiencies were over 99% for all the target controls and correlation coefficients were over 0.998. The amplification of the Multiplex Control Mix is shown in FIG. 18 and respective C_{q} values for target controls are shown in Table 13.

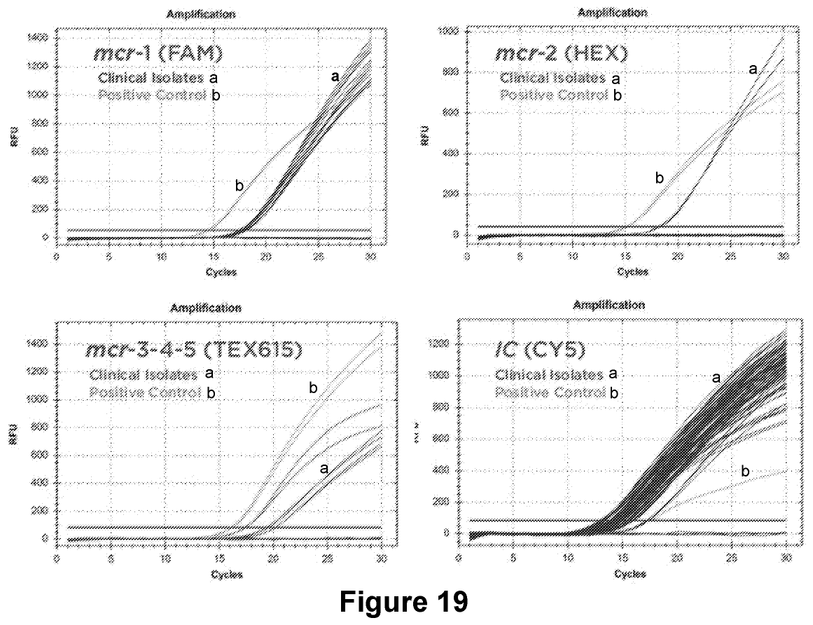

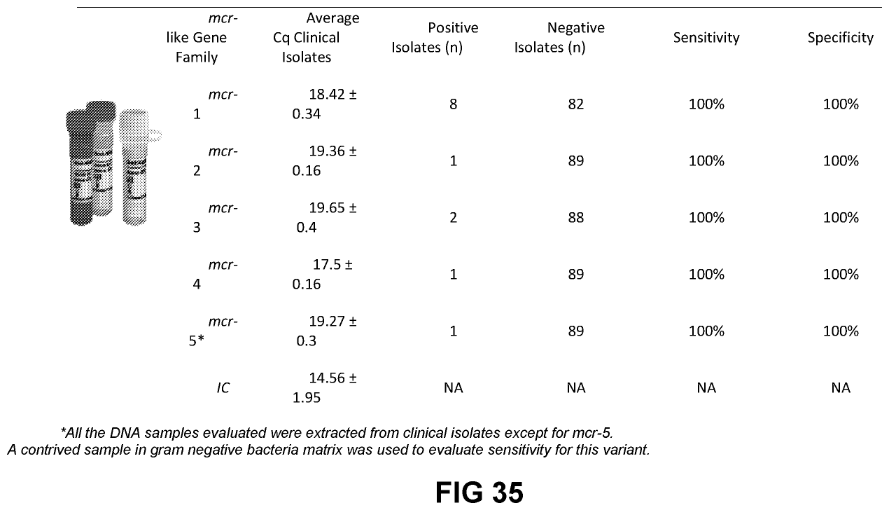

Out of the 90 isolates evaluated, 12 expressed mcr gene families. Representative amplification data of mcr-positive isolates with respect to positive controls is shown in FIG. 19. The mcr positive isolates amplified within 20 cycles of the PCR run (Table 14 and FIG. 35). The clinical isolates were correctly identified by a representative MCR kit of the disclosure and the sensitivity and specificity were 100%.

TABLE 13
C_{q} values of MCR Control Mix from a representative kit of the disclosure.

| mcr-like gene family | Fluorophore | Average Cq value of positive control (n = 2) |
|---|---|---|
| mcr-1 | FAM | 15.10 ± 0.13 |
| mcr-2 | HEX | 16.47 ± 0.05 |
| mcr-3-4-5 | TEX615 | 16.18 ± 0 |
| IC | CY5 | 14.64 ± 0.02 |

TABLE 14
MCR-Clinical Isolate Testing using a
representative kit of the disclosure.

| mcr- | Average | | | | |
|---|---|---|---|---|---|
| like | Cq | Positive | Negative | | |
| Gene | Clinical | Isolates | Isolates | | |
| Family | Isolates | (n) | (n) | Sensitivity | Specificity |
| mcr-1 | 18.42 ± | 8 | 82 | 100% | 100% |
| | 0.34 | | | | |
| mcr-2 | 19.36 ± | 1 | 89 | 100% | 100% |
| | 0.16 | | | | |
| mcr-3 | 19.65 ± | 2 | 88 | 100% | 100% |
| | 0.4 | | | | |
| mcr-4 | 17.5 ± | 1 | 89 | 100% | 100% |
| | 0.16 | | | | |
| mcr-5* | 19.27 ± | 1 | 89 | 100% | 100% |
| | 0.3 | | | | |
| IC | 14.56 ± | NA | NA | NA | NA |
| | 1.95 | | | | |

*All the DNA samples evaluated were extracted from clinical isolates except for mcr-5.
A contrived sample in gram negative bacteria matrix was used to evaluate sensitivity for this variant.

The genes covered in MCR kits of the disclosure were differentiated using target-specific hydrolysis probes, chemically linked to different fluorescent dyes. The gene variants covered by the assay are shown in Table 15. PCR master mix preparation and PCR cycling are shown in Tables 16 and 17, respectively. The efficiency and correlation coefficients were determined for each target by the amplification of serial dilutions of the Control Mix. The sensitivity and specificity of the assay was evaluated with DNA purified from mcr-negative and mcr-positive overnight bacterial cultures (n=90). Positive isolates for mcr gene targets were obtained from the CDC & FDA Antibiotic Resistance (AR) isolate bank panel with new or novel antibiotic resistance (AR Bank #0346, 0349, 0350, 0493, 0494, 0495, 0496, 0497, 0538, 0539, 0540, 0635). DNA was isolated using the Qiagen® DNeasy® Blood and Tissue Kit or Exiprep Dx Bacteria Genomic DNA kit per the manufacturer's instructions. Duplicate reactions were run for all clinical isolates and controls.

TABLE 15
Gene targets covered by the MCR kits of the disclosure.

| Gene Family | Gene Targets |
|---|---|
| mcr-1 | mcr-1.1, 1.2, 1.3, 1.4, 1.5, 1.6, 1.7, 1.8, 1.9, 1.11, 1.12, 1.13, 1.14, |
| | 1.15 |
| mcr-2 | mcr-2.1 |
| mcr-3 | mcr-3.1, 3.2, 3.3, 3.4, 3.5, 3.6, 3.7, 3.8, 3.9, 3.10, 3.11, 3.12, 3.13, |
| | 3.14, 3.15, 3.16, 3.18, 3.19, 3.20, 3.21, 3.22, 3.23, 3.24, 3.25 |
| mcr-4 | mcr-4.1, 4.2, 4.3, 4.4, 4.5, 4.6 |
| mcr-5* | mcr-5.1, 5.2, 5.3 |
| IC | 16S rRNA |

TABLE 16
Master Mix Preparation.

| Source | Component | 25 μL Reaction | Final Concentration |
|---|---|---|---|
| Lab supplied | Nuclease free water | 9 μL | — |
| Streck 2X Supermix | — | 12.5 μL | 1X |
| Streck ARM-D Kit | 10X PCR Mix | 2.5 μL | — |

Distribute Master Mix into PCR wells or tubes as appropriate before sample

| | | | |
|---|---|---|---|
| Lab supplied or | Template Unknown | 1 μL | — |
| Streck ARM-D Kit | or NTC | | |
| | -or- | | |
| | Template-Control | | |

TABLE 17
PCR Cycling Conditions.

| Step | Temp (° C.) | Duration (sec) | Cycles |
|---|---|---|---|
| Enzyme Activation | 98 | 30 | Hold |
| Denature | 98 | 5 | |
| Anneal | 60 | 10 | 30 |
| Extension/Capture | 72 | 10 | |

Representative MCR kits of the disclosure:
Identified and differentiated mcr-1, mcr-2 and mcr-3-4-5 gene families with 100% sensitivity and 100% specificity.
Can detect up to 48 out of 51 mcr allelic variants within the described gene families.
Represents a surveillance tool to track the spread of mobilized colistin resistance.

### Example 6

In further aspects and embodiments of the disclosure, kits and methods for rapid detection of OXA β-lactamases by nucleic acid amplification (e.g., multiplex real-time PCR) are provided.

Background: The OXA β-lactamases have evolved to metabolize cephalosporins and carbapenems in addition to the penicillins, making them a growing problem when selecting effective antibiotic therapies. These enzymes are often associated with Acinetobacter spp., but due to the mobility of these genes, other organisms have acquired resistance to this class of enzymes, facilitating the spread of this type of antibiotic resistance. As such, assays that identify these resistance mechanisms are needed for faster detection of resistance-associated genes. These data can be used to supplement phenotypic test results and promote improved antimicrobial stewardship and surveillance. In this study, a multiplex real-time PCR assay is described that successfully discriminated 6 genetically similar OXA β-lactamase gene families and utilized an external positive control that fully mimics a patient sample. OXA and mcr have been identified by the CDC as emerging and serious public health threats.

Methods: The NCBI GenBank database was used to identify sequences for each OXA gene family. Sequences for each of the 6 groups were aligned to identify genetic variation between each OXA group, which guided primer and probe design to improve discrimination of each OXA family within the multiplex PCR reaction. A custom 2×qPCR MasterMix was run with thermal cycling conditions as disclosed herein on the BioRad CFX96 Real-Time PCR System for the multiplex reactions. An internal control (IC), which targets a conserved region in Gram-negative bacteria, was included to reduce false-negative results [Poirel L, Naas T, Nordmann P. Diversity, Epidemiology, and Genetics of Class D β-Lactamases. Antimicrobial Agents and Chemotherapy. 2010; 54(1):24-38. doi:10.1128/AAC.01512-08; Vázquez-Ucha J C, Maneiro M, Martínez-Guitián M, et al. Activity of the β-Lactamase Inhibitor LN-1-255 against Carbapenem-Hydrolyzing Class D β-Lactamases from Acinetobacter baumannii. Antimicrobial Agents and Chemotherapy. 2017; 61 (11):e01172-17. doi:10.1128/AAC.01172-17]. External positive control samples containing several of the targets amplified by this kit were prepared via proprietary methods and run on the kit in conjunction with a positive control and clinical isolates.

Results: Together, the 6 oligo sets in this assay amplified a total of 224 OXA-like variants without cross reactivity between the OXA subgroups. Positive samples were identified within the first 22 cycles of PCR. Sensitivity and specificity for the control DNA tested in this assay was greater than or equal to 95% in each case. The external positive controls were positive for the expected targets, and performed comparably to the clinical isolates.

Conclusions: β-lactamases are a major mechanism of antibiotic resistance in Gram-negative bacteria, which continues to threaten health care facilities by reducing the available treatment options. Because there are many genes associated with antibiotic resistance, it is critical that tests such as these are developed to comprehensively detect these mechanisms. The assays described herein provide a rapid detection strategy for genotypic monitoring of oxacillinase-based antibiotic resistance in Gram-negative bacteria. Inclusion of an external positive control allows the entire analytical process for a clinical specimen to be monitored during testing. More rapid identification of these genes provides an added tool to improve antibiotic stewardship practices and active surveillance of resistance mechanisms.

### Example 7

Background: The OXA Real-Time PCR assay was developed to address the continual need to improve resistance testing and monitoring. The OXA-like gene families detected by this assay are 6 of the OXA families that are as classified as Carbapenemase Hydrolyzing Class D β-lactamases (CHDL) which are clinically significant for their ability to produce resistance to antibiotics of last resort. This is a rapidly growing class of β-lactamase enzymes with more than 500 reported enzymes to date [Poirel L, Naas T, Nordmann P. Diversity, Epidemiology, and Genetics of Class D β-Lactamases. Antimicrobial Agents and Chemotherapy. 2010; 54(1):24-38. doi:10.1128/AAC.01512-08; Vázquez-Ucha J C, Maneiro M, Martínez-Guitián M, et al. Activity of the β-Lactamase Inhibitor LN-1-255 against Carbapenem-Hydrolyzing Class D β-Lactamases from Acinetobacter baumannii. Antimicrobial Agents and Chemotherapy. 2017; 61 (11):e01172-17. doi:10.1128/AAC.01172-17].

This assay is capable of rapid identification and discrimination between the 6 OXA families. This information is valuable as resistance and reduced susceptibility for certain drugs varies between the different enzymes and may rely on the presence of addition resistance mechanisms [Evans B A, Amyes S G B. OXA β-Lactamases. Clinical Microbiology Reviews. 2014; 27(2):241-263. doi:10.1128/CMR.00117-13; Antunes N T, Fisher J F. Acquired Class D β-Lactamases. Antibiotics. 2014; 3(3):398-434. doi:10.3390/antibiotics3030398]. Existing assays are designed to identify the more common OXA R-lactamases, such as OXA-48, and may not be able to detect these clinically significant variants [Vázquez-Ucha J C, Maneiro M, Martínez-Guitián M, et al. Activity of the β-Lactamase Inhibitor LN-1-255 against Carbapenem-Hydrolyzing Class D β-Lactamases from Acinetobacter baumannii. Antimicrobial Agents and Chemotherapy. 2017; 61 (11):e01172-17. doi:10.1128/AAC.01172-17; Antunes N T, Fisher J F. Acquired Class D β-Lactamases. Antibiotics. 2014; 3(3):398-434. doi:10.3390/antibiotics3030398].

In addition, the OXA Real-Time PCR assay was developed to incorporate external positive controls as a part of the test kit. These controls are needed to meet the requirements described by CAP, ISO, and the CFR for controls used in molecular diagnostic testing. Each of these entities describes specific guidelines to ensure consistent and reliable practices for patient testing. The external positive control included in the OXA Real-Time PCR assay contains stabilized microorganisms and mimics patient samples thereby monitoring the entire testing system as described by the regulatory agencies. See also Example 4, herein above.

TABLE 18
OXA β-lactamase gene families identified in each
master mix of the OXA Real-Time PCR Assay.
The same internal control (IC) is included in each master mix.
Table 18: Targets Detected by the OXA Real-Time PCR Assay

| | OXA-like | |
|---|---|---|
| Master Mix | Gene Family | Fluorophore |
| PCR Mix #1 | OXA-143 | FAM |
| | OXA-48 | HEX |
| | OXA-24/40 | TEX615 |
| | IC | TYE665 |
| PCR Mix #2 | OXA-58 | FAM |
| | OXA-51 | HEX |
| | OXA-23 | TEX615 |
| | IC | TYE665 |

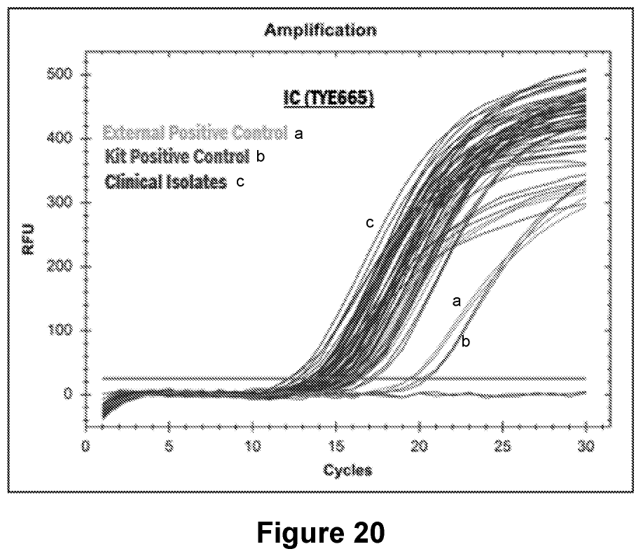

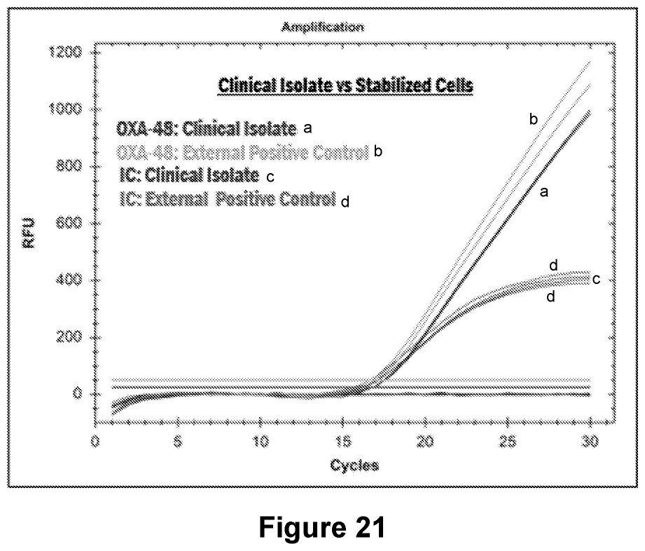

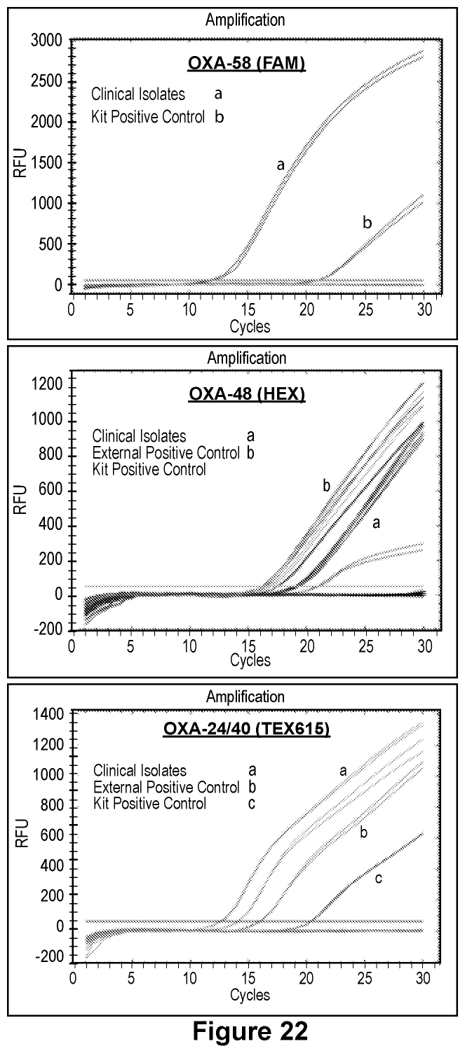

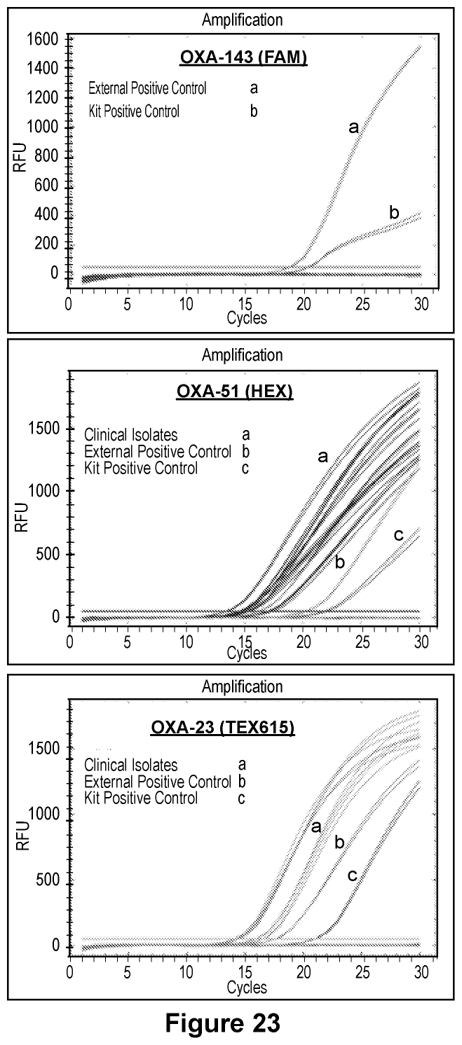

Results: Data in FIGS. 20-23 illustrate the amplification of 6 OXA β-lactamase gene families detected by the OXA Real-Time PCR assay, and the comparable performance of external positive controls to clinical isolates. The assay correctly identified the 17 isolates that carried one or more OXA β-lactamase genes, and 4 additional isolates were identified as positive that will be further characterized by sequencing, a total of 58 isolates were tested. The assay also correctly identified the resistance genes carried by the four external positive control specimens. As demonstrated in a previous study, DNA extracted from cells stabilized using this methodology produced Cq values that correlated to the number of cells in the extraction. A summary of the results is shown in Table 8, which shows the average quantification cycle (Cq) values±the standard deviation are shown for each target source across each of the targeted gene families. The data set (n) in each of the Average Cq columns represents the number of PCR reactions used to generate these values. All specimens and controls were run in duplicate.

### Example 8

Materials and Methods: Clinical isolates that had been previously characterized by singleplex PCR and/or sequencing were used to evaluate the performance of the assay. The Qiagen® DNeasy® Blood and Tissue Kit was used to extract nucleic acid from a set of overnight bacterial cultures (n=58) per the manufacturer's instructions. A subset of these isolates carried various OXA β-lactamase resistance genes.

The instructions for use from the Streck ARM-D kits was used to run the OXA Real-Time PCR assay on the BioRad CFX96 Real-Time PCR System. Each 25 μL PCR reaction was comprised of 12.5 μL of a custom 2×qPCR master mix, 2.5 μL of a 10× oligo mix, 9 μL of molecular grade water, and 1 μL of nucleic acid.

Duplicate reactions were run for all specimens and controls. Four Gram-negative organisms derived from clinical specimens (2 Acinetobacter baumannii, 1 Acinetobacter spp., and 1 Klebsiella pneumonia), containing various OXA R-lactamase resistance genes, were chemically stabilized in a cellular suspension according to proprietary procedures. These stabilized cells served as external positive controls for the OXA Real-Time PCR assay. DNA was extracted from each control using the QuickGene DNA tissue kit S and the QuickGene-810 system according to previously published procedures [QuickGene Series Application Guide. Genomic DNA extraction from Pseudomonas aeruginosa. No. 37]. Two of the isolates used to prepare the external positive controls were also part of the set of clinical isolates evaluated by the assay allowing for a side-by-side comparison of the control with an unknown sample. The other control isolates contained different targets across both mastermixes. Amplicons generated by the clinical isolates were compared to the positive controls and used to determine the specificity and sensitivity of the assay based on the correct identification of specimens that carried resistance genes versus specimens that did not.

TABLE 19
PCR cycling Conditions used for the OXA Real-Time PCR Assay.
Table 19: PCR Cycling Conditions

| Step | Temp (° C.) | Duration | Cycles |
|---|---|---|---|
| Enzyme Activation | 98 | 30 s | Hold |
| Denature | 98 | 5 s | 30 |
| Anneal | 60 | 10 s | |
| Extension/Capture | 72 | 20 s | |

### Summary and Conclusions

Identified and differentiated 6 OXA β-lactamase gene families with 100% sensitivity and specificity.
Can detect up to 224 OXA β-lactamase allelic variants within the described gene families.
One of the first assays available to detect and discriminate OXA carbapenemases.
May promote improved antimicrobial resistance stewardship through reduced use of inappropriate antibiotics.
The external positive control ensured reliable and consistent analytical performance in the clinical setting by mimicking patient specimens.

### Example 9

Further kits provided by the disclosure are described below.

In various embodiments, the kit includes:
10×PCR mixes with primers and probes for real-time PCR detection
Control mixes that serve as external positive controls
2× Supermix vials containing PhilisaFAST® DNA polymerase, MgCl₂, dNTPs, buffer

The kits utilize an internal control to avoid false negatives of unknown samples, and utilize thermal cycling conditions as set out in Table 20.

TABLE 20
PCR Cycling Conditions.

| Step | Temp (° C.) | Duration | Cycles |
|---|---|---|---|
| Enzyme Activation | 98 | 30 s | Hold |
| Denature | 98 | 5 s | 30 |
| Anneal | 60 | 10 s | |
| Extension/Capture | 72 | 20 s | |

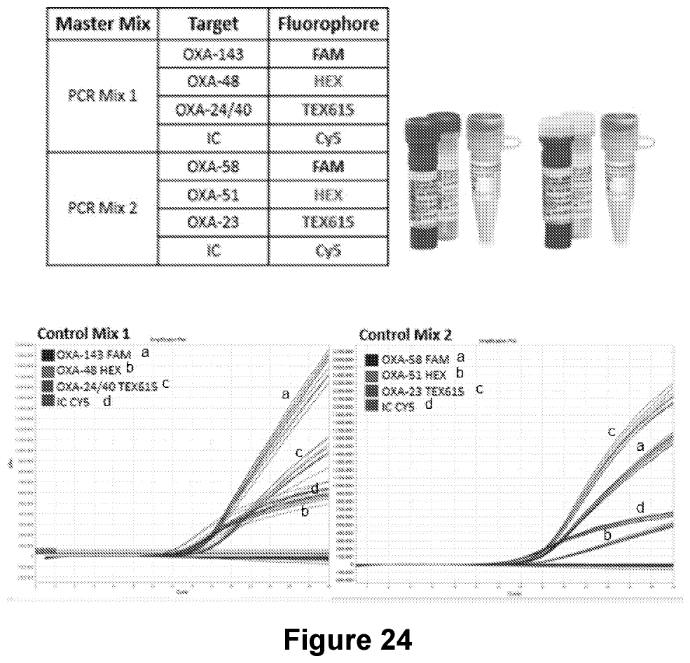

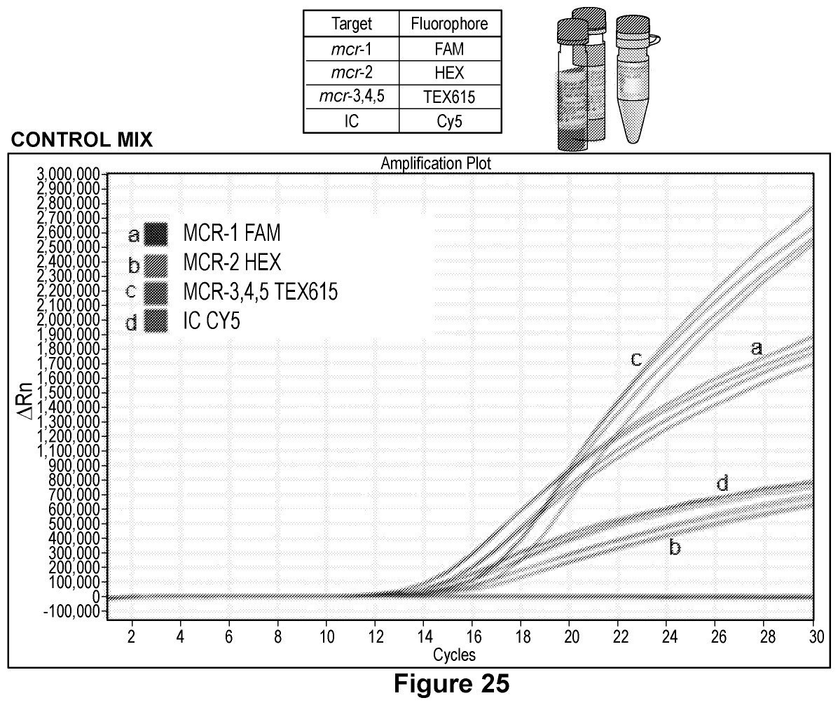

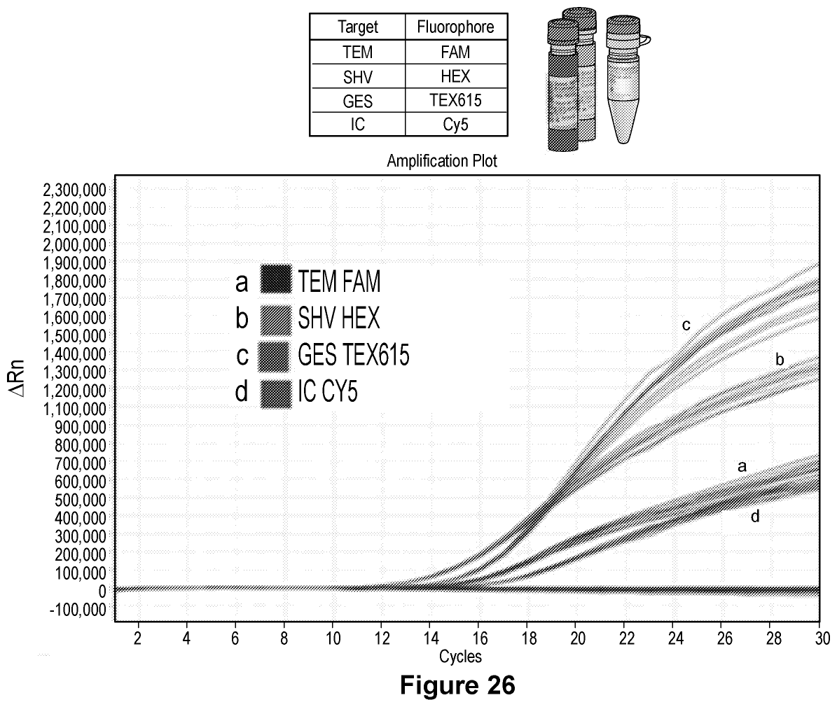

Results generated from additional experiments using kits as generally described herein are shown in FIGS. 24-26.

Additional sequences contemplated for use in the kits and methods of the disclosure include, but are not limited to, the following:

| Name | 5′-3′ Sequence | SEQ ID NO: |
|---|---|---|
| IMP-F′ v1 | TTTATCCAGGCCCAGGGCACA | 296 |
| IMP-F′ v2 | TTTTTTATCCAGGCCCAGGG | 297 |
| IMP-F′ v3 | ACGGGGTTAGTTATTGGCTGG | 298 |
| IMP-R′ | GGCCAAGCTTCTAAATTTGCG | 299 |
| IMP-HEX v1 | 5HEX/CCGGACGGT/ZEN/CTTGGTAATTTGGGT/ | 87 |
| | 3IABKFQ | |
| IMP-HEX v2 | 5HEX/CCGTACGGT/ZEN/TTAGGCAATTTGGGT/ | 88 |
| | 3IABKFQ | |
| IMP-HEX v3 | 5HEX/CCTCACGGC/ZEN/CTTGGTAATTTGGGT/ | 300 |
| | 3IABKFQ | |
| IMP-HEX v4 | 5HEX/ACCGTATGG/ZEN/TCTAGGTAATTTGGGTG/ | 301 |
| | 3IABKFQ | |
| IMP-HEX v5 | 5HEX/CCTCACGGT/ZEN/CTTGGCAATTTAGGT/ | 302 |
| | 3IABKFQ | |
| MCR-3 Fwd | 5′-GAT GTT CGT TCG TGT GGG AC-3′ | 305 |
| MCR-3 Rev | 5′-GAA GGA CAA CCT CGT CAT AGC AT-3′ | 306 |
| MCR-3 TEX615 | 5′-/5TEX615/AAA GGC GTC TGC GAC CGA | 307 |
| | GT/3IABRQSP/-3′ | |
| MCR-4 Fwd | 5′-AGG CGT TAC ATT GTC CCT ACC-3′ | 308 |
| MCR-4 Rev | 5′-AGC ACG GCG AGG ATC ATA-3′ | 309 |
| MCR-4 TEX615 | 5′-/5Tex615/TCT GCC CGC CCC ATT CGT | 310 |
| | GAA AAC/3IABRQSP/-3′ | |
| MCR-5 Fwd | 5′-GAC TAC GAC GAA CGC CAG ATT-3′ | 311 |
| MCR-5 Rev | 5′-TCG GTA GCT TGC GGG ATA G-3′ | 312 |
| MCR-5 TEX615 | 5′-/5TEX615/AGT CGG GCT GTA AAG GCG | 313 |
| | TCT GT/3IABRQSP/3′ | |
| TEM Fwd | 5′-CGG TCG CCG CAT ACA CTA TT-3′ | 314 |
| TEM Rev | 5′-CAG TGC TGC AAT GAT ACC GC-3′ | 315 |
| TEM FAM | 5′-/56-FAM/ATG AAG CCA/ZEN/TAC CAA | 148 |
| | ACG ACG AGC/3IABKFQ/-3′ | |
| SHV Fwd | 5′-CGC CAT TAC CAT GAG CGA TAA-3′ | 316 |
| SHV Rev | 5′-GGA AGC GCC TCA TTC AGT T-3′ | 317 |
| SHV-HEX | 5′-/5HEX/ACA ACG TCA/ZEN/CCC GCC TTG | 318 |
| | AC/3IABKFQ/-3′ | |
| GES Fwd | 5′-GCTGATCGGAAACCAAACGG-3′ | 319 |
| GES Rev | 5′-ACTTGACCGACAGAGGCAAC-3′ | 320 |
| GES TEX615 | 5′- | 321 |
| | /TEX615/AAACCAATGTCGTTCCGGCCC/3IABKFQ/- | |
| | 3′ | |
| OXA-143 F′ | 5′-GGT AAT AAC CTG GTA CGA GCA CAT | 322 |
| | AC-3′ | |
| OXA-143 R′ | 5′-ACC AAA ACC AAC CCG TTT AAC TTC | 323 |
| | T-3′ | |
| OXA-143 FAM | 5′-/56-FAM/CCA TGG CAC/ZEN/TTT CAG | 324 |
| | CAG TTC CTG T/3IABKFQ/-3′ | |
| OXA-48 F′ | 5′-AAT CAC AGG GCG TAG TTG TG-3′ | 79 |
| OXA-48 R′ | 5′-ACC CAC CAG CCA ATC TTA GG-3′ | 80 |
| OXA-48 -HEX | 5′-/5HEX/TAG CTT GAT CGC CCT CGA TTT | 130 |
| | GGG/3BHQ_1/-3′ | |
| OXA-24/40 F′ | 5′-CTT AGC ACC TAT GGT AAT GCT CTT | 328 |
| | GC-3′ | |
| OXA-24/40 R′ | 5′-TTC TGC ATT AGC TCT AGG CCA G-3′ | 329 |
| OXA-24/40 TEX615 | 5′-/5TEX615/ACT TTA GGT GAG GCA ATG | 330 |
| | GCA TTG TCA GC/3IAbRQSp/-3′ | |
| OXA-58 F′ | 5′-CAT CGA TCA GAA TGT TCA AGC GC-3′ | 331 |
| OXA-58 R′ | 5′-CCA ATA CGA CGT GCC AAT TCT TG-3′ | 332 |
| OXA-58 FAM | 5′-/56-FAM/TGG CAC GCA/ZEN/TTT AGA | 333 |
| | CCG AGC AAA AAC AG/3IABKFQ/-3′ | |
| OXA-51 F′ | 5′-CTC GTG CTT CGA CCG AGT ATG-3′ | 334 |
| OXA-51 R′ | 5′-TTA ACC AGC CTA CTT GTG GGT-3′ | 335 |
| OXA-51 HEX | 5′-/5HEX/CCT GCT TCG/ZEN/ACC TTC | 336 |
| | AAA ATG CTT AAT GCT/3IABKFQ/-3′ | |
| OXA-23 F′ | 5′-TAC AGA ATA TGT GCC AGC CTC TAC- | 337 |
| | 3′ | |
| OXA-23 R′ | 5′-GCA TGA GAT CAA GAC CGA TAC GTC- | 338 |
| | 3′ | |
| OXA-23 TEX615 | 5′-/5TEX615/TGC CCT GAT CGG ATT GGA | 339 |
| | GAA CCA/3IAbRQSp/-3′ | |

Unless otherwise stated, any numerical values recited herein include all values from the lower value to the upper value in increments of one unit provided that there is a separation of at least 2 units between any lower value and any higher value. As an example, if it is stated that the amount of a component, a property, or a value of a process variable such as, for example, temperature, pressure, time and the like is, for example, from 1 to 90, preferably from 20 to 80, more preferably from 30 to 70, it is intended that intermediate range values such as (for example, 15 to 85, 22 to 68, 43 to 51, 30 to 32 etc.) are within the teachings of this specification. Likewise, individual intermediate values are also within the present teachings. For values which are less than one, one unit is considered to be 0.0001, 0.001, 0.01, or 0.1 as appropriate. These are only examples of what is specifically intended and all possible combinations of numerical values between the lowest value and the highest value enumerated are to be considered to be expressly stated in this application in a similar manner. As can be seen, the teaching of amounts expressed as “parts by weight” herein also contemplates the same ranges expressed in terms of percent by weight. Thus, an expression in the of a range in terms of “at least ‘x’ parts by weight of the resulting composition” also contemplates a teaching of ranges of same recited amount of “x” in percent by weight of the resulting composition.”

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The disclosures of all articles and references, including patent applications and publications, are incorporated by reference for ail purposes. The term “consisting essentially of to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist of, or consist essentially of the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

It is understood that the above description is intended to be illustrative and not restrictive. Many embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

1. A kit comprising one or more primers and/or one or more probes for the identification of one or more genes associated with antibiotic resistance, wherein the genes are:
(A) Imipenem-resistant carbapenemase (IMP), wherein the primers are SEQ ID NO: 296-299 and the probes are SEQ ID NO: 354-356;
(B) Mobilized colistin resistance (MCR), wherein the primers are SEQ ID NO: 305-306, 308-309, and 311-312, and the probes are SEQ ID NO: 357-361;
(C) Temoniera (TEM), wherein the primers are SEQ ID NO: 314-315;
(D) Sulfhydral reagents variable (SHV), wherein the primers are SEQ ID NO: 316-317, and the probe is SEQ ID NO: 362;
(E) Guiana extended-spectrum β-lactamase (GES), wherein the primers are SEQ ID NO: 319-320, and the probe is SEQ ID NO: 363;
(F) Oxacillinase-type β-lactamase (OXA), wherein the primers are SEQ ID NO: 322-323, 328-329, 331-332, 334-335, and 337-338, and the probes are SEQ ID NO: 364-369,
or a combination thereof.

2. The kit of claim 1, wherein (A) comprises each of the primers having sequences as set out in SEQ ID NO: 296-299 and each of the probes having sequences as set out in SEQ ID NO: 354-356.

3. The kit of claim 1, wherein the kit comprises (A) and further comprises:
(i) primers having SEQ ID NOs: 67-68, 70-71, 73-74, 76-77, 79-80, 89-90, 92-93, 95-96, and 98-99; and
(ii) probes having SEQ ID NOs: 69, 72, 75, 78, 81, 87-88, 91, 94, 97, and 100.

4. The kit of claim 3, further comprising (iii) control sequences having SEQ ID NOs: 261-267 and 269-271.

5. The kit of claim 1(A), wherein one or more probes comprises a label.

6. (canceled)

7. The kit of claim 5, wherein:
SEQ ID NO: 354, as labeled, is as set forth in SEQ ID NO: 300;
SEQ ID NO: 355, as labeled, is as set forth in SEQ ID NO: 301; and
SEQ ID NO: 356, as labeled, is as set forth in SEQ ID NO: 302.

8. The kit of claim 1, wherein (B) comprises each of the primers having sequences as set out in SEQ ID NO: 305-306, 308-309, and 311-312, and each of the probes having sequences as set out in SEQ ID NO: 357-361.

9. The kit of claim 1, wherein the kit comprises (B) and further comprises:
(i) primers having SEQ ID NOs: 252, 141, 143, 144, 76, and 77; and
(ii) probe having SEQ ID NO: 340.

10. The kit of claim 9, further comprising (iii) control sequences having SEQ ID NOs: 341-345 and 264.

11. The kit of claim 1(B), wherein one or more probes comprises a label.

12. (canceled)

13. The kit of claim 11, wherein:
SEQ ID NO: 357, as labeled, is as set forth in SEQ ID NO: 303;
SEQ ID NO: 358, as labeled, is as set forth in SEQ ID NO: 304;
SEQ ID NO: 359, as labeled, is as set forth in SEQ ID NO: 307;
SEQ ID NO: 360, as labeled, is as set forth in SEQ ID NO: 310; and
SEQ ID NO: 361, as labeled, is as set forth in SEQ ID NO: 313.

14. The kit of claim 1, wherein:
(C) comprises each of the primers having sequences as set out in SEQ ID NO: 314-315;
(D) comprises each of the primers having sequences as set out in SEQ ID NO: 316-317, and probe having a sequence as set out in SEQ ID NO: 362; and
(E) comprises each of the primers having sequences as set out in SEQ ID NO: 319-320, and probe having a sequence as set out in SEQ ID NO: 363.

15. The kit of claim 1, wherein the kit comprises (C), (D), and (E), and further comprises:
(i) primers having SEQ ID NOs: 76 and 77; and
(ii) probes having SEQ ID NOs: 148 and 340.

16. The kit of claim 15, further comprising (iii) control sequences having SEQ ID NOs: 346-348, and 264.

17. The kit of claim 16, wherein one or more probes comprises a label.

18. (canceled)

19. The kit of claim 17, wherein:
SEQ ID NO: 362, as labeled, is as set forth in SEQ ID NO: 318; and
SEQ ID NO: 363, as labeled, is as set forth in SEQ ID NO: 321.

20. The kit of claim 1, wherein (F) comprises each of the primers having sequences set out in SEQ ID NO: 322-323, 328-329, 331-332, 334-335, and 337-338, and each of the probes having sequences as set out in SEQ ID NO: 364-369.

21. The kit of claim 1, wherein the kit comprises (F) and further comprises:
(i) primers having SEQ ID NOs: 79-80 and 76-77; and
(ii) probes having SEQ ID NOs: 370 and 340.

22. The kit of claim 21, further comprising (iii) control sequences having SEQ ID NOs: 58, 349-353, and 264.

23. The kit of claim 1(F), wherein one or more probes comprises a label.

24. (canceled)

25. The kit of claim 23, wherein:
SEQ ID NO: 364, as labeled, is as set forth in SEQ ID NO: 324;
SEQ ID NO: 365, as labeled, is as set forth in SEQ ID NO: 330;
SEQ ID NO: 366, as labeled, is as set forth in SEQ ID NO: 333;
SEQ ID NO: 367, as labeled, is as set forth in SEQ ID NO: 336;
SEQ ID NO: 368, as labeled, is as set forth in SEQ ID NO: 339; and
SEQ ID NO: 369, as labeled, is as set forth in SEQ ID NO: 340.

26. A method of detecting one or more genes associated with antibiotic resistance comprising:
(a) amplifying at least a portion of a target nucleic acid from a biological sample using the kit of claim 1 to produce an amplified target nucleic acid; and
(b) analyzing the amplified target nucleic acid to detect the one or more genes associated with antibiotic resistance.

27. The method of claim 26, wherein the amplifying is performed by polymerase chain reaction (PCR).

28. (canceled)

29. (canceled)

30. (canceled)

31. (canceled)

32. The method of claim 26, wherein the analyzing is performed by fluorescence and/or infra-red probe-based detection chemistries.

33. The method of claim 26, wherein the biological sample is blood, a blood culture, urine, plasma, feces, a fecal swab, a peri-rectal/peri-anal swab, sputum, and/or a bacterial culture.

34. (canceled)

35. The method of claim 26, wherein the one or more genes associated with antibiotic resistance comprise IMP, MCR, TEM, SHV, GES, and/or OXA.

36. (canceled)

37. (canceled)

38. (canceled)

39. (canceled)


SEQUENCE LISTING (370 sequences)

SEQ ID NO: 1 - DNA, 18 bp, Artificial Sequence
  MOX F′
agaccctgttcgagatag

SEQ ID NO: 2 - DNA, 18 bp, Artificial Sequence
  MOX R′
atggtgatgctgtcaaag

SEQ ID NO: 3 - DNA, 20 bp, Artificial Sequence
  MOX-FAM
cgtgagcaagaccctgactg

SEQ ID NO: 4 - DNA, 20 bp, Artificial Sequence
  FOX F′
actatttcaactatggggtt

SEQ ID NO: 5 - DNA, 18 bp, Artificial Sequence
  TTG TCA TCC AGC TCA AAG
ttgtcatccagctcaaag

SEQ ID NO: 6 - DNA, 18 bp, Artificial Sequence
  FOX-TEX
tgaccgcagcataggcac

SEQ ID NO: 7 - DNA, 18 bp, Artificial Sequence
  EBC F′
gtggcggtgatttatgag

SEQ ID NO: 8 - DNA, 23 bp, Artificial Sequence
  EBC R′
cggtgaaggttttacttatagaa

SEQ ID NO: 9 - DNA, 19 bp, Artificial Sequence
  EBC-HEX
cagccgcactacttcacct

SEQ ID NO: 10 - DNA, 21 bp, Artificial Sequence
  DHA F′
tgcgtacggttatgagaacaa

SEQ ID NO: 11 - DNA, 18 bp, Artificial Sequence
  DHA R′
cccagcgcagcatatctt

SEQ ID NO: 12 - DNA, 24 bp, Artificial Sequence
  DHA-FAM
atgcggaatcttacggcgtggaat

SEQ ID NO: 13 - DNA, 19 bp, Artificial Sequence
  CMY F′
tccagcgttattgatatgg

SEQ ID NO: 14 - DNA, 18 bp, Artificial Sequence
  CMY R′
catctcccagcctaatcc

SEQ ID NO: 15 - DNA, 21 bp, Artificial Sequence
  CMY-TEX
acatatcgccaatacgccagt

SEQ ID NO: 16 - DNA, 20 bp, Artificial Sequence
  ACC F′
gccgctgatgcagaagaata

SEQ ID NO: 17 - DNA, 20 bp, Artificial Sequence
  ACC R′
tttgccgctaacccatagtt

SEQ ID NO: 18 - DNA, 11 bp, Artificial Sequence
  ACC-HEX
ccgacataccg

SEQ ID NO: 19 - DNA, 20 bp, Artificial Sequence
  IC F′
gagaggatgaccagccacac

SEQ ID NO: 20 - DNA, 21 bp, Artificial Sequence
  IC R′
agtactttacaacccgaaggc

SEQ ID NO: 21 - DNA, 25 bp, Artificial Sequence
  IC-TYE
tgagacacggtccagactcctacgg

SEQ ID NO: 22 - DNA, 18 bp, Artificial Sequence
  CTX-M-14 F′
ttggtgacgtggctcaaa

SEQ ID NO: 23 - DNA, 21 bp, Artificial Sequence
  CTX-M-14 R′
atatcattggtggtgccgtag

SEQ ID NO: 24 - DNA, 24 bp, Artificial Sequence
  CTX-M-14-FAM
cgtggactgtgggtgataagaccg

SEQ ID NO: 25 - DNA, 21 bp, Artificial Sequence
  CTX-M-15 F′
gtcacgctgttgttaggaagt

SEQ ID NO: 26 - DNA, 21 bp, Artificial Sequence
  CTX-M-15 R′
taatcaatgccacacccagtc

SEQ ID NO: 27 - DNA, 24 bp, Artificial Sequence
  CTX-M-15-TEX615
aacttgccgaattagagcggcagt

SEQ ID NO: 28 - DNA, 24 bp, Artificial Sequence
  OXA48-F′
agcagcaaggatttaccaataatc

SEQ ID NO: 29 - DNA, 21 bp, Artificial Sequence
  OXA48-R′
cgtctgtccatcccacttaaa

SEQ ID NO: 30 - DNA, 24 bp, Artificial Sequence
  OXA48-HEX
tagcttgatcgccctcgatttggg

SEQ ID NO: 31 - DNA, 19 bp, Artificial Sequence
  CMY F′
tccagcgttattgatatgg

SEQ ID NO: 32 - DNA, 18 bp, Artificial Sequence
  CMY R′
catctcccagcctaatcc

SEQ ID NO: 33 - DNA, 21 bp, Artificial Sequence
  CMY-TxR
acatatcgccaatacgccagt

SEQ ID NO: 34 - DNA, 19 bp, Artificial Sequence
  NDM F′
tttgatcgtcagggatggc

SEQ ID NO: 35 - DNA, 21 bp, Artificial Sequence
  NDM R′
caggttgatctcctgcttgat

SEQ ID NO: 36 - DNA, 22 bp, Artificial Sequence
  NDM-HEX
agaccgcccagatcctcaactg

SEQ ID NO: 37 - DNA, 21 bp, Artificial Sequence
  KPC F′
cgctaaactcgaacaggactt

SEQ ID NO: 38 - DNA, 21 bp, Artificial Sequence
  KPC R′
taacttacagttgcgcctgag

SEQ ID NO: 39 - DNA, 24 bp, Artificial Sequence
  KPC-FAM
atcggtgtgtacgcgatggatacc

SEQ ID NO: 40 - DNA, 19 bp, Artificial Sequence
  VIM F′
cattcgaccgacaacttag

SEQ ID NO: 41 - DNA, 17 bp, Artificial Sequence
  VIM R′
cgtgcgtgacaactcat

SEQ ID NO: 42 - DNA, 24 bp, Artificial Sequence
  VIM-TEX
tgtgctctatggtggttgtgcgat

SEQ ID NO: 43 - DNA, 21 bp, Artificial Sequence
  DHA F′
tgcgtacggttatgagaacaa

SEQ ID NO: 44 - DNA, 18 bp, Artificial Sequence
  DHA R′
cccagcgcagcatatctt

SEQ ID NO: 45 - DNA, 24 bp, Artificial Sequence
  DHA-FAM
atgcggaatcttacggcgtgaaat

SEQ ID NO: 46 - DNA, 21 bp, Artificial Sequence
  IMP F′
acgtagtggtttggttacctg

SEQ ID NO: 47 - DNA, 22 bp, Artificial Sequence
  IMP R′
aagcttctaaatttgcgtcacc

SEQ ID NO: 48 - DNA, 24 bp, Artificial Sequence
  IMP-TYE705
tttgttaaaccggacggtcttggt

SEQ ID NO: 49 - DNA, 201 bp, Artificial Sequence
  MOX
aaccgggagagcggggccagcgtcagcgagcagaccctgttcgagataggatccgtgagcaagaccctgactgcgaccctgggggcctatgcggtggtcaagggagcgatgcagctggatgacaaggcgagccggcacgcgccctggctcaagggatccgtctttgacagcatcaccatgggggagcttgccacctacagc

SEQ ID NO: 50 - DNA, 240 bp, Artificial Sequence
  FOX
ggggatggcggtcgccgtgctgaaagatggcaaggcccactatttcaactatggggttgccaaccgcgagagtggtcagcgcgtcagcgagcagaccctgttcgagattggctcggtcagcaagaccctgaccgcgaccctcggtgcctatgctgcggtcaaggggggctttgagctggatgacaaggtgagccagcacgccccctggctcaaaggttccgcctttgatggtgtgaccat

SEQ ID NO: 51 - DNA, 220 bp, Artificial Sequence
  EBC
ggaccgttacgccgctgatgaaagcgcaggccattccgggtatggcggtggcggtgatttatgagggtcagccgcactacttcaccttcggtaaagccgatgttgcggcgaacaaacctgtcactccacaaaccttgttcgaactgggttctataagtaaaaccttcaccggcgtactcggtggcgatgccattgctcgcggtgaaatatcgctgggcga

SEQ ID NO: 52 - DNA, 220 bp, Artificial Sequence
  DHA
gactgcacggatcctggcaccgctggggttatctcacacctttattactgtgccggaaagtgcgcaaagccagtatgcgtacggttatgagaacaaaaaaccggtccgcgtgtcgccgggacagcttgatgcggaatcttacggcgtggaatccgcctcaaaagatatgctgcgctgggcggaaatgaatatggagccgtcacgggccggtaatgcggat

SEQ ID NO: 53 - DNA, 260 bp, Artificial Sequence
  CMY
gcctgtacacgtttctccgggacaacttgacgccgaagcctatggcgtgaaatccagcgttattgatatggcccgctgggttcaggtcaacatggacgccagccgcgttcaggagaaaacgctccagcagggcattgcgcttgcgcagtctcgctactggcgtattggcgatatgtaccagggattaggctgggagatgctgaactggccgctgaaagctgattcgatcatcaacggtagcgacagcaaagtggcattgg

SEQ ID NO: 54 - DNA, 150 bp, Artificial Sequence
  ACC
gagagcaaaattaaagacaccgttgatgacctgatccagccgctgatgcagaagaataatattcccggtatgtcggtcgcagtgaccgtcaacggtaaaaactacatttataactatgggttagcggcaaaacagcctcagcagccggtt

SEQ ID NO: 55 - DNA, 270 bp, Artificial Sequence
  IC
agcttgttggtggggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccg

SEQ ID NO: 56 - DNA, 321 bp, Artificial Sequence
  CTX-M-14
cgtttcgtctggatcgcactgaacctacgctgaataccgccattcccggcgacccgagagacaccaccacgccgcgggcgatggcgcagacgttgcgtcagcttacgctgggtcatgcgctgggcgaaacccagcgggcgcagttggtgacgtggctcaaaggcaatacgaccggcgcagccagcattcgggccggcttaccgacgtcgtggactgtgggtgataagaccggcagcggcgactacggcaccaccaatgatattgcggtgatctggccgcagggtcgtgcgccgctggttctggtgacctattttacccagc

SEQ ID NO: 57 - DNA, 121 bp, Artificial Sequence
  CTX-M-15
ccgtcacgctgttgttaggaagtgtgccgctgtatgcgcaaacggcggacgtacagcaaaaacttgccgaattagagcggcagtcgggaggcagactgggtgtggcattgattaacacagc

SEQ ID NO: 58 - DNA, 553 bp, Artificial Sequence
  OXA
aatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgcgatatcgccacttggaatcgcgatcataatctaatcaccgcgatgaaatattcagttgtgcctgtttatcaagaatttgcccgccaaattggcgaggcacgtatgagcaagatgctacatgctttcgattatggtaatgaggacatttcgggcaatgtagacagtttctggctcgacggtggtattcgaatttcggccacggagcaaatcagctttttaagaaagctgtatcacaataagttacacgtatcggagcgcagccagcgtattgtcaaacaagccatgctgaccgaagccaatggtgactaattattcgggctaaaactggatactcgactagaatcgaacctaagattggctggctgggt

SEQ ID NO: 59 - DNA, 270 bp, Artificial Sequence
  IC
cggagttagccggtgcttcttctgcgggtaacgtcaatgagcaaaggtattaactttactcccttcctccccgctgaaagtactttacaacccgaaggccttcttcatacacgcggcatggctgcatcaggcttgcgcccattgtgcaatattccccactgctgcctcccgtaggagtctggaccgtgtctcagttccagtgtggctggtcatcctctcagaccagctagggatcgtcgccttggtgagccgttaccccaccaacaagct

SEQ ID NO: 60 - DNA, 260 bp, Artificial Sequence; identical to SEQ ID NO: 53
  CMY

SEQ ID NO: 61 - DNA, 263 bp, Artificial Sequence
  NDM
ggcgaaagtcaggctgtgttgcgccgcaaccatcccctcttgcggggcaagctggttcgacaacgcattggcataagtcgcaatccccgccgcatgcagcgcgtccataccgcccatcttgtcctgatgcgcgtgagtcaccaccgccagcgcgaccggcaggttgatctcctgcttgatccagttgaggatctgggcggtctggtcatcggtccaggcggtatcgaccaccagcacgcggccgccatccctgacgatcaaac

SEQ ID NO: 62 - DNA, 209 bp, Artificial Sequence
  KPC
gtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagg

SEQ ID NO: 63 - DNA, 292 bp, Artificial Sequence
  VIM
ccattcagccagatcggcatcggccacgttccccgcagacgtgcgtgacaactcatgaatcgcacaaccaccatagagcacactcgcagacgggacgtacacaactaagttgtcggtcgaatgcgcagcaccaggatagaagagttctactggaccgaagcgcactgcgtccccgctcgagtccttctagagagtgcgtgggaatctcgttcccctctacctcggctagccggcgtgtcgacggtgatgcgtacgttgccaccccagccgcccgaaggacatcaacgccgcc

SEQ ID NO: 64 - DNA, 220 bp, Artificial Sequence
  DHA
gactgcacggatcctggcaccgctggggttatctcacacctttattactgtgccggaaagtgcgcaaagccagtatgcgtacggttatgagaacaaaaaaccggtccgcgtgtcgccgggacagcttgatgcggaatcttacggcgtgaaatccgcctcaaaagatatgctgcgctgggcggaaatgaatatggagccgtcacgggccggtaatgcggat

SEQ ID NO: 65 - DNA, 270 bp, Artificial Sequence; identical to SEQ ID NO: 59
  IC

SEQ ID NO: 66 - DNA, 177 bp, Artificial Sequence
  IMP
gcggagttagttattggctagttaaaaataaaattgaagttttttatcccggcccggggcacactcaagataacgtagtggtttggttacctgaaaagaaaattttattcggtggttgttttgttaaaccggacggtcttgrvgtaatttgggtgacgcaaatttagaagcttggcc

SEQ ID NO: 67 - DNA, 21 bp, Artificial Sequence
  PRIMER/PROBE
tggccagaactgacaggcaaa

SEQ ID NO: 68 - DNA, 21 bp, Artificial Sequence
  PRIMER/PROBE
tttctcctgaacgtggctggc

SEQ ID NO: 69 - DNA, 24 bp, Artificial Sequence
  PRIMER/PROBE
acgctaactccagcattggtctgt

SEQ ID NO: 70 - DNA, 19 bp, Artificial Sequence
  PRIMER/PROBE
ccgtcacgctgttgttagg

SEQ ID NO: 71 - DNA, 22 bp, Artificial Sequence
  PRIMER/PROBE
gctgtgttaatcaatgccacac

SEQ ID NO: 72 - DNA, 24 bp, Artificial Sequence
  PRIMER/PROBE
aacttgccgaattagagcrgcagt

SEQ ID NO: 73 - DNA, 19 bp, Artificial Sequence
  PRIMER/PROBE
cgtttcgtctggatcgcac

SEQ ID NO: 74 - DNA, 20 bp, Artificial Sequence
  PRIMER/PROBE
gctgggtaaaataggtcacc

SEQ ID NO: 75 - DNA, 24 bp, Artificial Sequence
  PRIMER/PROBE
tatcattggtggtgccgtagtcgc

SEQ ID NO: 76 - DNA, 20 bp, Artificial Sequence
  PRIMER/PROBE
gagaggatgaycagccacac

SEQ ID NO: 77 - DNA, 20 bp, Artificial Sequence
  PRIMER/PROBE
cgcccattgtscaatattcc

SEQ ID NO: 78 - DNA, 24 bp, Artificial Sequence
  PRIMER/PROBE
tgagacacggtccagactcctacg

SEQ ID NO: 79 - DNA, 20 bp, Artificial Sequence
  PRIMER/PROBE
aatcacagggcgtagttgtg

SEQ ID NO: 80 - DNA, 20 bp, Artificial Sequence
  PRIMER/PROBE
acccaccagccaatcttagg

SEQ ID NO: 81 - DNA, 24 bp, Artificial Sequence
  PRIMER/PROBE
tagcttgatcgccctcgatttggg

SEQ ID NO: 82 - DNA, 21 bp, Artificial Sequence
  PRIMER/PROBE
gcggagttaactattggctag

SEQ ID NO: 83 - DNA, 21 bp, Artificial Sequence
  PRIMER/PROBE
ggccaagcttctatatttgcg

SEQ ID NO: 84 - DNA, 23 bp, Artificial Sequence
  PRIMER/PROBE
ttrttyggtggttgytttrttaa

SEQ ID NO: 85 - DNA, 21 bp, Artificial Sequence
  PRIMER/PROBE
gcggagttarytattggctag

SEQ ID NO: 86 - DNA, 21 bp, Artificial Sequence
  PRIMER/PROBE
ggccaagcytctawatttgcg

SEQ ID NO: 87 - DNA, 24 bp, Artificial Sequence
  PRIMER/PROBE
ccggacggtcttggtaatttgggt

SEQ ID NO: 88 - DNA, 24 bp, Artificial Sequence
  PRIMER/PROBE
ccgtacggtttaggcaatttgggt

SEQ ID NO: 89 - DNA, 20 bp, Artificial Sequence
  PRIMER/PROBE
ggcggcgttgatgtccttcg

SEQ ID NO: 90 - DNA, 21 bp, Artificial Sequence
  PRIMER/PROBE
ccattcagccagatcggcatc

SEQ ID NO: 91 - DNA, 23 bp, Artificial Sequence
  PRIMER/PROBE
agctcttctatcctggtgctgcg

SEQ ID NO: 92 - DNA, 22 bp, Artificial Sequence
  PRIMER/PROBE
aactttcacaggtgtgctgggt

SEQ ID NO: 93 - DNA, 21 bp, Artificial Sequence
  PRIMER/PROBE
ccgtacgcatactggctttgc

SEQ ID NO: 94 - DNA, 24 bp, Artificial Sequence
  PRIMER/PROBE
aaaccgggcgatatgcgtctgtat

SEQ ID NO: 95 - DNA, 20 bp, Artificial Sequence
  PRIMER/PROBE
gtatcgccgtctagttctgc

SEQ ID NO: 96 - DNA, 22 bp, Artificial Sequence
  PRIMER/PROBE
ccttgaatgagctgcacagtgg

SEQ ID NO: 97 - DNA, 23 bp, Artificial Sequence
  PRIMER/PROBE
tcgtcgcggaaccattcgctaaa

SEQ ID NO: 98 - DNA, 20 bp, Artificial Sequence
  PRIMER/PROBE
gtttgatcgtcagggatggc

SEQ ID NO: 99 - DNA, 18 bp, Artificial Sequence
  PRIMER/PROBE
ggcgaaagtcaggctgtg

SEQ ID NO: 100 - DNA, 24 bp, Artificial Sequence
  PRIMER/PROBE
catcaggacaagatgggcggtatg

SEQ ID NO: 101 - DNA, 21 bp, Artificial Sequence
  PRIMER/PROBE
gctgctcaaggagcacaggat

SEQ ID NO: 102 - DNA, 22 bp, Artificial Sequence
  PRIMER/PROBE
cacattgacataggtgtggtgc

SEQ ID NO: 103 - DNA, 24 bp, Artificial Sequence
  PRIMER/PROBE
aggatggcaaggcccactatttca

SEQ ID NO: 104 - DNA, 21 bp, Artificial Sequence
  PRIMER/PROBES
aacagcctcagcagccggtta

SEQ ID NO: 105 - DNA, 21 bp, Artificial Sequence
  PRIMER/PROBE
ttcgccgcaatcatccctagc

SEQ ID NO: 106 - DNA, 24 bp, Artificial Sequence
  PRIMER/PROBE
agccattacgttccagagttgcgt

SEQ ID NO: 107 - DNA, 21 bp, Artificial Sequence
  PRIMER/PROBE
gccgaggcttacgggatcaag

SEQ ID NO: 108 - DNA, 21 bp, Artificial Sequence
  PRIMER/PROBE
caaagcgcgtaaccggattgg

SEQ ID NO: 109 - DNA, 24 bp, Artificial Sequence
  PRIMER/PROBE
tctgctgaagtttrycgaggcmaa

SEQ ID NO: 110 - DNA, 22 bp, Artificial Sequence
  PRIMER/PROBE
aactttcacaggtgtgctgggt

SEQ ID NO: 111 - DNA, 21 bp, Artificial Sequence
  PRIMER/PROBE
ccgtacgcatactggctttgc

SEQ ID NO: 112 - DNA, 24 bp, Artificial Sequence
  PRIMER/PROBE
aaaccgggcgatatgcgtctgtat

SEQ ID NO: 113 - DNA, 29 bp, Artificial Sequence
  PRIMER/PROBE
ctgggttctataagtaaaaccttcaccgg

SEQ ID NO: 114 - DNA, 21 bp, Artificial Sequence
  PRIMER/PROBE
cttccactgcggctgccagtt

SEQ ID NO: 115 - DNA, 23 bp, Artificial Sequence
  PRIMER/PROBE
gatgccattgcycgsggtgaaat

SEQ ID NO: 116 - DNA, 23 bp, Artificial Sequence
  PRIMER/PROBE
ccgaagcctatggcgtgaaatcc

SEQ ID NO: 117 - DNA, 19 bp, Artificial Sequence
  PRIMER/PROBE
gcaatgccctgctggagcg

SEQ ID NO: 118 - DNA, 20 bp, Artificial Sequence
  PRIMER/PROBE
atgttggcctgaacccagcg

SEQ ID NO: 119 - DNA, 24 bp, Artificial Sequence
  PRIMER/PROBE
agcacatacagaatatgtccctgc

SEQ ID NO: 120 - DNA, 25 bp, Artificial Sequence
  PRIMER/PROBE
acctgttaaccaacctacttgaggg

SEQ ID NO: 121 - DNA, 24 bp, Artificial Sequence
  PRIMER/PROBE
ttgcaagacggactggcttagacc

SEQ ID NO: 122 - DNA, 20 bp, Artificial Sequence
  PRIMER/PROBE
cctgatcggattggagaacc

SEQ ID NO: 123 - DNA, 23 bp, Artificial Sequence
  PRIMER/PROBE
ctacctcttgaataggcgtaacc

SEQ ID NO: 124 - DNA, 24 bp, Artificial Sequence
  PRIMER/PROBE
acgtcgcgcaagttcctgatagac

SEQ ID NO: 125 - DNA, 25 bp, Artificial Sequence
  PRIMER/PROBE
tagtgactgctaatccaaatcacag

SEQ ID NO: 126 - DNA, 25 bp, Artificial Sequence
  PRIMER/PROBE
gcacgagcaagatcattaccatagc

SEQ ID NO: 127 - DNA, 27 bp, Artificial Sequence
  PRIMER/PROBE
agttatccaacaaggccaaactcaaca

SEQ ID NO: 128 - DNA, 20 bp, Artificial Sequence
  PRIMER/PROBE
aatcacagggcgtagttgtg

SEQ ID NO: 129 - DNA, 20 bp, Artificial Sequence
  PRIMER/PROBE
acccaccagccaatcttagg

SEQ ID NO: 130 - DNA, 24 bp, Artificial Sequence
  PRIMER/PROBE
tagcttgatcgccctcgatttggg

SEQ ID NO: 131 - DNA, 18 bp, Artificial Sequence
  PRIMER/PROBE
gtgggatggaaagccacg

SEQ ID NO: 132 - DNA, 18 bp, Artificial Sequence
  PRIMER/PROBE
cacttgcgggtctacagc

SEQ ID NO: 133 - DNA, 24 bp, Artificial Sequence
  PRIMER/PROBE
ttactttgggcgaagccatgcaag

SEQ ID NO: 134 - DNA, 21 bp, Artificial Sequence
  PRIMER/PROBE
cacctatggtaatgctcttgc

SEQ ID NO: 135 - DNA, 20 bp, Artificial Sequence
  PRIMER/PROBE
ctggaactgctgacaatgcc

SEQ ID NO: 136 - DNA, 30 bp, Artificial Sequence
  PRIMER/PROBE
tgggagaaagatatgactttaggtgaggca

SEQ ID NO: 137 - DNA, 18 bp, Artificial Sequence
  PRIMER/PROBES
ccgtgtatgttcagctat

SEQ ID NO: 138 - DNA, 18 bp, Artificial Sequence
  PRIMER/PROBE
cttatccatcacgccttt

SEQ ID NO: 139 - DNA, 26 bp, Artificial Sequence
  PRIMER/PROBES
tatgatgtcgataccgccaaatacca

SEQ ID NO: 140 - DNA, 18 bp, Artificial Sequence
  PRIMER/PROBE
ctgtatgtcagcgatcat

SEQ ID NO: 141 - DNA, 19 bp, Artificial Sequence
  PRIMER/PROBE
gatgccagtttgcttatcc

SEQ ID NO: 142 - DNA, 25 bp, Artificial Sequence
  PRIMER/PROBE
aagtctgggtgagaacggtgtctat

SEQ ID NO: 143 - DNA, 19 bp, Artificial Sequence
  PRIMER/PROBE
cagtcagtatgcgagtttc

SEQ ID NO: 144 - DNA, 18 bp, Artificial Sequence
  PRIMER/PROBE
aaaattcgccaagccatc

SEQ ID NO: 145 - DNA, 25 bp, Artificial Sequence
  PRIMER/PROBE
tgcataagccagtgcgtttttatat

SEQ ID NO: 146 - DNA, 18 bp, Artificial Sequence
  PRIMER/PROBE
agatcagttgggtgcacg

SEQ ID NO: 147 - DNA, 20 bp, Artificial Sequence
  PRIMER/PROBE
tgcttaatcagtgaggcacc

SEQ ID NO: 148 - DNA, 24 bp, Artificial Sequence
  PRIMER/PROBE
atgaagccataccaaacgacgagc

SEQ ID NO: 149 - DNA, 20 bp, Artificial Sequence
  PRIMER/PROBE
ctggagcgaaagatccacta

SEQ ID NO: 150 - DNA, 18 bp, Artificial Sequence
  PRIMER/PROBE
atcgtccaccatccactg

SEQ ID NO: 151 - DNA, 22 bp, Artificial Sequence
  PRIMER/PROBE
ccagatcggcgacaacgtcacc

SEQ ID NO: 152 - DNA, 21 bp, Artificial Sequence
  PRIMER/PROBE
tggccagaactgacaggcaaa

SEQ ID NO: 153 - DNA, 21 bp, Artificial Sequence
  PRIMER/PROBE
tttctcctgaacgtggctggc

SEQ ID NO: 154 - DNA, 24 bp, Artificial Sequence
  PRIMER/PROBE
acgctaactccagcattggtctgt

SEQ ID NO: 155 - DNA, 19 bp, Artificial Sequence
  PRIMER/PROBE
ccgtcacgctgttgttagg

SEQ ID NO: 156 - DNA, 22 bp, Artificial Sequence
  PRIMER/PROBE
gctgtgttaatcaatgccacac

SEQ ID NO: 157 - DNA, 24 bp, Artificial Sequence
  PRIMER/PROBE
aacttgccgaattagagcrgcagt

SEQ ID NO: 158 - DNA, 19 bp, Artificial Sequence
  PRIMER/PROBE
cgtttcgtctggatcgcac

SEQ ID NO: 159 - DNA, 20 bp, Artificial Sequence
  PRIMER/PROBE
gctgggtaaaataggtcacc

SEQ ID NO: 160 - DNA, 24 bp, Artificial Sequence
  PRIMER/PROBE
tatcattggtggtgccgtagtcgc

SEQ ID NO: 161 - DNA, 20 bp, Artificial Sequence
  PRIMER/PROBE
gagaggatgaycagccacac

SEQ ID NO: 162 - DNA, 20 bp, Artificial Sequence
  PRIMER/PROBE
cgcccattgtscaatattcc

SEQ ID NO: 163 - DNA, 24 bp, Artificial Sequence
  PRIMER/PROBE
tgagacacggtccagactcctacg

SEQ ID NO: 164 - DNA, 20 bp, Artificial Sequence
  PRIMER/PROBE
aatcacagggcgtagttgtg

SEQ ID NO: 165 - DNA, 20 bp, Artificial Sequence
  PRIMER/PROBE
acccaccagccaatcttagg

SEQ ID NO: 166 - DNA, 24 bp, Artificial Sequence
  PRIMER/PROBE
tagcttgatcgccctcgatttggg

SEQ ID NO: 167 - DNA, 21 bp, Artificial Sequence
  PRIMER/PROBE
gcggagttaactattggctag

SEQ ID NO: 168 - DNA, 21 bp, Artificial Sequence
  PRIMER/PROBE
ggccaagcttctatatttgcg

SEQ ID NO: 169 - DNA, 23 bp, Artificial Sequence
  PRIMER/PROBE
ttrttyggtggttgytttrttaa

SEQ ID NO: 170 - DNA, 21 bp, Artificial Sequence
  PRIMER/PROBE
gcggagttarytattggctag

SEQ ID NO: 171 - DNA, 21 bp, Artificial Sequence
  PRIMER/PROBE
ggccaagcytctawatttgcg

SEQ ID NO: 172 - DNA, 24 bp, Artificial Sequence
  PRIMER/PROBE
ccggacggtcttggtaatttgggt

SEQ ID NO: 173 - DNA, 24 bp, Artificial Sequence
  PRIMER/PROBE
ccgtacggtttaggcaatttgggt

SEQ ID NO: 174 - DNA, 20 bp, Artificial Sequence
  PRIMER/PROBE
ggcggcgttgatgtccttcg

SEQ ID NO: 175 - DNA, 21 bp, Artificial Sequence
  PRIMER/PROBE
ccattcagccagatcggcatc

SEQ ID NO: 176 - DNA, 23 bp, Artificial Sequence
  PRIMER/PROBE
agctcttctatcctggtgctgcg

SEQ ID NO: 177 - DNA, 20 bp, Artificial Sequence
  PRIMER/PROBE
gagaggatgaycagccacac

SEQ ID NO: 178 - DNA, 20 bp, Artificial Sequence
  PRIMER/PROBE
cgcccattgtscaatattcc

SEQ ID NO: 179 - DNA, 24 bp, Artificial Sequence
  PRIMER/PROBE
tgagacacggtccagactcctacg

SEQ ID NO: 180 - DNA, 22 bp, Artificial Sequence
  PRIMER/PROBE
aactttcacaggtgtgctgggt

SEQ ID NO: 181 - DNA, 21 bp, Artificial Sequence
  PRIMER/PROBE
ccgtacgcatactggctttgc

SEQ ID NO: 182 - DNA, 24 bp, Artificial Sequence
  PRIMER/PROBE
aaaccgggcgatatgcgtctgtat

SEQ ID NO: 183 - DNA, 20 bp, Artificial Sequence
  PRIMER/PROBE
gtatcgccgtctagttctgc

SEQ ID NO: 184 - DNA, 22 bp, Artificial Sequence
  PRIMER/PROBE
ccttgaatgagctgcacagtgg

SEQ ID NO: 185 - DNA, 23 bp, Artificial Sequence
  PRIMER/PROBE
tcgtcgcggaaccattcgctaaa

SEQ ID NO: 186 - DNA, 20 bp, Artificial Sequence
  PRIMER/PROBE
gtttgatcgtcagggatggc

SEQ ID NO: 187 - DNA, 18 bp, Artificial Sequence
  PRIMER/PROBE
ggcgaaagtcaggctgtg

SEQ ID NO: 188 - DNA, 24 bp, Artificial Sequence
  PRIMER/PROBE
catcaggacaagatgggcggtatg

SEQ ID NO: 189 - DNA, 20 bp, Artificial Sequence
  PRIMER/PROBE
gagaggatgaycagccacac

SEQ ID NO: 190 - DNA, 20 bp, Artificial Sequence
  PRIMER/PROBE
cgcccattgtscaatattcc

SEQ ID NO: 191 - DNA, 24 bp, Artificial Sequence
  PRIMER/PROBE
tgagacacggtccagactcctacg

SEQ ID NO: 192 - DNA, 21 bp, Artificial Sequence
  PRIMER/PROBE
gctgctcaaggagcacaggat

SEQ ID NO: 193 - DNA, 22 bp, Artificial Sequence
  PRIMER/PROBE
cacattgacataggtgtggtgc

SEQ ID NO: 194 - DNA, 24 bp, Artificial Sequence
  PRIMER/PROBE
aggatggcaaggcccactatttca

SEQ ID NO: 195 - DNA, 21 bp, Artificial Sequence
  PRIMER/PROBE
aacagcctcagcagccggtta

SEQ ID NO: 196 - DNA, 21 bp, Artificial Sequence
  PRIMER/PROBE
ttcgccgcaatcatccctagc

SEQ ID NO: 197 - DNA, 24 bp, Artificial Sequence
  PRIMER/PROBE
agccattacgttccagagttgcgt

SEQ ID NO: 198 - DNA, 21 bp, Artificial Sequence
  PRIMER/PROBE
gccgaggcttacgggatcaag

SEQ ID NO: 199 - DNA, 21 bp, Artificial Sequence
  PRIMER/PROBE
caaagcgcgtaaccggattgg

SEQ ID NO: 200 - DNA, 24 bp, Artificial Sequence
  PRIMER/PROBE
tctgctgaagtttrycgaggcmaa

SEQ ID NO: 201 - DNA, 20 bp, Artificial Sequence
  PRIMER/PROBE
gagaggatgaycagccacac

SEQ ID NO: 202 - DNA, 20 bp, Artificial Sequence
  PRIMER/PROBE
cgcccattgtscaatattcc

SEQ ID NO: 203 - DNA, 24 bp, Artificial Sequence
  PRIMER/PROBE
tgagacacggtccagactcctacg

SEQ ID NO: 204 - DNA, 22 bp, Artificial Sequence
  PRIMER/PROBE
aactttcacaggtgtgctgggt

SEQ ID NO: 205 - DNA, 21 bp, Artificial Sequence
  PRIMER/PROBE
ccgtacgcatactggctttgc

SEQ ID NO: 206 - DNA, 24 bp, Artificial Sequence
  PRIMER/PROBE
aaaccgggcgatatgcgtctgtat

SEQ ID NO: 207 - DNA, 29 bp, Artificial Sequence
  PRIMER/PROBE
ctgggttctataagtaaaaccttcaccgg

SEQ ID NO: 208 - DNA, 21 bp, Artificial Sequence
  PRIMER/PROBE
cttccactgcggctgccagtt

SEQ ID NO: 209 - DNA, 23 bp, Artificial Sequence
  PRIMER/PROBE
gatgccattgcycgsggtgaaat

SEQ ID NO: 210 - DNA, 23 bp, Artificial Sequence
  PRIMER/PROBE
ccgaagcctatggcgtgaaatcc

SEQ ID NO: 211 - DNA, 19 bp, Artificial Sequence
  PRIMER/PROBE
gcaatgccctgctggagcg

SEQ ID NO: 212 - DNA, 20 bp, Artificial Sequence
  PRIMER/PROBE
atgttggcctgaacccagcg

SEQ ID NO: 213 - DNA, 20 bp, Artificial Sequence
  PRIMER/PROBE
gagaggatgaycagccacac

SEQ ID NO: 214 - DNA, 20 bp, Artificial Sequence
  PRIMER/PROBE
cgcccattgtscaatattcc

SEQ ID NO: 215 - DNA, 24 bp, Artificial Sequence
  PRIMER/PROBE
tgagacacggtccagactcctacg

SEQ ID NO: 216 - DNA, 24 bp, Artificial Sequence
  PRIMER/PROBE
agcacatacagaatatgtccctgc

SEQ ID NO: 217 - DNA, 25 bp, Artificial Sequence
  PRIMER/PROBE
acctgttaaccaacctacttgaggg

SEQ ID NO: 218 - DNA, 24 bp, Artificial Sequence
  PRIMER/PROBE
ttgcaagacggactggcttagacc

SEQ ID NO: 219 - DNA, 20 bp, Artificial Sequence
  PRIMER/PROBE
cctgatcggattggagaacc

SEQ ID NO: 220 - DNA, 23 bp, Artificial Sequence
  PRIMER/PROBE
ctacctcttgaataggcgtaacc

SEQ ID NO: 221 - DNA, 24 bp, Artificial Sequence
  PRIMER/PROBE
acgtcgcgcaagttcctgatagac

SEQ ID NO: 222 - DNA, 25 bp, Artificial Sequence
  PRIMER/PROBE
tagtgactgctaatccaaatcacag

SEQ ID NO: 223 - DNA, 25 bp, Artificial Sequence
  PRIMER/PROBE
gcacgagcaagatcattaccatagc

SEQ ID NO: 224 - DNA, 27 bp, Artificial Sequence
  PRIMER/PROBE
agttatccaacaaggccaaactcaaca

SEQ ID NO: 225 - DNA, 20 bp, Artificial Sequence
  PRIMER/PROBE
gagaggatgaycagccacac

SEQ ID NO: 226 - DNA, 20 bp, Artificial Sequence
  PRIMER/PROBE
cgcccattgtscaatattcc

SEQ ID NO: 227 - DNA, 24 bp, Artificial Sequence
  PRIMER/PROBE
tgagacacggtccagactcctacg

SEQ ID NO: 228 - DNA, 20 bp, Artificial Sequence
  PRIMER/PROBE
aatcacagggcgtagttgtg

SEQ ID NO: 229 - DNA, 20 bp, Artificial Sequence
  PRIMER/PROBE
acccaccagccaatcttagg

SEQ ID NO: 230 - DNA, 24 bp, Artificial Sequence
  PRIMER/PROBE
tagcttgatcgccctcgatttggg

SEQ ID NO: 231 - DNA, 18 bp, Artificial Sequence
  PRIMER/PROBE
gtgggatggaaagccacg

SEQ ID NO: 232 - DNA, 18 bp, Artificial Sequence
  PRIMER/PROBE
cacttgcgggtctacagc

SEQ ID NO: 233 - DNA, 24 bp, Artificial Sequence
  PRIMER/PROBE
ttactttgggcgaagccatgcaag

SEQ ID NO: 234 - DNA, 21 bp, Artificial Sequence
  PRIMER/PROBE
cacctatggtaatgctcttgc

SEQ ID NO: 235 - DNA, 20 bp, Artificial Sequence
  PRIMER/PROBE
ctggaactgctgacaatgcc

SEQ ID NO: 236 - DNA, 30 bp, Artificial Sequence; identical to SEQ ID NO: 136
  PRIMER/PROBE

SEQ ID NO: 237 - DNA, 20 bp, Artificial Sequence
  PRIMER/PROBE
gagaggatgaycagccacac

SEQ ID NO: 238 - DNA, 20 bp, Artificial Sequence
  PRIMER/PROBE
cgcccattgtscaatattcc

SEQ ID NO: 239 - DNA, 24 bp, Artificial Sequence
  PRIMER/PROBE
tgagacacggtccagactcctacg

SEQ ID NO: 240 - DNA, 18 bp, Artificial Sequence
  PRIMER/PROBE
agatcagttgggtgcacg

SEQ ID NO: 241 - DNA, 20 bp, Artificial Sequence
  PRIMER/PROBE
tgcttaatcagtgaggcacc

SEQ ID NO: 242 - DNA, 24 bp, Artificial Sequence
  PRIMER/PROBE
atgaagccataccaaacgacgagc

SEQ ID NO: 243 - DNA, 20 bp, Artificial Sequence
  PRIMER/PROBE
ctggagcgaaagatccacta

SEQ ID NO: 244 - DNA, 18 bp, Artificial Sequence
  PRIMER/PROBE
atcgtccaccatccactg

SEQ ID NO: 245 - DNA, 22 bp, Artificial Sequence
  PRIMER/PROBE
ccagatcggcgacaacgtcacc

SEQ ID NO: 246 - DNA, 20 bp, Artificial Sequence
  PRIMER/PROBE
gagaggatgaycagccacac

SEQ ID NO: 247 - DNA, 20 bp, Artificial Sequence
  PRIMER/PROBE
cgcccattgtscaatattcc

SEQ ID NO: 248 - DNA, 24 bp, Artificial Sequence
  PRIMER/PROBE
tgagacacggtccagactcctacg

SEQ ID NO: 249 - DNA, 18 bp, Artificial Sequence
  PRIMER/PROBE
ccgtgtatgttcagctat

SEQ ID NO: 250 - DNA, 18 bp, Artificial Sequence
  PRIMER/PROBE
cttatccatcacgccttt

SEQ ID NO: 251 - DNA, 26 bp, Artificial Sequence
  PRIMER/PROBE
tatgatgtcgataccgccaaatacca

SEQ ID NO: 252 - DNA, 18 bp, Artificial Sequence
  PRIMER/PROBE
ctgtatgtcagcgatcat

SEQ ID NO: 253 - DNA, 19 bp, Artificial Sequence
  PRIMER/PROBE
gatgccagtttgcttatcc

SEQ ID NO: 254 - DNA, 25 bp, Artificial Sequence
  PRIMER/PROBE
aagtctgggtgagaacggtgtctat

SEQ ID NO: 255 - DNA, 19 bp, Artificial Sequence
  PRIMER/PROBE
cagtcagtatgcgagtttc

SEQ ID NO: 256 - DNA, 18 bp, Artificial Sequence
  PRIMER/PROBE
aaaattcgccaagccatc

SEQ ID NO: 257 - DNA, 25 bp, Artificial Sequence
  PRIMER/PROBE
tgcataagccagtgcgtttttatat

SEQ ID NO: 258 - DNA, 20 bp, Artificial Sequence
  PRIMER/PROBE
gagaggatgaycagccacac

SEQ ID NO: 259 - DNA, 20 bp, Artificial Sequence
  PRIMER/PROBE
cgcccattgtscaatattcc

SEQ ID NO: 260 - DNA, 24 bp, Artificial Sequence
  PRIMER/PROBE
tgagacacggtccagactcctacg

SEQ ID NO: 261 - DNA, 462 bp, Artificial Sequence
  CONTROL MIX
tggccagaactgacaggcaaacagtggcagggtatccgcctgctgcacttagccacctatacggcaggcggcctaccgctgcagatccccgatgacgttagggataaagccgcattactgcatttttatcaaaactggcagccgcaatggactccgggcgctaagcgactttacgctaactccagcattggtctgtttggcgcgctggcggtgaaaccctcaggaatgagttacgaagaggcaatgaccagacgcgtcctgcaaccattaaaactggcgcatacctggattacggttccgcagaacgaacaaaaagattatgcctggggctatcgcgaagggaagcccgtacacgtttctccgggacaacttgacgccgaagcctatggcgtgaaatccagcgttattgatatggcccgctgggttcaggccaacatggatgccagccacgttcaggagaaa

SEQ ID NO: 262 - DNA, 121 bp, Artificial Sequence; identical to SEQ ID NO: 57
  CONTROL MIX

SEQ ID NO: 263 - DNA, 321 bp, Artificial Sequence; identical to SEQ ID NO: 56
  CONTROL MIX

SEQ ID NO: 264 - DNA, 84 bp, Artificial Sequence
  CONTROL MIX
gagaggatgaccagccacactggaactgagacacggtccagactcctacgggaggcagcagtggggaatattgcacaatgggcg

SEQ ID NO: 265 - DNA, 553 bp, Artificial Sequence
  CONTROL MIX
aatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgcgatatcgccacttggaatcgcgatcataatctaatcaccgcgatgaaatattcagttgtgcctgtttatcaagaatttgcccgccaaattggcgaggcacgtatgagcaagatgctacatgctttcgattatggtaatgaggacatttcgggcaatgtagacagtttctggctcgacggtggtattcgaatttcggccacggagcaaatcagctttttaagaaagctgtatcacaataagttacacgtatcggagcgcagccagcgtattgtcaaacaagccatgctgaccgaagccaatggtgactatattattcgggctaaaactggatactcgactagaatcgaacctaagattggctggtgggt

SEQ ID NO: 266 - DNA, 175 bp, Artificial Sequence
  CONTROL MIX
gcggagttagttattggctagttaaaaataaaattgaagttttttatcccggcccggggcacactcaagataacgtagtggtttggttacctgaaaagaaaattttattcggtggttgttttgttaaaccggacggtcttggtaatttgggtgacgcaaatttagaagcttggcc

SEQ ID NO: 267 - DNA, 297 bp, Artificial Sequence
  CONTROL MIX
ggcggcgttgatgtccttcgggcggctggggtggcaacgtacgcatcaccgtcgacacgccggctagccgaggtagaggggaacgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgattcatgagttgtcacgcacgtctgcggggaacgtggccgatgccgatctggctgaatgg

SEQ ID NO: 268 - DNA, 84 bp, Artificial Sequence; identical to SEQ ID NO: 264
  CONTROL MIX

SEQ ID NO: 269 - DNA, 405 bp, Artificial Sequence
  CONTROL MIX
aactttcacaggtgtgctgggtgcggtttctgtggcgaaaaaagagatggcgctgaatgatccggcggcaaaataccagccggagctggctctgccgcagtggaaggggatcacattgctggatctggctacctataccgcaggcggactgccgttacaggtgccggatgcggtaaaaagccgtgcggatctgctgaatttctatcagcagtggcagccgtcccggaaaccgggcgatatgcgtctgtatgcaaacagcagtatcggcctgtttggtgctctgaccgcaaacgcggcggggatgccgtatgagcagttgctgactgcacggatcctggcaccgctggggttatctcacacctttattactgtgccggaaagtgcgcaaagccagtatgcgtacgg

SEQ ID NO: 270 - DNA, 209 bp, Artificial Sequence; identical to SEQ ID NO: 62
  CONTROL MIX

SEQ ID NO: 271 - DNA, 263 bp, Artificial Sequence
  CONTROL MIX
gtttgatcgtcagggatggcggccgcgtgctggtggtcgataccgcctggaccgatgaccagaccgcccagatcctcaactggatcaagcaggagatcaacctgccggtcgcgctggcggtggtgactcacgcgcatcaggacaagatgggcggtatggacgcgctgcatgcggcggggattgcgacttatgccaatgcgttgtcgaaccagcttgccccgcaagaggggatggttgcggcgcaacacagcctgactttcgcc

SEQ ID NO: 272 - DNA, 84 bp, Artificial Sequence; identical to SEQ ID NO: 264
  CONTROL MIX

SEQ ID NO: 273 - DNA, 520 bp, Artificial Sequence
  CONTROL MIX
gctgctcaaggagcacaggatcccgggcatggcggtggccgtgctcaaggatggcaaggcccactatttcaattacggggtggccaaccgggagagcggggccagcgtcagcgagcagaccctgttcgagataggatccgtgagcaagaccctgactgcgaccctgggggcctatgcggtggtcaagggagcgatgcagctggatgacaaggcgagccggcacgcgccctggctcaagggatccgtctttgacagcatcaccatgggggagcttgccacctacagcgccggaggcctgccactgcaattccccgaggaggtggattcatccgagaagatgcgcgcctactaccgccagtgggcccctgtctattcgccgggctcccatcgccagtactccaaccccagcatagggctgttcggccacctggcggcgagcagcctgaagcagccatttgcccagttgatggagcagaccctgctgcccgggctcggcatgcaccacacctatgtcaatgtg

SEQ ID NO: 274 - DNA, 346 bp, Artificial Sequence
  CONTROL MIX
aacagcctcagcagccggttacggaaaatacgttatttgaagtgggttcgctgagtaaaacgtttgctgccaccttggcgtcctatgcgcaggtgagcggtaagctgtctttggatcaaagcgttagccattacgttccagagttgcgtggcagcagctttgaccacgttagcgtactcaatgtgggcacgcatacctcaggcctacagctatttatgccggaagatattaaaaataccacacagctgatggcttatctaaaagcatggaaacctgccgatgcggctggaacccatcgcgtttattccaatatcggtactggtttgctagggatgattgcggcgaa

SEQ ID NO: 275 - DNA, 247 bp, Artificial Sequence
  CONTROL MIX
gccgaggcttacgggatcaagaccggctcggcggatctgctgaagtttaccgaggccaacatggggtatcagggagatgccgcgctaaaaacgcggatcgcgctgacccataccggtttctactcggtgggagacatgactcaggggctgggttgggagagctacgcctatccgttgaccgagcaggcgctgctggcgggcaactccccggcggtgagcttccaggccaatccggttacgcgctttg

SEQ ID NO: 276 - DNA, 84 bp, Artificial Sequence; identical to SEQ ID NO: 264
  CONTROL MIX

SEQ ID NO: 277 - DNA, 405 bp, Artificial Sequence; identical to SEQ ID NO: 269
  CONTROL MIX

SEQ ID NO: 278 - DNA, 302 bp, Artificial Sequence
  CONTROL MIX
tcggtaaagccgatgttgcggcgaacaaacccgtcaccccgcaaaccctgtttgagctgggctctataagtaaaaccttcaccggcgtactgggcggcgatgccattgcccggggtgaaatagcgctgggcgatccggtagcaaaatactggcctgagctcacgggcaagcagtggcagggcattcgcatgctggatctggcaacctataccgcaggcggtctgccgttacaggtgccggatgaggtcacggataccgcctctctgctgcgcttttatcaaaactggcagccgcagtggaag

SEQ ID NO: 279 - DNA, 106 bp, Artificial Sequence
  CONTROL MIX
ccgaagcctatggcgtgaaatccagcgttattgatatggcccgctgggttcaggccaacatggatgccagccacgttcaggagaaaacgctccagcagggcattgc

SEQ ID NO: 280 - DNA, 84 bp, Artificial Sequence; identical to SEQ ID NO: 264
  CONTROL MIX

SEQ ID NO: 281 - DNA, 487 bp, Artificial Sequence
  CONTROL MIX
agcacatacagaatatgtccctgcatcaacatttaagatgctaaatgccttaattggactagaaaatcataaagctacaacaactgagattttcaaatgggacggtaaaaagagatcttatcccatgtgggaaaaagatatgactttaggtgatgccatggcactttcagcagttcctgtatatcaagaacttgcaagacggactggcttagacctaatgcaaaaagaagttaaacgggttggttttggtaatatgaacattggaacacaagttgataacttctggttggttggccccctcaagattacaccaatacaagaggttaattttgccgatgattttgcaaataatcgattaccctttaaattagagactcaagaagaagttaaaaaaatgcttctgattaaagaattcaatggtagtaaaatttatgcaaaaagcggctggggaatggatgtaacccctcaagtaggttggttaacaggt

SEQ ID NO: 282 - DNA, 278 bp, Artificial Sequence
  CONTROL MIX
cctgatcggattggagaaccagaaaacggatattaatgaaatatttaaatggaagggcgagaaaaggtcatttaccgcttgggaaaaagacatgacactaggagaagccatgaagctttctgcagtcccagtctatcaggaacttgcgcgacgtatcggtcttgatctcatgcaaaaagaagtaaaacgtattggtttcggtaatgctgaaattggacagcaggttgataatttctggttggtaggaccattaaaggttacgcctattcaagaggtag

SEQ ID NO: 283 - DNA, 151 bp, Artificial Sequence
  CONTROL MIX
tagtgactgctaatccaaatcacagcgcttcaaaatctgatgaaaaagcagagaaaattaaaaatttatttaacgaagtacacactacgggtgttttagttatccaacaaggccaaactcaacaaagctatggtaatgatcttgctcgtgc

SEQ ID NO: 284 - DNA, 84 bp, Artificial Sequence; identical to SEQ ID NO: 264
  CONTROL MIX

SEQ ID NO: 285 - DNA, 553 bp, Artificial Sequence; identical to SEQ ID NO: 265
  CONTROL MIX

SEQ ID NO: 286 - DNA, 376 bp, Artificial Sequence
  CONTROL MIX
gtgggatggaaagccacgtttttttaaagcatgggacaaagattttactttgggcgaagccatgcaagcatctacagtgcctgtatatcaagaattggcacgtcgtattggtccaagcttaatgcaaagtgaattgcaacgtattggttatggcaatatgcaaataggcacggaagttgatcaattttggttgaaagggcctttgacaattacacctatacaagaagtaaagtttgtgtatgatttagcccaagggcaattgccttttaaacctgaagttcagcaacaagtgaaagagatgttgtatgtagagcgcagaggggagaatcgtctatatgctaaaagtggctggggaatggctgtagacccgcaagtg

SEQ ID NO: 287 - DNA, 376 bp, Artificial Sequence
  CONTROL MIX
cacttgcgggtctacagccattccccagccacttttagcatatagacgattctcccctctgcgctctacatacaacatctctttcacttgttgctgaacttcaggtttaaaaggcaattgcccttgggctaaatcatacacaaactttacttcttgtataggtgtaattgtcaaaggccctttcaaccaaaattgatcaacttccgtgcctatttgcatattgccataaccaatacgttgcaattcactttgcattaagcttggaccaatacgacgtgccaattcttgatatacaggcactgtagatgcttgcatggcttcgcccaaagtaaaatctttgtcccatgctttaaaaaaacgtggctttccatcccac

SEQ ID NO: 288 - DNA, 203 bp, Artificial Sequence
  CONTROL MIX
cacctatggtaatgctcttgcacgagcaaataaagaatatgtccctgcatcaacatttaagatgctaaatgctttaatcgggctagaaaatcataaagcaacaacaaatgagattttcaaatgggatggtaaaaaaagaacttatcctatgtgggagaaagatatgactttaggtgaggcaatggcattgtcagcagttccag

SEQ ID NO: 289 - DNA, 84 bp, Artificial Sequence; identical to SEQ ID NO: 264
  CONTROL MIX

SEQ ID NO: 290 - DNA, 1625 bp, Artificial Sequence
  CONTROL MIX
atgatgcagcatacttctgtgtggtaccgacgctcggtcagtccgtttgttcttgtgggagtgttgccgttttcttgaccgcgaccgccaatcttaccttttttgataaaatcagccaaacctatcccatcgcggacaatctcggctttgtgctgacgatcgctgtcgtgctctttggcgcgatgctactgatcaccacgctgttatcatcgtatcgctatgtgctaaagcctgtgttgattttgctattaatcatgggcgcggtgaccagttattttactgacacttatggcacggtctatgatacgaccatgctccaaaatgccctacagaccgaccaagccgagaccaaggatctattaaacgcagcgtttatcatgcgtatcattggtttgggtgtgctaccaagtttgcttgtggcttttgttaaggtggattatccgacttggggcaagggtttgatgcgccgattgggcttgatcgtggcaagtcttgcgctgattttactgcctgtggtggcgttcagcagtcattatgccagtttctttcgcgtgcataagccgctgcgtagctatgtcaatccgatcatgccaatctactcggtgggtaagcttgccagtattgagtataaaaaagccagtgcgccaaaagataccatttatcacgccaaagacgcggtacaagcaaccaagcctgatatgcgtaagccacgcctagtggtgttcgtcgtcggtgagacggcacgcgccgatcatgtcagcttcaatggctatgagcgcgatactttcccacagcttgccaagatcgatggcgtgaccaattttagcaatgtcacatcgtgcggcacatcgacggcgtattctgtgccgtgtatgttcagctatctgggcgcggatgagtatgatgtcgataccgccaaataccaagaaaatgtgctggatacgctggatcgcttgggcgtaagtatcttgtggcgtgataataattcggactcaaaaggcgtgatggataagctgccaaaagcgcaatttgccgattataaatccgcgaccaacaacgccatctgcaacaccaatccttataacgaatgccgcgatgtcggtatgctcgttggcttagatgactttgtcgctgccaataacggcaaagatatgctgatcatgctgcaccaaatgggcaatcacgggcctgcgtattttaagcgatatgatgaaaagtttgccaaattcacgccagtgtgtgaaggtaatgagcttgccaagtgcgaacatcagtccttgatcaatgcttatgacaatgccttgcttgccaccgatgatttcatcgctcaaagtatccagtggctgcagacgcacagcaatgcctatgatgtctcaatgctgtatgtcagcgatcatggcgaaagtctgggtgagaacggtgtctatctacatggtatgccaaatgcctttgcaccaaaagaacagcgcagtgtgcctgcatttttctggacggataagcaaactggcatcacgccaatggcaaccgataccgtcctgacccatgacgcgatcacgccgacattattaaagctgtttgatgtcaccgcggacaaagtcaaagaccgcaccgcattcatccgctga

SEQ ID NO: 291 - DNA, 1617 bp, Artificial Sequence
  CONTROL MIX
atgacatcacatcactcttggtatcgctattctatcaatccttttgtgctgatgggtttggtggcgttatttttggcagcgacagcgaacctgacattttttgaaaaagcgatggcggtctatcctgtatcggataacttaggctttatcatctcaatggcggtggcggtgatgggtgctatgctactgattgtcgtgctgttatcctatcgctatgtgctaaagcctgtcctgattttgctactgattatgggtgcggtgacgagctattttaccgatacttatggcacggtctatgacaccaccatgctccaaaatgccatgcaaaccgaccaagccgagtctaaggacttgatgaatttggcgttttttgtgcgaattatcgggcttggcgtgttgccaagtgtgttggtcgcagttgccaaagtcaattatccaacatggggcaaaggtctgattcagcgtgcgatgacatggggtgtcagccttgtgctgttgcttgtgccgattggactatttagcagtcagtatgcgagtttctttcgggtgcataagccagtgcgtttttatatcaacccgattacgccgatttattcggtgggtaagcttgccagtatcgagtacaaaaaagccactgcgccaacagacaccatctatcatgccaaagacgccgtgcagaccaccaagccgagcgagcgtaagccacgcctagtggtgttcgtcgtcggtgagacggcgcgtgctgaccatgtgcagttcaatggctatggccgtgagactttcccgcagcttgccaaagttgatggcttggcgaattttagccaagtgacatcgtgtggcacatcgacggcgtattctgtgccgtgtatgttcagctatttgggtcaagatgactatgatgtcgataccgccaaataccaagaaaatgtgctagatacgcttgaccgcttgggtgtgggtatcttgtggcgtgataataattcagactcaaaaggcgtgatggataagctacctgccacgcagtattttgattataaatcagcaaccaacaataccatctgtaacaccaatccctataacgaatgccgtgatgtcggtatgcttgtcgggctagatgactatgtcagcgccaataatggcaaagatatgctcatcatgctacaccaaatgggcaatcatgggccggcgtactttaagcgttatgatgagcaatttgccaaattcacccccgtgtgcgaaggcaacgagcttgccaaatgcgaacaccaatcactcatcaatgcctatgacaatgcgctacttgcgactgatgattttatcgccaaaagcatcgattggctaaaaacgcatgaagcgaactacgatgtcgccatgctctatgtcagtgaccacggcgagagcttgggcgaaaatggtgtctatctgcatggtatgccaaatgcctttgcaccaaaagaacagcgagctgtgcctgcgtttttttggtcaaataatacgacattcaagccaactgccagcgatactgtgctgacgcatgatgcgattacgccaacactgcttaagctgtttgatgtcacagcgggcaaggtcaaagaccgcgcggcatttatccagtaa

SEQ ID NO: 292 - DNA, 84 bp, Artificial Sequence; identical to SEQ ID NO: 264
  CONTROL MIX

SEQ ID NO: 293 - DNA, 750 bp, Artificial Sequence
  CONTROL MIX
agatcagttgggtgcacgagtgggttacatcgaactggatctcaacagcggtaagatccttgagagttttcgccccgaagaacgttttccaatgatgagcacttttaaagttctgctatgtggtgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgacgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccagtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagca

SEQ ID NO: 294 - DNA, 372 bp, Artificial Sequence
  CONTROL MIX
ctggagcgaaagatccactatcgccagcaggatctggtggactactcgccggtcagcgaaaaacaccttgccgacggcatgacggtcggcgaactctgcgccgccgccattaccatgagcgataacagcgccgccaatctgctgctggccaccgtcggcggccccgcaggattgactgcctttttgcgccagatcggcgacaacgtcacccgccttgaccgctgggaaacggaactgaatgaggcgcttcccggcgacgcccgcgacaccactaccccggccagcatggccgcgaccctgcgcaagctgctgaccagccagcgtctgagcgcccgttcgcaacggcagctgctgcagtggatggtggacgat

SEQ ID NO: 295 - DNA, 84 bp, Artificial Sequence; identical to SEQ ID NO: 264
  CONTROL MIX

SEQ ID NO: 296 - DNA, 21 bp, Artificial Sequence
  Synthetic polynucleotide
tttatccaggcccagggcaca

SEQ ID NO: 297 - DNA, 20 bp, Artificial Sequence
  Synthetic polynucleotide
ttttttatccaggcccaggg

SEQ ID NO: 298 - DNA, 21 bp, Artificial Sequence
  Synthetic polynucleotide
acggggttagttattggctgg

SEQ ID NO: 299 - DNA, 21 bp, Artificial Sequence
  Synthetic polynucleotide
ggccaagcttctaaatttgcg

SEQ ID NO: 300 - DNA, 24 bp, Artificial Sequence
  Synthetic polynucleotide
cctcacggccttggtaatttgggt

SEQ ID NO: 301 - DNA, 26 bp, Artificial Sequence
  Synthetic polynucleotide
accgtatggtctaggtaatttgggtg

SEQ ID NO: 302 - DNA, 24 bp, Artificial Sequence
  Synthetic polynucleotide
cctcacggtcttggcaatttaggt

SEQ ID NO: 303 - DNA, 18 bp, Artificial Sequence
  Synthetic polynucleotide
aagtctgggtgagaacgg

SEQ ID NO: 304 - DNA, 21 bp, Artificial Sequence
  Synthetic polynucleotide
tgcataagccagtgcgttttt

SEQ ID NO: 305 - DNA, 20 bp, Artificial Sequence
  Synthetic polynucleotide
gatgttcgttcgtgtgggac

SEQ ID NO: 306 - DNA, 23 bp, Artificial Sequence
  Synthetic polynucleotide
gaaggacaacctcgtcatagcat

SEQ ID NO: 307 - DNA, 20 bp, Artificial Sequence
  Synthetic polynucleotide
aaaggcgtctgcgaccgagt

SEQ ID NO: 308 - DNA, 21 bp, Artificial Sequence
  Synthetic polynucleotide
aggcgttacattgtccctacc

SEQ ID NO: 309 - DNA, 18 bp, Artificial Sequence
  Synthetic polynucleotide
agcacggcgaggatcata

SEQ ID NO: 310 - DNA, 24 bp, Artificial Sequence
  Synthetic polynucleotide
tctgcccgccccattcgtgaaaac

SEQ ID NO: 311 - DNA, 21 bp, Artificial Sequence
  Synthetic polynucleotide
gactacgacgaacgccagatt

SEQ ID NO: 312 - DNA, 19 bp, Artificial Sequence
  Synthetic polynucleotide
tcggtagcttgcgggatag

SEQ ID NO: 313 - DNA, 23 bp, Artificial Sequence
  Synthetic polynucleotide
agtcgggctgtaaaggcgtctgt

SEQ ID NO: 314 - DNA, 20 bp, Artificial Sequence
  Synthetic polynucleotide
cggtcgccgcatacactatt

SEQ ID NO: 315 - DNA, 20 bp, Artificial Sequence
  Synthetic polynucleotide
cagtgctgcaatgataccgc

SEQ ID NO: 316 - DNA, 21 bp, Artificial Sequence
  Synthetic polynucleotide
cgccattaccatgagcgataa

SEQ ID NO: 317 - DNA, 19 bp, Artificial Sequence
  Synthetic polynucleotide
ggaagcgcctcattcagtt

SEQ ID NO: 318 - DNA, 20 bp, Artificial Sequence
  Synthetic polynucleotide
acaacgtcacccgccttgac

SEQ ID NO: 319 - DNA, 20 bp, Artificial Sequence
  Synthetic polynucleotide
gctgatcggaaaccaaacgg

SEQ ID NO: 320 - DNA, 20 bp, Artificial Sequence
  Synthetic polynucleotide
acttgaccgacagaggcaac

SEQ ID NO: 321 - DNA, 21 bp, Artificial Sequence
  Synthetic polynucleotide
aaaccaatgtcgttccggccc

SEQ ID NO: 322 - DNA, 26 bp, Artificial Sequence
  Synthetic polynucleotide
ggtaataacctggtacgagcacatac

SEQ ID NO: 323 - DNA, 25 bp, Artificial Sequence
  Synthetic polynucleotide
accaaaaccaacccgtttaacttct

SEQ ID NO: 324 - DNA, 25 bp, Artificial Sequence
  Synthetic polynucleotide
ccatggcactttcagcagttcctgt

SEQ ID NO: 325-327 - skipped (3 entries)

SEQ ID NO: 328 - DNA, 26 bp, Artificial Sequence
  Synthetic polynucleotide
cttagcacctatggtaatgctcttgc

SEQ ID NO: 329 - DNA, 22 bp, Artificial Sequence
  Synthetic polynucleotide
ttctgcattagctctaggccag

SEQ ID NO: 330 - DNA, 29 bp, Artificial Sequence
  Synthetic polynucleotide
actttaggtgaggcaatggcattgtcagc

SEQ ID NO: 331 - DNA, 23 bp, Artificial Sequence
  Synthetic polynucleotide
catcgatcagaatgttcaagcgc

SEQ ID NO: 332 - DNA, 23 bp, Artificial Sequence
  Synthetic polynucleotide
ccaatacgacgtgccaattcttg

SEQ ID NO: 333 - DNA, 29 bp, Artificial Sequence
  Synthetic polynucleotide
tggcacgcatttagaccgagcaaaaacag

SEQ ID NO: 334 - DNA, 21 bp, Artificial Sequence
  Synthetic polynucleotide
ctcgtgcttcgaccgagtatg

SEQ ID NO: 335 - DNA, 21 bp, Artificial Sequence
  Synthetic polynucleotide
ttaaccagcctacttgtgggt

SEQ ID NO: 336 - DNA, 30 bp, Artificial Sequence
  Synthetic polynucleotide
cctgcttcgaccttcaaaatgcttaatgct

SEQ ID NO: 337 - DNA, 24 bp, Artificial Sequence
  Synthetic polynucleotide
tacagaatatgtgccagcctctac

SEQ ID NO: 338 - DNA, 24 bp, Artificial Sequence
  Synthetic polynucleotide
gcatgagatcaagaccgatacgtc

SEQ ID NO: 339 - DNA, 24 bp, Artificial Sequence
  Synthetic polynucleotide
tgccctgatcggattggagaacca

SEQ ID NO: 340 - DNA, 26 bp, Artificial Sequence
  Synthetic polynucleotide
tgagacacgtagtccagactcctacg

SEQ ID NO: 341 - DNA, 132 bp, Artificial Sequence
  Synthetic polynucleotide
ctgtatgtcagcgatcatggcgaaagtctgggtgagaacggtgtctatctacatggtatgccaaatgcctttgcaccaaaagaacagcgcagtgtgcctgcatttttctggacggataagcaaactggcatc

SEQ ID NO: 342 - DNA, 297 bp, Artificial Sequence
  Synthetic polynucleotide
cagtcagtatgcgagtttctttcgggtgcataagccagtgcgtttttatatcaacccgattacgccgatttattcggtgggtaagcttgccagtatcgagtacaaaaaagccactgcgccaacagacaccatctatcatgccaaagacgccgtgcagccaccaagccgagcgagcgtaagccacgcctagtggtgttcgtcgtcggtgagacggcgcgtgctgaccatgtgcagttcaatggctatggccgtgagactttcccgcagcttgccaaagttgatggcttggcgaatttt

SEQ ID NO: 343 - DNA, 279 bp, Artificial Sequence
  Synthetic polynucleotide
tttaatgatgttcgttcgtgtgggactgcaaccgctgtatccgtcccctgcatgttctccaatatggggagaaaggagtttgatgataatcgcgctcgcaatagcgagggcctgctagatgtgttgcaaaaaacggggatctccattttttggaaggagaacgatggaggctgcaaaggcgtctgcgaccgagtacctaacatcgaaatcgaaccaaaggatcaccctaagttctgcgataaaaacacatgctatgacgaggttgtccttcaagacctc

SEQ ID NO: 344 - DNA, 356 bp, Artificial Sequence
  Synthetic polynucleotide
tgagttaaggcgttacattgtccctacctattttgtcagtagtgcatctaaatatctcaatgagcactatttgcagacgcccatggaataccaacaacttggcctagatgcgaagaatgccagtcgtaacccgaacactaaacctaacttattagtggttgttgtgggtgaaactgcgcgctcaatgagctatcaatattatggatataacaagccaaccaatgctcatacccaaaatcaggggctgattgcgtttaacgatactagctcatgcggcacggccacggcggtgtctctaccctgtatgttttcacgaatggggcgggcagactatgatcctcgccgtgctaatgctc

SEQ ID NO: 345 - DNA, 319 bp, Artificial Sequence
  Synthetic polynucleotide
tcggcgcgactacgacgaacgccagattcgtcggcgcgagtccgtgctgcacgttttaaaccgtagtgacgtcaacattctctggcgcgataaccagtcgggctgtaaaggcgtctgtgatggactgccctttgaaaacctgtcttcggcaggccatcccacactgtgccatggcgtgcgctgcctggatgaaattctgctcgaagggttggccgagaagataacaacaagccgcagcgatatgctgatcgttctgcatatgctgggcaatcacggcccagcgtatttcctgcgctatcccgcaagctaccgacgctgg

SEQ ID NO: 346 - DNA, 475 bp, Artificial Sequence
  Synthetic polynucleotide
cggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgacgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccagtgagcgtgggtctcgcggtatcattgcagcactg

SEQ ID NO: 347 - DNA, 147 bp, Artificial Sequence
  Synthetic polynucleotide
cgccattaccatgagcgataacagcgccgccaatctgctgctggccaccgtcggcggccccgcaggattgactgcctttttgcgccagatcggcgacaacgtcacccgccttgaccgctgggaaacggaactgaatgaggcgcttcc

SEQ ID NO: 348 - DNA, 216 bp, Artificial Sequence
  Synthetic polynucleotide
gctgatcggaaaccaaacgggagacgcgacactacgagcgggttttcctaaagattgggttgttggagagaaaactggtacctgcgccaacgggggccggaacgacattggtttttttaaagcccaggagagagattacgctgtagcggtgtatacaacggccccgaaactatcggccgtagaacgtgacgaattagttgcctctgtcggtcaagt

SEQ ID NO: 349 - DNA, 273 bp, Artificial Sequence
  Synthetic polynucleotide
tatggtaataacctggtacgagcacatacagaatatgtccctgcgtcaacatttaagatgctaaatgccttaattggattagaaaatcataaagctacaacaactgagattttcaaatgggatggtaaaaaaagatcttatcctatgtgggaaaaagatatgactttaggtgatgccatggcactttcagcagttcctgtatatcaagaacttgcaagacggactggcttagatctaatgcaaaaagaagttaaacgggttggttttggtaat

SEQ ID NO: 350 - DNA, 267 bp, Artificial Sequence
  Synthetic polynucleotide
aatcttagcacctatggtaatgctcttgcacgagcaaataaagaatatgtccctgcatcaacatttaagatgctaaatgctttaatcgggctagaaaatcgcgggcaacaacaaatgagattttcaaatgggatggtaaaaaaagaacttatcctatgtgggagaaagatatgactttaggtgaggcaatggcattgtcagcagttccagtatatcaagagcttgcaagacggactggcctagagctaatgcagaaagaagtaaagc

SEQ ID NO: 351 - DNA, 332 bp, Artificial Sequence
  Synthetic polynucleotide
tcaatcatcgatcagaatgttcaagcgctttttaatgaaatctcagctgatgctgtgtttgtcacatatgatggtcaaaatattaaaaaatatggcacgcatttagaccgagcaaaaacagcttatattcctgcatctacatttaaaattgccaatgcactaattggtttagaaaatcataaagcaacatctacagaaatatttaagtgggatggaaagccacgtttttttaaagcatgggacaaagattttactttgggcgaagccatgcaagcatctacagtgcctgtatatcaagaattggcacgtcgtattggtccaagcttaatgca

SEQ ID NO: 352 - DNA, 507 bp, Artificial Sequence
  Synthetic polynucleotide
gtaatgatcttgctcgtgcttcgaccgagtatgtacctgcttcgaccttcaaaatgcttaatgctttgatcggccttgagcaccataaggcaaccaccacagaagtatttaagtgggacgggcaaaaaaggctattcccagaatgggaaaaggacatgaccctaggcgatgctatgaaagcttccgctattccggtttatcaagatttagctcgtcgtattggacttgaactcatgtctaaggaagtgaagcgtgttggttatggcaatgcagatatcggtacccaagtcgataatttttggctggtgggtcctttaaaaattactcctcagcaagaggcacagtttgcttacaagctagctaataaaacgcttccatttagcccaaaagtccaagatgaagtgcaatccatgttattcatagaagaaaagaatggaaataaaatatacgcaaaaagtggttggggatgggatgtagacccacaagtaggctggttaactggatggg

SEQ ID NO: 353 - DNA, 225 bp, Artificial Sequence
  Synthetic polynucleotide
caaatacagaatatgtgccagcctctacatttaaaatgttgaatgccctgatcggattggagaaccagaaaacggatattaatgaaatatttaaatggaagggcgagaaaaggtcatttaccgcttgggaaaaagacatgacactaggagaagccatgaagctttctgcagtcccagtctatcaggaacttgcgcgacgtatcggtcttgatctcatgcaaaaag

SEQ ID NO: 354 - DNA, 24 bp, Artificial Sequence
  Synthetic polynucleotide
cctcacggccttggtaatttgggt

SEQ ID NO: 355 - DNA, 26 bp, Artificial Sequence
  Synthetic polynucleotide
accgtatggtctaggtaatttgggtg

SEQ ID NO: 356 - DNA, 24 bp, Artificial Sequence
  Synthetic polynucleotide
cctcacggtcttggcaatttaggt

SEQ ID NO: 357 - DNA, 18 bp, Artificial Sequence
  Synthetic polynucleotide
aagtctgggtgagaacgg

SEQ ID NO: 358 - DNA, 21 bp, Artificial Sequence
  Synthetic polynucleotide
tgcataagccagtgcgttttt

SEQ ID NO: 359 - DNA, 20 bp, Artificial Sequence
  Synthetic polynucleotide
aaaggcgtctgcgaccgagt

SEQ ID NO: 360 - DNA, 24 bp, Artificial Sequence
  Synthetic polynucleotide
tctgcccgccccattcgtgaaaac

SEQ ID NO: 361 - DNA, 23 bp, Artificial Sequence
  Synthetic polynucleotide
agtcgggctgtaaaggcgtctgt

SEQ ID NO: 362 - DNA, 20 bp, Artificial Sequence
  Synthetic polynucleotide
acaacgtcacccgccttgac

SEQ ID NO: 363 - DNA, 21 bp, Artificial Sequence
  Synthetic polynucleotide
aaaccaatgtcgttccggccc

SEQ ID NO: 364 - DNA, 25 bp, Artificial Sequence
  Synthetic polynucleotide
ccatggcactttcagcagttcctgt

SEQ ID NO: 365 - DNA, 29 bp, Artificial Sequence
  Synthetic polynucleotide
actttaggtgaggcaatggcattgtcagc

SEQ ID NO: 366 - DNA, 29 bp, Artificial Sequence
  Synthetic polynucleotide
tggcacgcatttagaccgagcaaaaacag

SEQ ID NO: 367 - DNA, 30 bp, Artificial Sequence; identical to SEQ ID NO: 336
  Synthetic polynucleotide

SEQ ID NO: 368 - DNA, 24 bp, Artificial Sequence
  Synthetic polynucleotide
tgccctgatcggattggagaacca

SEQ ID NO: 369 - DNA, 24 bp, Artificial Sequence
  Synthetic polynucleotide
tgagacacggtccagactcctacg

SEQ ID NO: 370 - DNA, 24 bp, Artificial Sequence
  Synthetic Polynucleotide
tagcttgatcgccctcgatttggg